(19) United States
(12) Patent Application Publication
(10) Pub. No.: US 2009/0075925 A1
(43) Pub. Date: Mar. 19, 2009

(54) Methods and Compositions Related to APOBEC-1 Expression

(76) Inventor: Harold C. Smith, South Rochester, NY (US)

Correspondence Address: CLARK G. SULLIVAN;ARNALL GOLDEN GREGORY LLP, 171 17TH STREET NW, SUITE 2100, ATLANTA, GA, 30363, US

(21) Appl. No.: 11/941,802

(22) Filed: Nov. 16, 2007

Related U.S. Application Data
(60) Provisional application No. 60/859,402, filed on Nov. 16, 2006.

(51) Int. Cl. C12N 15/85; C07K 14/00; C12N 5/06; C12P 21/02; C40B 30/06; G01N 33/53; A61K 31/7088; C12N 15/11

(52) U.S. Cl. 514/44; 530/350; 536/23.1; 435/320.1; 435/455; 435/375; 435/325; 435/69.1; 435/7.1; 506/8

(57) ABSTRACT

Disclosed are methods and compositions related to ACF and to APOBEC-1.

This invention was made with government support under Public Health Services Grant DK43739, NIH Grant DK43739. The government has certain rights in the invention.

### BACKGROUND OF THE INVENTION

ApoB mRNA editing involves the site-specific deamination of cytidine 6666 to uridine within a glutamine codon (CAA) thereby creating an in-frame translation stop codon (Smith et al. RNA 3:1105-23 (1997)). Consequently, two apoB protein variants are expressed, full-length apoB100 and the truncated protein apoB48, both of which participate in lipid transport, but with markedly different roles as atherogenic risk factors (Smith et al. (1997)). Minimally, apoB mRNA editing requires the cytidine deaminase APOBEC-1 as a homodimer (Lau et al. J Biol Chem 266:30550-4 (1991), Navaratnam et al. J Mol Biol 275:695-714 (1998), Oka et al. J Biol Chem 272:1456-60 (1997), Xie et al. Proc Natl Acad Sci USA 101:8114-9 (2004)), APOBEC-1 Complementation Factor (ACF) (Harris et al. J Biol Chem 268:7382-92 (1993), Lellek et al. J Biol Chem 275:19848-56 (2000), Mehta et al. Mol Cell Biol 18:4426-32 (1998), Sowden et al. J Biol Chem 279:197-206 (2004)) and the tripartite editing motif within the mRNA substrate (Backus and Smith Nucleic Acids Res 20:6007-14 (1992), Shah et al. J Biol Chem 266:16301-4 (1991), Smith et al. Proc Natl Acad Sci USA 88:1489-93 (1991)). ACF is the mooring sequence-specific RNA binding protein that directs site-specific editing (Harris et al. (1993), Lellek et al. (2000), Mehta et al. (1998), Sowden et al. (2004), Dance et al. J Biol Chem 277:12703-12709 (2002)).

Limited tissue expression of APOBEC-1 and apoB mRNA restricts editing in humans to the small intestine (≧85% editing), but apoB mRNA editing also occurs in the liver of several species (Navaratnam et al. (1998), Chen et al. Science 238:363-6 (1987), Greeve et al. J Lipid Res 34:1367-83 (1993), Powell et al. Cell 50:831-40 (1987)). Hepatic editing is modulated by fasting and refeeding in part due to an insulin-dependent increase in APOBEC-1 expression (von Wronski et al. Metabolism 47:869-73 (1998)). Hepatic editing is also regulated independently of changes in APOBEC-1 expression levels by developmental, hormonal, and nutritional perturbations (von Wronski et al. (1998), Chen et al. Biochem Biophys Res Commun 277:221-7 (2000), Funahashi et al. J Lipid Res 36:414-428 (1995), Inui et al. J Lipid Res 33:1843-56 (1992), Lau et al. J Lipid Res 36:2069-78 (1995), Phung et al. Metabolism 45:1056-8 (1996), Van Mater et al. Biochem Biophys Res Commun 252:334-9 (1998)). The mechanism for this form of editing activity involves the nuclear trafficking of editing factors (Sowden et al. J Cell Science 115:1027-1039 (2002), Yang, Y and Smith, H C Proc Natl Acad Sci USA 94:13075-80 (1997), Blanc et al. J Bio Chem (2003), Chester et al. Embo J22:3971-82 (2003)).

ApoB mRNA editing occurs primarily on spliced and polyadenylated RNA in the nucleus (Lau et al. (1991), Sowden et al. (2002), Yang and Smith (1997), Sowden, M P and Smith, H C Biochem J359:697-705 (2001), Yang et al. J Biol Chem 275:22663-9 (2000), Yang et al. Exp Cell Res 267:153-64 (2001)). Despite this, APOBEC-1 and ACF are distributed in both the cytoplasm and nucleus (Sowden et al. (2002), Blanc et al. (2003), Yang et al. (2000), Yang et al. (2001), Chester et al. Rna 10:1399-41 (2004)). In nuclear extracts, APOBEC-1 and ACF co-sedimented in 27S, editing-competent complexes, but as inactive 60S complexes in cytoplasmic extracts (Harris et al. (1993), Sowden et al. (2002)). Under in vitro editing conditions, 60S complexes reorganized to active 27S complexes on reporter RNAs (Harris et al. (1993), Sowden et al. (2002)). Furthermore, localization studies demonstrated that ACF and APOBEC-1 traffic between the cytoplasm and the nucleus (Blanc et al. (2003), Chester et al. (2003)). In support of trafficking as a regulatory mechanism, ethanol, insulin and thyroid hormone stimulation of hepatocyte editing activity promoted an increase in nuclear localization of ACF (Sowden et al. (2002), Yang et al. (2001), Mukhopadhyay et al. Endocrinology 144:711-9 (2003)).

What are needed in the art are compositions and methods related to ACF-induced apoB mRNA transport from the nucleus. The present invention is directed to overcoming these and other deficiencies in the art.

### SUMMARY OF THE INVENTION

In accordance with the purposes of this invention, as embodied and broadly described herein, this invention, in one aspect, relates to methods of expressing ACF in a cell, comprising bringing into contact a cell and a vector comprising a nucleic acid, wherein the nucleic acid encodes a polypeptide comprising a chimeric ACF sequence, a secretion sequence and a transduction sequence; whereby the nucleic acid produces the polypeptide, thereby expressing ACF in the cell.

Also disclosed are vectors comprising a nucleic acid, wherein the nucleic acid encodes a chimeric polypeptide comprising an ACF sequence, a secretion sequence and a transduction sequence.

Further disclosed are methods comprising administering to a cell a chimeric

polypeptide comprising an ACF sequence and a transduction sequence, wherein the ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated.

Also disclosed are methods of expressing ACF in a cell, comprising bringing into contact a cell and a vector comprising a nucleic acid, wherein the nucleic acid encodes a chimeric polypeptide comprising an ACF sequence; whereby the nucleic acid produces the polypeptide, thereby expressing ACF in the cell, wherein the chimeric ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated.

Disclosed herein are methods of screening for a compound that modulates phosphorylation of ACF, comprising: contacting a cell expressing ACF with a test compound, detecting the level of phosphorylated ACF using ACF phosphorylation site-specific antibodies, wherein a change in the level of phosphorylated ACF compared to the level of phosphorylated ACF in a control cell expressing ACF not exposed to the test compound indicates that the test compound is a compound that modulates phosphorylation of ACF.

Further disclosed are methods of screening for a compound that increases expression of ACF, comprising: contacting a cell with a test compound, detecting the level of ACF expression in the cell, wherein an increased level of ACF expression compared to the level of ACF expression in a control cell not exposed to the test compound indicates that the test compound is a compound that increases expression of ACF.

Also disclosed are compounds identified by the screening methods herein.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows protein phosphorylation is important for the interaction of ACF with APOBEC-1. Nuclear and cytoplasmic S100 extracts were prepared from a stable McArdle cell line that overexpresses HA-tagged APOBEC-1. Endogenous ACF was immunoprecipitated using ACF C-terminal peptide specific polyclonal antibodies, the complexes were washed with 850 mM NaCl to remove nonspecific adsorbed proteins (ACF's interaction with APOBEC-1 is stable up to 1 M NaCl, (Yang et al. (1997), Lau et al. (1991)) and then resolved by PAGE and western blotted with either anti-HA or ACF N-terminal, peptide specific antibodies. Blotting of a fractionated cell extract (starting material) demonstrated ACF64 and APOBEC-1 were present in the nucleus and the cytoplasm. However, following ACF specific immunoprecipitation, APOBEC-1 was only detected in the nuclear extract. Therefore, although ACF and APOBEC-1 co-sediment as 60S complexes from cytoplasmic extracts they apparently do not form stable complexes, consistent with the lack of cytoplasmic editing activity (Yang et al. (2000)). In contrast, phosphorylated ACF and APOBEC-1 form stable complexes in nuclear extracts consistent with the presence of 27S complexes and nuclear editing activity.

FIG. 2 shows phosphatase treatment inhibits apoB mRNA editing. FIG. 2A shows co-immunoprecipitation: The HA-tagged APOBEC-1 overexpressing McArdle cell line was treated with ethanol for 4 hours and fractionated. Nuclear extracts were immunoprecipitated with ACF CT antibody and processed as described in FIG. 1. For CIAP treatment extracts were adjusted to 5 mM MgCl₂, 3 mM CaCl₂, 0.1 mM ZnCl₂ and 25% glycerol and then incubated with 5U CIAP for 1 hour at 30° C. Control extracts were treated similarly but lacked phosphatase. The ratios of APOBEC-1:ACF were determined by scanning densitometry and quantitation using ImageJ Software. HC, position of 1g heavy chain. (n=4, standard deviation+/−0.2). FIG. 2B shows in vitro editing activity: Liver nuclear extract was treated with CIAP as described above. In vitro editing activity was determined using the poisoned-primer extension assay. The percent editing was quantified by PhoshorImager (Molecular Dynamics) scanning densitometry. Data shown is representative of 3 independent experiments. *Statistical significance was determined to be P≦0.01 by unpaired t-test relative to control. FIG. 2C shows RNA binding: ACF RNA binding activity was determined by ultraviolet light induced cross-linking of liver nuclear extracts. Quantitation of relative amounts of ACF bound was performed using PhoshorImager scanning densitometry. Data shown are representative of 4 independent experiments. *Statistical significance was determined to be P≦0.01 by unpaired t-test relative to control.

FIG. 3 shows hepatic ACF is a phosphoprotein. FIG. 3A shows hepatic ACF is phosphorylated in vivo: ACF was immunopurified from radiolabeled 27S containing glycerol gradient fractions (i.e. editosomes) and incubated with 100 U CIAP or buffer alone (control). Membranes were exposed to X-ray film (autorad) and subsequently reacted with ACF NT antibody (western). HC, position of Ig heavy chain. Data shown are representative of 3 independent experiments. FIG. 3B shows nuclear ACF contains more than one phosphorylated residue. Rat liver nuclear extracts (150 μg), incubated with or without 100 U CIAP were resolved by equilibrium 2D gel electrophoresis. The applicable range of the gel is shown corresponding to pH 8.3-9.3. The boxed area delineates the acidic isoform of ACF that was not observed following CIAP treatment. Nuclear extract was run on the end of the 2^{nd} dimension gel to verify the relative migration (Mr) of ACF and is delineated by a vertical bar. Data shown are representative of 2 independent experiments. FIG. 3C shows ACF is phosphorylated on serine residue(s): ACF was immunoprecipitated from control or 0.9% ethanol treated radiolabeled rat hepatocytes and transferred to nitrocellulose. The radiolabeled band corresponding to ACF western blot reactivity was excised from the blot, acid hydrolyzed and the products from control and ethanol-treated hepatocytes resolved in parallel by 2D thin layer electrophoresis. The migration of phosphoamino acids was determined from ninhydrin staining of known standards spiked into the samples and is indicated and the thin layer plate was autoradiographed. Data shown are representative of 3 independent experiments.

FIG. 4 shows protein phosphatase I inhibitors modulate apoB mRNA editing and ACF phosphorylation: FIG. 5A shows RNA Editing: Rat primary hepatocytes were incubated with concentrations of cantharidin ranging from 47 nM (IC₅₀ of PP2A) to 4.7 μM (10 times IC₅₀ PP1) and 0.45% ethanol where indicated. In vivo editing activity was determined on endogenous apoB mRNA using apoB specific RT-PCR followed by poisoned primer extension (Sowden et al. (1996)). *Statistical significance was determined to be P≦0.01 by unpaired t-test relative to DMSO control n>5. FIG. 5B shows ACF phosphorylation and specific activity: In vivo ACF ³²P incorporation was determined by PhosphorImager scanning of ACF immunoprecipitates prepared from rat hepatocytes treated with 470 nM cantharidin. ACF specific activity (relative to control hepatocytes) was calculated as the ACF ³²P density (PhosphorImager) divided by the recovery of ACF determined from densitometric scanning of ACF western blots (Image J). ACF immunopurified from control hepatocytes was arbitrarily assigned a value of 1 (n=3). Exp. 1 and Exp. 2 denote independent experiments 1 and 2.

FIG. 5 shows higher resolution analysis afforded by immunoelectron microscopy of rat liver demonstrated nuclear (Nu) ACF in perichromatin distribution and the bulk of cytoplasmic ACF localized to the cytoplasmic surface of the endoplasmic reticulum (ER). The right panel, arrows point to NT-Ab reactivity.

FIG. 6 shows PAGE analysis and autoradiography demonstrated incorporation of ³²P on ACF under the conditions of acute insulin and short labeling period, showing a relatively high turnover site of phosphorylation (FIG. 6A). CIAP treatment of nuclear extracts did not affect the recovery of IP'ed ACF but markedly reduced the amount of isotopic label recovered with ACF (FIG. 6A, CIAP). Western blots of nuclear extract resolved by two dimensional (2D) PAGE revealed an ACF isoform at pI 8.3 (FIG. 6B; representing 15-20% of the total nuclear protein) with the majority of ACF focusing at pI 8.8 (FIG. 6B, arrow; the isoelectric point predicted for unmodified ACF). CIAP reduced the abundance of the pI 8.3 isoform and increased the amount of the pI 8.8 isoform (FIG. 6B, lower panel). The difference in charge between the two ACF isoforms is consistent with an acidic shift due to approximately two phosphates (0.2-0.3 pH units per phosphate).

FIG. 7 shows that to determine the relevance of ACF phosphorylation to these complexes, nuclear and cytoplasmic extracts from basal insulin or 10 nM insulin (post-prandial level) treated rat primary hepatocytes were sedimented through 10%-50% glycerol gradients. Relevant fractions (indicated across the top of panels A & B in FIG. 7) were IP'ed with CT-Ab (‘IP’), resolved by PAGE, western blotted and autoradiographed (‘Autorad’) and then the blots were reacted with the NT-Ab to visualize ACF. FIG. 7C shows that despite 6-fold more cytoplasmic extract protein applied to the glycerol gradient (compared to nuclear protein), no evidence of cytoplasmic phosphoACF was found. Insulin stimulated ACF phosphorylation and nuclear restriction can also be demonstrated in the cells.

FIG. 8 shows the amino acids subject to insulin regulated phosphorylation were determined by 2D phoshoamino acid electrophoresis of protein acid hydrozolates. ACF was IP'ed from nuclear extracts of human primary hepatocytes treated with 0 or 0.1 nM insulin for four hours in the presence of 32Pi and transferred to nylon membrane for acid hydrolysis. The 2D profiles demonstrate ninhydrin stained co-electrophoresed standards (pair of panels in A) and showed that ACF IP'ed from insulin treated hepatocytes only contained phosphoserine (panel B, insulin). Low or no radiolabeled phosphoamino acids were obtained with rat ACF from insulin or ethanol treated primary hepatocytes.

FIG. 9 shows MS analysis detected >80% sequence coverage when compared to the ACF (top panel) and ions consistent with the peptide TKKREEILSEMK (SEQ ID NO: 42) containing a phosphorylated S154 were readily observed (lower left panel). The data also indicated phosphoserine within the peptide GHLSNRALIR (SEQ ID NO: 43) containing a phosphorylated S368 (lower right panel). All of these sites were predicted computationally.

FIG. 10 shows the results of representative substitution mutations on basal and ethanol stimulated editing activity. The central vertical line indicates the percent editing of wild type (WT) McArdle cells. To calibrate the system cells were incubated in the presence of ethanol for 4 hours and the amount of edited apoB mRNA determined. Ethanol induced the anticipated increase (52%) in editing activity (21% editing to 32%) (Yang et al. (2000), Van Mater et al. Biochem Biophys Res Commun 252:334-339 (1998)). Stable ectopic expression of ACF64 (WT ACF) stimulated editing slightly (19%) compared to untransfected McArdle cells and the addition of ethanol to this cell line induced a further 3-fold stimulation.

FIG. 11 shows cantharidin treatment at the IC₅₀ of PP2A, 47 nM had little effect on the recovery of phosphorylated ACF (as well as apoB mRNA editing) An increased recovery of phosphorylated ACF was first apparent at the IC₅₀ of PP1 and became markedly elevated following complete inhibition of PP1 (10-times the IC₅₀ or 4.7 μM). These data were observed with several hepatocyte preparations (n=5).

FIG. 12 shows the effect of cantharidin on the distribution of ACF within primary hepatocytes was also evaluated by fractionating cells into cytoplasmic and nuclear extracts as described above and immunoblotting each fraction with the ACF CT antibody.

FIG. 13 shows the reduction of intracellular apoB protein in hepatocyte cytoplasmic extracts and apoB protein secreted into the media as VLDL by treatment with cantharidin and inhibition of PP1 quantified as ng apoB protein per mg total protein by radioimmunoassay using a monoclonal antibody against apoB as described previously (Chirieac et al. (2000), Au et al. (2004), Sparks, J D and Sparks, C E J Biol Chem 265:8854-62 (1990)).

FIG. 14 shows the ability to use RNAi selective for human ACF to knockdown expression of human ACF protein in HepG2 cells without affect rat ACF expression from a transfected cDNA encoding rat ACF. 72 hours post-transfection total cell lysates were western blotted with ACF N-terminal peptide specific and actin antibodies; the latter to ensure that an equivalent amount of cell material was compared. In two separate experiments (left and right pairs) human ACF (hACF) abundance was significantly reduced compared to control RNAi (Luci) treated cells.

FIG. 15 shows the efficiency of adenoviral GFP delivery into primary mouse hepatocytes has been evaluated. The fluorescent images of primary hepatocytes demonstrate increasing expression of GFP in mouse primary hepatocytes with increasing adenoviral m.o.i. (from left to right) and with increasing time subsequent to infection (compare corresponding 18 h and 24 h post-infection images in upper and lower panels).

FIG. 16 shows in situ hyper phosphorylation of ACF64 was demonstrated by ³²P labeling of rat primary hepatocytes under basal insulin (Basal 0.1 nM insulin), post-prandial insulin (10 nM) or acute ethanol exposure (0.45% ethanol with 0.1 nM insulin). Using sub-saturating amounts of ACF C-terminal specific antibody, ACF was immunoprecipitated from glycerol gradient fractions containing either nuclear (A) or cytoplasmic (B) S100 extracts. The data demonstrated a 2 and 3.5-fold stimulation of ACF phosphorylation by insulin or ethanol respectively. Phosphorylated ACF selectively co-sedimented with 27S nuclear editosomes and was not detected in pre-editosomal 60S complexes (FIG. 16A) or in cytoplasmic extracts (FIG. 16B).

FIG. 17 shows alignment of HuD RRM1-RRM2 sequence with those of human and rat ACF64, showing this region of HuD was an appropriate model for comparative modeling of the comparable region in ACF64. First, there is 94% amino acid identity (98% similarity) between human and rat ACF. Second, 23% of the aligned HuD residues were identical to ACF (shown in black) with 51% similarity. Finally, the selection of HuD versus other RRM containing structures was based on UV crosslinking and RNA binding competition analyses that indicated a preference of ACF64/65 for binding to AU-rich RNA, an observation in accordance with the AU rich nature of the RNA sequence in the vicinity of the apoB RNA editing site Harris et al. (1993), Mehta et al. (1996), Blanc et al. (2001), Mehta, A and Driscoll, D M RNA 8:69-82 (2002), Backus, J W and Smith, H C Biochim Biophys Acta 1217:65-73 (1994), Backus et al. Biochim Biophys Acta 1219:1-14 (1994)). Given these similarities, the HuD structure (Wang and Hall 92001), Kielkopf et al. (2004)).

FIG. 18 shows a typical RRM-containing protein, wherein RNA recognition occurs through the β-strands and flanking loops, whereas protein-protein interactions can occur at the a helices or β-strands. In HuD, the β-sheets of RRM1 and RRM2 bind AU-rich ssRNA whereas the α helices face outward, exposed to solvent, thereby making them accessible for protein-protein interactions (Wang and Hall (2001, Kieldopf et al. (2004)). The model was calculated by use of the program MODELLER.

FIG. 19 shows induction of apobec-1 mRNA expression (FIG. 25A) and apoB mRNA editing activity (FIG. 19B). Intestinal ACF becomes constitutively phosphorylated at serine or threonine residues as cells differentiate into enterocytes. The apobec-1 data revealed a low level of expression by 7 days that increased upon a further 2 weeks of differentiation. Apobec-1 mRNA expression was insufficient to induce a significant increase in editing activity, compared to that in proliferating cells until 2 weeks of differentiation (FIG. 19B). Editing activity increased upon further differentiation concomitant with an increase in apobec-1 mRNA expression. Similar semi-quantitative RT-PCR analyses revealed that proliferating Caco2 cells expressed acf mRNA and the levels of the alternatively spliced RNA variants acf64 and acf65 were almost equivalent (FIG. 25C). After 2-3 weeks of differentiation acf64 mRNA was the prominent spliced variant (acf64:acf65 ratio of 1.8:1) consistent with the finding that acf64 mRNA is the predominant spliced variant in fetal and adult human intestine (Henderson et al. (2001)) as well as in rat liver, rat intestine and HepG2 human hepatoma cells (Sowden et al. (2004)).

FIG. 20 shows that APOBEC-1 expression alone is not sufficient. Transient transfection of EGFP using FuGene 6 (Roche) indicated that up to 50% of proliferating Caco2 cells can be transfected. Stable cell lines were also established that expressed C-terminal V5-tagged rat ACF64 and ACF65 (FIG. 20, left panel). Editing activity, quantified by poisoned primer extension on endogenous apoB mRNA, was low in proliferating Caco2 cells as well as in cells transiently transfected with HA-tagged APOBEC-1 (FIG. 20, right panel).

FIG. 21 shows 72 hours post-transfection total cell lysates were western blotted with ACF N-terminal peptide specific and actin antibodies; the latter to ensure that an equivalent amount of cell material was compared. In two separate experiments (left and right pairs) human ACF (hACF) abundance was significantly reduced compared to control RNAi (Luci) treated cells. To determine the species specificity of the RNAi SMARTpool, McArdle cells were transfected and rACF expression determined. Significantly, rACF was refractory to RNAi knock down (lower two panels).

FIG. 22 shows that while phosphatase treatment of nuclear extracts inhibited in vitro editing activity, it did not significantly affect UV crosslinking of ACF variants to apoB RNA.

FIG. 23 shows cell extracts were immunoprecipitated with the ACF C-terminal specific antibody and analyzed by PAGE and autoradiography. At the IC₅₀ of PKA A3 had a modest effect on the phosphorylation of ACF (20% decrease) relative to untreated cells. However, at and above the WC₅₀ of PKC, phosphorylation of ACF64 was inhibited by 90% and ˜100%.

FIG. 24 shows the co-localization of ACF by western blotting with the C-terminal anti-ACF antibody (panel C) as enriched in cytoplasmic membrane fractions that contain the bulk of apoB mRNA (panel A) and apoB protein (B).

FIG. 25 shows inhibition of protein phosphatase with cantharadin at the IC₅₀ and higher of corresponding to concentrations known to be high enough to inhibit protein phosphate 1 (PP1) in primary hepatocytes, increased the recovery of phosphorylated ACF (A). Cantharadin did not block ethanol stimulate ACF phosphorylation (B) and stimulated apoB mRNa editing (C).

FIG. 26 shows modulation of apoB mRNA editing in rat primary hepatocytes treated with insulin. Stimulation of hepatic editing was associated with: (i) increased expression of APOBEC-1, (ii) enhanced nuclear abundance of ACF65/64 and (iii) increased abundance of acf65 mRNA relative to acf64 mRNA.

FIG. 27 shows hepatic apoB mRNA editing increased in ethanol consuming rats as well as in ethanol treated rat primary hepatocytes and McArdle hepatoma cells. Ethanol stimulated editing without increasing APOBEC-1 expression but promoted increased nuclear abundance of ACF64/65. Simultaneous amplification of acf64 and acf65 mRNAs and RT-PCR ratio analysis showed that ethanol treatment increased acf64 mRNA relative to acf65. (FIG. 27B) The effects were reversible following 12 hours of ethanol withdrawal (washout).

FIG. 28 shows the utility of the ACF65 specific antibody (peptide specific polyclonal antibody raised against the 8 amino acid insert in ACF65; EIYMNVPVG, SEQ ID NO: 36). ACF64 (ACF; lanes 1&2) and ACF65 (ASP; lanes 5&6) were detected by western blot analysis of 35 μg of cytoplasmic (C) or nuclear (N) protein. The data showed that ACF64 was more abundant than ACF65 in both subcellular fractions. Cytoplasmic (320 μg) and nuclear (60 μg) extract proteins were immunoprecipitated (IP'ed) with ACF C-terminal antibody and complexes precipitated with Protein A agarose. IPs were western blotted and reacted with anti-ACF (lanes 3&4) or anti-ASP (lanes 7&8). The data demonstrated efficient recovery of ACF64 and ACF65 from cytoplasmic and nuclear extracts. Primary antibody Ig heavy chain (HC) co-eluted with the antigen because it could not be crosslinked to beads without a loss of activity. ACF45 has a sufficiently long and unique C-terminus (CSRTPSIYLCFLTAVHAGVHHI HVQ, SEQ ID NO: 37) to serve as an epitope. It has been observed that N-terminally conjugated peptides elicit immunoreactivity to the C-terminal amino acids, showing that a peptide consisting of 9-11 ACF43/45 common amino acids plus the C-terminal ACF43 three unique residues (NIS) can elicit ACF43-specific antibodies.

FIG. 29 shows HepG2 human hepatoma (A) and human primary hepatocytes (B) expressing high levels of GFP after transduction with BacMam-GFP virus. Replacement of the polyhedron promoter and 3′ RNA processing signals in pFastBac-1 (Invitrogen) with an RNA polymerase III promoter (e.g. U6) is planned for the expression of ACF isoform specific siRNA cassettes.

FIG. 30 shows an RNAi specific to GFP reduced editing nearly 20-fold, showing that GFP-APOBEC-1 abundance had been reduced.

FIG. 31 shows phosphoserine and phosphothreonine specific antibodies (Zymed), but not anti-phosphotyrosine were reactive with phosphorylated ACF further indicating that ACF64/65 is a phosphoprotein.

FIG. 32 shows two immunoprecipitations resolved by PAGE (Coomassie) and ACF64/65 identified (western). Comparison to mass standards (Mr, BioRad Precision Plus) of 100 kDa and 50 kDa (representing 150 ng and 750 ng of protein respectively) indicated that >400 ng of ACF64/65 (total of >5000 Cerenkov cpms) were obtained.

FIG. 33 shows the specific recovery of a radiolabeled apoB transcript, but not of RNA that lacked the mooring sequence (WT-1), from an in vitro editosome assembly by IP with ACF64 antibody (33A). Western blotting with the ACF N-terminal antibody demonstrated good recovery of the endogenous ACF64 from liver extract or recombinant ACF64 (47B). The recovery of endogenous or recombinant ACF64 crosslinked to radiolabeled apoB RNA was virtually eliminated by 100-fold molar excess of competing cold apoB RNA but was not affected by competing WT-1 RNA (33C). The assembly reactions (A and C) used 50 fmols of radiolabeled apoB RNA, detected readily by RT-PCR-RT, minus reverse transcriptase control) (33D).

FIG. 34 shows co-expression of ACF isoforms with different functional properties showed that ACF isoforms are interactive components of a regulatory network controlling the amount of edited apoB mRNA. A cartoon of this model depicts four metabolic states in rat hepatocytes: (i) basal (˜0.1% insulin and ˜65% editing) (ii) fasted (<0.1% insulin and ˜30% editing) (iii) 10-25 nM insulin, high sucrose refed (˜80% editing) and (iv)>0.1% ethanol treated (˜95% editing). Also shown are the relative abundance of editing factors in arbitrary units estimated from scanning densitometry of western blots, and for APOBEC-1, alterations in mRNA expression levels. Also listed are the relative APOBEC-1 binding, apoB UV crosslinking activities and complementation activities of each ACF isoform.

FIG. 35 shows the overall expression and subcellular localization of epitope tagged ACF isoforms is established by western blotting of nuclear and cytoplasmic extracts (prepared using NE-PER system; Pierce). These biochemical studies are complemented by immunofluorescence microscopy of paraformaldehyde fixed cells reacted with epitope tag or ACF isoform-specific antibodies. In pilot experiments varying levels of HA-tagged ACF43 and ACF45 were expressed by transfecting differing amounts of cDNA into McArdle or McAPOBEC cells (FIGS. 35A & C, western blots). The level of exogenously expressed HA-tagged APOBEC-1 in McAPOBEC cells was high and unaffected by ACF isoform expression (FIG. 35C). Sufficient endogenous ACF was expressed in McAPOBEC cells to support 61% editing (FIG. 35D, poisoned primer extension) given the level of APOBEC-1 overexpression (FIG. 35C). ACF43 expression in McArdle cells did not affect editing whereas ACF45 inhibited editing over two-fold (FIG. 35B, poisoned primer extension). In contrast, ACF43 stimulated editing activity in McAPOBEC cells while ACF45 had no affect (FIG. 35D).

FIG. 36 shows UV cross-linking of an equivalent amount of nuclear and cytoplasmic protein, which demonstrated that while the concentration of ACF64/65 was less in the cytoplasm, its RNA binding activity was significantly more than that in the nuclear extract. Fasting reduced the nuclear abundance of ACF64/65 and increased their abundance in the cytoplasm. Correspondingly ACF64/65 UV crosslinking activity increased in the cytoplasm.

### DETAILED DESCRIPTION

The polypeptides and nucleic acids described herein are useful in treating various diseases and disorders associated with apoB mRNA transport from the nucleus, and with lipid clearance from the liver.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

“Optional” or “optionally” means that the subsequently described event or circumstance may or may not occur, and that the description includes instances where said event or circumstance occurs and instances where it does not.

The terms “higher,” “increases,” “elevates,” “enhances,” or “elevation” refer to increases as compared to a control level. The terms “low,” “lower,” “reduces,” “suppresses” or “reduction” refers to decreases as compared to a control level. Control levels can be normal in vivo levels prior to, or in the absence of, treatment. Thus, the control can be from the same subject prior to treatment or can be an untreated control subject or group thereof.

By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

The terms “control levels” or “control cells” are defined as the standard by which a change is measured, for example, the controls are not subjected to the experiment, but are instead subjected to a defined set of parameters, or the controls are based on pre- or post-treatment levels.

By “contacting” is meant an instance of exposure of at least one substance to another substance. For example, contacting can include contacting a substance, such as a cell, or cell to a test compound described herein. A cell can be contacted with the test compound, for example, by adding the protein or small molecule to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). The duration of contact with a cell or group of cells is determined by the time the test compound is present at physiologically effective levels or at presumed physiologically effective levels in the medium or extracellular fluid bathing the cell.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

By “effective amount” is meant a therapeutic amount needed to achieve the desired result or results, e.g., reducing atherogenic risk, blunting physiological functions, altering the qualitative or quantitative nature of the proteins expressed by cell or tissues, and eliminating or reducing disease causing molecules and/or the mRNA or DNA that encodes them.

Herein, “inhibition” or “suppression” means to reduce activity as compared to a control (e.g., activity in the absence of such inhibition). It is understood that inhibition or suppression can mean a slight reduction in activity to the complete ablation of all activity. An “inhibitor” or “suppressor” can be anything that reduces the targeted activity.

Many methods disclosed herein refer to “systems.” It is understood that systems can be, for example, cells, columns, or batch processing containers (e.g., culture plates). A system is a set of components, any set of components that allows for the steps of the method to performed. Typically a system will comprise one or more components, such as a protein(s) or reagent(s).

The term “ACF polypeptide” or “APOBEC-1 polypeptide” or “ACF nucleic acid” or “APOBEC-1 nucleic acid” refers to not only the polypeptide or nucleic acid of APOBEC-1 or ACF itself, but can also refer to any associated transduction or signal sequences associated therewith.

General

RNA editing is the co- or post-transcriptional alteration of genomically encoded RNA sequence by nucleotide insertion, deletion, substitution or modification (Gott, et al. Annu Rev Genet. 34, 499-531 (2000), Wedekind et al. Trends Genet. 19, 207-216 (2003)). Editing of mRNA can alter codon sense enabling expression of proteins with different functions or, in some cases, production of a functional protein is strictly dependent on editing.

Apolipoprotein B is a non-exchangeable structural component of intestinally derived chylomicrons and of liver derived very low density lipoprotein particles (VLDL). ApoB is translated from a 14 kb mRNA that is transcribed from a single copy gene located on human chromosome 2 (Scott, J. Mol. Biol Med 6:65-80 (1989)). A large (apoB100) and small (apoB48) isoform of apoB lipoprotein are expressed by post-transcriptional RNA editing (Chen et al. Science 238:363-366 (1987), Powell et al. Cell 50:831-840 (1987)), which involves a site-specific hydrolytic deamination of cytidine at nucleotide 6666 (C6666) to form uridine (Johnson et al. Biochem Biophys Res Commun 195:1204-1210 (1993)), thereby creating an in-frame translation stop codon, UAA, from a glutamine codon, CAA (Chen et al (1987), Powell et al. (1987)). The mammalian small intestine constitutively edits ≧85% of apoB mRNA and stores apoB48 for the assembly and secretion of chylomicrons containing dietary lipids (Chen et al (1987), Powell et al. (1987), Greeve et al. A J Lipid Res 34:1367-1383 (1993), Backus et al. Biochem Biophys Res Commun 170:513-518.((1990)).

In several mammals, with the important exception of humans, apoB mRNA editing also occurs in liver (Greeve et al. (1993)). Rodent liver regulates apoB mRNA editing thereby modulating the proportion of edited apoB mRNA and the proportion of B48 versus B100 secreted as VLDL (Greeve et al. (1993, Sparks et al. Can J Biochem 59:693-699 (1981)). Hepatic VLDL are assembled co-translationally with apoB48 or apoB100 and are secreted into the blood from where they are cleared by peripheral tissues and the liver. B100 VLDL are converted by hepatic and peripheral lipases to low density lipoprotein particles (LDL) (an atherogenic risk factor (Corsetti et al. Athersclerosis 171:351-358 (2003)) whereas B48 VLDL that lack the LDL receptor binding domain are cleared more rapidly via chylomicron remnant or apoE surface receptors and are not converted to LDL. Hence hepatic editing is associated with a reduced risk of atherosclerosis (Sparks et al. J Lipid Res 22:519-527 (1981); Greeve et al. Oncogene 18:6357-6366 (1999)).

Many factors contribute to atherogenic disease (Corsetti et al. (2003)) however, studies identified elevated LDL (hypercholesterolemia) as the primary atherosclerotic risk factor in 25% of the population. Significantly, lipoprotein analyses revealed that apoB editing in mammalian liver reduces the VLDL+LDL to HDL ratio (Greeve et al. (1993), which is associated with reduced atherogenesis. Human liver expresses all the factors necessary for apoB mRNA editing except the enzyme APOBEC-1 (Navaratnam et al. Proc Natl Acad Sci USA 90:222-226 (1993)) and consequently cannot synthesize apoB48.

APOBEC-1−/− knockout mice expressing a human apoB transgene expressed only B100 in their intestine and liver resulting in elevated serum LDL levels, low triglyceride secretion rates and larger lipoprotein size (Nakamuta et al. J. Biol. Chem. 271: 25981-25988 (1996), Hirano et al. J. Biol. Chem. 271:9887-9890 (1996) Xie et al. Am J Physiol Gastrointest Liver Physiol 285:G735-746 (2003)). Control ratios of B48:B100 in serum VLDL and the proportion of serum VLDL and LDL lipoproteins was restored by apobec-1 gene transfer without adverse side effects (Nakamuta et al. (1996), Qian et al. Arterioscler Thromb Vasc Biol 18:1013-1020 (1998), Hughes et al. Hum Gene Ther 7:39-49 (1996), Teng et al. J. Biol. Chem. 269:29395-29404 (1994)). These findings demonstrated not only that APOBEC-1 is the sole enzyme capable of editing apoB mRNA but also suggested the exciting possibility of apobec-1 gene therapy for atherogenic disease.

The RNA sequence directing C6666 site-specific apoB mRNA editing has been extensively characterized (Smith, et al. RNA 3:1105-1123 (1997), Smith, H. C. Semin Cell Biol 4:267-278 (1993)). The most important element for cytidine deamination was the ‘mooring sequence’ (UGAUCAGUAUA: nts 6671-6681, SEQ ID NO: 44) that is the 3′ most element of a 21 nt tripartite motif that also comprises an enhancer element (immediately 5′ of C6666) and a spacer element between C6666 and the mooring sequence. The significance of the tripartite motif for editing site utilization has been proven by in vitro mutagenesis, transient transfection, in transgenic mice and underscored by chicken apoB mRNA that is not edited in any species because of a degenerate motif (Sowden and Smith (1996), Backus and Smith, Nucleic Acids Research 19:6781-6786 (1991), Backus and Smith, Nucleic Acids Research 20:6007-6014 (1992), Shah, et al. J Biol Chem 266:16301-16304 (1991), Driscoll, et al. Mol Cell Biol 13:7288-7294 (1993)).

Analysis of nuclear apoB mRNAs at various stages of RNA processing demonstrated that editing occurred coincident with, or immediately following, splicing (Lau, et al. J. Biol. Chem. 266:20550-20554 (1991)). Chimeric RNAs comprising the Adenovirus late leader RNA splicing site linked to apoB sequence containing the tripartite editing motif were edited poorly in McArdle cells (Sowden and Smith, (1996)). Splice site mutants demonstrated that splicing of the premRNA mediated editing suppression but splice site suppression diminished as the distance of splice junctions from the editing site was increased (Sowden and Smith, Biochem J 359:697-705 (2001)). Editing was abolished when the tripartite editing motif was within an intron unless commitment of this RNA to the splicing pathway was bypassed by the inclusion of a cis-acting Rev Responsive Element and co-expression of the HIV-1 Rev. Unspliced RNAs were edited and exported from the nucleus (Sowden and Smith (2001)). Splicing and editing factors interact or compete for similar or proximal RNA binding sites.

The accepted mechanism of site-specific C to U apoB mRNA editing is the requirement for a macromolecular assembly of proteins (editosome) that minimally contains a catalytic subunit, the cytidine deaminase APOBEC-1 (27 kDa), and an essential complementation factor, ACF (64 kDa) (Smith, et al. Proc Natl Acad Sci USA 88:1489-1493 (1991)). The understanding of the minimal editosome is that a dimer of APOBEC-1 is targeted to C6666 by an interaction with ACF which is bound selectively to the apoB mRNA ‘mooring sequence’ (Mehta, et al. Mol Cell Biol 20:1846-1854 (2000)). These proteins can be isolated from editing competent nuclear extracts of liver and intestinal cells as 27S (˜300-500 kDa), a complexity indicative of multimers of minimal editosomes and/or associated RNA substrate and/or additional proteins (Backus and Smith (1991), Backus and Smith (1992), Smith et al. (1991), Harris, et al. J. Biol. Chem. 268:7382-7392 (1993), Smith, H. C. Methods 15:27-39 (1998), Steinburg, et al. Biochem. Biophys. Res. Co mun. 263:81-86 (1999), Sowden, et al. J Cell Science 115:1027-1039 (2002)). 60S cytoplasmic aggregates containing APOBEC-1 and ACF were identified in rat liver and intestine that are postulated to contain inactive editing factors (Harris et al. (1993), Sowden et al. (2002)).

The amino acid sequence of APOBEC-1 from several organisms (Teng et al. (1994), Yamanaka et al. J Biol Chem 269:21725-21734 (1994), Nakamuta et al. J Biol Chem 270:1042-13056 (1995), Lau et al. Proc Natl Acad Sci USA 91:8522-8526 (1994), Hadjiagapiou et al. Nucl Acids Res 22:1874-1879 (1994)) is highly conserved. It is a zinc dependent enzyme whose catalytic domain shares similarity with a variety of nucleoside/nucleotide deaminases (Wedekind et al. (2003), Navaratnam et al. (1993), Anant et al. Biol Chem 379:1075-1081 (1998), Mian et al. J Comput Biol 5:57-72 (1998)). His61, Pro92, Cys93 and Cys96 within the catalytic domain of APOBEC-1 are essential for zinc coordination and editing and Glu63 is required for proton shuttling during catalysis (Anat et al. J Biol Chem 270:14762-14767 (1995), MacGinnitie et al. J Biol Chem 270:14768-14775 (1995), Teng et al. J Lipid Res 40:623-635 (1999), Driscoll et al. J Biol Chem 269:19843-19847 (1994)). Residues within the catalytic domain, particularly H61, E63, F66, F87 and C93 are also required for APOBEC-1's low affinity (Kd˜450 mM, (Anant and Davidson Mol Cell Biol 20:1982-1992 (2000)) and nonspecific RNA binding activity. Separated from the N-terminal catalytic domain by a functionally important linker region is a C-terminal Non-Functional Catalytic Domain (NFCD) (Wedekind et al. (2003), Jannuz et al. GenomicI 79:285-296 (2002)). The NFCD does not contain all the residues necessary for zinc coordination but is required for the function of the N-terminal catalytic domain. This region also contains a Nuclear Export/Cytoplasmic Retention Signal (NES/CRS) that was dominant over the Nuclear Localization Signal (NLS) of SV40T antigen in chimeric reporter proteins (Chester et al. (2003), Yang et al. Exp. Cell Res. 267:153-164 (2001), Yang and Smith Proc Natl Acad Sci USA 94:13075-13080 (1997)). A stretch of basic amino acids in the N-terminus of APOBEC-1 is similar to the NLS of SV40T antigen. The NLS and a novel M-domain (residues 97-172) were both required for nuclear import (Yang et al. (2001), Yang et al. J Biol Chem 272:27700-27706 (1997)). Point mutations in this region inhibited in vitro editing activity, showing that in addition to trafficking, interactions involving the N-terminus of APOBEC-1 were fundamental to editosome function (Teng et al. (1999)).

The discovery that a cytidine deaminase from Saccharomyces cerevisiae (ScCDD1) mediated mooring sequence dependent editing of reporter apoB mRNA expressed in yeast (Dance et al. Nucleic Acids Res 29:1772-1780 (2001)) provided a tenable connection between the enzymes of pyrimidine metabolism and APOBEC-1. Unlike APOBEC-1, soluble CDD1 was readily expressed in E. coli and crystals were grown that diffracted to 2.0 Å. From the structural determination of CDD1 and amino acid alignments of known oligomeric cytidine deaminase crystal structures, including CDD1, a comparative model for APOBEC-1 was calculated (Xie et al. Proc Natl Acad Sci USA 101:8114-8119 (2004)). A requirement for dimerization of APOBEC-1 for catalytic activity was first suggested by experiments in which dimerization competent, but catalytically inactive mutants of APOBEC-1 exhibited a dominant negative effect when transfected into cells (Oka et al. J Biol Chem 272:1456-1460 (1997)). C-terminal leucines between residues 169-184 (PQYPPLWMMLYLALEL, SEQ ID NO: 45) were required for homodimerization (Lau et al. (1994), Teng et al. (1999), Navaratnam et al. J Mol Biol 275:695-714 (1998)). The model explains this need for dimerization (Lau (1994), Oka et al. (1997)) as it reveals how two polypeptide chains interact to form a single catalytic domain. Head to head dimerization assures that the N-terminal catalytic domain of one monomer receives essential trans-acting residues contained in a flexible loop domain from the linker region of the other monomer. By analogy to known cytidine deaminase structures these intermolecular structural elements are essential for ribose binding and catalytic activity (Xie et al. (2004).

The controversy of whether apoB mRNA editing was mediated by a lone catalytic subunit or a macromolecular assembly (editosome) was resolved by the identification of APOBEC-1 (Smith et al. (1991), Greeve et al. (1991)). Under physiological conditions APOBEC-1 could not edit apoB mRNA unless cell extracts were added (Teng et al. Science 260:1816-1819 (1993)). This finding led to the proposal of the requirement for auxiliary proteins to complement APOBEC-1 and provided strong support for the ‘mooring sequence hypothesis’. This hypothesis, which has now been proven, was based on native gel shift analyses and glycerol gradient sedimentation which predicted a 27S ‘C/U editosome’ consisting of multiple proteins serving the function of apoB mRNA recognition, catalysis and regulation (Backus and Smith (1991), Backus and Smith (1992), Smith, H. C. (1998)). Only 27S complexes contained both unedited and edited apoB RNA (Smith et al. (1991)). The essential auxiliary proteins, now referred to as ACF64/65 (Mehta et al. (2000), Sowden et al. (2002), Lellek et al. J Biol Chem 275:19848-19856 (2000)) and ACF43/45 (Sowden et al. (2002)) selectively interacted with the mooring-sequence (Navaratnam et al. (1993), Shah et al. (1991) Driscoll et al. (1993), Harris et al. (1993), Mehta et al. (1996), Mehta and Driscoll Mol Cell Biol 18:4426-4423 (1998)), co-purified with editosomes (Harris et al. (1993), Smith, H C. (1998)) and were co-selected when APOBEC-1 was affinity purified from cell extracts Yang et al. (1997)). In contrast to the tissue specificity of editing activity (Greeve et al. (1993)) these auxiliary proteins are expressed in a variety of tissue and cell types (Teng et al. (1993), Inui et al. J Lipid Res 35 (1994), Giannoni et al. J Biol Chem 269:5932-5936 (1994)).

A p66-like, UV crosslinking protein from baboon kidney was purified by biochemical means. Peptide sequencing and database comparisons identified two human Expressed Sequence Tags (ESTs) that facilitated cloning of ACF cDNA. Comparative analyses revealed that the mooring sequence-selective, RNA binding protein known as p66 (Harris et al. (1993)) is in fact ACF64/65 (Sowden et al. (2002)). ACF64 was necessary and sufficient for complementing APOBEC-1 in vitro editing activity. It contained three RNA Recognition Motifs (RRM) and interacted with APOBEC-1. A second complementation factor, APOBEC-1 Stimulating Protein (ASP), was identified in rat liver, and a corresponding human cDNA cloned (Lellek et al. (2000)). ASP was identical to ACF except for an 8 amino acid insertion that is predicted to encode a tyrosine kinase phosphorylation site. Human ACF and ASP are encoded by a single gene on chromosome 10 and arise through insulin-regulated alternative premRNA splicing of acf mRNA promoted by the splicing factor SRp40 (Dance et al. J Biol Chem 277:12703-12703 (2002), Henderson et al. Biochim Biophys Acta 1522:22-30 (2001)). These variants were renamed ACF64 and ACF65 based on their predicted molecular masses. The N-terminus of ACF64 contained the domains required for RNA and APOBEC-1 binding/complementing activity; (i) amino acids 1-129 were required for APOBEC-1 binding, (ii) RRM1 (58-123) and RRM2 (138-208) contributed the most to apoB mRNA binding, (iii) RRM3 (208-315) enhanced apoB RNA binding activity and (iv) sequence following RRM3 (331-385) contains a novel nuclear localization signal and is required for wild type levels of apoB RNA binding (Blanc et al. (2003), Mehta and Driscoll RNA 8:69-82 (2002), Blanc et al. J Biol Chem 276:46386-46393 (2001)).

Alternatively spliced acf mRNA variants were identified in human EST databases and validated (Dance et al. (2002), Henderson et al. (2001), Chester et al. Biochim Biophys Acta 1494:1-13 (2000)). Two additional rat specific variants arising from alternative acf premRNA splicing and 3′ end formation were uniquely expressed in liver and intestine (Sowden et al. J Biol Chem 278:197-206 (2004). These mRNAs encoded 43 kDa and 45 kDa proteins (henceforth termed ACF43 and ACF45) identical to the N-terminal two thirds of ACF64/65 (380 of 586 amino acids) and consequently, ACF43/45 bound apoB mRNA, interacted with APOBEC-1, were localized to the nucleus and complemented editing activity (Sowden et al. (2002)). ACF43/45 represent the minimal functional auxiliary proteins that are sufficient to complement APOBEC-1 in apoB mRNA editing.

The cellular localization of endogenous APOBEC-1 can be determined by assaying subcellular fractions for editing activity. The data indicate a nuclear and cytoplasmic distribution for APOBEC-1 (Anant and Davidson (2000)). Transfected cell studies in rat (McArdle) and human (HepG2) hepatoma cells, as well as heterokaryon analyses also showed a cytoplasmic and nuclear distribution of APOBEC-1 (Sowden and Smith (2001), Chester et al. (2003), Yang et al. (2001), Yang et al. (1997), Blanc et al. (2003)). Nuclear localization or export signal mutants of APOBEC-1 distributed nearly quantitatively to the cytoplasm or nucleus (respectively) of transfected McArdle cells (Yang et al. (2001), Yang and Smith (1997)) showing that overexpressed APOBEC-1 assumed the biologically representative subcellular distribution.

Following APOBEC-1 overexpression, apoB mRNA editing was stimulated on cytoplasmic mRNA as well as on the normal nuclear apoB mRNAs (Yang et al. (2000)). Moreover, editing site fidelity was compromised by high levels of APOBEC-1 overexpression in cell lines (Yang et al. (2000), Sowden et al. (1996), Sowden et al. (1998), Siddiqui et al. (1999), Sowden and Smith (3002)) and transgenic animals (Yamanaka et al. (1996), Yamanaka et al. (1997), Yamanaka et al. Proc Natl Acad Sci USA 92:8483-8487 (1995)). Lower levels of expression in transgenic animals (Nakamuta et al. (1996), Hughes et al. (1996), Teng et al. (1994)) or via TAT-dependent transduction of APOBEC-1 protein directly into hepatocytes Yang et al. (2002)) stimulated editing without compromising site specificity.

Endogenous ACF64/65 protein expression can be detected with peptide-specific antibodies (Sowden et al. (2002)). Immunocytochemical analyses in McArdle cells revealed that native ACF was both nuclear and cytoplasmic (Sowden et al. (2002)). Subcellular fractionation and western blotting of rat hepatocytes demonstrated a similar distribution of ACF which was corroborated by immunoelectron microscopy of rat liver thin sections (Sowden et al. (2002)). Importantly, HA-tagged ACF64 distributed to the cytoplasm and nucleus of transfected McArdle cells (Yang et al. (2000)) and complemented APOBEC-1 editing activity but, unlike APOBEC-1 overexpression, did not compromise site specificity (Sowden et al. (2002)).

Metabolic studies in rats and with rat primary hepatocytes demonstrated that the proportion of cellular ACF in the nucleus increased when apoB mRNA editing was stimulated by ethanol or insulin and returned to basal levels when the stimuli were withdrawn (Yang et al. (2000), Sowden et al. (2002)). This showed that nuclear trafficking is a control point for the regulation of editing activity. Using heterokaryon analyses, it was demonstrated that ACF trafficks between the cytoplasm and nucleus (Chester et al. (2003), Blanc et al. (2003)). These studies also showed that APOBEC-1 was required for nuclear import of ACF. It was also found that APOBEC-1 had a weak nuclear localization signal but did not import into the nucleus of cells that did not express ACF (Yang et al. (2001), Yang and Smith (1997)). These findings were confirmed and extended by showing that an interaction of ACF with transportin-2 was required for APOBEC-1 nuclear import (Blanc et al. (2003)).

Splicing competent and translatable chimeric mRNAs containing the apoB RNA editing site were expressed in HeLa cells with or without transfected editing factors (Chester et al. (2003)). In the absence of editing these chimeric mRNAs were stable. Pre-edited (i.e. containing a PTC) or APOBEC-1 edited RNAs were also stable but only in the presence of co-expressed ACF. Expression of APOBEC-1 alone or an RNA-binding defective mutant of ACF64 resulted in NMD of the PTC-containing reporter RNA (Chester et al. (2003)). A role for ACF in stabilizing edited apoB mRNA for translation shows how mammalian intestinal cells can edit >85% of their apoB mRNA and, despite the presence of a PTC, express high levels of apoB48. The data show a significant role for cytoplasmic ACF in regulating apoB protein synthesis and correspondingly lipoprotein production. ACF has a high affinity for the mooring sequence (8 nM Kd) (Mehta and Driscoll (2002)) and demonstrated equal affinity for unedited or edited apoB mRNA (Harris et al. (1993)). Nuclear apoB mRNA associated ACF appears to remain associated with edited and unedited apoB mRNA during its export to the cytoplasm. Immunoelectron microscopy localization of ACF to the external surface of the endoplasmic reticulum (the site of apoB translation and lipoprotein assembly) is consistent with an interaction of ACF with apoB mRNA (Sowden et al. (2002)).

Rat liver regulation of ACF trafficking through reversible phosphorylation and its role in regulating apoB mRNA nuclear export are relevant to humans as they are key elements that determine the availability of apoB mRNA for translation of the atherogenic risk factor apoB100 which regulates lipoprotein particle assembly and secretion into the blood (Chirieac et al. Am J Physiol Endocrinol Metab 279:D-1003-1011 (2000), Au et al. Metabolism 53:228-235 (2004)). It is disclosed herein that the regulatory control of ACF can enable safe and effective expression of APOBEC-1 dependent editing in human liver and/or the prevention or treatment of hypercholesterolemia, particularly where reduced serum lipoprotein clearance is part of the etiology (Whitfield et al. Clin Chem 50:1725-1732 (2004)).

The regulation of ACF in human liver is also significant in relation to the increasing prevalence of obesity and insulin resistance leading to type II diabetes as well as in nonalcoholic fatty liver disease from which 14% to 24% of the US population currently suffer. Frequently patients with this condition progress from simple hepatic steatosis to cirrhosis and require liver transplantation (Browning, J D and Horton, J D J Clin Invest 114:147-152 (2004), Festi et al. Obes Rev 5:27-42 (2004)). Fatty infiltrates are characteristic of the hyperinsulinemic and insulin resistant state can arise from increased hepatic gluconeogenesis, increased hepatic uptake of fatty acids due to elevated lipolysis in adipose tissue and the inability of the liver to process triglycerides through lipoprotein production and secretion. Factors contributing to progression to steato-hepatitis can involve enhanced susceptibility to oxidative stress and inflammation (Browning and Horton (2004)) as is frequently the case with steatosis in alcoholics (Keegan et al. J Hepatol 23:591-600 (1995)) and following viral infections (Negro, F. J Hepatol 40:533-535 (2004)). Studies of rats fed polyunsaturated fats show this can be due to oxidative stress induced degradation of B100 (Pan et al. J Clin Invest 113:1277-1287 ((2004)). Hepatic synthesis of apoB protein regulates VLDL assembly and secretion. Therefore, the regulated trafficking of ACF and its interaction with apoB mRNA can determine apoB mRNA stability and translation efficiency and as such, could serve as a one of the control points for hepatic VLDL assembly and clearance. Therapeutic targeting of this process can increase hepatic triglyceride clearance through increased B100 production that can, if a corresponding and problematic rise in serum LDL was observed, be treated with available hyperlipidemia reduction therapies. However, apoB48 was not degraded in livers undergoing oxidative stress (Pan et al. (2004)) and as apoB48 has a higher capacity to transport lipids and apoB48 VLDL do not contribute to LDL, the therapeutic induction of apoB mRNA editing can reduce fatty liver infiltrates without contributing to vascular disease.

Ethanol activates several serine/threonine protein kinases (PK) including PKA, PKC, casein kinase JI and calcium-calmodulin kinase II (CaMKII) (Hoek, J B and Kholodenko, B N Alcohol Clin Exp Res 22:224 S-230S (1998)). Insulin binding to its cognate receptor tyrosine kinase results in the rapid stimulation of multiple serine/threonine protein kinases for example PKC, GSK3β, PI-dependent protein kinase, PI3K, B/AKT, MAPK, p90RSK and p70 S6 kinase (Czech et al. J Biol Chem 263:11017-11020 (1988), Werner et al. J Biol Chem 279:35298-35305 (2004)). Protein phosphatases are also activated by ethanol (Higashi et al. Alcohol Alcohol 29 Suppl 1:53-59 (1994)) and insulin (Ugi et al. Mol Cell Biol 24:8778-9890 (2004), Shi et al. J Biochem (Tokyo) 136:89-96 (2004)). These facts indicate a complex pattern in which site specific phosphorylation or dephosphorylation is finely orchestrated to regulate cell signaling, gene expression and protein function. Phosphorylation of proteins can either activate or inactivate these processes and frequently proteins are phosphorylated at multiple sites (hyper-phosphorylated), revealing that many exist in a range of hyper- or hypo-phosphorylated states.

Ethanol and insulin regulation involves overlapping protein kinase and protein phosphatase activities but ethanol abuse is antagonistic to insulin regulation (Mokuda et al. Ann Nutr Metab 48:276-280 (2004), Rubin et al. National Institutes of Alocohol Abuse and Alcoholism, Bethesda, Md. (2000)) and can induce insulin resistance (Yi, S J and Jhun, B H J Med Food 7:24-30 (2004), Kraus et al. Kidney Int 65:881-887 (2004)) that may lead to type II diabetes with associated atherogenic disease (Hanley et al. Diabetes 53:2623-2632 (2004)). Complementary effects of low level or acute ethanol consumption on insulin regulation have been described (Avogaro et al. Diabetes Care 27:1369-1374 (2004)). Both ethanol and insulin stimulate apoB mRNA editing and increase the nuclear abundance of ACF (Sowden et al. (2004)) and significantly it can be shown that both agents stimulated ACF phosphorylation.

Observation indicates that serines 47 and 72 of APOBEC-1 are differentially phosphorylated (Chen et al. Biochem J 357:661-672 (2001)). Mutants S47D or S72A stimulated apoB mRNA editing in rat hepatoma cells whereas as S47A or S72D inhibited editing. These residues were predicted protein kinase C sites and, although the C₀ isoform is predominantly expressed in skeletal muscle, its overexpression in hepatoma cells stimulated editing activity (Chen et al. (2001)).

The induction of TAT-ACF expression containing phosphorylation site mutations (serine to alanine, for example), in a limited number of hepatocytes in situ through viral delivery and its secretion into the local hepatic circulation results in the local uptake of TAT-ACF by liver cells and the induction of apoB100 mRNA mobilization. As discussed above, unlike rodent liver, human liver cannot edit apoB mRNA and consequently all of the lipoproteins assembled and secreted by human liver contain apoB100, which although mobilize lipid from the liver, have the potential to become LDL, a risk factor for atherogenesis, cardiovascular disease, and stroke, for example. Combination of the TAT-ACF technology with TAT-APOBEC-1 circumvents this risk.

### Compositions

### ACF Polypeptides and Nucleic Acids

Disclosed herein are polypeptides comprising an ACF sequence, a secretion sequence and a transduction sequence. Also disclosed herein is a vector comprising a nucleic acid, wherein the nucleic acid encodes a polypeptide comprising an ACF sequence, a secretion sequence and a transduction sequence. In one example, the polypeptide can be secreted from the cell from which it was produced, and the polypeptide can transduce a second cell, thereby delivering the polypeptide to the second cell. The polypeptide can be delivered to the second cell in vivo or in vitro. When the polypeptide is delivered in vitro, it can be delivered in culture, for example. This can be accomplished via transduction. Delivery of the polypeptide to the second cell can increase transport of apoB mRNA from the nucleus to the cytoplasm of the second cell.

The ACF sequence can be selected from the group consisting of an ACF65 sequence, an ACF64 sequence, an ACF45 sequence, and an ACF43 sequence.

The ACF sequence can have a mutation at one or more sites where ACF is phosphorylated. For example, the ACF sequence can have a serine to alanine or aspartic acid substitution at one or more sites where ACF is phosphorylated. For instance, the amino acid substitution can comprise a serine to alanine or aspartic acid substitution at the amino acid residue corresponding to amino acid residue 154 in SEQ ID NO:1.

In another example, the amino acid substitution can comprise a serine to alanine or aspartic acid substitution at the amino acid residue corresponding to amino acid residue 368 in SEQ ID NO:1. In another example, both of these mutations can be present simulataneously.

The full-length rat ACF has an amino acid sequence according to SEQ ID NO: 25:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Pro Gly Trp Asp
Thr Thr Pro Pro Glu Arg Gly Cys Glu Ile Phe Ile
Gly Lys Leu Pro Arg Asp Leu Phe Glu Asp Glu Leu
Ile Pro Leu Cys Glu Lys Ile Gly Lys Ile Tyr Glu
Met Arg Met Met Met Asp Phe Asn Gly Asn Asn Arg
Gly Tyr Ala Phe Val Thr Phe Ser Asn Lys Gln Glu
Ala Lys Asn Ala Ile Lys Gln Len Asn Asn Tyr Glu
Ile Arg Asn Gly Arg Leu Leu Gly Val Cys Ala Ser
Val Asp Asn Cys Arg Leu Phe Val Gly Gly Ile Pro
Lys Thr Lye Lys Arg Glu Glu Ile Leu Ser Glu Met
Lys Lye Val Thr Glu Gly Val Val Asp Val Ile Val
Tyr Pro Ser Ala Ala Asp Lys Thr Lys Asn Arg Gly
Phe Ala Phe Val Glu Tyr Glu Ser His Arg Ala Ala
Ala Met Ala Arg Arg Arg Leu Leu Pro Gly Arg Ile
Gln Leu Trp Gly His Pro Ile Ala Val Asp Trp Ala
Glu Pro Glu Val Glu Val Asp Glu Asp Thr Met Ser
Ser Val Lys Ile Leu Tyr Val Arg Asn Leu Met Leu
Ser Thr Ser Glu Glu Met Ile Glu Lys Glu Phe Asn
Ser Ile Lys Pro Gly Ala Val Glu Arg Val Lys Lys
Ile Arg Asp Tyr Ala Phe Val His Phe Ser Asn Arg
Glu Asp Ala Val Glu Ala Met Lys Ala Leu Asn Gly
Lys Val Leu Asp Gly Ser Pro Ile Glu Val Thr Leu
Ala Lys Pro Val Asp Lys Asp Ser Tyr Val Arg Tyr
Thr Arg Gly Thr Gly Gly Arg Asn Thr Met Leu Gln
Glu Tyr Thr Tyr Pro Leu Ser His Val Tyr Asp Pro
Thr Thr Thr Tyr Leu Gly Ala Pro Val Phe Tyr Thr
Pro Gln Ala Tyr Ala Ala Ile Pro Ser Leu His Phe
Pro Ala Thr Lys Gly His Leu Ser Asn Arg Ala Leu
Ile Arg Thr Pro Ser Val Arg Glu Ile Tyr Met Asn
Val Pro Val Gly Ala Ala Gly Val Arg Gly Leu Gly
Gly Arg Gly Tyr Leu Ala Tyr Thr Gly Leu Gly Arg
Gly Tyr Gln Val Lys Gly Asp Lys Arg Gln Asp Lys
Leu Tyr Asp Leu Leu Pro Gly Met Glu Leu Thr Pro
Met Asn Thr Ile Ser Leu Lys Pro Gln Gly Val Lys
Leu Ala Pro Gln Ile Leu Glu Glu Ile Cys Gln Lys
Asn Asn Trp Gly Gln Pro Val Tyr Gln Leu His Ser
Ala Ile Gly Gln Asp Gln Arg Gln Leu Phe Leu Tyr
Lys Val Thr Ile Pro Ala Leu Ala Ser Gln Asn Pro
Ala Ile His Pro Phe Thr Pro Pro Lys Leu Ser Ala
Tyr Val Asp Glu Ala Lys Arg Tyr Ala Ala Glu His
Thr Leu Gln Thr Leu Gly Ile Pro Thr Glu Gly Gly
Asp Ala Gly Thr Thr Ala Pro Thr Ala Thr Ser Ala
Thr Val Phe Pro Gly Tyr Ala Val Pro Ser Ala Thr
Ala Pro Val Ser Thr Ala Gln Leu Lys Gln Ala Val
Thr Leu Gly Gln Asp Leu Ala Ala Tyr Thr Thr Tyr
Glu Val Tyr Pro Thr Phe Ala Val Thr Thr Arg Gly
Asp Gly Tyr Gly Thr Phe

A DNA molecule encoding the full length rat ACF has a nucleotide sequence according to SEQ ID No: 26 as follows:

atggaatcaa atcacaaatc cggggatgga ttgagcggca
cccagaagga agcagcactc cgcgcactgg tccagcgcac
aggatatagc ttggtccagg aaaatggaca aagaaaatat
ggtggtcctc caccaggctg ggatactaca cccccagaaa
ggggctgcga gattttcatt gggaaacttc cccgggacct
ttttgaggat gaactcatac cattgtgtga aaaaattggt
aaaatttatg aaaigagaat gatgatggat ttcaatggga
acaacagagg ctatgcattt gtaaccttct caaataagca
ggaagccaag aatgcaatca agcaacttaa taattatgaa
attcggaatg gccgtctcct gggcgtctgt gccagtgtgg
acaactgccg gttgtttgtg gggggaatcc ccaaaaccaa
aaagagagaa gaaatcttgt cagagatgaa aaaggtcact
gaaggagttg ttgatgtcat tgtctaccca agcgctgccg
ataaaaccaa aaaccggggg tttgcctttg tggaatatga
gagtcaccgc gcagccgcca tggctaggcg gaggctgctg
ccaggaagaa ttcagttgtg gggacatcct atcgcagtag
actgggcaga gccagaagtc gaagttgacg aagacacaat
gtcttccgtg aaaatcctgt acgtaaggaa ccttatgctg
tctacctcgg aagagatgat tgagaaggaa ttcaacagta
ttaaaccagg tgctgtggaa cgggtgaaga agatccgaga
ctatgctttt gtgcatttca gtaaccgaga agatgcagtt
gaagccatga aggctttgaa tggcaaggtg ctggatggtt
ccccaataga agtgaccttg gccaagccag tggacaagga
cagttacgtt aggtacaccc ggggcaccgg gggcaggaac
accatgctgc aagaatacac ctaccctctg agccatgttt
atgaccctac cacaacctac cttggagctc ctgtcttcta
tactccccaa gcctacgcag ccattccaag tcttcatttc
ccagetacca aaggacatct cagcaacaga gctctcatcc
ggaccccttc tgtcagagaa atttacatga atgtccctgt
aggggctgcg ggcgtgagag gactgggcgg ccgtgggtat
ttggcatata caggcctggg tcgaggatac caggtcaaag
gagacaagag acaagacaaa ctctatgacc ttctgcctgg
gatggagctc accccgatga atactatctc tttaaaacca
caaggagtta aacttgctcc tcagatatta gaagaaatct
gtcagaaaaa taactgggga cagccagtgt accagctgca
ctctgccatt ggacaagacc aaagacagtt attcctatac
aaagtaacta tcccagcgct ggccagccag aatcctgcga
tccacccttt cacaccccca aagctaagcg cctacgtgga
tgaagcaaag aggtacgccg cagagcacac cctacagaca
ctaggcatcc ccacagaagg aggggacgct gggactacag
cacccactgc cacatccgcc actgtgtttc caggatacgc
tgtccccagt gccaccgctc ctgtgtctac agcccagctc
aagcaagcag tgacacttgg acaagactta gcagcatata
caacctatga ggtctaccct acttttgcag tgaccacccg
aggtgatgga tatggcacct tctga

The amino acid sequence and nucleotide sequence for the full length rat ACF65 is reported at Genbank Accession Nos. AAK50145 and AY028945, respectively, each of which is hereby incorporated by reference in its entirety. In addition, it should be noted that a short isoform of rat ACF64 exists, as identified at Genbank Accession No. AF290984, which is hereby incorporated by reference in its entirety.

The full length human ACF has an amino acid sequence according to SEQ ID No: 27 as follows:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Pro Gly Trp Asp
Ala Ala Pro Pro Glu Arg Gly Cys Glu Ile Phe Ile
Gly Lys Leu Pro Arg Asp Leu Phe Glu Asp Glu Leu
Ile Pro Leu Cys Glu Lys Ile Gly Lys Ile Tyr Glu
Met Arg Met Met Met Asp Phe Asn Gly Asn Asn Arg
Gly Tyr Ala Phe Val Thr Phe Ser Asn Lys Val Glu
Ala Lys Asn Ala Ile Lys Gln Leu Asn Asn Tyr Glu
Ile Arg Asn Gly Arg Leu Leu Gly Val Cys Ala Ser
Val Asp Asn Cys Arg Leu Phe Val Gly Gly Ile Pro
Lys Thr Lys Lys Arg Glu Glu Ile Leu Ser Glu Met
Lys Lys Val Thr Glu Gly Val Val Asp Val Ile Val
Tyr Pro Ser Ala Ala Asp Lys Thr Lys Asn Arg Gly
Phe Ala Phe Val Glu Tyr Glu Ser His Arg Ala Ala
Ala Met Ala Arg Arg Lys Leu Len Pro Gly Arg Ile
Gln Leu Trp Gly His Gly Ile Ala Val Asp Trp Ala
Glu Pro Glu Val Glu Val Asp Glu Asp Thr Met Ser
Ser Val Lys Ile Leu Tyr Val Arg Asn Leu Met Leu
Ser Thr Ser Glu Glu Met Ile Glu Lys Glu Phe Asn
Asn Ile Lys Pro Gly Ala Val Glu Arg Val Lys Lys
Ile Arg Asp Tyr Ala Phe Val His Phe Ser Asn Arg
Lys Asp Ala Val Glu Ala Met Lys Ala Leu Asn Gly
Lys Val Len Asp Gly Ser Pro Ile Glu Val Thr Leu
Ala Lys Pro Val Asp Lys Asp Ser Tyr Val Arg Tyr
Thr Arg Gly Thr Gly Gly Arg Gly Thr Met Leu Gln
Gly Glu Tyr Thr Tyr Ser Leu Gly Glu Val Tyr Asp
Pro Thr Thr Thr Tyr Leu Gly Ala Pro Val Phe Tyr
Ala Pro Gln Thr Tyr Ala Ala Ile Pro Ser Leu His
Phe Pro Ala Thr Lys Gly His Leu Ser Asn Arg Ala
Ile Ile Arg Ala Pro Ser Val Arg Gly Ala Ala Gly
Val Arg Gly Leu Gly Gly Arg Gly Tyr Leu Ala Tyr
Thr Gly Leu Gly Arg Gly Tyr Gln Val Lys Gly Asp
Lys Arg Glu Asp Lys Leu Tyr Asp Ile Leu Pro Gly
Met Glu Leu Thr Pro Met Asn Pro Val Thr Leu Lys
Pro Gln Gly Ile Lys Leu Ala Pro Gln Ile Leu Glu
Glu Ile Cys Gln Lys Asn Asn Trp Gly Gln Pro Val
Tyr Gln Leu His Ser Ala Ile Gly Gln Asp Gln Arg
Gln Leu Phe Leu Tyr Lys Ile Thr Ile Pro Ala Leu
Ala Ser Gln Asn Pro Ala Ile His Pro Phe Thr Pro
Pro Lys Leu Ser Ala Phe Val Asp Glu Ala Lys Thr
Tyr Ala Ala Glu Tyr Thr Leu Gln Thr Leu Gly Ile
Pro Thr Asp Gly Gly Asp Gly Thr Met Ala Thr Ala
Ala Ala Ala Ala Thr Ala Phe Pro Gly Tyr Ala Val
Pro Asn Ala Thr Ala Pro Val Ser Ala Ala Gln Leu
Lys Gln Ala Val Thr Leu Gly Gln Asp Leu Ala Ala
Tyr Thr Thr Tyr Glu Val Tyr Pro Thr Phe Ala Val
Thr Ala Arg Gly Asp Gly Tyr Gly Thr Phe

A DNA molecule encoding the full length human ACF has a nucleotide sequence according to SEQ ID No: 28 as follows:

atggaatcaa atcacaaatc cggggatgga ttgagcggca
ctcagaagga agcagccctc cgcgcactgg tccagcgcac
aggatatagc ttggtccagg aaaatggaca aagaaaatat
ggtggccctc cacctggttg ggatgctgca ccccctgaaa
ggggctgtga aatttttatt ggaaaacttc cccgagacct
ttttgaggat gagcttatac cattatgtga aaaaatcggt
aaaatttatg aaatgagaat gatgatggat tttaatggca
acaatagagg atatgcattt gtaacatttt caaataaagt
ggaagccaag aatgcaatca agcaacttaa taattatgaa
attagaaatg ggcgcctctt aggggtttgt gccagtgtgg
acaactgccg attatttgtt gggggcatcc caaaaaccaa
aaagagagaa gaaatcttat cggagatgaa aaaggttact
gaaggtgttg tcgatgtcat cgtctaccca agcgctgcag
ataaaaccaa aaaccgaggc tttgccttcg tggagtatga
gagtcatcga gcagctgcca tggcgaggag gaaactgcta
ccaggaagaa ttcagttatg gggacatggt attgcagtag
actgggcaga gccagaagta gaagttgatg aagatacaat
gtcttcagtg aaaatcctat atgtaagaaa tcttatgctg
tctacctctg aagagatgat tgaaaaggaa ttcaacaata
tcaaaccagg tgctgtggag agggtgaaga aaattcgaga
ctatgctttt gtgcacttca gtaaccgaaa agatgcagtt
gaggctatga aagctttaaa tggcaaggtg ctggatggtt
cccccattga agtcacccta gcaaaaccag tggacaagga
cagttatgtt aggtataccc gaggcacagg tggaaggggc
accatgctgc aaggagagta tacctactct ttgggccaag
tttatgatcc caccacaacc taccttggag ctcctgtctt
ctatgccccc cagacctatg cagcaattcc cagtcttcat 1080
ttcccagcca ccaaaggaca tctcagcaac agagccatta
tccgagcccc ttctgttaga ggggctgcgg gagtgagagg
actgggcggc cgtggctatt tggcatacac aggcctgggt
cgaggatacc aggtcaaagg agacaaaaga gaagacaaac
tctatgacat tttacctggg atggagctca ccccaatgaa
tcctgtcaca ttaaaacccc aaggaattaa actcgctccc
cagatattag aagagatttg tcagaaaaat aactggggac
agccagtgta ccagctgcac tctgctattg gacaagacca
aagacagcta ttcttgtaca aaataactat tcctgctcta
gccagccaga atcctgcaat ccaccctttc acacctccaa
agctgagtgc ctttgtggat gaagcaaaga cgtatgcagc
cgaatacacc ctgcagaccc tgggcatccc cactgatgga
ggcgatggca ccatggctac tgctgctgct gctgctactg
ctttcccagg atatgctgtc cctaatgcaa ctgcacccgt
gtctgcagcc cagctcaagc aagcggtaac ccttggacaa
gacttagcag catatacaac ctatgaggtc tacccaactt
ttgcagtgac tgcccgaggg gatggatatg gcaccttctg a

The amino acid sequence and nucleotide sequence for the full length human ACF is reported at Genbank Accession Nos. AAF76221 and AF271789, respectively, each of which is hereby incorporated by reference in its entirety.

As mentioned above, disclosed herein are variants of ACF, such as ACF65, ACF64, ACF45, and ACF43. ACF65.

The ACF65 sequence can comprise the amino acid sequence SEQ ID NO:17, an amino acid sequence at least about 90% identical to the amino acid sequence of SEQ ID NO:17, the amino acid sequence SEQ ID NO:17 having one or more conservative amino acid substitutions, or the amino acid sequence SEQ ID NO:17 having one or more mutations. The full length of ACF65 has an amino acid sequence according to SEQ ID NO:17 as follows:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Asn Pro Gly Trp
Asp Ala Ala Pro Pro Glu Arg Gly Cys Glu Ile Phe
Ile Gly Lys Leu Pro Arg Asp Leu Phe Glu Asp Glu
Leu Ile Pro Leu Cys Glu Lys Ile Gly Lys Ile Tyr
Glu Met Arg Met Met Met Asp Phe Asn Gly Asn Asn
Arg Gly Tyr Ala Phe Val Thr Asn Phe Ser Asn Lys
Val Glu Ala Lys Asn Ala Ile Lys Gln Leu Asn Asn
Tyr Glu Ile Arg Asn Gly Arg Leu Leu Gly Val Cys
Ala Ser Val Asp Asn Cys Arg Leu Phe Val Gly Gly
Ile Pro Lys Thr Lys Lys Arg Glu Glu Ile Leu Ser
Glu Met Lys Lys Val Thr Asn Glu Gly Val Val Asp
Val Ile Val Tyr Pro Ser Ala Ala Asp Lys Thr Lys
Asn Arg Gly Phe Ala Phe Val Glu Tyr Glu Ser His
Arg Ala Ala Ala Met Ala Arg Arg Lys Leu Leu Pro
Gly Arg Ile Gln Leu Trp Gly His Gly Ile Ala Val
Asp Trp Ala Glu Pro Asn Glu Val Glu Val Asp Glu
Asp Thr Met Ser Ser Val Lys Ile Leu Tyr Val Arg
Asn Leu Met Leu Ser Thr Ser Glu Glu Met Ile Glu
Lys Glu Phe Asn Asn Ile Lys Pro Gly Ala Val Glu
Arg Val Lys Lys Ile Arg Asp Tyr Ala Phe Val His
Phe Ser Asn Arg Asn Glu Asp Ala Val Glu Ala Met
Lys Ala Leu Asn Gly Lys Val Leu Asp Gly Ser Pro
Ile Glu Val Thr Leu Ala Lys Pro Val Asp Lys Asp
Ser Tyr Val Arg Tyr Thr Arg Gly Thr Gly Gly Arg
Gly Thr Met Leu Gln Gly Glu Tyr Thr Tyr Ser Leu
Gly Gln Val Asn Tyr Asp Pro Thr Thr Thr Tyr Leu
Gly Ala Pro Val Phe Tyr Ala Pro Gln Thr Tyr Ala
Ala Ile Pro Ser Leu His Phe Pro Ala Thr Lys Gly
His Leu Ser Asn Arg Ala Ile Ile Arg Ala Pro Ser
Val Arg Gly Ala Ala Gly Val Arg Gly Leu Gly Gly
Arg Gly Asn Tyr Leu Ala Tyr Thr Gly Leu Gly Arg
Gly Tyr Gln Val Lys Gly Asp Lys Arg Glu Asp Lys
Leu Tyr Asp Ile Leu Pro Gly Met Glu Leu Thr Pro
Met Asn Pro Val Thr Leu Lys Pro Gln Gly Ile Lys
Leu Ala Pro Gln Ile Leu Glu Glu Ile Cys Gln Lys
Asn Asn Asn Trp Gly Gln Pro Val Tyr Gln Leu His
Ser Ala Ile Gly Gln Asp Gln Arg Gln Leu Phe Leu
Tyr Lys Ile Thr Ile Pro Ala Leu Ala Ser Gln Asn
Pro Ala Ile His Pro Phe Thr Pro Pro Lys Leu Ser
Ala Phe Val Asp Glu Ala Lys Thr Tyr Ala Ala Glu
Asn Tyr Thr Leu Gln Thr Leu Gly Ile Pro Thr Asp
Gly Gly Asp Gly Thr Met Ala Thr Ala Ala Ala Ala
Ala Thr Ala Phe Pro Gly Tyr Ala Val Pro Asn Ala
Thr Ala Pro Val Ser Ala Ala Gln Leu Lys Gln Ala
Val Thr Leu Gly Gln Asp Leu Ala Ala Tyr Thr Asn
Thr Tyr Glu Val Tyr Pro Thr Phe Ala Val Thr Ala
Arg Gly Asp Gly Tyr Gly Thr Phe

A DNA molecule encoding ACF65 has a nucleotide sequence according to SEQ ID NO: 20 as follows:

ctcaatggaa tcaaatcaca aatccgggga tggattgagc
ggcactcaga aggaagcagc cctccgcgca ctggtccagc
gcacaggata tagcttggtc caggaaaatg gacaaagaaa
atatggtggc cctccacctg gttgggatgc tgcaccccct
gaaaggggct gtgaaatttt tattggaaaa cttccccgag
acctttttga ggatgagctt ataccattat gtgaaaaaat
cggtaaaatt tatgaaatga gaatgatgat ggattttaat
ggcaacaata gaggatatgc atttgtaaca ttttcaaata
aagtggaagc caagaatgca atcaagcaac ttaataatta
tgaaattaga aatgggcgcc tcttaggggt ttgtgccagt
gtggacaact gccgattatt tgttgggggc atcccaaaaa
ccaaaaagag agaagaaatc ttatcggaga tgaaaaaggt
tactgaaggt gttgtcgatg tcatcgtcta cccaagcgct
gcagataaaa ccaaaaaccg aggctttgcc ttcgtggagt
atgagagtca tcgagcagct gccatggcga ggaggaaact
gctaccagga agaattcagt tatggggaca tggtattgca
gtagactggg cagagccaga agtagaagtt gatgaagata
caatgtcttc agtgaaaatc ctatatgtaa gaaatcttat
gctgtctacc tctgaagaga tgattgaaaa ggaattcaac
aatatcaaac caggtgctgt ggagagggtg aagaaaattc
gagactatgc ttttgtgcac ttcagtaacc gagaagatgc
agttgaggct atgaaagctt taaatggcaa ggtgctggat
ggttccccca ttgaagtcac cctagcaaaa ccagtggaca
aggacagtta tgttaggtat acccgaggca caggtggaag
gggcaccatg ctgcaaggag agtataccta ctctttgggc
caagtttatg atcccaccac aacctacctt ggagctcctg
tcttctatgc cccccagacc tatgcagcaa ttcccagtct
tcatttccca gccaccaaag gacatctcag caacagagcc
attatccgag ccccttctgt tagaggggct gcgggagtga
gaggactggg cggccgtggc tatttggcat acacaggcct
gggtcgagga taccaggtca aaggagacaa aagagaagac
aaactctatg acattttacc tgggatggag ctcaccccaa
tgaatcctgt cacattaaaa ccccaaggaa ttaaactcgc
tccccagata ttagaagaga tttgtcagaa aaataactgg
ggacagccag tgtaccagct gcactctgct attggacaag
accaaagaca gctattcttg tacaaaataa ctattcctgc
tctagccagc cagaatcctg caatccaccc tttcacacct
ccaaagctga gtgcctttgt ggatgaagca aagacgtatg
cagccgaata caccctgcag accctgggca tccccactga
tggaggcgat ggcaccatgg ctactgctgc tgctgctgct
actgctttcc caggatatgc tgtccctaat gcaactgcac
ccgtgtctgc agcccagctc aagcaagcgg taacccttgg
acaagactta gcagcatata caacctatga ggtctaccca
acttttgcag tgactgcccg aggggatgga tatggcacct
tctgaagatg

The ACF64 sequence can comprise the amino acid sequence SEQ ID NO:1, an amino acid sequence at least about 90% identical to the amino acid sequence of SEQ ID NO:1, the amino acid sequence SEQ ID NO:1 having one or more conservative amino acid substitutions, or the amino acid sequence SEQ ID NO:1 having one or more mutations. The full length of ACF64 has an amino acid sequence according to SEQ ID NO:1 as follows:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Pro Gly Trp Asp
Ala Ala Pro Pro Glu Arg Gly Cys Glu Ile Phe Ile
Gly Lys Leu Pro Arg Asp Leu Phe Glu Asp Glu Leu
Ile Pro Leu Cys Glu Lys Ile Gly Lys Ile Tyr Glu
Met Arg Met Met Met Asp Phe Asn Gly Asn Asn Arg
Gly Tyr Ala Phe Val Thr Phe Ser Asn Lys Val Glu
Ala Lys Asn Ala Ile Lys Gln Leu Asn Asn Tyr Glu
Ile Arg Asn Gly Arg Leu Leu Gly Val Cys Ala Ser
Val Asp Asn Cys Arg Leu Phe Val Gly Gly Ile Pro
Lys Thr Lys Lys Arg Glu Glu Ile Leu Ser Glu Met
Lys Lys Val Thr Asn Glu Gly Val Val Asp Val Ile
Val Tyr Pro Ser Ala Ala Asp Lys Thr Lys Asn Arg
Gly Phe Ala Phe Val Glu Tyr Glu Ser His Arg Thr
Ala Ala Met Ala Arg Arg Lys Leu Leu Pro Gly Arg
Ile Gln Leu Trp Gly His Gly Ile Ala Val Asp Trp
Ala Glu Pro Glu Val Glu Val Asp Glu Asp Thr Met
Ser Ser Val Lys Ile Leu Tyr Val Arg Asn Leu Met
Leu Ser Thr Ser Glu Glu Met Ile Glu Lys Glu Phe
Asn Asn Ile Lys Pro Gly Ala Val Glu Arg Val Lys
Lys Ile Arg Asp Tyr Ala Phe Val His Phe Ser Asn
Arg Lys Asp Ala Val Glu Ala Met Lys Ala Leu Asn
Gly Lys Val Leu Asp Gly Ser Pro Ile Glu Val Thr
Leu Ala Lys Pro Val Asp Lys Asp Ser Tyr Val Arg
Tyr Thr Arg Gly Thr Gly Gly Arg Gly Thr Met Leu
Gln Gly Glu Tyr Thr Tyr Ser Leu Gly Gln Val Tyr
Asp Pro Thr Thr Thr Tyr Leu Gly Ala Pro Val Phe
Tyr Ala Pro Gln Thr Tyr Ala Ala Ile Pro Ser Leu
His Phe Pro Ala Thr Lys Gly His Leu Ser Asn Arg
Ala Ile Ile Arg Ala Pro Ser Val Arg Gly Ala Ala
Gly Val Arg Gly Leu Gly Gly Arg Gly Tyr Leu Ala
Tyr Thr Gly Leu Gly Arg Gly Tyr Gln Val Lys Gly
Asp Lys Arg Glu Asp Lys Leu Tyr Asp Ile Leu Pro
Gly Met Glu Leu Thr Pro Met Asn Pro Val Thr Leu
Lys Pro Gln Gly Ile Lys Leu Ala Pro Gln Ile Leu
Glu Glu Ile Cys Gln Lys Asn Asn Trp Gly Gln Pro
Val Tyr Gln Leu His Ser Ala Ile Gly Gln Asp Gln
Arg Gln Leu Phe Leu Tyr Lys Ile Thr Ile Pro Ala
Leu Ala Ser Gln Asn Pro Ala Ile His Pro Phe Thr
Pro Pro Lys Leu Ser Ala Phe Val Asp Glu Ala Lys
Thr Tyr Ala Ala Glu Asn Tyr Thr Leu Gln Thr Leu
Gly Ile Pro Thr Asp Gly Gly Asp Gly Thr Met Ala
Thr Ala Ala Ala Ala Ala Thr Ala Phe Pro Gly Tyr
Ala Val Pro Asn Ala Thr Ala Pro Val Ser Ala Ala
Gln Leu Lys Gln Ala Val Thr Leu Gly Gln Asp Leu
Ala Ala Tyr Thr Asn Thr Tyr Glu Val Tyr Pro Thr
Phe Ala Val Thr Ala Arg Gly Asp Gly Tyr Gly Thr
Phe

The ACF45 sequence can comprise the amino acid sequence SEQ ID NO:18, an amino acid sequence at least about 90% identical to the amino acid sequence of SEQ ID NO:18, the amino acid sequence SEQ ID NO:18 having one or more conservative amino acid substitutions, or the amino acid sequence SEQ ID NO:18 having one or more mutations. A DNA molecule encoding ACF45 has a nucleotide sequence according to SEQ ID NO:5 as follows:

gataatcaag gaaacctttt ccgggtgggg atctctgaaa
ttactcagat acagtgctgtgccaaaaacc tgtggatttt
ctctacaaaa attattgagc aaccctaatt aacctgattt
tttgctgata atcactctca atggaatcaa atcacaaatc
cggggatgga ttgagcggca ctcagaagga agcagccctc
cgcgcactgg tccagcgcac aggatatagc ttggtccagg
aaaatggaca aagaaaatat ggtggccctc cacctggttg
ggatgctgca ccccctgaaa ggggctgtga aatttttatt
ggaaaacttc cccgagacct ttttgaggat gagcttatac
cattatgtga aaaaatcggt aaaatttatg aaatgagaat
gatgatggat tttaatggca acaatagagg atatgcattt
gtaacatttt caaataaagt ggaagccaag aatgcaatca
agcaacttaa taattatgaa attagaaatg ggcgcctctt
aggggtttgt gccagtgtgg acaactgccg attatttgtt
gggggcatcc caaaaaccaa aaagagagaa gaaatcttat
cggagatgaa aaaggttact gaaggtgttg tcgatgtcat
cgtctaccca agcgctgcag ataaaaccaa aaaccgaggc
tttgccttcg tggagtatga gagtcatcga acagctgcca
tggcgaggag gaaactgcta ccaggaagaa ttcagttatg
gggacatggt attgcagtag actgggcaga gccagaagta
gaagttgatg aagatacaat gtcttcagtg aaaatcctat
atgtaagaaa tcttatgctg tctacctctg aagagatgat
tgaaaaggaa ttcaacaata tcaaaccagg tgctgtggag
agggtgaaga aaattcgaga ctatgctttt gtgcacttca
gtaaccgaaa agatgcagtt gaggctatga aagctttaaa
tggcaaggtg ctggatggtt cccccattga agtcacccta
gcaaaaccag tggacaagga cagttatgtt aggtataccc
gaggcacagg tggaaggggc accatgctgc aaggagagta
tacctactct ttgggccaag tttatgatcc caccacaacc
taccttggag ctcctgtctt ctatgccccc cagacctatg
cagcaattcc cagtcttcat ttcccagcca ccaaaggaca
tctcagcaac agagccatta tccgagcccc ttctgttaga
ggggctgcgg gagtgagagg actgggcggc cgtggctatt
tggcatacac aggcctgggt cgaggatacc aggtcaaagg
agacaaaaga gaagacaaac tctatgacat tttacctggg
atggagctca ccccaatgaa tcctgtcaca ttaaaacccc
aaggaattaa actcgctccc cagatattag aagagatttg
tcagaaaaat aactggggac agccagtgta ccagctgcac
tctgctattg gacaagacca aagacagcta ttcttgtaca
aaataactat tcctgctcta gccagccaga atcctgcaat
ccaccctttc acacctccaa agctgagtgc ctttgtggat
gaagcaaaga cgtatgcagc cgaatacacc ctgcagaccc
tgggcatccc cactgatgga ggcgatggca ccatggctac
tgctgctgct gctgctactg ctttcccagg atatgctgtc
cctaatgcaa ctgcacccgt gtctgcagcc cagctcaagc
aagcggtaac ccttggacaa gacttagcag catatacaac
ctatgaggtc tacccaactt ttgcagtgac tgcccgaggg
gatggatatg gcaccttctg aagatgcttt tttaaattta
agaataagac acacaaaact ctatt

ACF45 has an amino acid sequence according to SEQ ID NO: 18 as follows:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Asn Pro Gly Trp
Asp Thr Thr Pro Pro Glu Arg Gly Cys Glu Ile Phe
Ile Gly Lys Leu Pro Arg Asp Leu Phe Glu Asp Glu
Leu Ile Pro Leu Cys Glu Lys Ile Gly Lys Ile Tyr
Glu Met Arg Met Met Met Asp Phe Asn Gly Asn Asn
Arg Gly Tyr Ala Phe Val Thr Asn Phe Ser Asn Lys
Gln Glu Ala Lys Asn Ala Ile Lys Gln Leu Asn Asn
Tyr Glu Ile Arg Asn Gly Arg Leu Leu Gly Val Cys
Ala Ser Val Asp Asn Cys Arg Leu Phe Val Gly Gly
Ile Pro Lys Thr Lys Lys Arg Glu Glu Ile Leu Ser
Glu Met Lys Lys Val Thr Asn Glu Gly Val Val Asp
Val Ile Val Tyr Pro Ser Ala Ala Asp Lys Thr Lys
Asn Arg Gly Phe Ala Phe Val Glu Tyr Glu Ser His
Arg Ala Ala Ala Met Ala Arg Arg Arg Leu Leu Pro
Gly Arg Ile Gln Leu Trp Gly His Pro Ile Ala Val
Asp Trp Ala Glu Pro Asn Glu Val Glu Val Asp Glu
Asp Thr Met Ser Ser Val Lys Ile Leu Tyr Val Arg
Asn Leu Met Leu Ser Thr Ser Glu Glu Met Ile Glu
Lys Glu Phe Asn Ser Ile Lys Pro Gly Ala Val Glu
Arg Val Lys Lys Ile Arg Asp Tyr Ala Phe Val His
Phe Ser Asn Arg Asn Glu Asp Ala Val Glu Ala Met
Lys Ala Leu Asn Gly Lys Val Leu Asp Gly Ser Pro
Ile Glu Val Thr Leu Ala Lys Pro Val Asp Lys Asp
Ser Tyr Val Arg Tyr Thr Arg Gly Thr Gly Gly Arg
Asn Thr Met Leu Gln Glu Tyr Thr Tyr Pro Leu Ser
His Val Tyr Asn Asp Pro Thr Thr Thr Tyr Leu Gly
Ala Pro Val Phe Tyr Ala Pro Gln Ala Tyr Ala Ala
Ile Pro Ser Leu His Phe Pro Ala Thr Lys Gly His
Leu Ser Asn Arg Ala Leu Ile Arg Thr Pro Ser Val
Arg Gly Cys Ser Arg Thr Pro Ser Ile Tyr Leu Cys
Phe Leu Asn Thr Ala Val His Ala Gly Val His His
Ile His Val Gln

A DNA molecule encoding ACF45 has a nucleotide sequence according to SEQ ID NO: 21 as follows:

gaattcacca taatcaagaa accttttccg ggtgggggaa
cctcagaaat tactcagtga gcaaccctaa ttaacctgat
tttttgctga taatcactct caatggaatc aaatcacaaa
tccggggatg gattgagcgg cacccagaag gaagcagcac
tccgcgcact ggtccagcgc acaggatata gcttggtcca
ggaaaatgga caaagaaaat atggtggtcc tccaccaggc
tgggatacta cacccccaga aaggggctgc gagattttca
ttgggaaact tccccgggac ctttttgagg atgaactcat
accattgtgt gaaaaaattg gtaaaattta tgaaatgaga
atgatgatgg atttcaatgg gaacaacaga ggctatgcat
ttgtaacctt ctcaaataag caggaagcca agaatgcaat
caagcaactt aataattatg aaattcggaa tggccgtctc
ctgggcgtct gtgccagtgt ggacaactgc cggttgtttg
tggggggaat ccccaaaacc aaaaagagag aagaaatctt
gtcagagatg aaaaaggtca ctgaaggagt tgttgatgtc
attgtctacc caagcgctgc cgataaaacc aaaaaccggg
ggtttgcctt tgtggaatat gagagtcacc gcgcagccgc
catggctagg cggaggctgc tgccaggaag aattcagttg
tggggacatc ctatcgcagt agactgggca gagccagaag
tcgaagttga cgaagacaca atgtcttccg tgaaaatcct
gtacgtaagg aaccttatgc tgtctacctc ggaagagatg
attgagaagg aattcaacag tattaaacca ggtgctgtgg
aacgggtgaa gaagattcga gactatgctt ttgtgcattt
cagtaaccga gaagatgcag ttgaagccat gaaggctttg
aatggcaagg tgctggatgg ttccccaata gaagtgacct
tggccaagcc agtggacaag gacagttacg ttaggtacac
ccggggcacc gggggcagga acaccatgct gcaagaatac
acctaccctc tgagccatgt ttatgaccct accacaacct
accttggagc tcctgtcttctatgctcccc aagcctacgc
agccattcca agtcttcatt tcccagctac caaaggacat
ctcagcaaca gagctctcat ccggacccct tctgtcagag
gctgctcaag aactcccagc atctaccttt gcttcctgac
tgctgtacat gcaggtgtgc accacatcca tgtgcagtaa
ctttgaaaaa acaaatcaaa caaggagatt aatattgctt
tagcataatg gaaaccaaat aaactaaaaa aaaa

The ACF43 sequence can comprise the amino acid sequence SEQ ID NO:19, an amino acid sequence at least about 90% identical to the amino acid sequence of SEQ ID NO:19, the amino acid sequence SEQ ID NO:19 having one or more conservative amino acid substitutions, or the amino acid sequence SEQ ID NO:19 having one or more mutations. ACF43 has an amino acid sequence according to SEQ ID NO:19 as follows:

Met Glu Ser Asn His Lys Ser Gly Asp Gly Leu Ser
Gly Thr Gln Lys Glu Ala Ala Leu Arg Ala Leu Val
Gln Arg Thr Gly Tyr Ser Leu Val Gln Glu Asn Gly
Gln Arg Lys Tyr Gly Gly Pro Pro Asn Pro Gly Trp
Asp Thr Thr Pro Pro Glu Arg Gly Cys Glu Ile Phe
Ile Gly Lys Leu Pro Arg Asp Leu Phe Glu Asp Glu
Leu Ile Pro Leu Cys Glu Lys Ile Gly Lys Ile Tyr
Glu Met Arg Met Met Met Asp Phe Asn Gly Asn Asn
Arg Gly Tyr Ala Phe Val Thr Asn Phe Ser Asn Lys
Gln Glu Ala Lys Asn Ala Ile Lys Gln Leu Asn Asn
Tyr Glu Ile Arg Asn Gly Arg Leu Leu Gly Val Cys
Ala Ser Val Asp Asn Cys Arg Leu Phe Val Gly Gly
Ile Pro Lys Thr Lys Lys Arg Glu Glu Ile Leu Ser
Glu Met Lys Lys Val Thr Asn Glu Gly Val Val Asp
Val Ile Val Tyr Pro Ser Ala Ala Asp Lys Thr Lys
Asn Arg Gly Phe Ala Phe Val Glu Tyr Glu Ser His
Arg Ala Ala Ala Met Ala Arg Arg Arg Leu Leu Pro
Gly Arg Ile Gln Leu Trp Gly His Pro Ile Ala Val
Asp Trp Ala Glu Pro Asn Glu Val Glu Val Asp Glu
Asp Thr Met Ser Ser Val Lys Ile Leu Tyr Val Arg
Asn Leu Met Leu Ser Thr Ser Glu Glu Met Ile Glu
Lys Glu Phe Asn Ser Ile Lys Pro Gly Ala Val Glu
Arg Val Lys Lys Ile Arg Asp Tyr Ala Phe Val His
Phe Ser Asn Arg Asn Glu Asp Ala Val Glu Ala Met
Lys Ala Leu Asn Gly Lys Val Leu Asp Gly Ser Pro
Ile Glu Val Thr Leu Ala Lys Pro Val Asp Lys Asp
Ser Tyr Val Arg Tyr Thr Arg Gly Thr Gly Gly Arg
Asn Thr Met Leu Gln Glu Tyr Thr Tyr Pro Leu Ser
His Val Tyr Asn Asp Pro Thr Thr Thr Tyr Leu Gly
Ala Pro Val Phe Tyr Ala Pro Gln Ala Tyr Ala Ala
Ile Pro Ser Leu His Phe Pro Ala Thr Lys Gly His
Leu Ser Asn Arg Ala Leu Ile Arg Thr Pro Ser Val
Arg Gly Asn Ile Ser

A DNA molecule encoding ACF43 has a nucleotide sequence according to SEQ ID NO: 22 as follows:

gaattcacca taatcaagaa accttttccg ggtgggggaa
cctcagaaat tactcagtga gcaaccctaa ttaacctgat
tttttgctga taatcactct caatggaatc aaatcacaaa
tccggggatg gattgagcgg cacccagaag gaagcagcac
tccgcgcact ggtccagcgc acaggatata gcttggtcca
ggaaaatgga caaagaaaat atggtggtcc tccaccaggc
tgggatacta cacccccaga aaggggctgc gagattttca
ttgggaaact tccccgggac ctttttgagg atgaactcat
accattgtgt gaaaaaattg gtaaaattta tgaaatgaga
atgatgatgg atttcaatgg gaacaacaga ggctatgcat
ttgtaacctt ctcaaataag caggaagcca agaatgcaat
caagcaactt aataattatg aaattcggaa tggccgtctc
ctgggcgtct gtgccagtgt ggacaactgc cggttgtttg
tggggggaat ccccaaaacc aaaaagagag aagaaatctt
gtcagagatg aaaaaggtca ctgaaggagt tgttgatgtc
attgtctacc caagcgctgc cgataaaacc aaaaaccggg
ggtttgcctt tgtggaatat gagagtcacc gcgcagccgc
catggctagg cggaggctgc tgccaggaag aattcagttg
tggggacatc ctatcgcagt agactgggca gagccagaag
tcgaagttga cgaagacaca atgtcttccg tgaaaatcct
gtacgtaagg aaccttatgc tgtctacctc ggaagagatg
attgagaagg aattcaacag tattaaacca ggtgctgtgg
aacgggtgaa gaagattcga gactatgctt ttgtgcattt
cagtaaccga gaagatgcag ttgaagccat gaaggctttg
aatggcaagg tgctggatgg ttccccaata gaagtgacct
tggccaagcc agtggacaag gacagttacg ttaggtacac
ccggggcacc gggggcagga acaccatgct gcaagaatac
acctaccctc tgagccatgt ttatgaccct accacaacct
accttggagc tcctgtcttc tatgctcccc aagcctacgc
agccattcca agtcttcatt tcccagctac caaaggacat
ctcagcaaca gagctctcat ccggacccct tctgtcagag
gtaacattag ctagctcttg tctctccata actccaaatt
tcaaatgact aataaacgag tttaaatgaa ctataaacta
tacttcagta gccttaaata gattggatgg aatcactggg
agaaaaaaaa gagtagaaaa ctgttaaaaa atcaactata
tcagtatctc ttgtcatata ggaatctatt ttttcacatt
acaactggtt attgaaaaat acttgtcaga acgtgatatg
aatgagccca gtccataagt gagattcttt tagtatataa
attggatcca caaaatatgc acacaatata accttgctct
tataaccatt gcctcttcct ccttattaaa ccaactagac
cagttacaga actttcaagg gagagcattg gggcctaatg
ggacctgcct caaatgaggc attctcatat aacctctctc
ctggatatgt gtgctatacc tttgctagcc catgtgtttg
tctgtatgtt ggtgacaagg gcttactata tattccaggc
tgctcaagaa ctcccagcat ctacctttgc ttcctgactg
ctgtacatgc aggtgtgcac cacatccatg tgcagtaact
ttgaaaaaac aaatcaaaca aggagattaa tattgcttta
gcataatgga aaccaaataa actaaaaaaa aaaaaa

### Apobec-1

Also disclosed herein are APOBEC-1 polypeptides and fragments thereof, which are capable of use with the methods disclosed herein. Specifically, compositions comprising APOBEC-1 polypeptides and nucleic acids can be used in conjunction with compositions comprising ACF polypeptides and nucleic acids. A more detailed description of the these methods can be found below.

The full length human APOBEC-1 has an amino acid sequence according to SEQ ID NO: 13 as follows:

Met Thr Ser Glu Lys Gly Pro Ser Thr Gly Asp Pro
Thr Leu Arg Arg Arg Ile Glu Pro Trp Glu Phe Asp
Val Phe Tyr Asp Pro Arg Glu Leu Arg Lys Glu Ala
Cys Leu Leu Tyr Glu Ile Lys Trp Gly Met Ser Arg
Lys Ile Trp Arg Ser Ser Gly Lys Asn Thr Thr Asn
His Val Glu Val Asn Phe Ile Lys Lys Phe Thr Ser
Glu Arg Asp Phe His Pro Ser Ile Ser Cys Ser Ile
Thr Trp Phe Leu Ser Trp Ser Pro Cys Trp Glu Cys
Ser Gln Ala Ile Arg Glu Phe Leu Ser Arg His Pro
Gly Val Thr Leu Val Ile Tyr Val Ala Arg Leu Phe
Trp His Met Asp Gln Gln Asn Arg Gln Gly Leu Arg
Asp Leu Val Asn Ser Gly Val Thr Ile Gln Ile Met
Arg Ala Ser Glu Tyr Tyr His Cys Trp Arg Asn Phe
Val Asn Tyr Pro Pro Gly Asp Glu Ala His Trp Pro
Gln Tyr Pro Pro Leu Trp Met Met Leu Tyr Ala Leu
Glu Leu His Cys Ile Ile Leu Ser Leu Pro Pro Cys
Leu Lys Ile Ser Arg Arg Trp Gln Asn His Leu Thr
Phe Phe Arg Leu His Leu Gln Asn Cys His Tyr Gln
Thr Ile Pro Pro His Ile Leu Leu Ala Thr Gly Leu
Ile His Pro Ser Val Ala Trp Arg.

This human APOBEC-1 sequence is reported at Genbank Accession No. NP_{—}13001635, which is hereby incorporated by reference in its entirety. The full length human APOBEC-1 includes a putative bipartite nuclear localization signal between amino acid residues 15-34, a catalytic center between amino acid residues 61-98, and a putative cytoplasmic retention signal between amino acid residues 173-229. A cDNA sequence which encodes the full length human APOBEC-1 is set forth as SEQ ID NO: 14 as follows:

atgacttctg agaaaggtcc ttcaaccggt gaccccactc
tgaggagaag aatcgaaccc tgggagtttg acgtcttcta
tgaccccaga gaacttcgta aagaggcctg tctgctctac
gaaatcaagt ggggcatgag ccggaagatc tggcgaagct
caggcaaaaa caccaccaat cacgtggaag ttaattttat
aaaaaaattt acgtcagaaa gagattttca cccatccatc
agctgctcca tcacctggtt cttgtcctgg agtccctgct
gggaatgctc ccaggctatt agagagtttc tgagtcggca
ccctggtgtg actctagtga tctacgtagc tcggcttttt
tggcacatgg atcaacaaaa tcggcaaggt ctcagggacc
ttgttaacag tggagtaact attcagatta tgagagcatc
agagtattat cactgctgga ggaattttgt caactaccca
cctggggatg aagctcactg gccacaatac ccacctctgt
ggatgatgtt gtacgcactg gagctgcact gcataattct
aagtcttcca ccctgtttaa agatttcaag aagatggcaa
aatcatctta catttttcag acttcatctt caaaactgcc
attaccaaac gattccgcca cacatccttt tagctacagg
gctgatacat ccttctgtgg cttggagatg a.

The full length rat APOBEC-1 has an amino acid sequence according to SEQ ID NO: 29 as follows:

Met Ser Ser Glu Thr Gly Pro Val Ala Val Asp Pro
Thr Leu Arg Arg Arg Ile Glu Pro His Glu Phe Glu
Val Phe Phe Asp Pro Arg Glu Leu Arg Lys Glu Thr
Cys Leu Leu Tyr Glu Ile Asn Trp Gly Gly Arg His
Ser Ile Trp Arg His Thr Ser Gln Asn Thr Asn Lys
His Val Glu Val Asn Phe Ile Glu Lys Phe Thr Thr
Glu Arg Tyr Phe Cys Pro Asn Thr Arg Cys Ser Ile
Thr Trp Phe Leu Ser Trp Ser Pro Cys Gly Glu Cys
Ser Arg Ala Ile Thr Glu Phe Leu Ser Arg Tyr Pro
His Val Thr Leu Phe Ile Tyr Ile Ala Arg Leu Tyr
His His Ala Asp Pro Arg Asn Arg Gln Gly Leu Arg
Asp Leu Ile Ser Ser Gly Val Thr Ile Gln Ile Met
Thr Glu Gln Glu Ser Gly Tyr Cys Trp Arg Asn Phe
Val Asn Tyr Ser Pro Ser Asn Glu Ala His Trp Pro
Arg Tyr Pro His Leu Trp Val Arg Leu Tyr Val Leu
Glu Leu Tyr Cys Ile Ile Leu Gly Leu Pro Pro Cys
Leu Asn Ile Leu Arg Arg Lys Gln Pro Gln Leu Thr
Phe Phe Thr Ile Ala Leu Gln Ser Cys His Tyr Gln
Arg Leu Pro Pro His Ile Leu Trp Ala Thr Gly Leu
Lys.

This rat APOBEC-1 sequence is reported at Genbank Accession No. P38483, which is hereby incorporated by reference in its entirety. Recombinant studies using rat APOBEC-1 have demonstrated that an N-terminal region, containing the putative nuclear localization signal, is required for nuclear distribution of APOBEC-1 while a C-terminal region, containing a putative cytoplasmic retention signal (Yang et al., Proc. Natl. Acad. Sci. USA 94:13075-13080 (1997), which is hereby incorporated by reference in its entirety.

A cDNA sequence which encodes the full length rat APOBEC-1 is set forth as SEQ ID NO: 30 as follows:

atgagttccg asacaggccc tgtagctgtt gatcccactc
tgaggagaag aattgagccc cacgagtttg aagtcttctt
tgacccccgg gaacttcgga aagagacctg tctgctgtat
gagatcaact gggaggaag gcacagcatc tggcgacaca
cgagccaaaa caccaacaaa cacgttgaag tcaatttcat
agaaaaattt actacagaaa gatacttttg tccaaacacc
agatgctcca ttacctggtt cctgtcctgg agtccctgtg
gggagtgctc cagggccatt acagaatttt tgagccgata
cccccatgta actctgttta tttatatagc acggctttat
caccacgcag atcctcgaaa tcggcaagga ctcagggacc
ttattagcag cggtgttact atccagatca tgacggagca
agagtctggc tactgctgga ggaattttgt caactactcc
ccttcgaatg aagctcattg gccaaggtac ccccatctgt
gggtgaggct gtacgtactg gaactctact gcatcatttt
aggacttcca ccctgtttaa atattttaag aagaaaacaa
cctcaactca cgtttttcac gattgctctt caaagctgcc
attaccaaag gctaccaccc cacatcctgt gggccacagg
gttgaaatga.

The cDNA molecule is reported at Genbank Accession No. L07114, which is hereby incorporated by reference in its entirety.

The full length mouse APOBEC-1 has an amino acid sequence according to SEQ ID NO: 31 as follows:

Met Ser Ser Glu Thr Gly Pro Val Ala Val Asp Pro
Thr Leu Arg Arg Arg Ile Glu Pro His Glu Phe Glu
Val Phe Phe Asp Pro Arg Glu Leu Arg Lys Glu Thr
Cys Leu Leu Tyr Glu Ile Asn Trp Gly Gly Arg His
Ser Val Trp Arg His Thr Ser Gln Asn Thr Ser Asn
His Val Glu Val Asn Phe Leu Glu Lys Phe Thr Thr
Glu Arg Tyr Phe Arg Pro Asn Thr Arg Cys Ser Ile
Thr Trp Phe Leu Ser Trp Ser Pro Cys Gly Glu Cys
Ser Arg Ala Ile Thr Glu Phe Leu Ser Arg His Pro
Tyr Val Thr Leu Phe Ile Tyr Ile Ala Arg Leu Tyr
His His Thr Asp Gln Arg Asn Arg Gln Gly Leu Arg
Asp Leu Ile Ser Ser Gly Val Thr Ile Gln Ile Met
Thr Glu Gln Glu Tyr Cys Tyr Cys Trp Arg Asn Phe
Val Asn Tyr Pro Pro Ser Asn Glu Ala Tyr Trp Pro
Arg Tyr Pro His Leu Trp Val Lys Leu Tyr Val Leu
Glu Leu Tyr Cys Ile Ile Leu Gly Leu Pro Pro Cys
Leu Lys Ile Leu Arg Arg Lys Gln Pro Gln Leu Thr
Phe Phe Thr Ile Thr Leu Gln Thr Cys His Tyr Gln
Arg Ile Pro Pro His Leu Leu Trp Ala Thr Gly Leu
Lys.

This mouse APOBEC-1 sequence is reported at Genbank Accession No. NP_{—}13112436, which is hereby incorporated by reference in its entirety. A cDNA sequence which encodes the full length mouse APOBEC-1 is set forth as SEQ ID NO: 32 as follows:

atgagttccg agacaggccc tgtagctgtt gatcccactc
tgaggagaag aattgagccc cacgagtttg aagtcttctt
tgacccccgg gagcttcgga aagagacctg tctgctgtat
gagatcaact ggggtggaag gcacagtgtc tggcgacaca
cgagccaaaa caccagcaac cacgttgaag tcaacttctt
agaaaaattt actacagaaa gatactttcg tccgaacacc
agatgctcca ttacctggtt cctgtcctgg agtccctgcg
gggagtgctc cagggccatt acagagtttc tgagccgaca
cccctatgt& actctgttta tttacatagc acggctttat
caccacacgg atcagcgaaa ccgccaagga ctcagggacc
ttattagcag cggtgtgact atccagatca tgacagagca
agagtattgt tactgctgga ggaatttcgt caactacccc
ccttcaaacg aagcttattg gccaaggtac ccccatctgt
gggtgaaact gtatgtattg gagctctact gcatcatttt
aggacttcca ccctgtttaa aaattttaag
aagaaagcaacctcaactca cgtttttcac aattactctt
caaacctgcc attaccaaag gataccaccc catctccttt
gggctacagg gttgaaatga.

The cDNA molecule is reported at Genbank Accession No. NM_{—}13031159, which is hereby incorporated by reference in its entirety.

Signal Sequences

Also disclosed herein are signal sequences, such as secretion sequences, useful with the ACF and APOBEC-1 polypeptides and nucleic acids disclosed herein. These sequences can be added to the N-terminal or the C-terminal amino acid sequences. These motifs are present in numerous secreted proteins and have varying abilities to direct a protein during translation to the endoplasmic reticulum and out of the cell. For example, the subject's own cells (hepatocytes, for example) produce and secrete TAT-ACF into the local circulation where it can transduce hepatocytes with the liver.

Various signal sequences can be used with the polypeptides disclosed herein. One example is an albumin signal sequence. Albumin is a soluble, monomeric protein that is produced by the liver and comprises about one-half of the blood serum protein. Albumin functions primarily as a carrier protein for steroids, fatty acids, and thyroid hormones and plays a role in stabilizing extracellular fluid volume. Mutations in this gene on chromosome 4 result in various anomalous proteins. Albumin is a globular unglycosylated serum protein of molecular weight 65,000. The human albumin gene is 16,961 nucleotides long from the putative ‘cap’ site to the first poly(A) addition site. It is split into 15 exons which are symmetrically placed within the 3 domains that are thought to have arisen by triplication of a single primordial domain. Albumin is synthesized in the liver as preproalbumin which has an N-terminal peptide that is removed before the nascent protein is released from the rough endoplasmic reticulum. The product, proalbumin, is in turn cleaved in the Golgi vesicles to produce the secreted albumin. An example of the albumin signal sequence can be found in SEQ ID NO: 24.

Other signal sequences are known to those of skill in the art, and can be found, for example, in the SIGPEP database. SigPep is a meta-database providing information on prokaryotic and eukaryotic signal peptides found in the public databases. The SigPep database provides information on signal peptide using information like accession number, sequence, and text search. Using relational database as the management system, coupled with a web interface, Sigpep facilitates efficient search of signal peptides. Data is extracted from high quality public databases and automatically updated when these databases are updated. SigPep can be accessed at http://proline.bic.nus.edu.sg/sigpep (von Heijne, G. Protein Seq Data Anal. 1987; 1 (1):41-2). Other secretion sequences can be used, which can readily be identified by one of skill in the art. For example, any protein that can be utilized by the liver to secrete TAT-APOBEC can also be used.

### Transduction Sequences

Various transduction sequences can be used with the polypeptides disclosed herein. By way of example, protein transduction domains from several known proteins can be employed, including without limitation, HIV-1 Tat protein, Drosophila homeotic transcription factor (ANTP), and HSV-1 VP22 transcription factor (Schwarze et al., TiPS 21:45-48 (2000), which is hereby incorporated by reference in its entirety).

A preferred protein transduction domain is the protein transduction domain of the human immunodeficiency virus (“HIV”) Tat protein. An exemplary HIV Tat protein transduction domain has an amino acid sequence of SEQ ID NO: 23 as follows:

Arg Lys Lys Arg Arg Gln Arg Arg Arg

This protein transduction domain has also been noted to be a nuclear translocation domain (HIV Sequence Compendium 2000, Kuiken et al. (eds.), Theoretical Biology and Biophysics Group, Los Alamos National Laboratory, which is hereby incorporated by reference in its entirety). One DNA molecule which encodes the HIV Tat protein transduction domain has a nucleotide sequence of SEQ ID NO: 14 as follows:

agaaaaaaaa gaagacaaag aagaaga

Variations of these Tat sequences can also be employed. Such sequence variants have been reported in HIV Sequence Compendium 2000, Kuiken et al. (eds.), Theoretical Biology and Biophysics Group, Los Alamos National Laboratory, which is hereby incorporated by reference in its entirety.

Other cellular uptake polypeptides and their use have been described in the literature, including membrane-permeable sequences of the SN50 peptide, the Grb2 SH2 domain, and integrin β3, β1, and αIIb cytoplasmic domains (Hawiger, Curr. Opin. Chem. Biol. 3:89-94 (1999), which is hereby incorporated by reference in its entirety).

### Sequence Homology/Identity

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

There are a variety of molecules disclosed herein that are nucleic acid based, including for example the nucleic acids that encode the polypeptides disclosed herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### Sequences

There are a variety of sequences related to, for example, ACF and APOBEC-1 polypeptides, as well as any other protein disclosed herein that are disclosed on Genbank, and these sequences and others are herein incorporated by reference in their entireties as well as for individual subsequences contained therein. The disclosed sequences can be naturally occurring sequences or fragments thereof, variants of sequences or fragments thereof, or modified forms of sequences or fragments thereof. Thus, the disclosed ACF and APOBEC-1 sequences can be naturally occurring sequences or fragments thereof or fragments thereof having RNA editing activity, variants of narurally occurring ACF and APOBEC-1 sequences or fragments thereof or fragments thereof having RNA editing activity, modified forms of narurally occurring ACF and APOBEC-1 sequences or fragments thereof or fragments thereof having RNA editing activity. Similarly, the disclosed secretion sequences can be naturally occurring secretion sequences or fragments thereof or fragments thereof having secretion activity, variants of narurally occurring secretion sequences or fragments thereof or fragments thereof having secretion activity, modified forms of narurally occurring secretion sequences or fragments thereof or fragments thereof having secretion activity. The disclosed transduction sequences can be naturally occurring transduction sequences or fragments thereof or fragments thereof having transduction activity, variants of narurally occurring transduction sequences or fragments thereof or fragments thereof having transduction activity, modified forms of narurally occurring transduction sequences or fragments thereof or fragments thereof having transduction activity. The disclosed ACF and APOBEC-1 sequences and polypeptides can comprise such variants, modified forms, and fragments. Variants, modifications and fragments of sequences are described below and elsewhere herein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

Disclosed are compositions including primers and probes, which are capable of interacting with the nucleic acids disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

In the methods disclosed herein, molecules such as ACF and APOBEC-1 polypeptides can be used in assays. These molecules can be, for example, chimeric proteins. By “chimeric protein” is meant any single polypeptide unit that comprises two distinct polypeptide domains joined by a peptide bond, optionally by means of an amino acid linker, or a non-peptide bond, wherein the two domains are not naturally occurring within the same polypeptide unit. Typically, such chimeric proteins are made by expression of a nucleic acid construct but could be made by protein synthesis methods known in the art. These chimeric proteins are useful in screening compounds, as well as with the compounds identified by the methods disclosed herein.

The compositions disclosed herein can also be fragments or derivatives of a naturally occurring deaminase or viral infectivity factor. A “fragment” is a polypeptide that is less than the full length of a particular protein or functional domain. By “derivative” or “variant” is meant a polypeptide having a particular sequence that differs at one or more positions from a reference sequence. The fragments or derivatives of a full length protein preferably retain at least one function of the full length protein. For example, a fragment or derivative of an ACF polypeptide includes a fragment of ACF that retains at least one function of the full length protein, such as its editing ability. The fragment or derivative can include conservative or non-conservative amino acid substitutions. The fragment or derivative optionally can form a homodimer or a homotetramer.

Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally can be made in accordance with the following Table 1 and are referred to as conservative substitutions.

Substantial changes in function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

TABLE 1
Amino Acid Substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

### Delivery of the Compositions to Cells

As discussed above, disclosed herein are polypeptides comprising an ACF sequence, a secretion sequence and a transduction sequence. Also disclosed herein are vectors comprising a nucleic acid, wherein the nucleic acid encodes a polypeptide comprising an ACF sequence, a secretion sequence and a transduction sequence. In one example, the polypeptide can be secreted from the cell from which it was produced, and the polypeptide can transduce a second cell, thereby delivering the polypeptide to the second cell.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as those encoding an ACF polypeptide, into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpes virus amplicon systems are also being used to package pieces of DNA >220 kb and to infect cells that can stably maintain DNA as episomes.

Other useful systems include, for example, replicating and host-restricted non-replicating vaccinia virus vectors.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### In Vivo/Ex Vivo

As described above, the ACF nucleic acids can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which may affect mRNA expression. These regions are transcribed as polyadenylated segments in the untranslated portion of the mRNA encoding tissue factor protein. The 3′ untranslated regions can also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### Markers

The viral vectors can include nucleic acid sequence encoding a marker product. This marker product can be used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### Therapeutic Uses

Effective dosages and schedules for administering the compositions, such as the ACF polypeptides, may be determined empirically, and making such determinations is within the skill in the art. The dosage ranges for the administration of the compositions are those large enough to produce the desired effect in which the symptoms or disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of nucleic acids. A typical daily dosage of the vectors disclosed herein might range from about 1 μg/kg to up to 100 mg/kg of body weight or more per day, depending on the factors mentioned above. For example, vector dosage can be given according to Naki et al., (J. Virol., 1999, 73:5438-47, herein incorporated by reference in its entirety). In one example, the dosage can be 5×10⁹, 5×10¹⁰ or 5×10¹¹ gene copies (GC)/injection/subject.

Following administration of a disclosed composition, such as an ACF nucleic acid, for treating, inhibiting, or preventing diseases or disorders associated with ApoB, the efficacy of the therapeutic nucleic acid can be assessed in various ways well known to the skilled practitioner. For instance, one of ordinary skill in the art will understand that a composition, such as a nucleic acid, disclosed herein is efficacious in reducing lipid levels in a cell by observing that the composition increases the transport of ApoB-mediated lipid transport from the cell, for example.

The compositions that reduce the level of lipids in the liver disclosed herein may be administered prophylactically to patients or subjects who are at risk for steatohepatitis, or who have insulin resistance, such as in type II diabetes. In subjects who are at risk for steatohepatitis, or those at risk for alcoholic hepatic steatosis but who have not yet experienced symptoms, efficacious treatment with an ACF nucleic acid or polypeptide can partially or completely inhibit the appearance of steatohepatitis and other diseases and disorders related to lipid clearance.

The disclosed compositions and methods can be used in combination with other compositions and methods. For example, there are many compositions and treatments that are available for treating the disorders and diseases disclosed herein related to ApoB100. Discussed herein is a combination therapy wherein APOBEC-1 is also delivered to a subject to reduce the risk of atherosclerosis and other related diseases and disorders.

Other examples of compositions that can be used in combination with those disclosed herein include statin drugs. Statins are drugs that improve cholesterol levels primarily by inhibiting the liver enzyme HMG C-A reductase. Statins have proven to be very effective in reducing cholesterol and in reducing the risk of heart attack and death. Statins include, but are not limited to, atorvastatin (Lipitor), fluvastatin (Lescol), lovastatin (Mevacor), pravastatin (Pravachol), simvastatin (Zocor), and rosuvastatin (Crestor).

The methods disclosed herein can also be used in combination with lipid-lowering bile-acid-binding resin therapy, which has shown efficacy via sequestering the bile acids in the intestine, thereby interrupting the enterohepatic circulation of bile acids and increasing the elimination of cholesterol from the body.

Other molecules that interact with ApoB or lipids which do not have a specific pharmaceutical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of liver-related diseases.

### Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

### Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Compositions Identified by Screening with Disclosed Compositions/Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed in SEQ ID NOS: 1-6, 13, 17-19, 25, 27, 29, 31, or portions thereof, are used as the target in a combinatorial or screening protocol.

It is understood that when using the disclosed compositions in combinatorial techniques or screening methods, molecules, such as macromolecular molecules, will be identified that have particular desired properties such as stimulation or the target molecule's function. The molecules identified and isolated when using the disclosed compositions such as the ACF polypeptides and nucleic acids are also disclosed. Thus, the products produced using the combinatorial or screening approaches that involve the disclosed compositions, such as ACF polypeptides and nucleic acids are also disclosed.

### Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include ACF or APOBEC-1 nucleic acids or polypeptides, such as those disclosed herein.

Compositions with Similar Functions

It is understood that the compositions disclosed herein have certain functions, such as increasing lipid clearance from the liver. Disclosed herein are certain structural requirements for performing the disclosed functions, and it is understood that there are a variety of structures which can perform the same function which are related to the disclosed structures, and that these structures will ultimately achieve the same result, for example variants of the polypeptides and nucleic acids disclosed herein.

### Methods of Making the Compositions

The compositions disclosed herein and the compositions necessary to perform the disclosed methods can be made using any method known to those of skill in the art for that particular reagent or compound unless otherwise specifically noted.

### Nucleic Acid Synthesis

For example, the nucleic acids, such as the ACF and APOBEC-1 nucleic acids, can be made using standard chemical synthesis methods or can be produced using enzymatic methods or any other known method. Such methods can range from standard enzymatic digestion followed by nucleotide fragment isolation (see for example, Sambrook et al., Molecular Cloning A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### Peptide Synthesis

One method of producing the disclosed proteins, such as SEQ ID NOS: 1-6, 13, 17-19, 25, 27, 29, and 31, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmseni L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Processes for making the Compositions

Disclosed are processes for making the compositions as well as making the intermediates leading to the compositions. For example, disclosed are nucleic acids in SEQ ID NOs: 5, 9, 10, 11, 12, 14, 20-22, 26, 28, 30, and 32. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NOS:, 9, 10, 11, 12, 14, 20-22, 26, 28, 30, and 32 and a sequence controlling the expression of the nucleic acid.

Also disclosed are nucleic acid molecules produced by the process comprising linking in an operative way a nucleic acid molecule comprising a sequence having 80% identity to a sequence set forth herein and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth herein and a sequence controlling the expression of the nucleic acid.

Disclosed are nucleic acid molecules produced by the process comprising linking in an operative way a nucleic acid molecule comprising a sequence encoding a peptide set forth herein and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth herein and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in any of SEQ ID NOS: 9, 10, 11, 12, 14, 20-22, 26, 28, 30, and 32, wherein any change from the disclosed sequence are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Such animals can be non-human animals, non-human mammals, or non-human primates.

Also disclosed are animals produced by the process of adding to the animal any of the cells disclosed herein.

### METHODS

Methods of using the Compositions

Disclosed herein are methods of expressing ACF in a cell, comprising bringing into contact a cell and a vector comprising a nucleic acid, wherein the nucleic acid encodes a polypeptide comprising an ACF sequence, a secretion sequence and a transduction sequence;

whereby the nucleic acid produces the polypeptide, thereby expressing ACF in the cell. Also disclosed herein are methods comprising administering to a cell a polypeptide comprising an ACF sequence and a transduction sequence, wherein the ACF sequence comprises the amino acid sequence SEQ ID NO: 17 having one or more mutations at one or more sites where ACF is phosphorylated, the amino acid sequence SEQ ID NO: 1 having one or more mutations at one or more sites where ACF is phosphorylated, the amino acid sequence SEQ ID NO: 18 having one or more mutations at one or more sites where ACF is phosphorylated, or the amino acid sequence SEQ ID NO: 19 having one or more mutations at one or more sites where ACF is phosphorylated.

Also disclosed herein are methods of expressing ACF in a cell, comprising bringing into contact a cell and a vector comprising a nucleic acid, wherein the nucleic acid encodes a polypeptide comprising an ACF sequence; whereby the nucleic acid produces the polypeptide, thereby expressing ACF in the cell, wherein the ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated. Expression of the polypeptide in the cell can increase transport of apoB mRNA from the nucleus to the cytoplasm of the cell. Expression of ApoB protein in the cytoplasm of the cell can also be increased. Increased expression of ApoB protein can lead to a reduction in the level of lipid in the cell.

Also disclosed herein are methods of expressing ACF in a cell, comprising bringing into contact a cell and a vector comprising a nucleic acid, wherein the nucleic acid encodes a polypeptide comprising an ACF sequence; whereby the nucleic acid produces the polypeptide, thereby expressing ACF in the cell, wherein the ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated. Expression of the polypeptide in the cell can increase transport of apoB mRNA from the nucleus to the cytoplasm of the cell. Expression of ApoB protein in the cytoplasm of the cell can also be increased. Increased expression of ApoB protein can lead to a reduction in the level of lipid in the cell.

In the methods above, the cell and the vector can be brought into contact in vivo or in vitro, such as in culture. The cell can be a liver cell, and the cell can be in a subject. The cell can also be any type of cell where ACF deficiency is indicated for RNAs other than apoB during embryonic development. There are various ACF nucleic acids their corresponding polypeptides that are useful with the methods disclosed herein. These nucleic acids and their variants are discussed above.

Delivery of the polypeptide to the second cell can increase transport of apoB mRNA from the nucleus to the cytoplasm of the second cell. Also, expression of ApoB protein in the cytoplasm of the second cell can be increased. Increased expression of ApoB protein can also lead to a reduction in the level of lipid in the second cell. In one example, the second cell can be a liver cell, and the cell can be in a subject.

The following diseases and disorders can be treated by the methods disclosed herein, and can be treated individually or can be treated simultaneously, meaning that one or more of them can be treated at the same time: nonalcoholic fatty liver disease (steatohepatitis), alcoholic fatty liver disease (alcoholic hepatic steatosis), viral induced steatohepatitis (hepatitis B infection), liver cirosis chronic hyperinsulinemia, type II diabetes, or obesity. The methods disclosed herein can also be used to treat birth defects due to low or no ACF or due to inappropriate regulation of ACF phosphorylation and its nuclear trafficking.

As discussed above, the methods disclosed herein can also be used in conjunction with APOBEC-1 to reduce the risk of atherogenesis. Dietary cholesterol is carried into blood from the intestine by a specific carrier protein called apolipoprotein B48 and from liver into the circulation for distribution to other tissues as apolipoprotein B100 or apoB100 and apolipoprotein B48 depending on the species. Apolipoprotein B is an integral and non-exchangeable structural component of lipoprotein particles referred to as chylomicrons, very low density lipoprotein (“VLDL”), and low density lipoprotein (“LDL”). Apolipoprotein B circulates in human plasma as two isoforms, apolipoprotein B100 and apolipoprotein B48. Apolipoprotein B48 is generated by an RNA editing mechanism which changes codon 2153 (CAA) to a translation stop codon (UAA) (Chen et al., Science 238:363-366 (1987); Powell et al., Cell 50:831-840 (1987)). Editing is a site-specific deamination event catalyzed by apolipoprotein B mRNA editing catalytic subunit 1 (known as APOBEC-1) (Teng et al., Science 260:18116-1819 (1993)) with the help of auxiliary factors (Teng et al., Science 260:18116-1819 (1993); Yang et al., J. Biol. Chem. 272:27700-27706 (1997); Yang et al., Proc. Natl. Acad. Sci. USA 94:13075-13080 (1997); Lellek et al., J. Biol. Chem. 275:19848-19856 (2000); Mehta et al., Mol. Cell. Biol. 20:1846-1854 (2000); Yang et al., J. Biol. Chem. 275:22663-22669 (2000); Blanc et al., J. Biol. Chem. 276:10272-10283 (2001)) as a holoenzyme or editosome (Smith et al. Proc. Natl. Acad. Sci. USA 88:1489-1493 (1991); Harris et al., J. Biol. Chem. 268:7382-7392 (1993)). Apolipoprotein B100 and apolipoprotein B48 play different roles in lipid metabolism, most importantly, apolipoprotein B100-associated lipoproteins (VLDL and LDL) are much more atherogenic than apolipoprotein B48-associated lipoproteins (chylomicrons and their remnants and VLDL).

Specifically, the apolipoprotein B48-associated lipoproteins are cleared from serum more rapidly than the apolipoprotein B100-associated lipoproteins. As a result, apolipoprotein B48-VLDL usually are not present in serum for an amount of time sufficient for serum lipases to convert the VLDL to LDL. In contrast, the apolipoprotein B100-VLDL are present in the serum for sufficient amounts of time, allowing serum lipases to convert the VLDL to LDL. Elevated serum levels of LDL are of particular biomedical significance as they are associated with an increased risk of atherogenic diseases or disorders. Lipoprotein analyses have shown that the ability of mammalian liver to edit results in a lowering of the VLDL+LDL:HDL ratio. Therefore modifying apolipoprotein B editing which favors an increase in the relative concentration of apolipoprotein B48 in proportion to apolipoprotein B100 (or total apolipoprotein concentration), thereby clearing a greater concentration of lipoproteins from serum and minimizing the atherogenic risks associated with high serum levels of VLDL and LDL is desirable.

Therefore, the methods and compositions disclosed herein can further comprise a second nucleic acid, wherein the second nucleic acid encodes a second polypeptide, wherein the second polypeptide comprises an APOBEC-1 sequence, a second secretion sequence and a second transduction sequence; whereby the nucleic acid produces the second polypeptide, thereby expressing APOBEC-1 in the cell. Also disclosed are bringing into contact the cell and a second vector, wherein the second vector comprises a second nucleic acid, wherein the second nucleic acid encodes a second polypeptide, wherein the second polypeptide comprises an APOBEC-1 sequence, a second secretion sequence and a second transduction sequence; whereby the nucleic acid produces the second polypeptide, thereby expressing APOBEC-1 in the cell.

The second polypeptide comprising APOBEC-1 can also be secreted from the cell, and the second polypeptide can also transduce a second cell, thereby delivering the second polypeptide to the second cell. The APOBEC-1 sequence can comprise the amino acid sequence SEQ ID NO: 8, an amino acid sequence at least about 90% identical to the amino acid sequence of SEQ ID NO: 8, or the amino acid sequence SEQ ID NO: 8 having one or more conservative amino acid substitutions, as discussed above.

The second transduction sequence comprising APOBEC-1 can also comprise a TAT sequence, for example, SEQ ID NO: 23. The second secretion sequence can be an albumin signal sequence, such as that comprising SEQ ID NO: 24. The second secretion sequence can selected from a signal sequence in the SIGPEP database (von Heijne, G. Protein Seq Data Anal. 1987; 1(1):41-2). The APOBEC-1 sequence, second secretion sequence, and second transduction sequence can be operably linked. For example, the APOBEC-1 sequence, second secretion sequence, and second transduction sequence can be contiguous.

### Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell can be transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein. Homologous recombination in mammalian cells may require the cells to be cultured, because the desired recombination event occurs at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### Methods of Screening

Disclosed herein are methods of screening for a compound that modulates phosphorylation of ACF, comprising contacting a cell expressing ACF with a test compound, detecting the level of phosphorylated ACF using ACF phosphorylation site-specific antibodies, wherein a change in the level of phosphorylated ACF compared to the level of phosphorylated ACF in a control cell expressing ACF not exposed to the test compound indicates that the test compound is a compound that modulates phosphorylation of ACF. The test compound can a phosphatase inhibitor, for example. A plurality of test compounds can be contacted with ACF in a high throughput cell-based assay system. The high throughput assay system can comprise an immobilized array of cells, for example, such as hepatocytes. Also, the level of phosphorylated ACF in the cells expressing ACF contacted with the test compound can be decreased compared to the level of phosphorylated ACF in the control cells, thereby identifying the test compound as a compound that decreases the level of phosphorylated ACF.

Also disclosed herein are methods of screening for a compound that increases expression of ACF, comprising: contacting a cell with a test compound, detecting the level of ACF expression in the cell, wherein an increased level of ACF expression compared to the level of ACF expression in a control cell not exposed to the test compound indicates that the test compound is a compound that increases expression of ACF. The cell can be a hepatocyte, for example. The level of ACF expression in the cell can be detected by detecting the level of ACF in the cell. Also, the cell can comprise a nucleic acid sequence comprising ACF expression control sequences operably linked to a sequence encoding a marker, wherein the level of ACF expression in the cell is detected by detecting the level of the marker. Also disclosed is

Also disclosed are methods of producing the compound identified in the screening methods above as modulating phosphorylation of ACF. Also disclosed are compounds identified by these methods.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Metabolic Regulation of ApoB mRNA Editing is Associated with Phosphorylation of APOBEC-1 Complementation Factor

It has been demonstrated that ACF was phosphorylated on one or more serine residues, and that ethanol and insulin induction of cipoB mRNA editing was accompanied by phosphorylation of ACF. PhosphoACF was only detected in the nucleus, and was selectively recovered with active 27S editosomes. Although ACF and APOBEC-1 are both present in the cytoplasm, APOBEC-1 co-immunoprecipitated with ACF only from nuclear extracts. Recovery of ACF/APOBEC-1 complexes and apoB mRNA editing activities were dependent on protein phosphorylation. Protein phosphatase inhibitor studies show that protein phosphatase I is involved in regulating editing activity, ACF phosphorylation, and ACF subcellular distribution. The significance of ACF phosphorylation for ACF trafficking to the nucleus, association with APOBEC-1 and assembly into 27S editosomes and the regulation of editing efficiency are discussed below.

Hepatic apoB mRNA editing is a regulated, nuclear process that requires the assembly of multi-protein editosomes. Reconstitution assays have identified the essential protein factors as the cytidine deaminase APOBEC-1 and the auxiliary factor ACF. It is herein shown that ACF is a phosphoprotein. ACF was phosphorylated on one or more serine residues under basal media conditions and the proportion of total cellular ACF that became phosphorylated increased upon ethanol or insulin treatment, stimuli both known to enhance editing activity. This shows that ACF phosphorylation is metabolically regulated and a part of the mechanism for activating apoB mRNA editing regardless of whether APOBEC-1 expression is increased (insulin treatment, (von Wronski et al. (1998), Phung et al. (1996)) or not (ethanol treatment, (Lau et al. (1995)).

Endogenous hepatic or intestinal cell editing activity is highly efficient. In fact, editing activity in vivo is regulated in a species- and tissue-specific manner, and inducible during development and in response to metabolic and hormonal perturbations (Chen et al. (1987), Powell et al. (1987)) (and reviewed in (Smith et al. (1997)). In this context, the data showed that phosphorylation of ACF optimized and/or stabilized the functional interaction with ACF in the nucleus leading to efficient editing activity. This mechanism explained how apoB mRNA editing activity can be activated metabolically or during development using pre-existing editing factors.

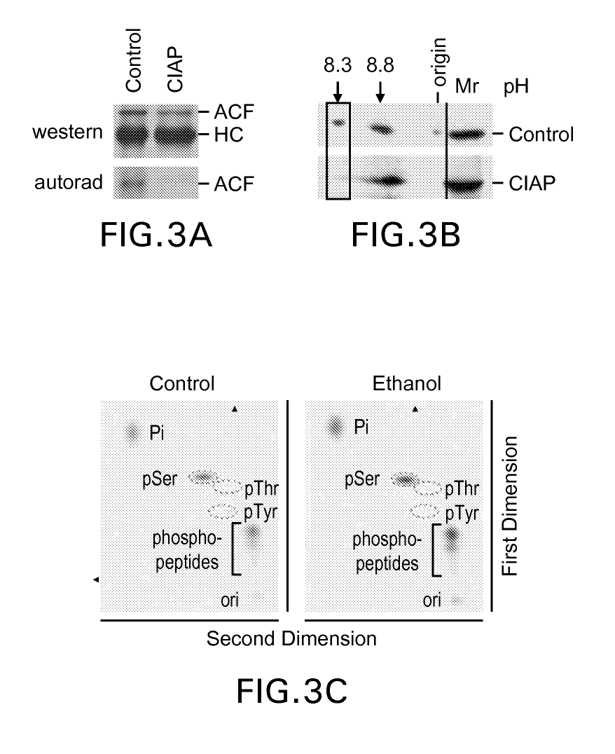

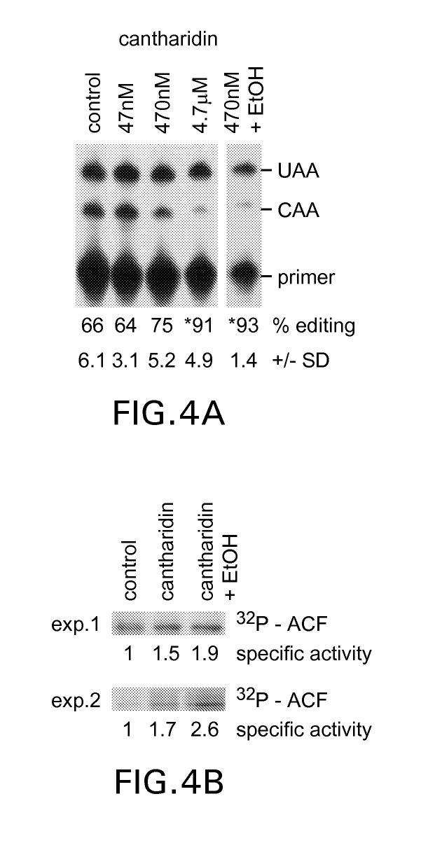

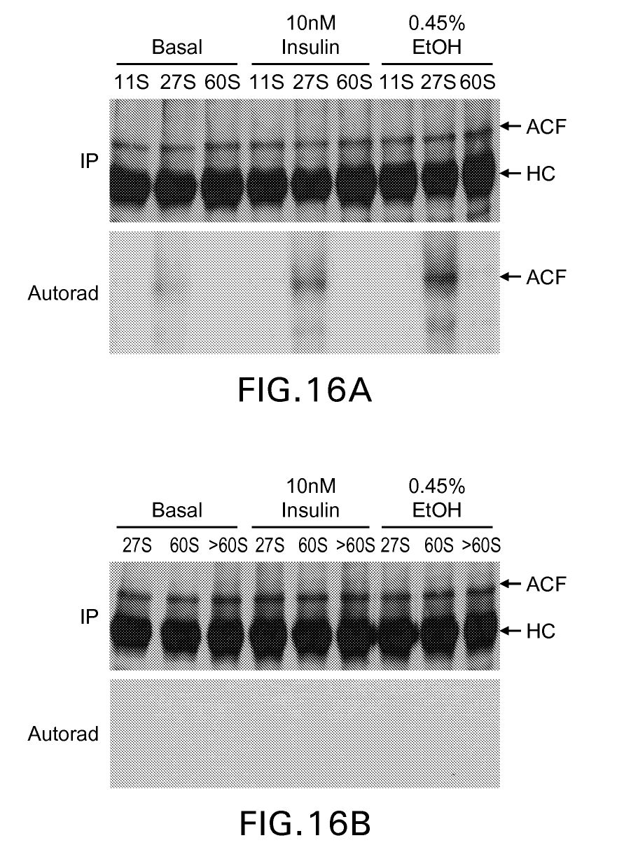

Under basal conditions, inhibition of PP 1 activity resulted in the nuclear retention and increased recovery of phosphoACF and increased apoB mRNA editing activity, indicating that ACF phosphorylation/dephosphorylation contributes to the modulation of editosome assembly and editing activity. Given that not all nuclear ACF is phosphorylated (FIGS. 3B and 4) and that not all ACF is assembled in 27S editosomes (FIG. 16 and Smith et al (1991) and Sowden et al. (2002)) the data show that at any given time not all of the cellular ACF is involved in editing. This shows that there is a pool of ACF that can be used to rapidly modulate editosome assembly upon metabolic or hormonal stimuli or that ACF has additional roles in the cell.

In addition to editosome structure and function, ACF plays an important role in the cellular regulation of apoB mRNA editing through its trafficking activity between the cytoplasm and the nucleus (Sowden et al. (2002), Yang and Smith (1997), Chester et al. (2003), Yang et al. (2001), Blanc et al. J Biol Chem 278:41198-204 (2003)). Although the site of apoB mRNA editing is within the cell nucleus (Lau et al. (1991), Yang (2000)) and takes place during or immediately after pre-mRNA splicing (Lau et al. (1991), Sowden and Smith (2001), Sowden et al. (1996)), APOBEC-1 and ACF are distributed in both the nucleus and cytoplasm (Sowden et al. (2004), Sowden et al. (2002), Chester et al. (2003), Yang et al. (2001), Blanc (2003)). The data shows that nuclear retention/import of ACF was increased in ethanol or insulin treated hepatocytes through ACF phosphorylation. Data also show that APOBEC-1 has strong cytoplasmic retention signals, and that its nuclear import is mediated by interactions with ACF (Yang and Smith (1997), Yang et al. Exp Cell Res 267:153-164 (2001)). ACF and APOBEC-1 are present in both the cytoplasm and nucleus of editing competent cells, but that they only co-immunopurify from nuclear extracts. The data show that nuclear retention/import of ACF is increased in ethanol or insulin treated hepatocytes through modulation of ACF phosphorylation state. This shows that phosphorylation of ACF results in its nuclear accumulation and enhances or stabilizes APOBEC-1 nuclear retention and ACF binding, leading to increased editing activity. In support of this, phospatase treatment of cell extracts was associated with reduced co-immunoprecipitation of APOBEC-1 with ACF and reduced editing activity.

A small proportion of nuclear ACF was phosphorylated in non-stimulated hepatocytes (0.1 nM insulin) (FIGS. 3B and 3C) which increased several-fold upon insulin and ethanol stimulation (FIGS. 3B and 3C). These data showed that a low level turnover of ACF phosphorylation can be required to maintain basal apoB mRNA editing activity. Significantly, protein phosphatase I inhibition stimulated editing activity even under basal media conditions. The turnover of editing complexes has been shown from studies that demonstrated nucleocytoplasmic shuttling of ACF and APOBEC-1 (Blanc et al. (2003), Chester et al. (2003), Yang et al. (1997), Yang et al. (2001)) and from in vitro studies of editosome assembly (Smith et al. (1991)). Addition of ethanol (or its catabolite, acetaldehyde) or chemicals affecting kinases and phosphatase to nuclear extracts did not affect in vitro editing activity or ACF phosphorylation. These data indicate that intact cell signal transduction cascades are required for the regulation of ACF phosphorylation and apoB mRNA editing. The identification of protein phosphatase I as a candidate phosphatase involved in regulating phosphate turnover on ACF relevant, as high levels of PP1 are present in rat hepatocyte nuclei (Kuret et al. FEBS Lett 203:197-202 (1986)) of which 90% was associated with chromatin (Beullens et al. J Biol Chem 267:16538-44 (1992)). Nuclear ACF is also associated with chromatin (Sowden et al. (2002)) placing it within the general domain of nuclear PP1.

The lack of labeled ACF in the cytoplasm also shows that ACF is dephosphorylated prior to or during nuclear export. These data show that if phosphorylation of ACF is restricted to the nucleus and associated with enhanced editing activity, then dephosphorylation of ACF can regulate its nuclear export. Given that ACF binds to both unedited and edited apoB mRNA (Harris et al. (1993)) and that dephosphorylated ACF binds to apoB RNA. ACF can remain bound to apoB mRNA and co-export to the cytoplasm following apoB mRNA editing and ACF dephosphorylation. Regulation of ACF dephosphorylation modulates apoB mRNA export to the cytoplasm in addition to protecting edited apoB mRNA from NMD) (Chester et al. (2004)). Under basal conditions, 2D gel electrophoresis analyses showed that ACF contained 2-3 phosphates. 2D phosphoamino acid analyses indicated serines were the residues phosphorylated following metabolic stimulation.

Regulation of hepatic apoB mRNA editing by ethanol and insulin promotes serine phosphorylation of ACF and its localization to active nuclear 27S editosomes. The data shows a role for the metabolic regulation of ACF phosphorylation that promotes its interaction with APOBEC-1, in editosome assembly, as well as ACF nuclear retention/import. Insulin stimulated hepatic apoB mRNA editing activity through an induction of APOBEC-1 expression as well as an enhanced nuclear accumulation of ACF (Sowden et al. (2004), Sowden et al. (2002)). Ethanol also stimulated hepatic apoB mRNA editing and ACF nuclear accumulation (Harris et al. (1993), Sowden et al. (2002), Yang et al. (2000)), but did not induce APOBEC-1 expression (Funahashi et al. (1995), Lau et al. (1995). In fact, ethanol stimulated apoB mRNA editing occurred selectively on nuclear mRNA (Yang et al. (2000)) and in the absence of de novo protein synthesis (Giangreco et al. (2001)) showing that post-translational modification of pre-existing editing factors can lead to their activation. Moreover, thyroid hormone administration to mice resulted in a marked redistribution of ACF to the nucleus and a concomitant increase in apoB mRNA editing (Mukhopadhyay et al. (2003)).

Materials and Methods

Animal care, primary hepatocyte isolation and hepatoma cell culture. Male Sprague-Dawley rats (275-325 g BW/Charles River Laboratories, Wilmington, Mass.) were housed under 12 hour light/dark cycles and fed normal rat chow (Purina, St. Louis, Mo.) ad libitum and euthanized between 9 and 10 AM. Primary hepatocytes were isolated (Van Mater et al. (1998)) and plated onto BIOCOAT type I collagen coated dishes (Becton Dickinson Labware, Franklin Lakes, N.J.) in Waymouth's 752/1 media (Sigma Chemical Co., St. Louis, Mo.) containing 0.1 nM porcine insulin (Sigma) for 12-16 hours prior to the onset of each experiment.

McArdle RH7777 cells (ATCC Manassas, Va.) stably expressing HA epitope-tagged APOBEC-1 (Yang et al. J Biol Chem 272:27700-6 (1997)) were treated for 4 hours with 0.9% ethanol and fractionated into nuclear extracts (Van Mater et al. (1998)).

In vivo phosphorylation of ACF. In vivo ³²P labeling was performed by intraperitoneal injection of rats with 12.5 mCi of orthophosphoric acid (10 mCi/ml ³²PO₄; NEN, Boston, Mass.) buffered with 50 mM HEPES, pH 7.0 and 150 mM NaCl. After 4 hours, rats were sacrificed and hepatic cytoplasmic and nuclear extracts prepared. Primary hepatocyte cultures in 60 mm dishes were incubated in phosphate-free Minimum Essential Eagle Media (Sigma) containing 0.1 nM porcine insulin for 6 hours and subsequently in fresh media containing 10 nM insulin (equivalent to that seen in post-prandial serum) or 0.45% to 0.9% ethanol (AAPER Alcohol and Chemical Co., Shelbyville, Ky.) (Van Mater et al. (1998), Yang et al. (2000)) plus 2.0 mCi orthophosphoric acid. Cultures were labeled for 4 hours prior to extract preparation.

Subcellular extract preparations. Livers were perfused in situ with 0.25 M sucrose, 50 mM Tris pH 7.0 and 5 mM MgCl₂ and protease inhibitors (Roche) (Harris et al. (1993), Sowden et al. (2002)). Cytoplasmic and nuclear extracts (Smith, H. C. Methods 15:27-39 (1998)) were supplemented with 10 mM NaF and fractionated through glycerol gradients.

Cultured primary hepatocytes and hepatoma cell lines were rinsed with 1× Tris-buffered saline (TBS; 10 mM Tris, 150 mM NaCl, pH 7.5) and scraped into TBS containing proteinase/phosphatase inhibitors (Roche). Nuclear and cytoplasmic extracts were prepared using the NE-PER kit (Pierce, Rockford, Ill.) supplemented with 10 mM NaF and proteinase inhibitors.

Glycerol gradient fractionation. Preparative (35 ml) 10-50% glycerol gradients containing 10 mM NaF were loaded with 20 mg or 2 mg of cytoplasmic or nuclear S100 extract respectively and sedimented at 100,000×g for 10 hours at 7° C. The distribution of 11S, 27S, and 60S complexes (fractions 3/4, 5/6, and 7/8) was determined by the sedimentation of bovine serum albumin (6S), catalase (11S) and 60S spliceosomes (Harris et al. (1993), Sowden et al. (2002)). Gradient fractions were treated with DNaseI and RNaseT1 to ensure the removal of DNA or RNA prior to the analysis of ACF ³²P labeling.

In vitro editing reactions and RNA binding assays. In vitro editing reactions and quantitative poisoned-primer extension assays were carried out using 80 μg of extract proteins as previously described (Shah et al. (1993), Smith, H. C. (1998)). ApoB RNA binding assays were performed by subjecting nuclear extract (80 μg) to in vitro editosome assembly and ultraviolet crosslinking (Smith, H. C. (1998)).

Protein phosphatase inhibitor studies. Primary hepatocytes were treated for 6 hours with cantharidin, endothall, or okadaic acid at concentrations encompassing their respective in viva IC₅₀ values as described by the manufacturer (Calbiochem, La Jolla, Calif.) and their suggested references. The compounds' effects on apoB mRNA editing were evaluated by RT-PCR and poisoned-primer extension analysis (Sowden et al. Rna 2:274-88 (1996)). The effect of 470 nM cantharidin on ACF subcellular distribution was investigated by treating primary hepatocyte cultures for 6 hours followed by nuclear and cytoplasmic extract preparation.

To evaluate the effect of phosphatase inhibition on ACF phosphorylation, cultures were pre-incubated for 2 hours with 470 nM cantharadin in phosphate-free Waymouth's media and subsequently supplemented with 0.5 mCi ³²pO₄ and incubated for an additional 4 hours. Cultures were harvested and subcellular extracts prepared.

Immunological techniques. Rabbit polyclonal peptide-specific antibodies were raised against ACF amino-terminal (NT) sequence (NHKSGDGLSGTQKE, SEQ ID NO: 34) and carboxy-terminal (CT) sequence (HTLQTLGIPTEGGD, SEQ ID NO: 35) (Sowden et al. (2002)), and affinity purified with the corresponding peptides (Bethyl Laboratories, Inc., TX). For immunoprecipitation analyses all radiolabeled extracts were adjusted to 5 mM MgCl₂ and digested with 100 units of DNase I (Promega), RNase T1 (Roche, Ind.), and RNase A (Sigma) for 1 hour on ice to remove radiolabeled nucleic acid. Where applicable, extracts were incubated overnight with ACF CT antibody at 4° C. and subsequently reacted with Protein A-agarose (Oncogene Research Products, Boston, Mass.) pre-washed with 1×TBS/10 mM NaF. The immuno-absorbed material was washed 3 times with 1×TBS/10 mM NaF, 3 times with 1×TBS/1 M NaCl (Yang et al. (1997)) and lastly 3 times with 1×TBS. Immuno-absorbed material to be treated with alkaline phosphatase was washed with 50 mM Tris, pH 8.4, 1 mM MgCl₂, 0.1 mM ZnCl₂, 25% glycerol and then incubated with 5 U CIAP for 1 hour at 30° C. and then washed 6 times with 1×TBS. ACF-antibody complexes were eluted with 3 M sodium thiocyanate, acetone precipitated and analyzed by 10.5% SDS-PAGE followed by autoradiography and/or western blotting with ACF NT antibody. For ACF immunoprecipitation from gradient fractions an equal volume of pooled gradient fractions from sedimentation zones of interest were reacted with sub-saturating amounts of ACF CT antibody to ensure that the recovery of phospho-ACF was not simply a reflection of ACF abundance in each zone.

Two-dimensional (2D) gel electrophoresis. Extracts isolated from hepatocytes were analyzed for ACF charge isoforms using the Protean isoelectric focusing (IEF) system (Bio-Rad Laboratories, Hercules, Calif.). Immobilized pH gradient (IPG) strips (Bio-Rad Laboratories, pH range 3-9.3) were hydrated with 150 μg nuclear or 340 μg cytoplasmic extract in 2D loading buffer (7.5 M urea, 1.0 M thiourea, 1% CHAPS, 58 mM DTT, 0.2% biolytes) and electrophoresed to equilibrium.

After completion of IEF the IPG strip was equilibrated in SDS buffer for 30 minutes and then in iodoacetamide buffer for an additional 30 minutes according to the manufacturer's recommendations. Proteins were resolved through a 10.5% Criterion gel (Bio-Rad Laboratories), transferred to nitrocellulose and reacted with ACF NT antibody.

Two-dimensional phosphoamino acid analysis. ACF was immunopurified using the ACF CT antibody from 27S enriched nuclear extracts of ³²P-labeled primary hepatocytes cultured in basal media (0.1 nM insulin) or in basal media containing 0.9% ethanol. Immunoprecipitates were resolved by SDS-PAGE, blotted onto PVDF membrane (BioRad, CA) and ACF was identified by autoradiography, excised and acid hydrolyzed in 5.7 N HCl at 110° C. for 1 hour (Blume-Jensen, P. and Hunter, T. Methods Mol Biol 124:49-65 (2001)). Lyophilized hydrolysates were spiked with unlabelled phosphoserine, phosphothreonine and phosphotyrosine (Sigma) and resolved on thin layer chromatography plates (Merck, Germany) by 2D electrophoresis using an HTLE 7000 peptide mapping system (CBS Scientific Co. Del Mar, Calif.) Blume-Jensen, P. and Hunter, T. Methods Mol Biol 124:49-65 (2001)). The migration of the unlabelled standards and ACF radiolabeled amino acid(s) were visualized by ninhydrin staining and phosphorimager scanning densitometry, respectively.

RATS: Livers of male Sprague-Dawley rats (250-275 g body weight) have provided the most consistently high yield of hepatocytes (Sowden et al. (2002), Giangreco et al. (2001) which are isolated once every 2 weeks on average and therefore 1-2 animals per month have been budgeted (total of 24 rats/yr). Consumption is anticipated to be highest after the first half of the proposed five years of funding. Rats are anesthetized with sodium pentobarbital (12 mg/kg) and attached to a recirculation system entering the animal via the hepatic portal vein and leaving via thoracic vena cava. Livers are perfused in situ with oxygenated buffer for 3 to 5 minutes to remove red blood cells. At this point the animal are terminated by cutting open the thorax. Buffered collagenase is perfused and recirculation initiated and continued until the lobes of the liver show signs of digestion, typically 10 to 15 minutes. The liver is removed to a Petri dish and the cells rinsed free from the liver capsule in sterile buffer washed three times in sterile buffer by gentle centrifugation (800×g, 3 min), viable hepatocytes counted by trypan blue exclusion and plated on rat tail collagen (Type I)-coated plastic 60 mm dishes (Beckton Dickinson) in two mls of Waymouth media containing 0.2% bovine serum albumin (Sigma) and 0.1 nM porcine insulin (Sigma) at a cell density of 1−2×10⁶ cells/ml.

APOBEC-1 KNOCKOUT MICE: A breeding pair of apobec1−/− mice (C57BL/6 background) [Nassir et al., (2004) Hepatic secretion of small lipoprotein particles in apobec-1−/− mice is regulated by the LDL receptor. J. Lipid Res. 45:1649-59] are used. Livers from adult male mice on normal diets or subjected to the fasting and refeeding regime are used for extract preparation. Alternatively male mice are used to prepare primary hepatocytes as described above. Primary hepatocytes are prepared once every 2 weeks on average and therefore 1-2 animals per month have been budgeted (total of 24 mice/yr). Livers from mice under the metabolic model are harvested from 9 mice once every 3 months (36 mice/yr).

Results

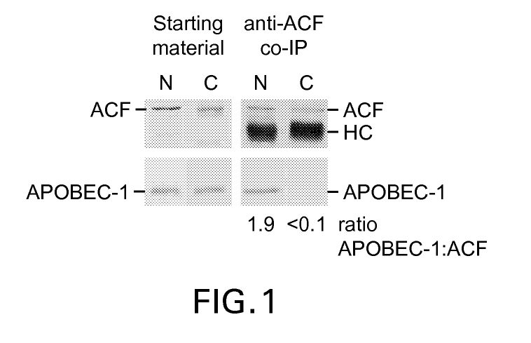

The minimal, functional editosome is composed of ACF, a homodimer of APOBEC-1 and the apoB RNA substrate (Mehta A. and Driscoll, D. M. RNA 8:69-82 (2002), Blanc et al. J Biol Chem 276:46386-93 (2001)). ACF and APOBEC-1 are distributed in the cytoplasm and nucleus of editing competent cells where they co-localize in macromolecular complexes of 60S and 27S, respectively (Harris et al. (1993), Sowden et al. (2002)). However, apoB mRNA editing is only associated with nuclear 27S complexes (Sowden et al. (2002), Yang et al. (2000)). The interaction of ACF with APOBEC-1 in the nucleus and cytoplasm was analyzed by co-immunoprecipitation from extracts prepared from McArdle cells that stably express HA-tagged APOBEC-1 (Yang and Smith (1997), Yang et al. (1997)). As anticipated, ACF and APOBEC-1 were abundant in both cytoplasmic and nuclear starting material (FIG. 1). However, APOBEC-1 was efficiently co-immunoprecipitated with ACF only from nuclear extracts.

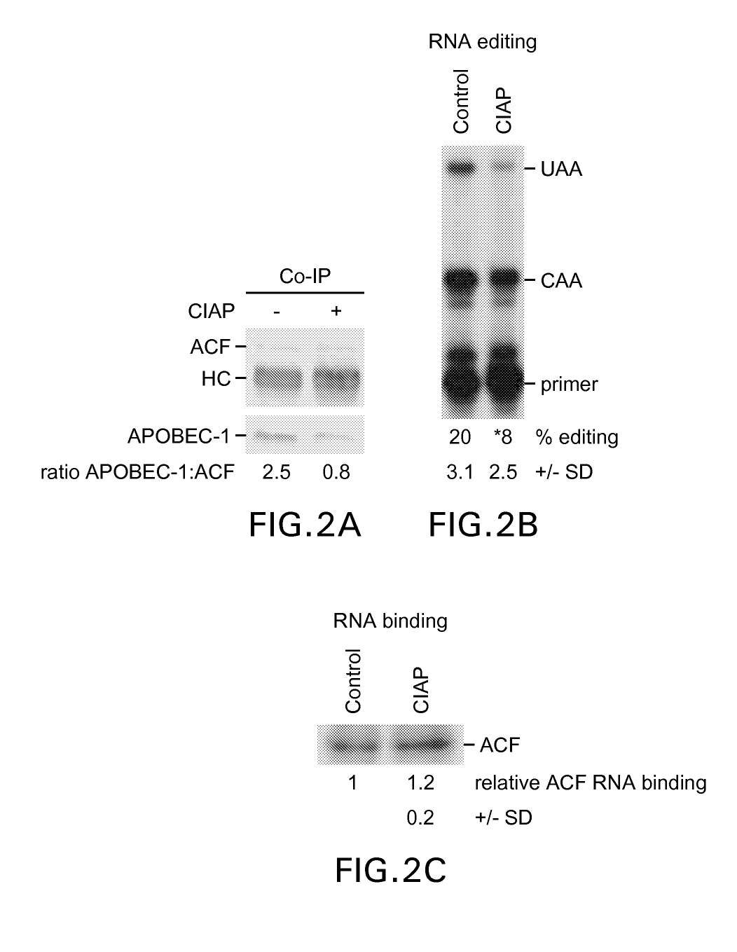

Given the selective recovery of APOBEC-1 with nuclear ACF (FIG. 1), the interaction between ACF and APOBEC-1 was explored in nuclear extracts was mediated by post-translational modifications such as protein phosphorylation. Treatment of nuclear extracts with alkaline phosphatase (IOU CIAP) resulted in a 3-fold reduction in HA-tagged APOBEC-1 recovered with immunoprecipitated ACF (FIG. 2A).

As an interaction between ACF and APOBEC-1 is critical for editing activity (Metha et al. Mol Cell Biol 20:1846-54 (2000)) the effect of alkaline phosphatase treatment on in vitro editing activity of hepatocyte extracts was investigated. ApoB mRNA editing activity in CIAP-treated nuclear extract was significantly inhibited 2.5-fold relative to control extracts (FIG. 2B). In contrast to the interaction with APOBEC-1, CIAP treatment (1, 5 or IOU) did not significantly increase ultraviolet light cross-linking of ACF to apoB mRNA (FIG. 2C). These data show that the reduction in editing activity in CIAP-treated extracts was the result of suppression of interactions between ACF and APOBEC-1 rather than an alteration in the binding affinity of ACF to apoB mRNA.

ACF is a phosphoprotein. Previous studies using site-directed mutagenesis of APOBEC-1 and overexpression of protein kinase Co implicated phosphorylation of APOBEC-1 as a mechanism for activating apoB mRNA editing (Chen et al. (2001)). To evaluate whether endogenous ACF is phosphorylated in vivo, rats were radiolabeled for 4 hours via an intraperitoneal injection of orthophosphoric acid in HEPES-buffered saline. Following extensive digestion of hepatic nuclear extracts with DNase I and RNase T1 to remove radiolabeled nucleic acids, ACF was immunoprecipitated with the CT antibody, resolved by SDS-PAGE, transferred to nitrocellulose and analyzed by autoradiography as well as immunoblotting with ACF NT antibody (FIG. 3A). A single band was detected by autoradiography that super-imposed with ACF-specific immunoblot reactivity. Greater than 90% of the ³²P-label was removed by CIAP treatment (FIG. 3B), consistent with the post-translational phosphorylation of protein. Cytoplasmic ACF did not become radiolabeled (FIG. 16). The amino acid sequence of rat ACF was predicted contain 23 serine/threonine and 7 tyrosine high probability sites of phosphorylation (http://www.cbs.dtu.dk/services.NetPhos and http://expasy.org/tools/scanprosite). To investigate the complexity of physiologically relevant ACF phosphorylation sites, liver extracts prepared from control rats were resolved by equilibrium 2D gel electrophoresis and immunoblotted with ACF NT antibody (Sowden et al. 2002)). The predicted isoelectric point (pI) of ACF is 8.8 (http://www.scripps.edu/cdputnam/protcalc.html) and the covalent addition of phosphate was expected to cause an acidic shift of the pI (O'Farrell, P. H. J Biol Chem 250:4007-21 (1975)). Nuclear ACF was detected as two predominant charge isoforms (FIG. 3B). The major isoform migrated at pI and 8.8 and is likely unmodified ACF or ACF containing both acidic and basic modifications. A second, less abundant, isoform migrated with a pI of 8.3 and is consistent with phosphorylation of ACF. To verify that the observed charge heterogeneity was due to protein phosphorylation, nuclear extracts were treated with CIAP which resulted in an almost complete loss of the acidic isoform (pI 8.3) and concomitant increase in the pI 8.8 isoform, which is consistent with the removal of 2-3 phosphates (0.2-0.3 pH units per phosphate) (O'Farrell, P. H. (1975)). Cytoplasmic ACF migrated as a single isoform at pT 8.8, which did not shift upon phosphatase treatment, further confirming that cytoplasmic ACF was not phosphorylated under these assay conditions.

To determine whether serine, threonine and/or tyrosine residues were the target of phosphorylation, primary hepatocytes were labeled to high specific activity with ³²PO₄ and ACF was immunoprecipitated from nuclear extracts. Two-dimensional thin layer electrophoresis of acid hydrolyzed ACF demonstrated that phosphorylation occurred on serine residues (FIG. 3C, left panel). The amount of phosphoserine detected increased (1.5-fold) in ACF immunopurified from an equivalent amount of nuclear extract isolated from primary hepatocytes incubated with 0.9% ethanol during the labeling period (FIG. 3C, right panel). The 1.5-fold change in phosphoserine abundance can represent a minimum as the radioactivity in partially hydrolyzed peptides (migrating along the first dimension) as the pSer spot shows greater ³²P incorporation (see Materials and Methods). These data show that threonine and tyrosine residues are not phosphorylated in rat ACF or that their phosphorylation exhibits a slow rate of turnover, preventing them from incorporating ³²P label during the experiment.

Phosphorylated ACF is only recovered with nuclear editosomes. If phosphorylated ACF is relevant to editing activity, it should be associated with nuclear 27S editosomes. To evaluate this, cytoplasmic and nuclear extracts from radiolabeled primary hepatocyte cultures were sedimented through 10%-50% glycerol gradients. ACF was immunoprecipitated with subsaturating quantities of the ACF CT antibody from pooled fractions corresponding to: (i) 11S (pre-editosomal ACF and APOBEC-1), (ii) the 27S editosome and (iii) 60S and greater. The ACF immunoprecipitates were resolved by SDS-PAGE, transferred to nitrocellulose, autoradiographed and subjected to phosphorimager scanning to detect and quantify radiolabeled proteins prior to immunoblotting with the ACF NT antibody. Consistent with prior analyses (Sowden et al. (2002)), ACF was recovered by immunopurification from all gradient fractions (FIG. 16). Although ACF was widely distributed in all nuclear fractions, phosphoACF was restricted to fractions containing the 27S editosome. Editing activity, indicative of the assembly of APOBEC-1 with ACF has only been observed in 27S gradient fractions (Harris et al. (1993)). Considering data presented in FIGS. 1 & 2A/B, the selective recovery of phosphorylated ACF in the nuclear 27S editosome fraction is highly shows this. Thus, a correlation linking phosphoACF to the physiologically relevant 27S editing complexes can be made.

Phosphorylation of ACF is metabolically regulated. ApoB mRNA editing is regulated by a variety of hormonal and dietary factors (Wedekind et al. Trends Genet. 19:207-16 (2003)). To determine if ACF phosphorylation is correlated with metabolic regulation of editing, rat primary hepatocytes were labeled with ³²PO₄ for 4 hours in the presence of either 0.45% ethanol or 10 nM insulin. A 3.5-fold increase of phosphoACF was associated with nuclear 27S editosomes isolated from ethanol treated hepatocytes. These data are consistent with the increase in serine phosphorylation observed following ethanol treatment (FIG. 3C). Moreover, phosphoACF was not detected in any cytoplasmic fractions despite a 10-fold higher protein load compared to nuclear extracts onto the gradients and a prolonged autoradiographic exposure (FIG. 16).

To determine if enhanced ACF phosphorylation is a more general mechanism associated with editing induction, the effect of insulin treatment on primary hepatocytes was investigated. Insulin, like ethanol, stimulates apoB mRNA editing (von Wronski et al. (1998), Sowden et al. (2002)) and the nuclear accumulation of ACF (Sowden et al. (2002)). Consistent with data from ethanol-treated primary hepatocytes, addition of 10 nM insulin during the 4-hour labeling period resulted in a 2.5-fold increase in the recovery of phosphorylated ACF in nuclear 27S editosomes (FIG. 16). No phosphoACF was detected in any cytoplasmic fractions of insulin stimulated hepatocytes. These data demonstrate that ACF phosphorylation is a general characteristic associated with modulation of apoB mRNA editing activity.

ApoB mRNA editing and ACF phosphorylation can be modulated by protein phosphatase inhibitors. If ACF phosphorylation is integral to the regulation of apoB mRNA editing activity, and ACF must become dephosphorylated in order to traffic to the cytoplasm, it was reasoned that inhibition of the appropriate protein phosphatase would result in a nuclear accumulation of phosphorylated ACF and stimulate apoB mRNA editing. To evaluate this, hepatocytes were treated with a series of protein phosphatase inhibitors and editing activity determined. Protein phosphatase inhibitors were selected such that a role for a specific class of protein phosphatases could be determined. Cantharidin is a protein phosphatase inhibitor with markedly different inhibitory concentrations for PP2A and PP1 (40 nM and 473 nM, respectively) (Hardie et al. Methods Enzymol 201:469-76 (1991), Honkanen, R. E. FEBS Lett 330:283-6 (1993)). When tested at 470 nM, the IC₅₀ for PP1, a reproducible, but not statistically significant increase in editing (FIG. 4A) was detected. Additional experiments using 4.7 μM, a concentration anticipated to inhibit >90% PP1 activity, editing increased from 66% to greater than 90% (FIG. 4A). Additionally, treatment of hepatocytes with both cantharidin and ethanol simultaneously, conditions proposed to both stimulate ACF nuclear localization and phosphorylation as well as inhibit dephosphorylation resulted in the largest stimulation of editing activity.

To further support the role of PP1 in editing regulation, two additional protein phosphatase inhibitors, okadaic acid (Hardie et al. (1991) and endothall (Li et al. Biochem Pharmacol 46:1435-43 (1993)) were tested. Editing was enhanced from 66% to greater than 90% (P≦0.01) in hepatocytes treated with okadaic acid at concentrations 10 times the IC₅₀ for PP1 (Table 2). Similarly, treatment with endothall at a concentration 10-times the IC₅₀ for PP1 stimulated editing to statistically significant levels (p≦0.01) (Table 2).

Since calcium levels have been implicated in the regulation of editing (Chen et al. (2000)), the effect of inhibition of the calcium-sensitive protein phosphatase 2B (PP2B) (Wera, S, and Hemmings, B. A. Biochem J 311 (Pt1), 17-29 (1995)) on editing was investigated. Treatment of hepatocytes with cyclosporin A (Liu et al. Cell 66:807-15 (1991)) and cypermethrin (Enan, E. and Matsumura, F. Biochem Pharmacol 43:1777-84 (1992)) did not affect significantly apoB mRNA editing (Table 2). In addition to ruling out PP2B, these data also demonstrate that enhanced editing in the presence of protein phosphatase inhibitors is not a nonspecific effect due to small molecule inhibitors; further illustrating that PP1 is involved in editing regulation.

In order to correlate the observed effects on editing with changes in ACF phosphorylation, ACF was immunoprecipitated from extracts isolated from hepatocytes treated with increasing concentrations of cantharidin (FIG. 4B). ACF phosphorylation was increased in hepatocytes treated with 470 nM cantharidin. Maximal ACF phosphorylation was observed when hepatocytes were treated with both ethanol and cantharidin, reflecting the editing activity data described in FIG. 4A. Taken together, these data demonstrate that the cellular effects of cantharidin that lead to enhanced editing activity are associated with increased ACF phosphorylation.

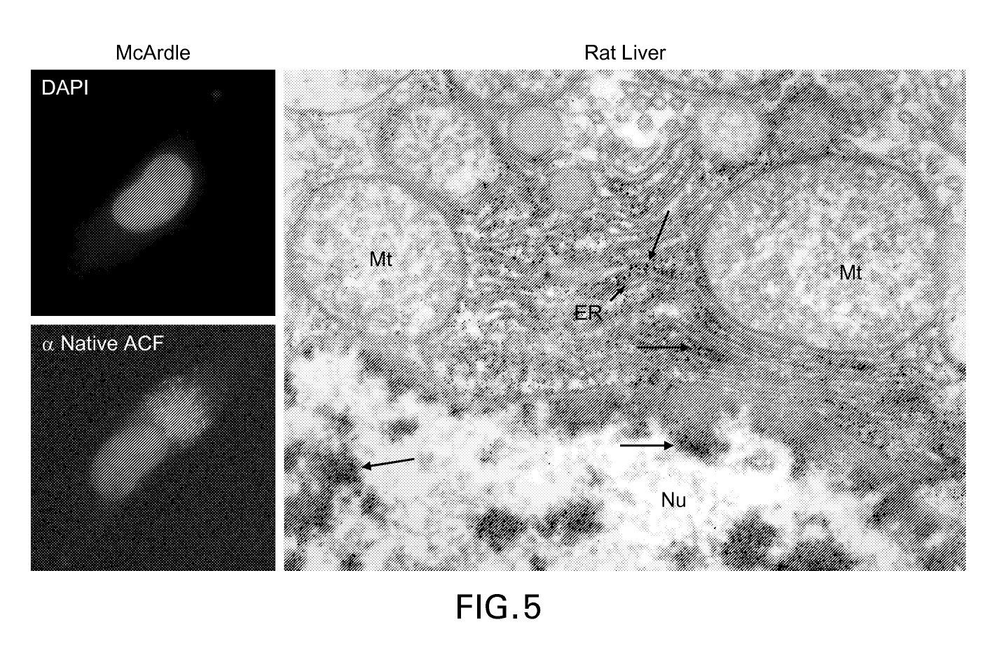

To evaluate the effect of PP 1 inhibition on the nuclear retention of ACF, hepatocytes were incubated with 470 nM cantharidin for 4 hours, fractionated into nuclear and cytoplasmic extracts, resolved by SDS-PAGE and immunoblotted for ACF. Subsequently, the blots were reprobed for actin and Histone H1 to verify cell fractionation quality and to serve as normalization standards for protein loading (FIG. 5). The normalized relative abundance of nuclear to cytoplasmic ACF was 1:1 in control cells. Upon incubation with cantharidin a five-fold increase in nuclear ACF was observed, showing that inhibition of ACF dephosphorylation prevents its nuclear export.

### Example 2

### PhosphoACF is Restricted to the Cell Nucleus

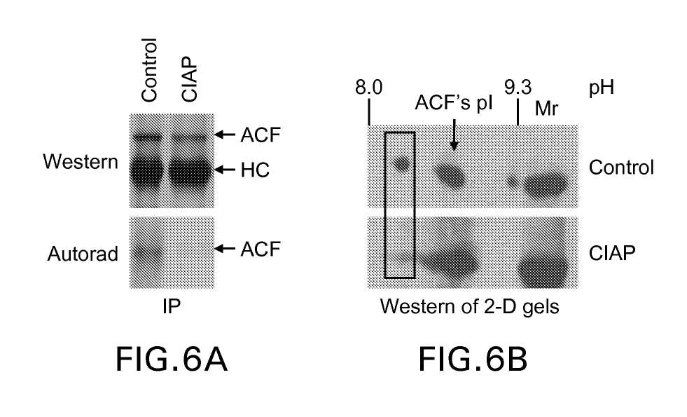

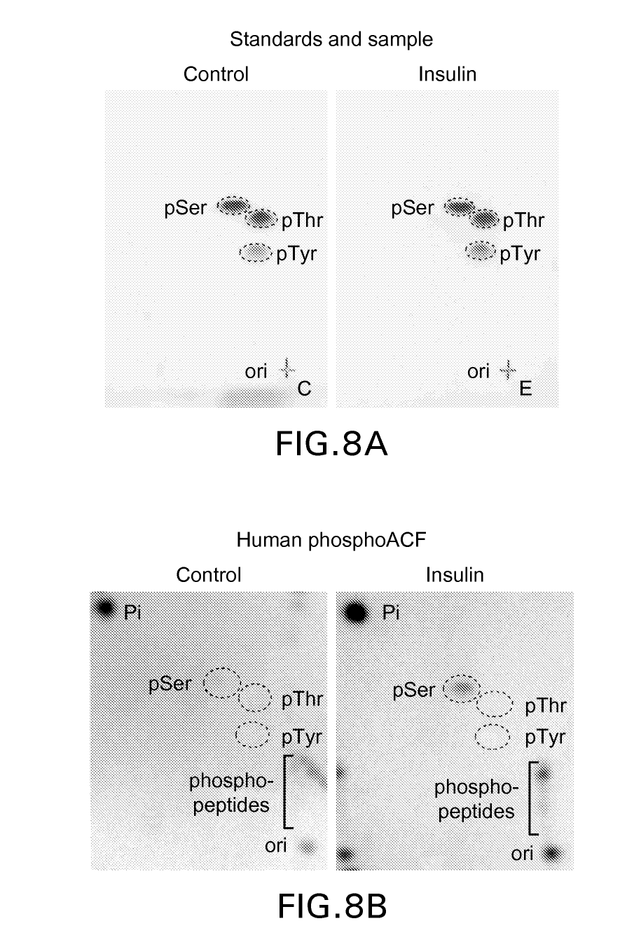

An evaluation of ACF phosphorylation was initiated because apoB mRNA editing activity could be induced without de novo protein synthesis (Giangreco et al. Biochem Biophys Res Commun 289:1162-7 (2001)). These data indicated that pre-existing editing factors such as ACF and APOBEC-1 could be activated (e.g. by post-translation modification) and/or mobilized (e.g. by nuclear import). To evaluate this, primary rat hepatocytes were cultured in basal insulin (0.1 nM) and labeled with 32Pi for 6 h and ACF immunoprecipitated (IP'ed) from nuclear extracts with ACF C-terminal peptide-specific rabbit polyclonal antibody (CT-Ab). PAGE analysis and autoradiography demonstrated incorporation of ³²P on ACF under these conditions (acute insulin and short labeling period), indicating a relatively high turnover site of phosphorylation (FIG. 6A). CIAP treatment of nuclear extracts did not affect the recovery of IP'ed ACF but markedly reduced the amount of isotopic label recovered with ACF (FIG. 6A, CIAP). Western blots of nuclear extract resolved by two dimensional (2D) PAGE revealed an ACF isoform at pI 8.3 (FIG. 6B; representing 15-20% of the total nuclear protein) with the majority of ACF focusing at pI 8.8 (FIG. 8B, arrow; the isoelectric point predicted for unmodified ACF). CIAP reduced the abundance of the pI 8.3 isoform and increased the amount of the pI 8.8 isoform (FIG. 6B, lower panel). The difference in charge between the two ACF isoforms is consistent with an acidic shift due to approximately two phosphates (0.2-0.3 pH units per phosphate, http://www.scansite.mit.edu/cgi-bin/calcpi).

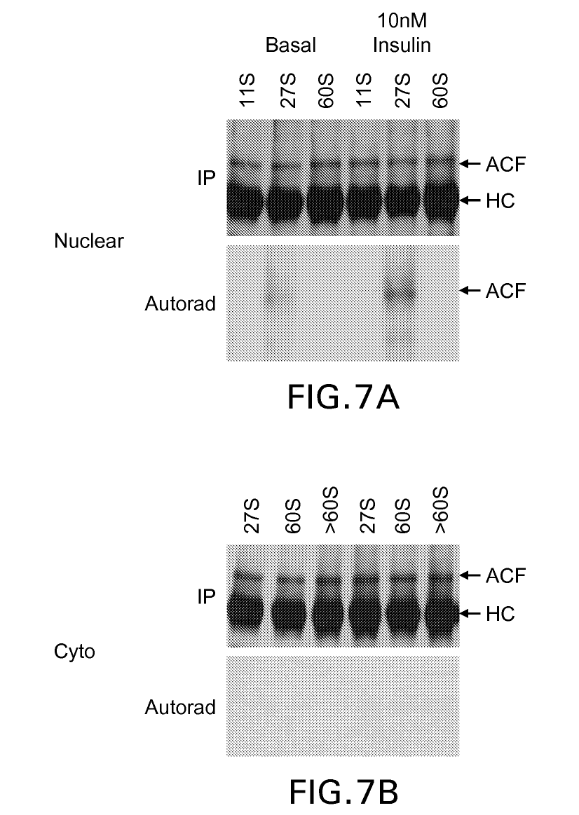

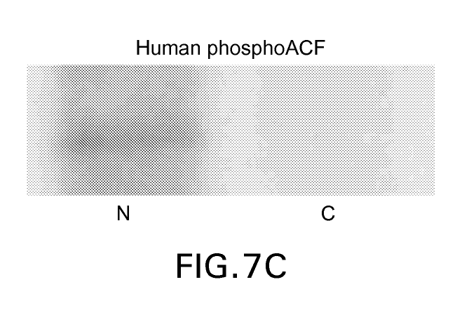

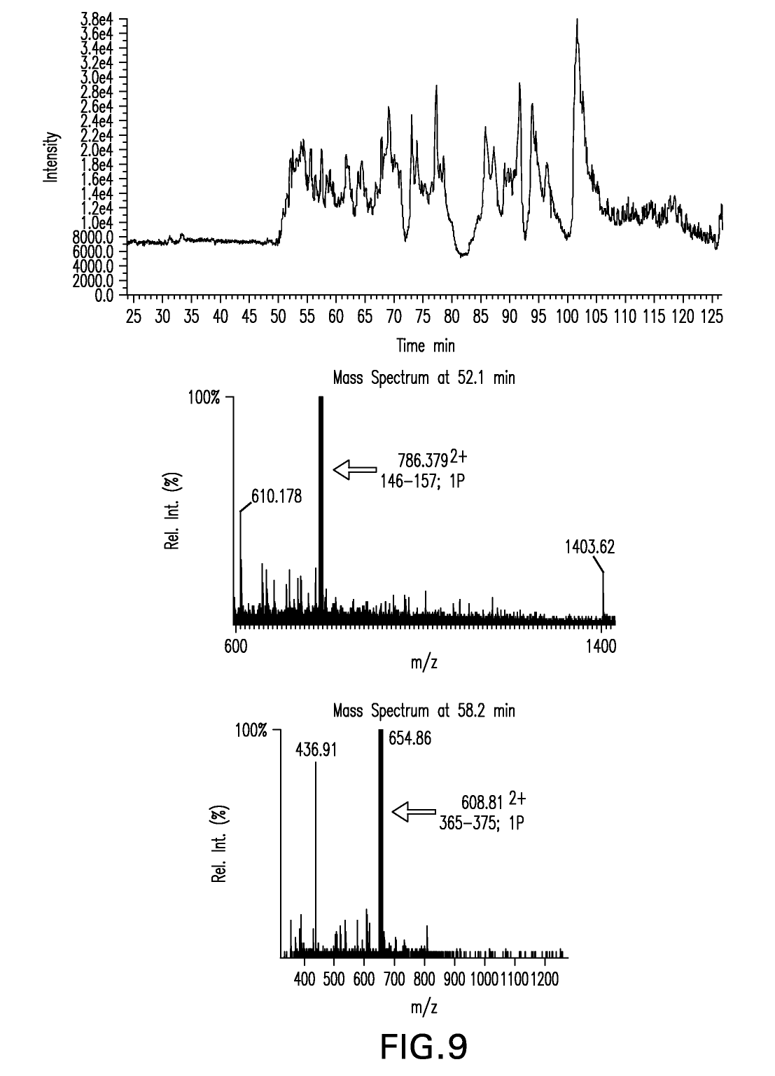

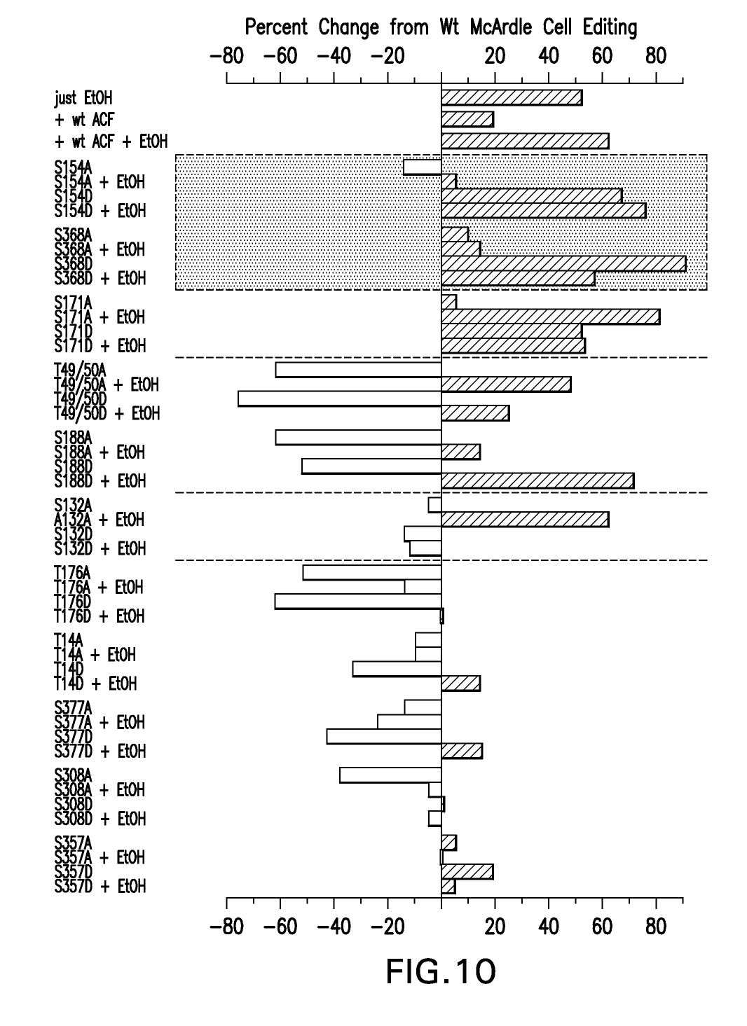

Previous analyses employing glycerol gradient sedimentation demonstrated that nuclear ACF was associated with APOBEC-1 in editing active 27S complexes and in the cytoplasm as 60S inactive complexes (the presumptive cytoplasmic reservoir for nuclear ACF) (Smith et al. (1991, Harris et al. (1993), Smith, H C (1998), Sowden et al. (2002)). To determine the relevance of ACF phosphorylation to these complexes, nuclear and cytoplasmic extracts from basal insulin or 10 nM insulin (post-prandial level) treated rat primary hepatocytes were sedimented through 10%-50% glycerol gradients. Relevant fractions (indicated across the top of panels A & B in FIG. 7) were IP'ed with CT-Ab (‘IP’), resolved by PAGE, western blotted and autoradiographed (‘Autorad’) and then the blots were reacted with the NT-Ab to visualize ACF. Although ACF could be IP'ed from all gradient fractions, phosphoACF was selectively recovered in nuclear 27S complexes (FIG. 7A). Despite 6-fold more cytoplasmic extract protein applied to the glycerol gradient (compared to nuclear protein), no evidence of cytoplasmic phosphoACF was found. It is apparent that insulin stimulated ACF phosphorylation and nuclear restriction can also be demonstrated in these cells (FIG. 9C). Primary hepatocytes were treated with insulin (0.1 nM) and ³²P for 6 h, fractionated into nuclear and cytoplasmic extracts and IP'ed with the CT-Ab, resolved by PAGE and autoradiographed. Collectively these data show that phosphoACF is sequestered in the nucleus of hepatocytes in humans and rats. Consequently, ACF phosphorylation is not dependent on its interaction with APOBEC-1.

### Example 3

### Protein Phosphatase 1 Activity as a Modulator of ACF Phosphorylation and Nuclear Retention

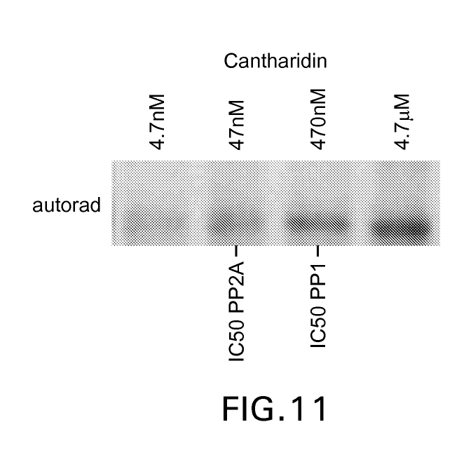

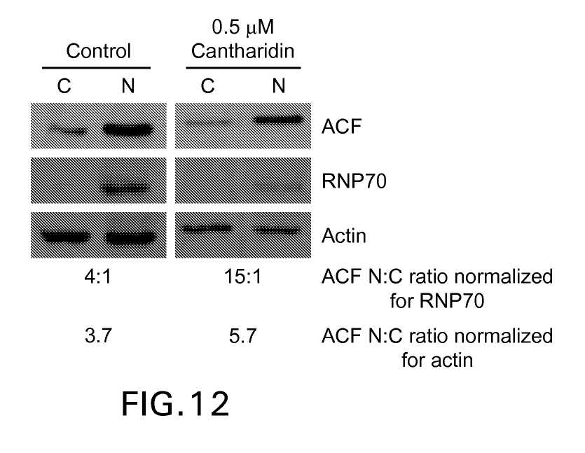

Chemical effectors of kinase and phosphatases were tested and demonstrated that Protein Kinase C activator (endothall) and Protein Phosphatase inhibitor (Cantharidin) stimulated apoB mRNA editing in primary rat hepatocytes. Cantharidin was evaluated further as a means of validating the role of ACF phosphorylation in its nuclear retention. Nuclear extracts of rat primary hepatocytes labeled with ³²P in the absence of presence of increase concentrations of Cantharidin for 6 hours were immunoprecipitated with ACF CT antibody and evaluated by PAGE and autoradiography (FIG. 11). Cantharidin treatment at the IC₅₀ of PP2A, 47 nM had little effect on the recovery of phosphorylated ACF (as well as apoB mRNA editing) An increased recovery of phosphorylated ACF was first apparent at the IC₅₀ of PP I and became markedly elevated following complete inhibition of PP1 (10-times the IC₅₀ or 4.7 μM). These data were observed with several hepatocyte preparations (n=5). The effect of Cantharidin on the distribution of ACF within primary hepatocytes was also evaluated by fractionating cells into cytoplasmic and nuclear extracts as described above and immunoblotting each fraction with the ACF CT antibody (FIG. 12). For each condition, a fixed percentage (3%) of the volume of cytoplasmic and nuclear extracts was resolved by PAGE; thereby ensuring that an equivalent amount of cellular starting material could be compared. Actin (which is found in both the cytoplasm and nucleus) under these loading conditions, immunoblotted with equivalent intensity in both fractions. RNP70 is a nuclear protein and its absence from the cytoplasmic fractions indicates that nuclear leaching during extract preparation was minimal. Scanning densitometry (of non-saturated film exposures, not shown) was used to determine reactivity of ACF CT antibody in each fraction and these values were normalized for actin (cytoplasmic fractions) or RNP70 (nuclear fractions) prior to calculating the N/C ratios. The data indicate a marked increase in nuclear ACF in hepatocytes treated with Cantharidin at the IC₅₀ of PP1 (0.5 μM) compared to untreated control hepatocytes. These data show trafficking of liver ACF in response to fasting-refeeding.

### Example 4

### Cantharidin Treatment Modulates the Relative Proportion of apoB mRNA Recovered with the Nucleus and the Amount of apoB Protein Produced

To evaluate changes in ACF phosphorylation and their modulation of apoB mRNA nuclear export, RNA was isolated from nuclear and cytoplasmic extracts of primary hepatocytes treated for 2 h with 10 nM insulin or 450 nM Cantharidin and quantified by real time PCR using Taqman technology and apoB primers flanking intron 27 and probe spanning the junction of exons 27/28. For these studies HPRT was quantified (CT value did not vary significantly between treatment groups) and was used to normalize the apoB mRNA data to equivalent number of input cells. Based on this, the recovery of total cellular apoB mRNA did not vary by more than 15% between treatment groups. The nuclear:cytoplasmic ratio of the apoB mRNA isolated from hepatocytes maintained in 0.1 nM insulin was 3:7. Treating hepatocytes with insulin or Cantharidin increased the amount of nuclear apoB mRNA such that nuclear:cytoplasmic ratios were 7:3 and 1:1 respectively. The simplest explanation for these data is that insulin and Cantharidin treatments lead a relative increased recovery of total cellular apoB mRNA in the nucleus by reducing nuclear export while nascent transcripts continued to accumulate.

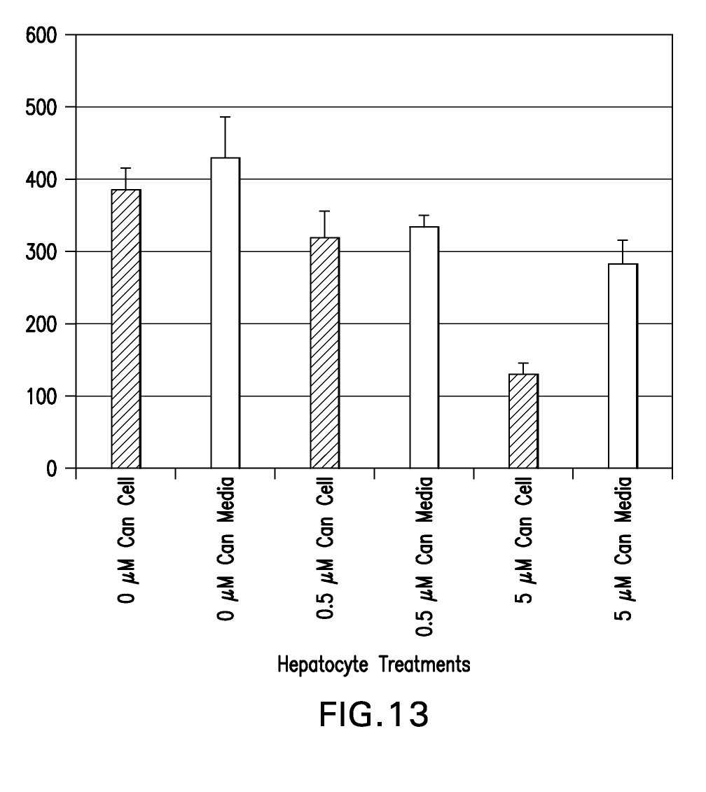

The effect of Cantharidin on the abundance of intracellular and secreted apoB protein was also evaluated. Primary hepatocyte cultures were treated with Cantharidin at the IC₅₀ of PP1 and 10-times higher (n=5 for each condition). Intracellular apoB protein in hepatocyte cytoplasmic extracts and apoB protein secreted into the media as VLDL was quantified as ng apoB protein per mg total protein in the Sparks' lab by radioimmunoassay using a monoclonal antibody against apoB as described previously (Chirieac et al. (2000), Au et al. (2004), Sparks, J D and Sparks, C E J Biol Chem 265:8854-62 (1990)) (FIG. 13). For these experiments, insulin was withdrawn from control and Cantharidin-treated cultures 24 hours prior to the addition of Cantharidin in order to isolate the effect of PP 1 inhibition in the absence of insulin stimulated kinase activity. A slight reduction in intracellular (cell) and secreted (media) apoB (22% and 17% respectively) was apparent at the IC₅₀ of PP1 (0.5 μM) relative to that observed in untreated hepatocytes (0 μM). A marked reduction in intracellular apoB (≧60%) was apparent in cells where PP1 was predicted to be completely inhibited (10-times the IC₅₀ or 5 μM). Secreted apoB was also reduced (≧30%) in cells treated with 5 μM Cantharidin. Taken together the data indicate a correlation between ACF phosphorylation/nuclear retention and suppression of apoB mRNA nuclear export and VLDL secretion.

### Example 5

### RNAi

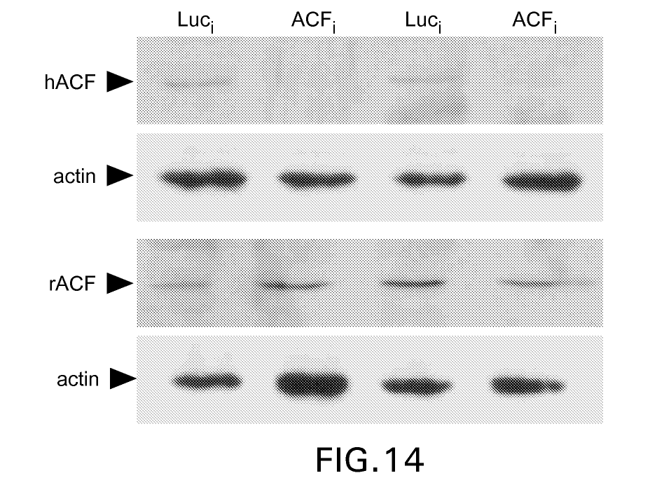

In one example, RNAi can be delivered by an adenovirus or adenoassociated virus 2 and can be used to reduce ACF where ACF is inappropriately overexpressed in disease or where one might want to affect an mRNA that ACF controls (other than apoB) for the purpose of down-regulating its protein expression. RNAi suppression of specific protein expression is required to demonstrate ACF-dependent nuclear export of apoB mRNA (Aim 1) and VLDL secretion and the role of PKC and PP 1 in regulating ACF phosphorylation-dependent changes in these two endpoints. Dharmacon have proven success with their RNA interference SMARTpool technology for the knock down of specific transcripts {described on the Dharmacon website publication}. A mixture of four double stranded RNAs was synthesized that was predicted to target specifically human ACF (ACFi). For these studies, a single dsRNA (Luci) specific for firefly luciferase and that targets no human transcript was purchased as a control for nonspecific effects of dsRNA transfection. The ability to knock down ACF expression was piloted in human HepG2 hepatoma cells. RNAi was transfected into cells in accordance with the manufacturer's recommendations using X-tremeGENE reagent (Roche). 72 hours post-transfection total cell lysates were western blotted with ACF N-terminal peptide specific and actin antibodies; the latter to ensure that an equivalent amount of cell material was compared (FIG. 14). In two separate experiments (left and right pairs) human ACF (hACF) abundance was significantly reduced compared to control RNAi (Luci) treated cells. To determine the species specificity of the RNAi SMARTpool, McArdle cells were transfected with human ACFᵢ and rACF expression determined. Significantly, rACF was refractory to RNAi knock down (lower two panels). Purchase of the individual dsRNAs in the SMARTpool also provided identification of their target ACF sequence. All four targeted sequence common to acf64/65 but each had 2-3 nucleotide mismatches for each site in rat ACF that explains the species specificity.

### Example 6

### Adenovirus Delivery System for Primary Hepatocytes

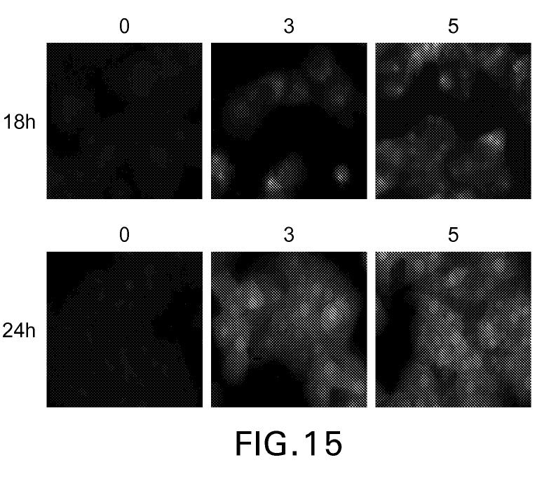

Primary hepatocytes transfect with very low efficiency (<5%) and, because they are not replicating cells, cannot be transduced with retroviral vectors. For this reason, adenovirus vectors and packaging systems are used for introducing cDNA and RNAi into primary hepatocytes. These systems are commercially available. The efficiency of adenoviral GFP delivery into primary mouse hepatocytes has been evaluated (FIG. 15). The fluorescent images of primary hepatocytes demonstrate increasing expression of GFP in mouse primary hepatocytes with increasing adenoviral m.o.i. (from left to right) and with increasing time subsequent to infection (compare corresponding 18 h and 24 h post-infection images in upper and lower panels). This adenoviral vector systems are widely available for introducing cDNAs (under the control of the CMV promoter) and RNAi (under the control of the mU6 promoter) into primary hepatocytes.

### Example 7

### A Role for ACF and Insulin-Dependent, Site-Specific ACF Phosphorylation in Modulating apoB mRNA Nuclear Export

ACF phosphorylation and nuclear retention are stimulated by insulin and these conditions are predicted to affect apoB mRNA nuclear export in hepatocytes. The occurrence of insulin-dependent phosphorylation of ACF and nuclear restriction of phosphoACF in human primary hepatocytes (which lack APOBEC-1) shows that ACF has other functions in addition to modulating editosome assembly and apoB mRNA editing. Changes in apoB mRNA abundance and export from the nucleus in response to insulin treatment are evaluated by real time RT-PCR. To this end, primary hepatocytes from apobec-1 KO mice (Hirano et al. (1996)) provide an ideal model for human hepatocytes.

Acute (2-6 h) and chronic (12-72 h) hyperinsulinemia (100 nM) are modeled in primary hepatocytes cultures. Primary culture enables an appropriate ‘n’ within treatment groups (6 plates per dose group) and replicates (3 repeats) for statistical analysis of variance (Student t-test). This is an appropriate and relevant experimental design as primary hepatocytes are not cell lines and their utility is based on the characteristic that for a limited time in culture (3-5 days), they retain the phenotype and regulatory pathways seen in liver in situ (Berry et al. Cell Biol Toxicol 13:223-33 (1997), Gomez-Lechon et al. Prog Mol Subcell boil 25:89-104 (2000), Neufeld, D S Methods Mol Biol 75:145-51 (1997), O'Brien et al. Chem Biol Interact 150:97-114 (2004)).

Quantification methods have been designed to evaluate fully processed nuclear and cytoplasmic apoB mRNA. RNA is isolated from primary hepatocytes as well as nuclear and cytoplasmic fractions using TriReagent® (MRC) and polyA+ mRNA purified with the MicroPoly (A) Purist kit (Ambion) following the manufacturer's protocols. Quantitative Real-Time RT-PCR will be performed in the Functional Genomics Center at the University of Rochester by a two-step reaction. Oligo-dT primed first-strand cDNAs are synthesized from polyA+ mRNA using AMV reverse transcriptase (Roche) according to the manufacturer's recommendations. Intron spanning primer combinations for apoB mRNA (forward 5′-TCAATGTGAAGTATAACGAAGATGG-3′ (SEQ ID NO: 38) and reverse 5′-ATGTCCAGATG AGCCTCTCC-3′ (SEQ ID NO: 39) and hprt mRNA (forward 5′-GACCGGTTCTGTCATGTCG-3′ SEQ ID NO: 40 and reverse 5′-ACCTGGTTCATCATCACTAA TCAC-3′, SEQ ID NO: 41) and TaqMan® hydrolysis probes (probe # 127 and 95, respectively) were selected from the Universal Probe library (Roche) using the vendor supplied software (http://www.univer salprobelibrary.com). Hprt mRNA is amplified in parallel in each sample as normalization control for cell number equivalents of mRNA evaluated. Reactions (10 μl) containing PCR Master Mix (ABI), primers (0.9 μM) and probe (0.1 μM) will be run on an ABI 7900 cycler using standard cycling parameters. Data will be analyzed using SDS software (ABI) and the relative quantification of apoB mRNA determined using the comparative Cₜ (2^{-ΔΔCt}) method.

Confirmation that the various treatments elicit the predicted phosphorylation of ACF and its nuclear retention are based on immunoprecipitation (IP) of ACF with ACF CT-specific antibodies from nuclear and cytoplasmic fractions of hepatocytes that have been radiolabeled with ³²P The IP'ed fractions are analyzed by PAGE and western blotting.

### Example 8

### The Function of ACF and Role of ACF Phosphorylation in Regulating Hepatic Secretion of VLDL

The role of insulin in the regulation of hepatic VLDL triglyceride export is complex. On the one hand, it has been established in the Sparks' lab (Sparks et al. (1990), by others (Patsch et al. J Clin Invest 71; 1161-1174 (1983), Patsch et al. J Biol Chem 261:9603-9606 (1986)) as well as reviewed (Sparks and Sparks (1994)) that insulin, during the immediate post-prandial period, inhibits the secretion of hepatic triglyceride by limiting newly synthesized apo B available for VLDL assembly. Thus, acute increases in insulin lead to transient storage of triglyceride in the liver. The limitation of apoB for lipoprotein synthesis by insulin occurs by two mechanisms. The first mechanism is related to the degradation of apo B that has failed to initiate full lipidation as VLDL. The second mechanism is related to insulin inhibition of B100 synthesis. It has been demonstrated that the inhibitory effect of insulin on B100 synthesis is not at the level of peptide elongation (ribosome transit studies), nor at the level of initiation (mRNP analysis in metrizamide gradients). These data show that insulin acts to sequester apoB mRNA in a non-translatable state. This shows that apoB mRNA can be non-translatable because it is sequestered in the nucleus through a mechanism dependent on the phosphorylation status of ACF.

The teleology of insulin inhibition of hepatic VLDL secretion is that it is the body's mechanism by which serum triglyceride levels are modulated during the immediate post-prandial and inter-prandial periods. Acute high levels of insulin that occur with meals, inhibit the synthesis and secretion of VLDL by the liver, allowing intestinal absorption of dietary fat in the formation of chylomicrons to proceed. As VLDL and chylomicrons share many catabolic pathways, the inhibition of liver VLDL production allows for efficient lipolysis of CM lipids targeted to fat stores while CM remnants are returned to the liver. As insulin levels fall during the inter-prandial period, apo B synthesis resumes, VLDL secretion is enhanced and hepatic lipids are cleared from the liver temporary storage pool.

Derangements of the insulin-apo B pathway are known to occur in obese, hyperinsulinemic rodent models (Sparks, J D and Sparks, C E Biochem Biophys Res Commun 205:417-422 (1994), Chireac et al. Amer J Physiol 287:E42-49 (2004)) and in streptozotocin induced diabetes (Sparks et al. J Clin Invest 82:37-43 (1988)), both states known to induce hepatic lipid accumulation. However, little has been known about the impact of ACF phosphorylation in determining the proportion of apoB mRNA capable of supporting apoB synthesis and VLDL assembly in these animal models. Retention of apoB mRNA in the nucleus by ACF and regulation of hepatic VLDL assembly in hyperinsulinemic states has a plausible role in the hepatic steatosis of type 2 diabetes.

The abundance of intracellular and secreted apoB are modulated in hepatocytes by ACF the treatments of primary hepatocyte are repeated except the endpoint is quantification of apoB protein. Although media from cell culture is collected and evaluated for VLDL content, the cells themselves have been processed for RNA isolation and as such, parallel cultures are needed for the quantification of intracellular apoB.

ApoB is quantified in culture supernatants harvested after various treatments by adjusting them to a density of 1.019 g/ml with NaBr and ultracentrifugation to ‘float’ VLDL and separate them from the infranatant containing LDL and HDL (Patsch et al. (1983)). Given that there is only one apoB protein molecule per VLDL (or LDL), immunological quantification of apoB protein in the gradient fractions of secreted lipoproteins is a reliable measure of the amount of VLDL produced. Relative to a standard curve composed of rat apoB VLDL, the amount of VLDL secreted in each culture is quantified by a radioimmunoassay based on an apoB polyclonal antibody (Sparks and Sparks (1990)).

Intracellular apoB is quantified by scrapping cells from the dishes into 0.25 M sucrose, 50 mM Tris pH 7.4, 5 mM MgCl₂ containing protease inhibitors (Complete®, Roche) following the various treatments. Cells are sheared by passage through 18, 20 and 26 guage needles. Cell lysates are cleared of debris by centrifugation (2000×g, 10 min 4C), adjusted to a density of 1.019 g/ml with NaBr and ultracentrifuged to separate lipoproteins. ApoB is quantified by RIA.

To determine the dependence of VLDL assembly and secretion on ACF, quantification of intracellular and secreted apoB is performed as described above at appropriate times following ACF RNAi with or without ACF cDNA rescue. The abundance of intracellular and secreted apoB in ACF knockdown hepatocytes and their ability to respond to insulin is assessed relative to that of hepatocytes that have not been treated with RNAi or treated with albumin RNAi and albumin cDNA rescue as negative controls.

The dependence of VLDL assembly and secretion on ACF phosphorylation status is determined by expressing ACF phosphorylation site-specific ala and asp mutants in cells and quantifying VLDL 48-72 h post-transfection or by quantifying intracellular and secreted apoB in cultures treated with ACF RNAi and rescued with ACF wild type and/or a phosphorylation site-specific ACF mutants as described above.

### Example 9

### The Kinase and Phosphatase Involved in Insulin-Regulated Phosphorylation of ACF

PKC and PP 1 have been implicated as responsible for modulating hepatic ACF phosphorylation in response to changes in insulin. It is critical for a mechanistic understanding of the regulatory processes that determine hepatic lipid accumulation and for the eventual development of therapeutics that ameliorate fatty liver disease that the enzyme activities required for site-specific ACF phosphorylation are determined. Kinase activators and inhibitors and phosphatase inhibitors have been evaluated for their effect on ACF phosphorylation and apoB mRNA editing. It has been demonstrated that the PKC inhibitor A3 (Calbiochem) (PKC IC₅₀=47 μM; PKA IC₅₀=4 μM) only inhibited ACF phosphorylation at 40 μM and higher in rat primary hepatocyte cultures treated with A3 and labeled with ³²Pi (500 μCi/ml) for 6 hours. This finding is consistent with the prediction based on computational analysis that S154 and S368 match PKC consensus phosphorylation sites. Moreover, among kinase activators, only Indolactam V (PKC activator) stimulated editing in hepatocytes.

The analysis with commercially available PKC isoform activities (Calbiochem) on recombinant ACF and ACF S154 and S368 alanine substitution mutants showed that PKC delta is responsible for ACF phosphorlyation. Consequently the initial RNAi analyses are with PKC delta knockdown in mouse primary hepatocytes. A limited number of other PKC isoforms or PKA (also affected by A3) are evaluated by RNAi knockdown to further demonstrate that PKC delta is the only relevant kinase.

Table 2 summarizes results of the effects of various phosphatase inhibitors on apoB mRNA editing in rat primary hepatocytes. These data indicate that inhibition of PP1 uniquely activates apoB mRNA editing and as indicated in FIGS. 11 and 12, inhibition of PP1 increased ACF phosphorylation and its nuclear retention; characteristics associate with enhanced editing activity. PP1 is focused on as a target for RNAi knockdown as the data indicate its involvement in ACF dephosphorylation and apoB regulation. Other phosphatase targets for knockdown with RNAi are evaluated (such as PP2A which is also inhibited with Cantharidin, see Table 2) to demonstrate that PP 1 is the only relevant phosphatase.

Validated short hairpin (sh) RNAi constructs that target 805 kinases and 35 phosphatases are available (http://www.openbiosystems.com) for probable candidates. Dharmacon and other commercial sources have, and continue to develop RNAi libraries from which kinase and phosphatase RNAi are obtained. Two to five different RNAi are typically recommended for each target mRNA and these are subcloned into Adenoviral vectors used to infect mouse primary hepatocytes. Isoform selective antibodies (Calbiochem) are used to evaluate the kinetics of kinase and phosphatase knockdown. Endpoints and their response to insulin treatment to that measured in hepatocytes with kinase or phosphatase knockdown can be measured. Rescue of RNAi knockdown using kinase or phosphatase cDNA Adenoviral delivery and cDNAs for the appropriate enzymes re obtained appropriately.

### Example 10

### Phosphorylation of ACF64 is Regulated In Vivo

In vivo phosphorylation of ACF associated with ethanol and insulin stimulation of hepatic apoB mRNA editing activity has been demonstrated. This was shown by data demonstrating that: (i) phosphoACF was selectively recovered with nuclei and co-sedimented with 27S editosomes and (ii) co-immunoprecipitation of APOBEC-1 with ACF was dependent on ACF phosphorylation status. Two-dimensional gel analyses revealed phosphatase sensitive and resistant isoforms of ACF and it can also be shown that ACF phosphorylation is regulated in both human and rat liver on a serine residue(s). Site directed mutagenesis showed that alanine replacement of serine 154 or serine 368 inhibited ethanol-stimulated editing activity but aspartic acid substitution fully stimulated editing activity in the absence of ethanol. Furthermore, mass spectroscopy data support phosphorylation of serine 154.

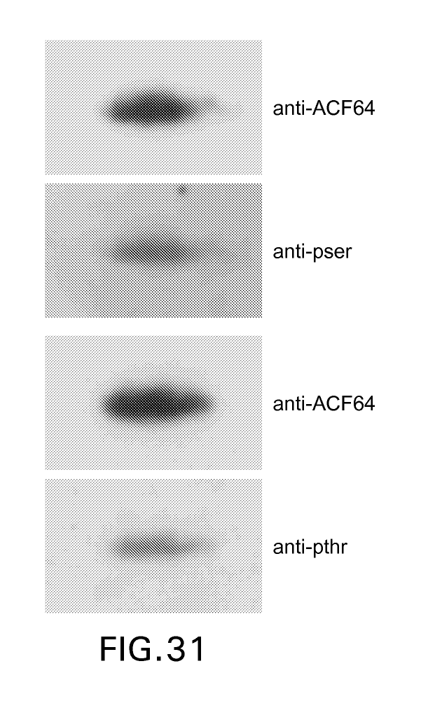

ACF specific antibodies were used to immunoprecipitate ACF from rat liver S100 nuclear extracts prepared from normal fed rats that were labeled with ³²P in vivo (20 mCi/350 gm body weight). Extracts were pretreated with RNase A/T1 as well as DNase I and then half of the material was treated with calf intestinal alkaline phosphatase (CIAP) prior to the addition of ACF C-terminal peptide specific, polyclonal antibodies (FIG. 3). The radiolabel was recovered by immunoprecipitation and was CIAP sensitive indicating protein phosphorylation (FIG. 3A). Western blots of 2D PAGE (FIG. 31) revealed two major charge isoforms of ACF64; one corresponding to the predicted pI of unphosphorylated ACF and a more acidic isoform (boxed region) calculated to contain 2-3 phosphates. The major band to the far right is ACF64 run on the second dimension gel as a positive control and size marker. Phosphatase treatment prior to PAGE virtually eliminated the acidic isoform. Phosphoamino acid specific antibody reactivity indicated that phosphoACF only contained phosphoserine and phosphothreonine (FIG. 31). Phosphatase treatment of rat liver nuclear extracts inhibited in vitro editing activity by >50% compared to buffer-treated control extracts (FIG. 2), showing that protein phosplhorylation was essential for optimal editing activity. In situ hyper phosphorylation of ACF64 was demonstrated by ³²P labeling of rat primary hepatocytes under basal insulin (Basal 0.1 nM insulin), post-prandial insulin (10 nM) or acute ethanol exposure (0.45% ethanol with 0.1 nM insulin). Using sub-saturating amounts of ACF C-terminal specific antibody, ACF was immunoprecipitated from glycerol gradient fractions containing either nuclear (A) or cytoplasmic (B) S100 extracts (FIG. 16). The data demonstrated a 2 and 3.5-fold stimulation of ACF phosphorylation by insulin or ethanol respectively. Phosphorylated ACF selectively co-sedimented with 27S nuclear editosomes and was not detected in pre-editosomal 60S complexes (FIG. 16A) or in cytoplasmic extracts (FIG. 16B). These showed that phosphorylation of ACF64 is regulated in vivo and based on selective recovery with 27S editosomes.

### Example 11

### Identification of Sites of ACF Phosphorylation using McArdle Cells

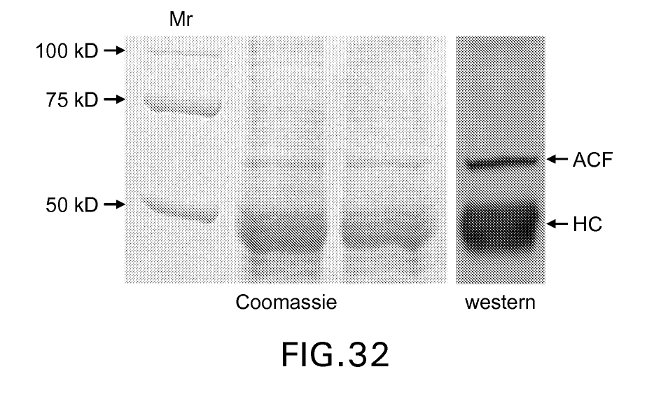

Radiolabeled ACF was immunopurified from 27S editosomes of ethanol treated, rat primary hepatocytes and analyzed by PAGE (FIG. 32 showed purity, specific activity and yield of ACF). The material submitted to ‘in-gel’ trypsin digestion, phosphopeptide purification and mass spectrometric analysis of phosphorylated ACF peptides. Substitution of a serine or threonine residue with alanine can prevent phosphorylation. If phosphorylation of these residues is required for a functional phenotype (in this case ethanol stimulated editing activity) then conversion to alanine can reduce or inhibit ethanol stimulation. Conversely, an aspartic acid substitution can mimic the charge resulting from serine or threonine phosphorylation. Therefore, if phosphorylation of these sites is important for activity, this substitution can activate the protein in the absence of ethanol stimulation. Some serines and/or threonines are functionally significant in the absence of modification (e.g. required for correct protein folding) and if changed, can adversely affect ACF function. Other sites are of less functional significance and alanine or aspartic acid substitutions can have no effect on function.

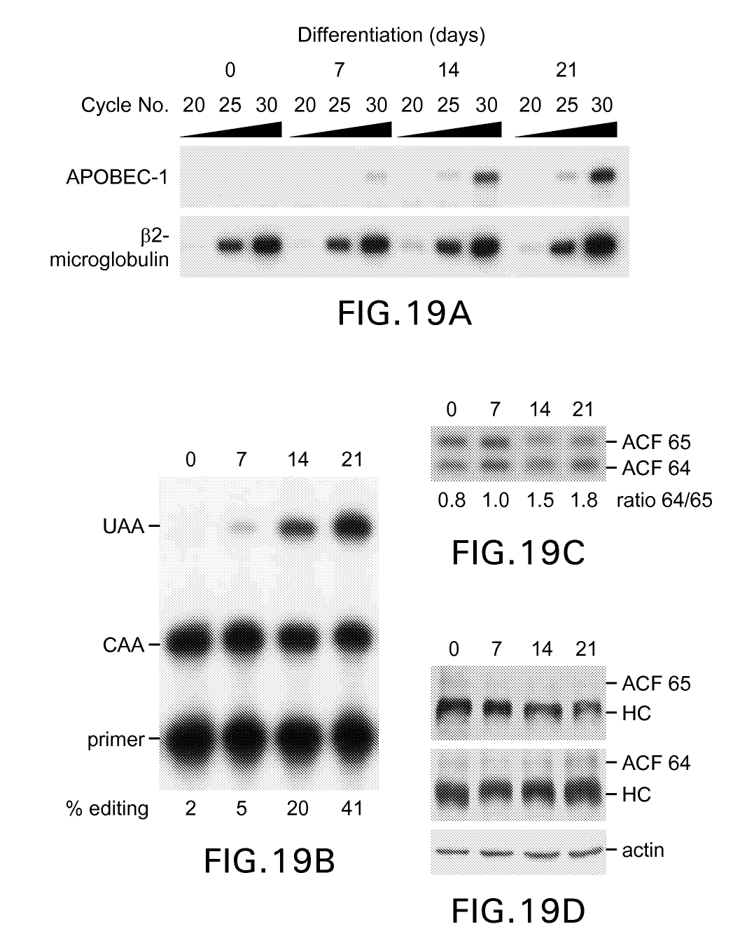

Screening phosphorylation sites in rat ACF64 involved creating alanine and aspartic acid substitutions for each of 20 serines or threonines predicted by NetPhos 2.0 and Prosite to be potential sites of phosphorylation. With the exception of one (S253; FIG. 19) these sites are conserved between rat and human ACF and are therefore more likely to be functionally significant. In total, the algorithms predicted 30 potential sites of phosphorylation of which only 12 serine, 11 threonine and 7 tyrosines had predictive scores greater that 0.5 (0-1.0 scale). 80% of these sites were localized to amino acids 3-377 of the 596 total in ACF and were considered to be of more potential significance because the N-terminal two-thirds of ACF is sufficient for nuclear localization, RNA binding and APOBEC-1 interaction (Sowden et al. (2004)). The predictive algorithms of NetPhos and Prosite have recognized deficiencies so the sites selected for mutagenesis were from both the high scoring (≧0.8), as well as the moderate (0.3-0.7) and low scoring sites (≦0.1). Each site was mutated to alanine and aspartic acid using the Quikchange system (Stratagene) and all mutants confirmed by DNA sequencing.

Another aspect involved isolation of stable McArdle cell lines that expressed equivalent levels of the mutant ACF proteins. McArdle cells were used for these studies because they (i) express APOBEC-1, (ii) their editing activity can be stimulated by ethanol (enabling mutant ACF to be evaluated in the context of a regulatory system and (iii) ectopic expression of wild type ACF has been characterized in these cells. Lines expressing only moderate levels (estimated by western blotting with ACF peptide specific antibodies to be 3- to 5-times that of endogenous ACF) were selected to minimize overexpression levels.

The results of representative substitution mutations on basal and ethanol stimulated editing activity are depicted in FIG. 13 (with the complete tabulation of the original data in Table 4). The central vertical line indicates the percent editing of wild type (WT) McArdle cells. To calibrate the system cells were incubated in the presence of ethanol for 4 hours and the amount of edited apoB mRNA determined. Ethanol induced the anticipated increase (52%) in editing activity (21% editing to 32%) (Yang et al. (2000), Van Mater et al. Biochem Biophys Res Commun 252:334-339 (1998)). Stable ectopic expression of ACF64 (WT ACF) stimulated editing slightly (19%) compared to untransfected McArdle cells and the addition of ethanol to this cell line induced a further 3-fold stimulation.

Editing activity in stable cell lines expressing S154A was inhibited compared to WT McArdle cells, showing a dominant negative phenotype. Cells expressing S368A had only slightly elevated editing activity but the response of both S368A and S1154A mutants to ethanol was marginal compared to that of WT ACF expressing cells. Importantly, aspartic acid substitutions at these sites stimulated editing activity to levels equal to or greater than those seen in ethanol treated WT ACF expressing or parental McArdle cells and ethanol did not increase this stimulation. The existence of more than one site of ACF phosphorylation is consistent with the mobility shift of ACF on 2D PAGE.

It is possible that alanine substitutions at these sites prevented an essential ACF64 function or interaction. For example, as S368 is within the nuclear localization motif (Blanc et al. (2003)) the dominant negative effect of this mutation could have been due to a failure to localize to the nucleus while interacting with and preventing APOBEC-1 nuclear co-import. S154 is in the APOBEC-1 interaction domain indicating S154A can be incapable of interacting with APOBEC-1 but retain nuclear importcapability and compete with the endogenous ACF for apoB mRNA binding.

The phenotype of S171A is equivalent to S154A and S368A in that it poorly stimulated editing relative to WT ACF but importantly, differed in that editing activity was stimulated by ethanol. This indicates that S171 can be important to ACF function but that it may not be a site of ethanol induced phosphorylation. The S171D mutation stimulated editing activity to levels near those seen with ethanol treated WT ACF and the addition of ethanol did not further enhance editing activity. This shows that S171 can activate editing if it became phosphorylated but that this might blunt the ethanol response by adversely affecting ethanol regulated phosphorylation at other sites, e.g. S154. Alternatively, a charged group at position 171 can be preferred over a serine (or alanine) and the ethanol response of S171D cannot be resolved from the background response of the endogenous ACF. In this regard S171 exemplifies the importance of evaluating select substitution mutants in the context of a regulatory cell system in which expression of the endogenous ACF has been reduced by ACF specific RNAi.

The remaining mutants have in common that both alanine and aspartic acid substitutions resulted in dominant negative effects on editing activity. This phenotype was predicted but presents as three categories of mutants (FIG. 13): (i) those for which ethanol treatment restored editing activity of the alanine and aspartate substitution (e.g. T49/T50 and S188), (ii) those for which only the alanine substitution was rescued by ethanol treatment (e.g. S132) and (iii) those whose editing activity never reached wild type levels. The data indicate that these sites maybe important as serine or threonine but that their phosphorylation did not contribute to ethanol stimulation of editing activity. Further analysis indicated aspartic acid substitution was more deleterious for editing activity than alanine (e.g. T14, T160, S253 and S377). Phosphorylation at these sites can reduce (but not eliminate) editing activity or inhibit phosphorylation at other sites such as S154 and S368. The role of an inhibitory mechanism in regulating editing activity has been discussed (Harris et al. (1993), Sowden et al. (2002), Blanc et al. (2003), Giangreco et al. Biochem Biophys Res Commun 289:1162-1167 (2001)). Re-evaluating the data in FIG. 13 in this context shows that negative regulation of editing can involve kinases acting at sites such as T160, S253 and/or S377 or phosphatases acting at S154 and/or S368. The converse is true when editing is activated. The ability of ethanol treatment to rescue T160D, S253D and S377D shows that phosphorylation at sites such as S154 and S368 can override negative regulation by phosphorylation at T160, S253 and/or S377.

### Example 12

### The Functional Significance of ACF64/65 Phosphorylation

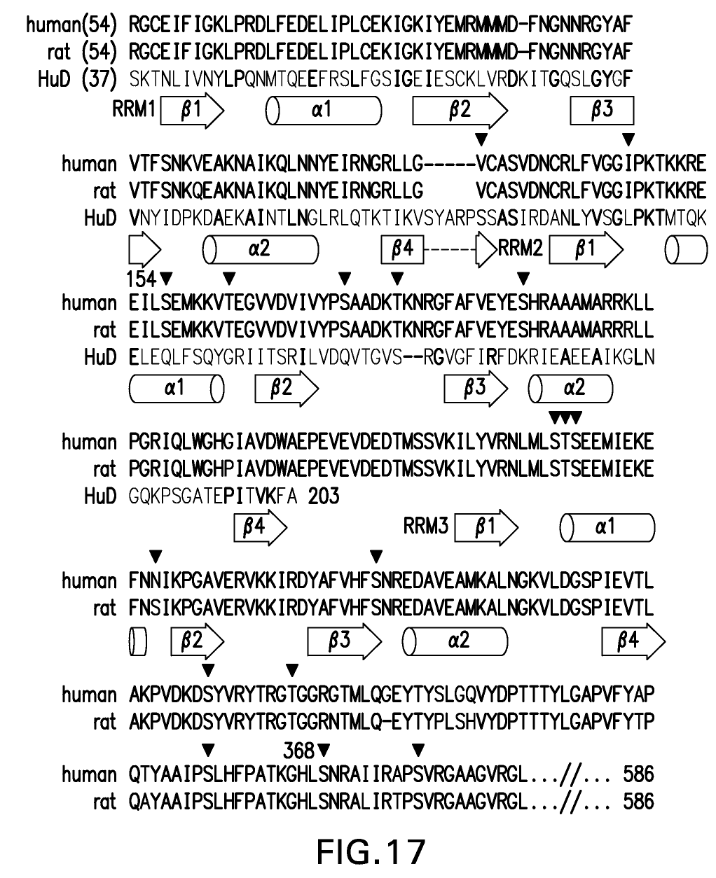

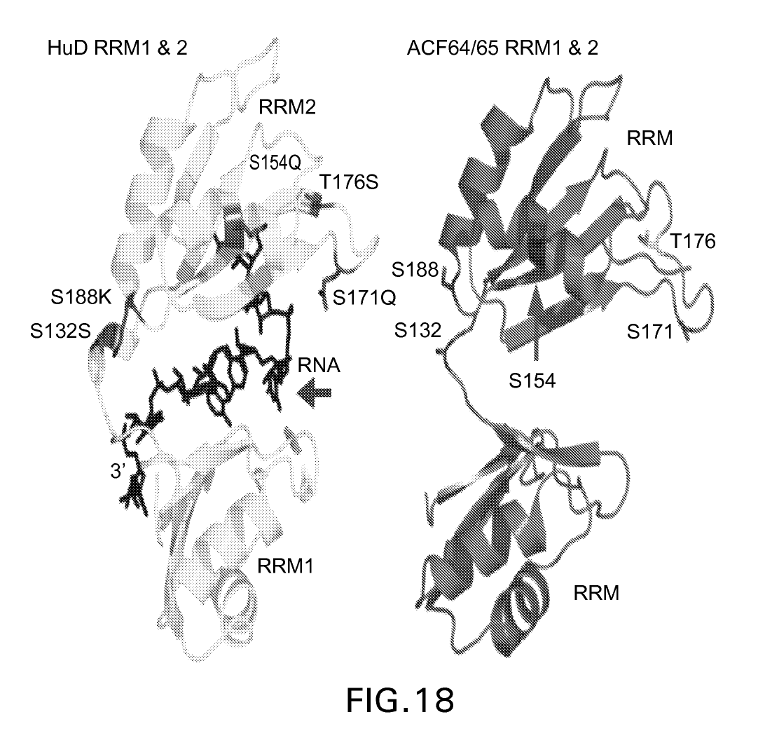

Comparative modeling based on known protein structures enables rational predictions that can guide the design of functional analyses of ACF phosphorylation sites. The RNA recognition motifs 1 (RRM1) and RRM2 (amino acids 55-223) of ACF64/65 was modeled by comparison to the 1.8 Å resolution crystal structure of the HuD protein comprising RRMs 1 & 2 in complex with an I nucleotide, AU-rich RNA (Wang et al. Nat Struct Biol 8; 141-145 (2001)). HuD is a member of the Elav/HelN1/HuR family of proteins that contain multiple copies of RRMs, which bind to AU-rich elements (AREs) in the 3′ UTR of mRNAs, where they regulate mRNA stability (Brennan, C M and Steitz, J A Cell Mol Life Sci 58:266-277 (2001), Burd, C G and Dreyfuss, G. Science 265:615-621 (1994), Kielkopf et al. Genes Dev 18:1513-1526 (2004)). Of significance is the analogous role of ACF in stabilizing edited apoB RNA to evade NMD. HuD RRM 1-RRM2 demonstrated a typical RRM structural fold comprising a four-stranded anti-parallel β sheet flanked by two ox helices (FIGS. 17 and 18). For a typical RRM-containing protein, RNA recognition occurs through the β-strands and flanking loops, whereas protein-protein interactions can occur at the α helices or β-strands. In HuD, the β-sheets of RRM1 and RRM2 bind AU-rich ssRNA (FIG. 17, HuD residues shown in a lighter font) whereas the (x helices face outward, exposed to solvent, thereby making them accessible for protein-protein interactions (Wang and Hall (2001, Kieldopf et al. (2004)) (FIG. 18).

Alignment of HuD RRM1-RRM2 sequence with those of human and rat ACF64 (FIG. 17) showed this region of HuD was an appropriate model for comparative modeling of the comparable region in ACF64. First, there is 94% amino acid identity (98% similarity) between human and rat ACF. Second, 23% of the aligned HuD residues were identical to ACF (shown in black) with 51% similarity. Finally, the selection of HuD versus other RRM containing structures was based on UV crosslinking and RNA binding competition analyses that indicated a preference of ACF64/65 for binding to AU-rich RNA, an observation in accordance with the AU rich nature of the RNA sequence in the vicinity of the apoB RNA editing site Harris et al. (1993), Mehta et al. (1996), Blanc et al. (2001), Mehta, A and Driscoll, D M RNA 8:69-82 (2002), Backus, J W and Smith, H C Biochim Biophys Acta 1217:65-73 (1994), Backus et al. Biochim Biophys Acta 1219:1-14 (1994)). Given these similarities, the HuD structure (Wang and Hall 92001), Kielkopf et al. (2004)) appears to be the most plausible structural template for ACF. The model (FIG. 18) was calculated by use of the program MODELLER.

The resulting model indicated that S154 in ACF64 (Q154 in HuD) resides in an α-helix and is exposed to solvent making it accessible to kinases. If the RRM1-RRM2 structure of ACF is functionally homologous to that of HuD and involved in protein-protein interactions, then phosphorylation of S154 is likely to affect the interaction of ACF with other proteins, but not RNA binding. This hypothesis is supported by mutagenesis of ACF64 indicating RRMs 1 and 2 are both required for optimal APOBEC-1 binding (Blanc et al. (2001), Mehta and Driscoll (2002)). Hence, structural modeling provides a rational explanation for why S154D enhanced editing (by 67%), a result of mimicking a salt-bridge necessary to promote a productive ACF-APOBEC-1 electrostatic interaction.

Conversely, Both S171 and T176 reside within a loop that joins two adjacent β-strands of RRM1. When each site was substituted with alanine, editing activity stayed the same or was reduced (by 50%), respectively (FIG. 13). Aspartic acid substitution at 171 stimulated editing but further inhibited editing at 176 consistent with a its structural role of this residue in negotiating a sharp turn in HuD. Hence, most substitutions (with the possible exception of Ser/Thr) are predicted to be detrimental to protein folding in this region. The location of S171 within a flexible loop is equivalent to Q171 of HuD, whose amide group is ˜4 Å from the RNA backbone. As such, S171 of ACF is not expected to contact RNA, nor is the S171D mutant expected to repel the RNA backbone significantly since Asp differs from Gln in lacking a methylene group (˜2 Å). Moreover, the side-chain of the D171 mutant is not conformationally restricted so that when RNA bound to ACF, the carboxylate of Asp could rotate away. As such, the substitution data are interpreted as indicating that neither S171 nor T176 are sites of physiologically relevant phosphorylation.

S132 and S188 also are unlikely to be ACF phosphorylation sites. In the model, these residues are positioned in the inter-domain linker and at the N-terminal end of an α-helix, respectively (FIG. 18). The localization of S132 within the linker (which has no effect as an Ala mutant but loss of activity as Asp), showing a potential structural perturbation due to the distance of S132 in HuD from the RNA.

S368 is within an identified NLS of ACF64/65 (amino acids 331-385) (Blanc et al. (2003)). Therefore phosphorylation at this site can affect interactions necessary for nuclear cytoplasmic shuttling (Zhu and Gulick (2004), Huang et al. (2004), Wilson et al. (2003, Maryland et al. (1993), Fung et al. (1997), Xiao and Manley (1997), Tacke et al. (1997), Li et al. (1997), Li et al. (1994), Imbert et al. (1996), Beg et al. (1992)).

Differentiation of Caco2 human carcinoma cells into intestinal cells (enterocytes) is an accepted model system for studying intestinal lipoprotein synthesis and apoB mRNA editing (Jiao et al. J Lipid Res 31:695-700 (1990)).

In characterizing the Caco2 cell model, apobec-1 mRNA expression was assessed during differentiation (0-21 days) by comparison to the housekeeping gene transcript β2 microglobulin by semi-quantitative RT-PCR amplification using a radiolabeled PCR primer. The β2 microglobulin results indicate an equivalent amount of total RNA was used in the RT-PCR reactions from each time point. The apobec-1 data revealed a low level of expression by 7 days that increased upon a further 2 weeks of differentiation. Apobec-1 mRNA expression was insufficient to induce a significant increase in editing activity, compared to that in proliferating cells until 2 weeks of differentiation (FIG. 19B). Editing activity increased upon further differentiation concomitant with an increase in apobec-1 mRNA expression.

Similar semi-quantitative RT-PCR analyses revealed that proliferating Caco2 cells expressed acf mRNA and the levels of the alternatively spliced RNA variants acf64 and acf65 were almost equivalent (FIG. 19C). After 2-3 weeks of differentiation acf64 mRNA was the prominent spliced variant (acf64:acf65 ratio of 1.8:1) consistent with the finding that acf64 mRNA is the predominant spliced variant in fetal and adult human intestine (Henderson et al. (2001)) as well as in rat liver, rat intestine and HepG2 human hepatoma cells (Sowden et al. (2004)).

To evaluate ACF protein expression during differentiation whole cell extracts from equivalent cell protein (based on actin normalization, FIG. 19) were immunoprecipitated with ACF C-terminal specific antibody, then blotted with either the N-terminal or anti-ACF65 specific antibody. Proliferating Caco2 cells expressed both ACF65 and ACF64 but expression of ACF65 waned while ACF64 remained during differentiation. This trend parallels that observed for the corresponding mRNAs showing that alternative mRNA splicing and protein variant translation can be regulated in differentiating Caco2 cells.

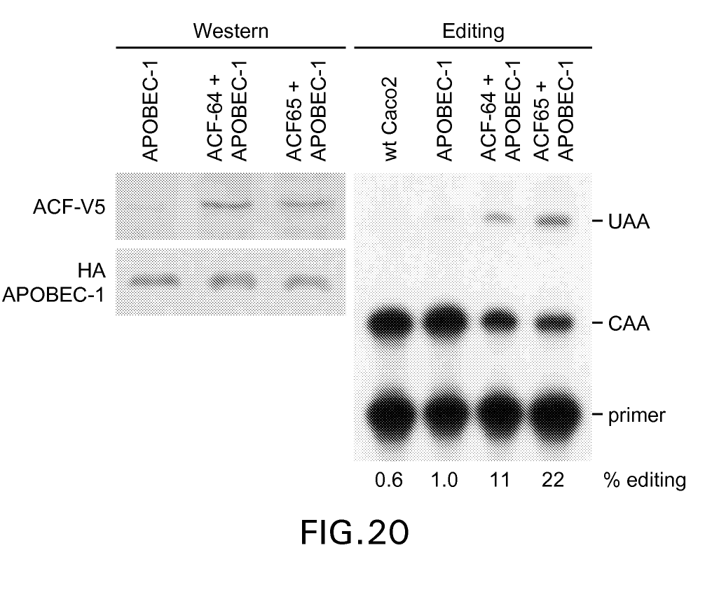

Of particular importance to the study of ACF phosphorylation as a regulatory mechanism is the question of whether induction of APOBEC-1 expression during differentiation is sufficient to stimulate apoB mRNA editing or if ACF phosphorylation is also required. It has been shown that APOBEC-1 expression alone is not sufficient. Transient transfection of EGFP using FuGene 6 (Roche) indicated that up to 50% of proliferating Caco2 cells can be transfected. Stable cell lines were also established that expressed C-terminal V5-tagged rat ACF64 and ACF65 (FIG. 20, left panel). Editing activity, quantified by poisoned primer extension on endogenous apoB mRNA, was low in proliferating Caco2 cells as well as in cells transiently transfected with HA-tagged APOBEC-1 (FIG. 20, right panel). In contrast, overexpression of APOBEC-1 in Caco2 cells stably expressing ACF64 or ACF65 induced much higher levels of editing. The data show that despite the expression of endogenous ACF64/65 in proliferating Caco2 cells, its abundance is too low or it is blocked from productive participation in the editing mechanism. Clearly however, APOBEC-1 expression was not sufficient to induce editing and therefore differentiation must involve additional regulatory processes. ACF phosphorylation is consistent with this type of regulation. Overexpression of ACF64 or ACF65 can overcome through mass action ACF regulation, enabling editing to become active.

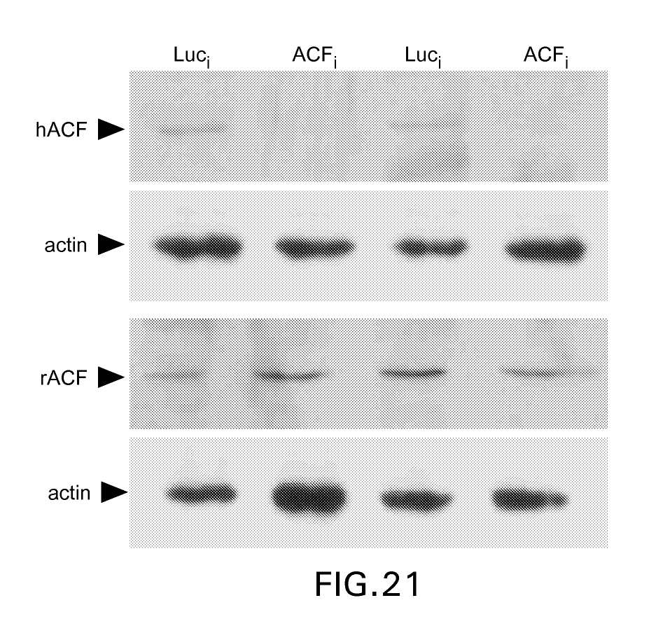

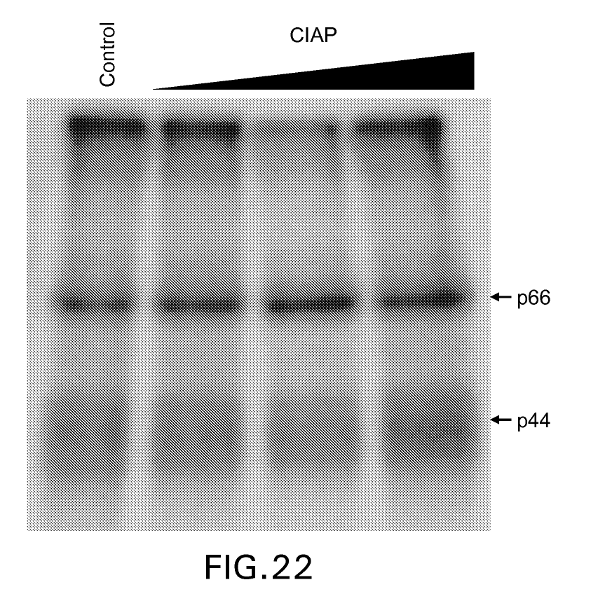

RNA interference SMARTpool technology for the knock down of specific transcripts has been shown to work (Dharmacon (2004)). A mixture of four double stranded RNAs was synthesized that was predicted to target specifically human ACF (ACFi). A single dsRNA (Luci) specific for firefly luciferase and that targets no human transcript was purchased as a control. RNAi was transfected into cells in accordance with the manufacturer's recommendations using X-tremeGENE reagent (Roche). 72 hours post-transfection total cell lysates were western blotted with ACF N-terminal peptide specific and actin antibodies; the latter to ensure that an equivalent amount of cell material was compared (FIG. 21). In two separate experiments (left and right pairs) human ACF (hACF) abundance was significantly reduced compared to control RNAi (Luci) treated cells. To determine the species specificity of the RNAi SMARTpool, McArdle cells were transfected and rACF expression determined. Significantly, rACF was refractory to RNAi knock down (lower two panels). Purchase of the individual dsRNAs in the SMARTpool also provided identification of their target ACF sequence. All four targeted sequence common to acf64/65 but each had 2-3 nucleotide mismatches for each site in rat ACF that explains the species specificity. RNAi treated HepG2 cells can have reduced ability to complement transfected APOBEC-1. The ability of ACF phosphorylation site mutants to rescue ethanol stimulation in McArdle cells or differentiation induced editing (Caco2 cells) is assessed in ACF RNAi treated cells.

Protein phosphorylation appears important for the interaction of ACF with APOBEC-1. Nuclear and cytoplasmic S100 extracts were prepared from a stable McArdle cell line that overexpresses HA-tagged APOBEC-1. Endogenous ACF was immunoprecipitated using ACF C-terminal peptide specific polyclonal antibodies, the complexes were washed with 850 mM NaCl to remove nonspecific adsorbed proteins (ACF's interaction with APOBEC-1 is stable up to 1 M NaCl, (Yang et al. (1997), Lau et al. (1991)) and then resolved by PAGE and western blotted with either anti-HA or ACF N-terminal, peptide specific antibodies. Blotting of a fractionated cell extract (starting material) demonstrated ACF64 and APOBEC-1 were present in the nucleus and the cytoplasm. However, following ACF specific immunoprecipitation, APOBEC-1 was only detected in the nuclear extract. Therefore, although ACF and APOBEC-1 co-sediment as 60S complexes from cytoplasmic extracts they apparently do not form stable complexes, consistent with the lack of cytoplasmic editing activity (Yang et al. (2000)). In contrast, phosphorylated ACF and APOBEC-1 form stable complexes in nuclear extracts consistent with the presence of 27S complexes and nuclear editing activity. Therefore co-immunoprecipitation assays can reveal whether a particular phosphorylation site mutant has lost the ability to traffick to the nucleus and/or interact with APOBEC-1.

### Example 13

### Identification of the Protein Kinase(s) and Phosphatase(s)

Mechanisms that target kinases to specific sites in proteins are key aspects of the fine tuning that makes reversible phosphorylation such a powerful regulatory process. In this regard there are examples, most notably in proteins involved in cell cycle regulation or those that respond to metabolic stimuli, of kinases that phosphorylate single sites within a protein and other examples where proteins are phosphorylated at multiple sites involving one or more kinases. Such proteins rarely exist in a state of complete phosphorylation or dephosphorylation but by the cellular regulation of kinase and phosphatase activities maintain various degrees of hyper- or hypo-phosphorylation (Morgan, D O Annual Reviews, Inc. Palo Alto, Calif. (1997)).

NetPhos and Prosite predicted S368 of ACF to be a site recognized by Protein Kinase C (PKC) (KGHLSNRAL, SEQ ID NO: 46) but a kinase for S154 was not specifically predicted (EEILSEMKK, SEQ ID NO: 47). Interestingly, many of the sites were predicted to be substrates for PKC, while a few were predicted substrates for PKA (T316), casein kinase II, CKII (S171, T49/50, S241, S243) and cAMP-dependent kinase (T160). It was reasoned that if phosphorylation of ACF regulated editing activity, treatment of rat primary hepatocytes with phosphatase inhibitors might stimulate apoB mRNA editing activity. Rat hepatocytes were incubated for 6 hours at concentrations of 10- to 100-times the IC₅₀ for various phosphatase inhibitors (Calbiochem) and the amount of endogenous apoB mRNA edited quantified by RT-PCR and poisoned primer extension (Table 3). The experiments (n=4-6) were done over a number of months and therefore represent primary hepatocytes from several rats.

Editing activity was increased at inhibitor concentrations that inhibit protein phosphatase 1 (PP1) but little change occurred at concentrations that inhibit either PP2a or PP2B (Table 2) showing that PP 1 can in part regulate editing activity.

The corollary to the these studies is the treatment of hepatocytes with protein kinase activators which can increase editing activity. Editing activity was quantified from primary hepatocytes incubated with 10-100-times the EC₅₀ of the various kinase activators (Calbiochem). Among those tested were Indolactam V, (PKC; EC₅₀=200 nM), 8-cpt camp, (PKA; EC₅₀=2 μM) and for activation of various cAMP dependent kinases (Forskolin, EC₅₀=4 μM; isoproterenol, EC₅₀=50 μM; glucagon, EC₅₀=50 nM). Only Indolactam V at 12 μM markedly stimulated editing activity compared to solvent treated controls (from 65% to 92% editing±SEM 4.8, n=2). The data implicate the activity of PKC as a regulator of ACF phosphorylation.

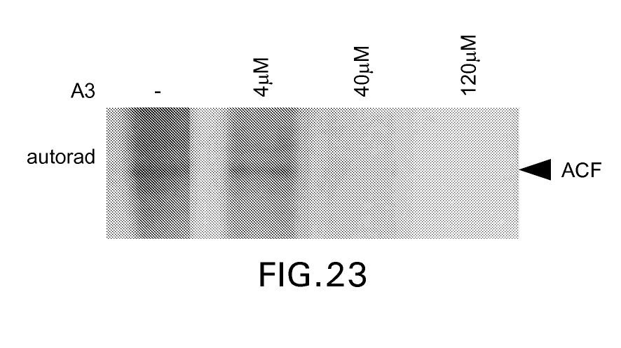

Numerous kinase inhibitors were evaluated but only those acting on Protein Kinase C affected editing activity. McArdle cells were pretreated with the PKC inhibitor A3 (Calbiochem) (PKC IC₅₀=47 μM; PKA IC₅₀=4 μM) at 4, 40 and 120 μM in phosphate free media before the addition of ³²Pi (500 μCi/ml), fresh A3 and a 6 hour incubation. Cell extracts were immunoprecipitated with the ACF C-terminal specific antibody and analyzed by PAGE and autoradiography (FIG. 23). At the IC₅₀ of PKA A3 had a modest effect on the phosphorylation of ACF (20% decrease) relative to untreated cells. However, at and above the IC₅₀ of PKC, phosphorylation of ACF64 was inhibited by 90% and 100%.

### Example 14

### ACF64/65

Human ACF64/65 were expressed from a single gene located on chromosome 10 by alternative splicing. The insulin regulated alternative splicing factor SRp40 modulated inclusion of the 5′ most 24 nucleotides of exon 12 thereby producing mRNA encoding ACF65. The study also revealed for that ACF64 and ACF65 had equivalent abilities to support APOBEC-1 dependent apoB mRNA editing. The isolation of rat homologs to ACF64 and ACF65 also identified two additional mRNA variants that encoded proteins named ACF43 and ACF45 based upon their predicted molecular masses. They arose by alternative polyadenylation and splicing of the acf pre-mRNA utilizing an exon not present in the human gene. The alternatively spliced mRNAs encoded the N-terminal two-thirds of ACF64/65 that included three RRMs followed by an amino acid sequence unique to ACF43 or ACF45. ACF43 and ACF45 together comprise at least part of the previously described p44 cluster of apoB RNA binding proteins. ACF43/45 were not proteolytic products of ACF64/65. The subcellular localization of the ACF isoforms was metabolically regulated.

High affinity, peptide specific polyclonal antibodies reactive with the N-terminus of ACF64/65 were produced that reacted with recombinant ACF43/45 and proteins of appropriate size in rat liver nuclear extracts. Western blots and RT-PCR analyses demonstrated that ACF65, ACF64, ACF45 and ACF43 were expressed simultaneously and co-localized in rat liver nuclei. Peptide-specific antibodies were raised also against a C-terminal motif common to ACF64/65 as well as against the eight amino acid motif that distinguishes ACF65 from ACF64. Both reacted with proteins of approximately 65 kDa in rat liver extracts but not with ACF43/45.

It has been demonstrated that ethanol stimulated apoB mRNA synthesis and editing in primary hepatocytes and McArdle cells. Stimulation of editing occurred within 5 minutes, remained elevated for at least 2 hours and was restricted to nuclear apoB mRNA. Ethanol increased editing without cle novo mRNA or protein synthesis and was accompanied by a marked increase in the abundance of nuclear ACF64/65 an observation recapitulated in insulin stimulated primary hepatocytes. The significance of these findings is that auxiliary proteins can be the specific targets of metabolic regulation because their subcellular distribution was altered regardless of whether APOBEC-1 abundance was (insulin) or was not (ethanol) altered. Moreover, pre-existing editing factors were sufficient to support the additional editing activity highlighting the role post-translational processes of phosphorylation and subcellular relocalization may play in regulating editing activity.

RT-PCR demonstrated that acf43/45 mRNA expression was restricted to liver and intestine, the two tissues that express APOBEC-1 and have demonstrable apoB mRNA editing. This contrasted with acf64/65 mRNAs that were expressed in all rat tissues examined and western blot reactivity of the ACF N-terminal specific antibody with appropriately sized proteins in rat primary hepatocytes, McArdle rat hepatoma, HepG2 human hepatoma, CaCo2 human intestinal, COS7 monkey kidney and in HeLa human cervical carcinoma cells. A lower molecular weight protein was observed in CHO Chinese hamster ovary cells. The Drosophila S2 cell was identified as a cell type that only supported apoB mRNA editing when transfected with apoB reporter RNA as well as APOBEC-1 and ACF isoforms.

In the course of establishing a yeast two-hybrid screen for auxiliary proteins a robust APOBEC-1 complementation activity was detected in stationary phase yeast grown in galactose. This resulted in the discovery that the yeast cytidine deaminase, CDD1, could edit ectopically expressed apoB mRNA and provided a valid premise for the modeling of APOBEC-1 on the crystal structure of CDD 1 (Using spatial restraints from known Cytidine Deaminase (CDA) structures, a comparative ‘APOBEC-1 Model’ was generated by the program Modeller. The model shows a structural organization consisting of a catalytic domain attached to a flexible linker that can function as a catalytic gate, followed by a structurally similar, but catalytically inactive pseudo catalytic domain.

The Drosophila S2 cell-based cipoB mRNA editing system demonstrated the APOBEC-1 complementing activity of ACF43/45 in the complete absence of ACF64/65. Both ACF43 and ACF45 complemented APOBEC-1 although not as effectively as ACF64 (ACF64>ACF43>ACF45). The minimal composition of the editosome, in terms of size, was therefore redefined as ACF43 plus APOBEC-1. Yeast two hybrid analyses between APOBEC-1 and the ACF isoforms revealed, based upon histidine prototrophy in the presence of increasing amounts of 3-aminotriazole, an order of interaction strength that was ACF64≧ACF43>>ACF45, consistent with the observed APOBEC-1 complementation activity for each protein. Conditions were established for the expression and purification from E. coli of high levels of soluble ACF variants. Each recombinant ACF isoform bound RNA preferentially to apoB RNA in vitro. RNA binding competition assays demonstrated that ACF45 displaced ACF43 and ACF64 from apoB mRNA whereas ACF43 facilitated ACF64 and ACF45 binding showing that the relative avidity of ACF isoforms for apoB mRNA was ACF45>>ACF64>>ACF43. The biological significance of these data was observed when a titration of ACF isoforms was transfected into McArdle cells or a cell line over-expressing APOBEC-1 that edits apoB mRNA at 60%.

The kinetics of the production of edited apoB mRNA in ethanol treated primary hepatocytes confirmed that apoB mRNA editing was a nuclear event. The nuclear and cytoplasmic localization of endogenous ACF was demonstrated by immunofluorescence in McArdle cells and by immunoelectron microscopy of rat liver. This localization was supported by co-sedimentation of APOBEC-1 and ACF in nuclear 27S and cytoplasmic 60S complexes. These studies established the ‘normal’ distribution of endogenous hepatic ACF.

Studies revealing that apoB mRNA editing occurred coincident with RNA splicing have inexorably linked the two processes but RNA splicing inhibited RNA editing. Splice site suppression of editing diminished as the distance of splice junctions from the editing site was increased. Splice site mutants demonstrated that splicing of the premRNA mediated editing suppression. Editing was completely inhibited when the tripartite editing cassette was located within an intron unless commitment of this RNA to the splicing pathway was bypassed by the inclusion of a cis-acting Rev Responsive Element and co-expression of the HIV Rev protein. Unspliced RNAs were edited and exported from the nucleus.

An amino-terminal 56 amino acids of APOBEC-1 and the M-domain (residues 97-152) were both necessary for APOBEC-1 nuclear import. These determinants only supported nuclear import of APOBEC-1 chimeras of less than 60 kDa. This showed that in liver cells APOBEC-1 can direct its own nuclear import, but is unlikely to be responsible for nuclear import of other editing factors or editosome complexes. The catalytic domain of APOBEC-1 was not required for its subcellular distribution.

The C-terminus of APOBEC-1 (173-196) contains a leucine-rich region (LRR) similar to the Nuclear Export Sequence (NES) of HIV Rev. The LRR exported heterologous proteins containing the SV40 T antigen NLS to the cytoplasm which showed APOBEC-1 nuclear export might be CRM 1-dependent. However, this region of APOBEC-1 did not complement an export defective Rev mutant. In transfected McArdle cells the nuclear and cytoplasmic distribution of tagged APOBEC-1, but not an APOBEC-1-REV chimera, was insensitive to Leptomycin B (LMB). This showed that although the CRM1-dependent pathway was functional in McArdle cells the cytoplasmic localization of APOBEC-1 was mediated by alternative mechanisms.

TAT-mediated protein transduction was developed as a mechanism for the delivery of recombinant APOBEC-1 directly into primary hepatocytes and McArdle cells on a transient basis. An E. coli expressed TAT-APOBEC-CMPK chimera stimulated editing activity in primary hepatocytes in a dose dependent manner within 6 hours and increased secretion of VLDL containing apoB48. The effect was transient and doses of TAT-APOBEC-CMPK could be achieved that stimulated 100% apoB mRNA editing without promiscuous editing.

### Example 15

### ACF Variants

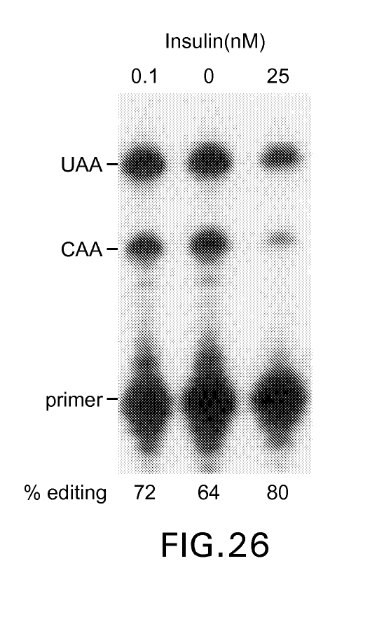

For the purpose of this example, ‘ACF65’, ‘ACF64’, ‘ACF45’ or ‘ACF43’ refers to each protein individually and ‘ACF64/65’ refers to both ACF64 and ACF65 and ‘ACF43/45’ refers to both ACF43 and ACF45. ‘ACF isoforms’ refer to all four of the above proteins. ‘APOBEC-1’ refers to the catalytic subunit but ‘editing factors’ refers to APOBEC-1 plus some or all of the ACF isoforms. The metabolic models: Fasting and refeeding of rats to modulate apoB mRNA editing was recapitulated in rat primary hepatocytes treated with insulin (FIG. 26). Stimulation of hepatic editing was associated with: (i) increased expression of APOBEC-1, (ii) enhanced nuclear abundance of ACF65/64 and (iii) increased abundance of acf65 mRNA relative to acf64 mRNA.

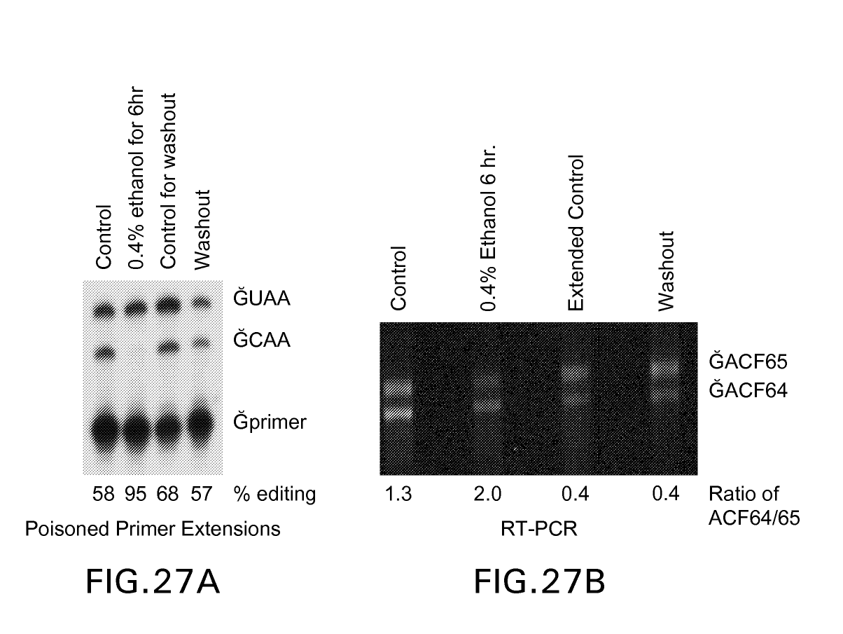

Hepatic apoB mRNA editing increased in ethanol consuming rats as well as in ethanol treated rat primary hepatocytes (FIG. 27A) and McArdle hepatoma cells. Ethanol stimulated editing without increasing APOBEC-1 expression but promoted increased nuclear abundance of ACF64/65. Simultaneous amplification of acf64 and acf65 mRNAs and RT-PCR ratio analysis showed that ethanol treatment increased acf64 mRNA relative to acf65. (FIG. 27B) The effects were reversible following 12 hours of ethanol withdrawal (washout). Taken together these data showed that ethanol and insulin have similar effects on auxiliary protein subcellular localization.

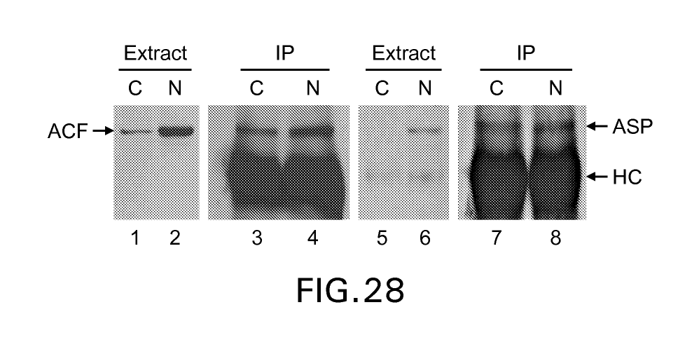

Specific reagents available: The expression of epitope tagged ACF isoforms in mammalian cells was confirmed by western blotting. RNA binding competent ACF isoforms have been purified from E. coli. Peptide specific, rabbit polyclonal antibodies prepared against an N-terminal sequence present in all ACF isoforms (NHKSGDGLSGTQKE (SEQ ID NO: 34)) and the C-tenninus unique to ACF64/65 (HTLQTLGIPTEGGD (SEQ ID NO: 35) were used in the identification of ACF43/45. The utility of the ACF65 specific antibody (peptide specific polyclonal antibody raised against the 8 amino acid insert in ACF65; EIYMNVPVG, SEQ ID NO: 36) is exemplified in FIG. 28 in which ACF64 (ACF; lanes 1&2) and ACF65 (ASP; lanes 5&6) were detected by western blot analysis of 351 g of cytoplasmic (C) or nuclear (N) protein. The data showed that ACF64 was more abundant than ACF65 in both subcellular fractions. Cytoplasmic (320 μg) and nuclear (60 μg) extract proteins were immunoprecipitated (IP'ed) with ACF C-terminal antibody and complexes precipitated with Protein A agarose. IPs were western blotted and reacted with anti-ACF (lanes 3&4) or anti-ASP (lanes 7&8). The data demonstrated efficient recovery of ACF64 and ACF65 from cytoplasmic and nuclear extracts. Primary antibody Ig heavy chain (HC) co-eluted with the antigen because it could not be crosslinked to beads without a loss of activity. ACF45 has a sufficiently long and unique C-tenninus (CSRTPSIYLCFLTAVHAGVHHI HVQ, SEQ ID NO: 37) to serve as an epitope. It has been observed that N-terminally conjugated peptides elicit immunoreactivity to the C-terminal amino acids, showing that a peptide consisting of 9-11 ACF43/45 common amino acids plus the C-terminal ACF43 three unique residues (NIS) can elicit ACF43-specific antibodies.

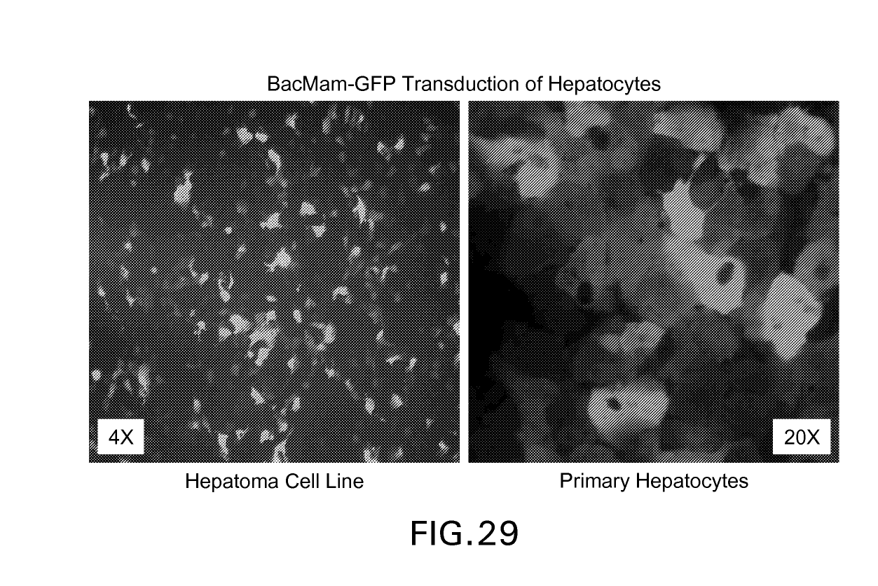

Expression of recombinant proteins and/or RNAI. All ACF isoforms can be expressed and detected in rat hepatoma and Drosophila S2 cells allowing the relative effect of each isoform, either alone or in combination, on RNA editing to be assessed. The metabolic regulation of editing observed in primary rat hepatocytes makes these cells the first choice for these studies but it has been found that primary hepatocytes transfect poorly (≦1%) with conventional lipophilic and non-cationic reagents. Therefore, a modification of baculovirus vector expression technology for high efficiency gene transfer into cultured primary rat hepatocytes can be used. The baculovirus Autographa californica multiple nuclear polyhedris virus (AcMNPV) can infect a wide variety of mammalian cells and when modified vectors carry promoters that initiate transcription efficiently in mammalian cells greater than 90% of cells can be infected and sustain long term (I 5 days) cDNA expression in the absence of cytotoxic or modulatory effects. This allows both ACF isoform expression and metabolic modulation of apoB mRNA editing to be evaluated in the context of primary hepatocytes. FIG. 29 shows HepG2 human hepatoma (A) and human primary hepatocytes (B) expressing high levels of GFP after transduction with BacMam-GFP virus. Replacement of the polyhedron promoter and 3′ RNA processing signals in pFastBac-1 (Invitrogen) with an RNA polymerase III promoter (e.g. U6) is planned for the expression of ACF isoform specific siRNA cassettes.

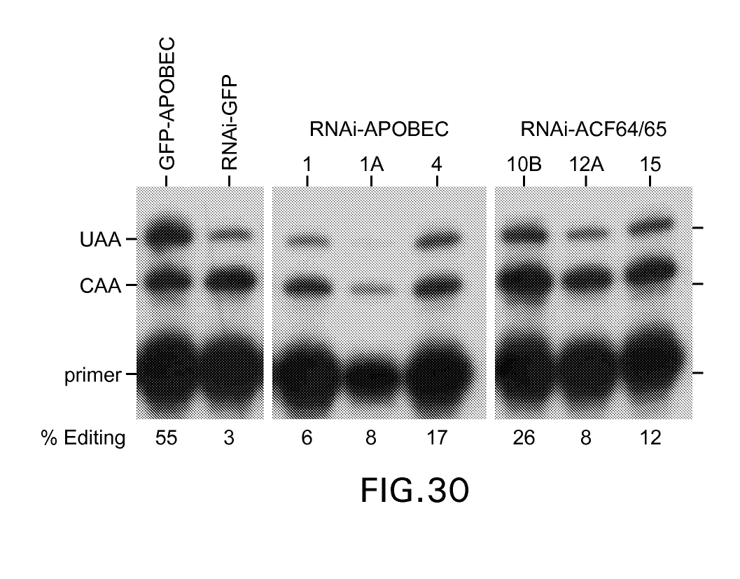

RNAi suppression of protein expression. RNAi has become a powerful methodology for the suppression of expression of specific proteins of interest. To evaluate the requirement of editing factors in the metabolic regulation of editing in hepatic cells, RNAi was used as a means of reducing APOBEC-1 and ACF64 abundance. To screen for the optimal sequences that could be effectively targeted by RNAi, PCR derived short RNAi hairpins were designed against several regions of APOBEC-1, ACF64/65 or GFP and cloned downstream of the human U6 promoter. These expression cassettes were transfected into a McArdle cell line that already stably expressed a GFP-APOBEC-1 chimera and the edits endogenous apoB mRNA at 50%. An RNAi specific to GFP reduced editing nearly 20-fold (FIG. 30), showing that GFP-APOBEC-1 abundance had been reduced. Three different APOBEC-1 specific RNAi hairpins also substantially reduced editing in the GFP-APOBEC stable cell line. As expected when the abundance of an essential auxiliary factor is reduced, three different RNAi hairpins targeted to the C-terminus unique to the ACF64/65 isoforms reduced editing. Several other RNAi hairpins tested were not as effective as those shown. Western blotting and UV crosslinking are performed to confirm RNAi-dependent reduction in protein abundance. RNAi can be used as a means of selectively reducing the abundance of each ACF isoform in transfected McArdle cells and in BacMam U6 vector transduced primary hepatocytes.

PROSITE (http://us.expasy.org/tools/scanprosite) and NetPhos (http://www.cbs.dtu.dk/services/NetPhos/) scans identified numerous high probability Casein Kinase II, Protein Kinase A, Protein Kinase C and CaM kinase IT sites within the N-terminal two-thirds of ACF64/65. The number of predicted sites indicated that inhibitors of protein phosphorylation/dephosphorylation can be useful in determining which protein kinase and/or phosphatase are involved, thereby restricting the number of specific phosphorylation site mutants that need to be created and assayed. Cell permeable and structurally unrelated inhibitors for protein kinases C, A and CaM kinase II (H89, A3 and KN93 respectively) were identified from the extensive database of Calbiochem and literature therein (www.calbiochem.com) and used at 10- to 100-fold below and above the suggested IC₅₀ for hepatocytes. Editing was stimulated in hepatocytes by all of these inhibitors. Myosin light chain kinase and MAP kinasekinase inhibitors (ML-7 and PD98059 respectively) that had no predicted target sites in ACF64/65, did not affect editing. Editing activity was unaffected by cyclosporin A (phosphatase 2B selective inhibitor) but was stimulated by protein phosphatase inhibitors okadaic acid and μM concentrations of endothall, showing a role for protein phosphatase 1.

Rat primary hepatocytes (sixty T-25 flasks) were cultured for 2 hours in phosphate-free media containing basal insulin (0.1 nM) and then pulsed for 4 hours with inorganic ³²P (1200 μCi/flask) in phosphate-free media containing basal, 10 nM insulin or 0.45% ethanol. Cells were harvested in Tris-buffered saline containing protease inhibitors and 50 mM NaF. Cytoplasmic and nuclear extracts were prepared with the NE-PER® system (Pierce) and digested with DNase I, RNase A and RNase T1 to remove labeled nucleic acids. Nuclear 11S and 27S complexes isolated by sedimentation through 10%-50% glycerol gradients contained more ACF64/65 than cyto-plasmic 60S complexes. To determine on a per μg protein basis where ACF64/65 phosphorylation was maximal, subsaturating quantities of ACF C-terminal antibody were used in the immunoprecipitation is. Western blots demonstrated similar recovery of ACF64/65 in gradient fractions from both nuclear (FIG. 16A) and cytoplasmic (FIG. 16B) extracts. Autoradiography of the western blot showed that: (i) only nuclear ACF64/65 was ³²P labeled, (ii) radiolabeled ACF64/65 was recovered primarily in the nuclear 27S editosomes fraction and (iii) insulin and ethanol stimulated the incorporation of ³²P into ACF64/65 recovered in the 27S fraction. Increasing the amount of protein analyzed and the length of autoradiographic exposure did not reveal a ³²P signal from cytoplasmic extracts. The lower molecular weight radiolabeled protein remains to be characterized; it does not correspond to ACF43 or ACF45 as the initial immunoprecipitation was performed with the ACF C-terminal antibody. These results show that phosphlorylation of ACF64/65 is metabolically regulated and that this can be essential for their nuclear distribution and editosome function.

To confirm ³²P incorporation into ACF64/65 protein, and not residual bound undigested ribonucleic acid, the immunoprecipitated radiolabeled ACF64/65 was treated with Calf Intestinal Alkaline Phosphatase (CIAP) (FIG. 1). Furthermore western blotting of 2D PAGE with the ACF N-terminal antibody revealed an acidic isoform of ACF64/65 (FIG. 1, ‘Control’ within the red box) whose abundance was reduced by CIAP and whose alkali mobility shift (˜0.4 pH units) was consistent with the loss of 2-3 phosphates (Mr.; migration of cellular ACF for size comparison). Phosphoserine and phosphothreonine specific antibodies (Zymed) (FIG. 31), but not anti-phosphotyrosine were reactive with phosphorylated ACF further indicating that ACF64/65 is a phosphoprotein.

The presence of phosphorylated ACF in nuclear 27S active editosomes and not 1 IS or 60S complexes nor cytoplasmic extracts (FIG. 16) shows phosphorylation was significant for RNA binding, for interaction with other editing factors or the deamination reaction. CIAP treatment of rat liver nuclear extract did not inhibit UV cross-linking of apoB RNA to p66 and p44; in fact phosphorimager quantification indicated that the yield of p66 increased by 40% compared to control with the highest CIAP treatment. This indicates that apoB RNA binding need not be dependent on ACF phosphorylation. To further investigate the role of phosphorylation, rat liver nuclear extract was treated with CIAP and assayed for in vitro apoB RNA editing activity. The data demonstrated that in vitro editing activity was inhibited by CIAP treatment, showing that phosphorylation of one or more editing factors was required for their function. Phosphorylation can play a role in the subcellular localization and/or stability of ACF. Cumulative evidence strongly indicates that ACF associated with active nuclear editosomes is a phosphoprotein. 5 pmoles (320 ng for ACF64) of radiolabeled protein containing 5000 Cerenkov cpm were necessary for the identification of phosphotryptic peptides and hence specific phosphorylation sites, by MALDI-TOF mass spectrometry. These yields were achieved by treatment of rat primary hepatocytes (40 T25 flasks) with 0.45% ethanol for four hours in the presence of 70 mCi of total inorganic ³²P followed by immunoprecipitation of ACF64/65 (FIG. 32). Two IPs were resolved by PAGE (FIG. 32, Coomassie) and ACF64/65 identified (western). Comparison to mass standards (Mr, BioRad Precision Plus) of 100 kDa and 50 kDa (representing 150 ng and 750 ng of protein respectively) indicated that ≧400 ng of ACF64/65 (total of >5000 Cerenkov cpmns) were obtained.

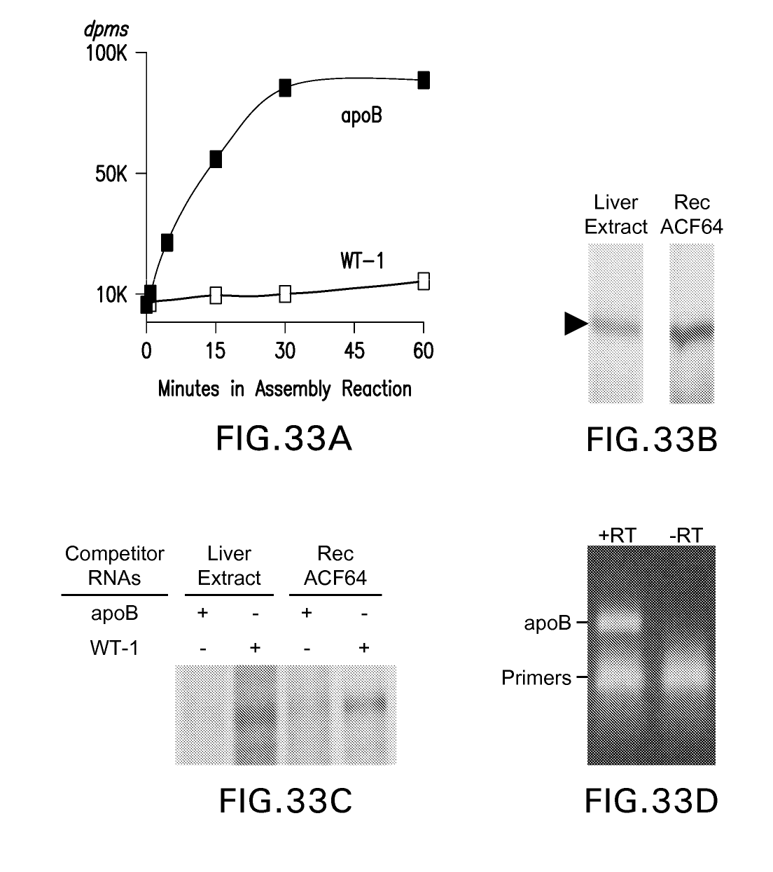

Mapping of the nucleotides involved in apoB RNA binding and determination of alterations in the ‘footprint’ due to competition between ACF variants are conducted. Editosome assembly reactions are performed with site-specifically labeled apoB RNA and various combinations of purified recombinant ACF isoforms. IP of ACF isoform-RNA complexes are necessary to permit the recovery of only those RNAs that were bound to ACF isoforms and mapping of each footprint. In piloting these IP studies FIG. 33A shows the specific recovery of a radiolabeled apoB transcript, but not of RNA that lacked the mooring sequence (WT-1), from an in vitro editosome assembly by IP with ACF64 antibody. Western blotting with the ACF N-terminal antibody demonstrated good recovery of the endogenous ACF64 from liver extract or recombinant ACF64 (47B). The recovery of endogenous or recombinant ACF64 crosslinked to radiolabeled apoB RNA was virtually eliminated by 100-fold molar excess of competing cold apoB RNA but was not affected by competing WT-1 RNA (47C). The assembly reactions (A and C) used 50 fmols of radiolabeled apoB RNA, detected readily by RT-PCR (-RT, minus reverse transcriptase control) (47D). These data demonstrated IP RNA yields sufficient for subsequent enzyme based analyses. Optimization for the amount of protein, RNA and antibody was performed.

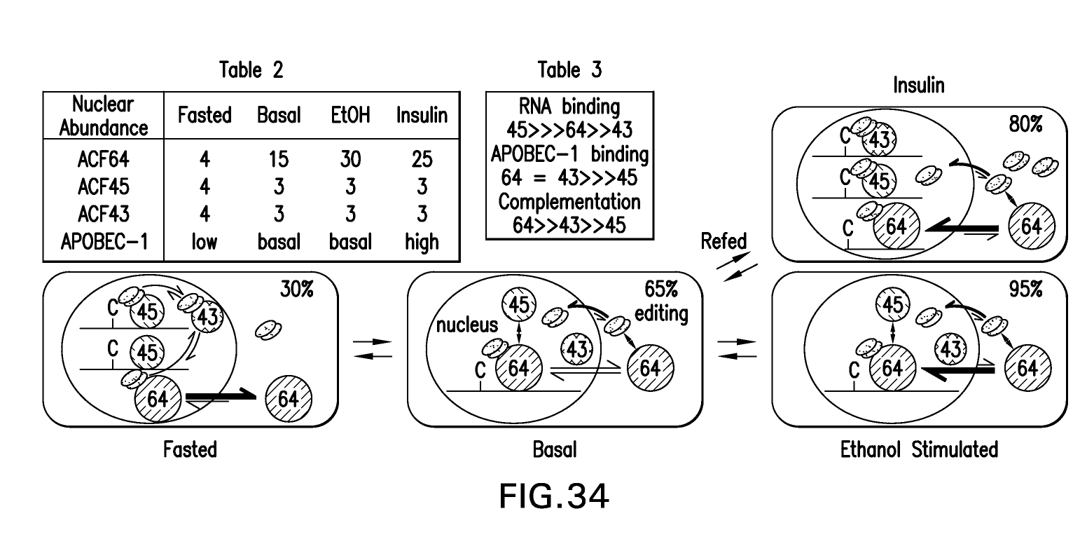

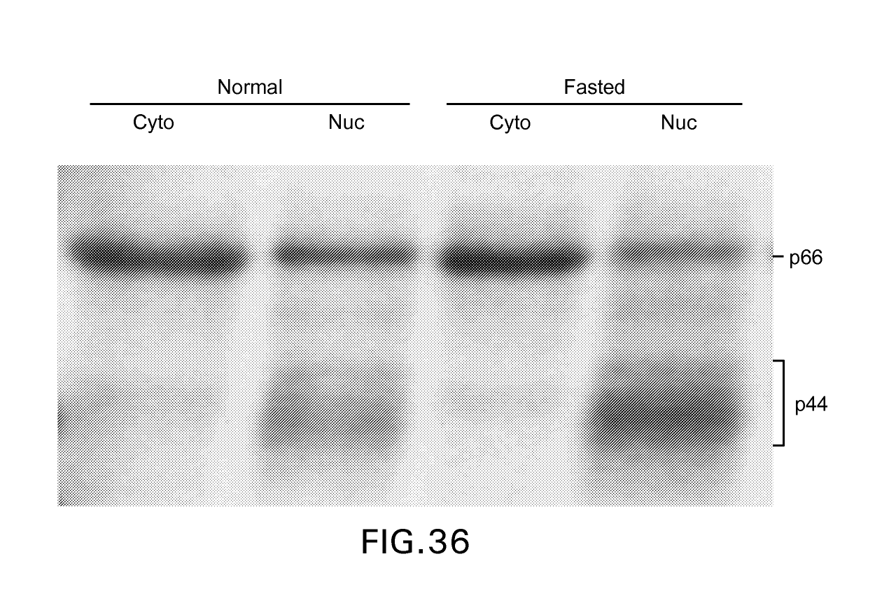

Co-expression of ACF isoforms with different functional properties showed that ACF isoforms are interactive components of a regulatory network controlling the amount of edited apoB mRNA. A cartoon of this model (FIG. 34) depicts four metabolic states in rat hepatocytes: (i) basal (˜0.1% insulin and ˜65% editing) (ii) fasted (<0.1% insulin and ˜30% editing) (iii) 10-25 nM insulin, high sucrose refed (˜80% editing) and (iv)>0.1% ethanol treated (˜95% editing). FIG. 34 lists the relative abundance of editing factors in arbitrary units estimated from scanning densitometry of western blots, and for APOBEC-1, alterations in mRNA expression levels. FIG. 34 also lists the relative APOBEC-1 binding, apoB UV crosslinking activities and complementation activities of each ACF isoform (FIG. 36). ACF64 and ACF65 have equal complementation activity when expressed at equivalent levels so for simplicity, ACF64/65 are shown as ACF64. In the model, a reduction in nuclear abundance of ACF64 (fasted or ethanol withdrawal) promotes editosome disassembly because ACF45 effectively competed to displace ACF-APOBEC-1 from apoB mRNA with facilitation from ACF43. ACF45 could only weakly complement editing because ACF43 could effectively compete with ACF45/64 for rate limiting quantities of APOBEC-1. ACF43-APOPBEC-1 complexes could however not efficiently bind to apoB mRNA in the presence of ACF45/64. In contrast, the model shows that an increase in nuclear abundance of ACF64 (ethanol or insulin) can shift the binding equilibrium to ACF64-APOBEC-1 complexes and hence promote editing. Insulin stimulated APOBEC-1 expression, enabling ACF43/45 to contribute to the overall editing efficiency by forming functional complexes with APOBEC-1 in addition to facilitating ACF64-APOBEC-1 turnover.

Co-expression of ACF isoforms with different functional properties can be essential for recycling editing factors to nascent apoB mRNAs following each catalytic event (editosome turnover). Given ACF64's high affinity for APOBEC-1 (which exceeded that of SV40T antigen interaction with p53 in yeast two hybrid analysis) and its strong binding to apoB mRNA (Kd-8 nM), editosomes can facilitate disassembly to carryout additional rounds of editing. The model also shows that metabolic stimulation of hepatocyte apoB mRNA editing can be blunted or inhibited if cells lacked ACF43/45 because ACF64/65-APOBEC-1 complexes have an impaired capacity to disengage and assemble onto nascent unedited apoB mRNA substrates (a Km affect).

Arrows between hepatocytes signify the reversibility of each metabolic state. Within each cell, nuclear abundance of ACF64 is determined by nucleocytoplasmic trafficking that is indicated by arrows crossing the nucleus. The length and thickness of each error signifies the steady state distribution. The tenet of the model is that alterations in the steady state nuclear abundance of ACF isoforms are driven by phosphorylation. The model does not exclude the possibility that ACF43/45 are phosphorylated and traffic. ACF isoform interactions with APOBEC-1 that can lead to co-import into the nucleus from the cytoplasm are indicated by arrows. Arrows are used to indicate ACF43/45 interactions with ACF64-APOBEC-1 complexes and apoB mRNA in net editosome disassembly (fasted) or editosome recycling (basal and ethanol).

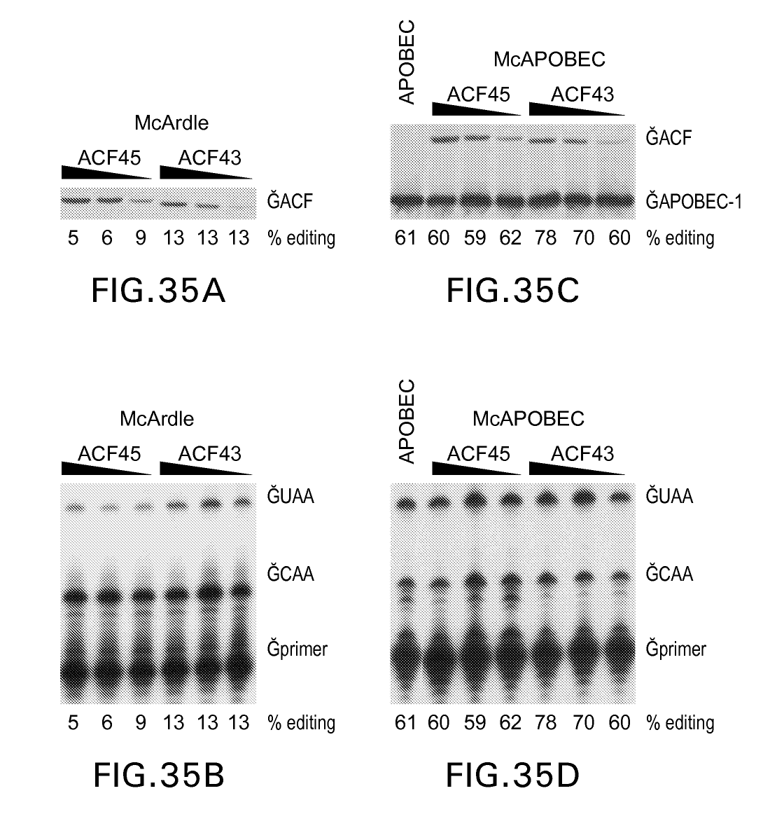

The overall expression and subcellular localization of epitope tagged ACF isoforms is established by western blotting of nuclear and cytoplasmic extracts (prepared using NE-PER system; Pierce). These biochemical studies are complemented by immunofluorescence microscopy of paraformaldehyde fixed cells reacted with epitope tag or ACF isoform-specific antibodies. In pilot experiments varying levels of HA-tagged ACF43 and ACF45 were expressed by transfecting differing amounts of cDNA into McArdle or McAPOBEC cells (FIGS. 35A & C, western blots). The level of exogenously expressed HA-tagged APOBEC-1 in McAPOBEC cells was high and unaffected by ACF isoform expression (FIG. 35C). Sufficient endogenous ACF was expressed in McAPOBEC cells to support 61% editing (FIG. 35D, poisoned primer extension) given the level of APOBEC-1 overexpression (FIG. 35C). ACF43 expression in McArdle cells did not affect editing whereas ACF45 inhibited editing over two-fold (FIG. 35B, poisoned primer extension). In contrast, ACF43 stimulated editing activity in McAPOBEC cells while ACF45 had no affect (FIG. 35D). These studies show that changes in the relative abundance of exogenous ACF43/45 isoforms can itself modulate editing activity in the presence of constant amounts of endogenous ACF64/65 and that this relationship will be affected by changes in the abundance of APOBEC-1. The data showed that inhibition of editing by overexpressing ACF45 only occurs when APOBEC-1 is rate limiting (analogous to FIG. 34, basal and fasted/washout). ACF43's facilitation of ACF64/65 binding to apoB mRNA enhanced editing activity only when APOBEC-1 abundance was not rate limiting (analogous to FIG. 34, insulin). The above transient transfection scenarios can be recapitulated in ethanol treated cells to evaluate the affect of ACF isoform abundance on cells ability to respond to metabolic perturbation. Ethanol treatment (0.45% for 2 to 12 hours) of McArdle and McAPOBEC can be used as this treatment stimulated editing activity and was not toxic. ACF expression levels, subcellular localization and editing activity are determined from replicate experiments and the relative affects of overexpressing each isoform compared in the absence (negative control) or presence of ethanol and to the response seen in wild type cells (positive control).

TABLE 2

| | Mean ± SEM |
|---|---|
| Okadaic acid | |
| control | 66 ± 2 |
| DMSO | 68 ± 2 |
| 100 nM | 91 ± 2 |
| 10 nM | 74 ± 4 |
| 1 nM | 72 ± 4 |
| IC₅₀PP1 = 10-15 nM; IC₅₀PP2A = 0.1 nM | |
| Endothall | |
| control | 65 ± 2 |
| DMSO | 70 ± 2 |
| 50 μM | 83 ± 2 |
| 5 μM | 64 ± 3 |
| 500 nM | 65 ± 2 |
| 100 nM | 66 ± 2 |
| IC₅₀PP2A = 90 nM; IC₅₀PP1 = 5 μM | |
| Cantharidin | |
| control | 66 ± 2 |
| DMSO | 65 ± 1 |
| 4.7 μM | 91 ± 1 |
| 470 nM | 75 ± 1 |
| 47 nM | 64 ± 1 |
| 4.7 nM | 62 ± 2 |
| IC₅₀PP2A = 40 nM; IC₅₀PP1 = 473 nM | |
| Cyclosporin A | |
| control | 66 ± 4 |
| DMSO | 68 ± 2 |
| 500 nM | 70 ± 4 |
| 50 nM | 66 ± 2 |
| 5 nM | 63 ± 3 |
| IC₅₀PP2B = “nM” | |
| Cypermethrin | |
| control | 65 ± 4 |
| DMSO | 63 ± 4 |
| 5 nM | 66 ± 4 |
| 50 pM | 69 ± 4 |

IC₅₀PP2B = 40 pM

TABLE 3
Effect of PKC Activators on Rat Primary Hepatocytes

| Indolactam V | Mean | STD | n | PMA | Mean | STD | n |
|---|---|---|---|---|---|---|---|
| Primary | 66.11 | 1.9 | 3 | Primary | 65.5 | 2.8 | 4 |
| hepatocytes | | | | hepatocytes | | | |
| DMSO | 75.62 | 0.8 | 3 | DMSO | 69.1 | 6.4 | 4 |
| 12 μM | 92.47 | 0.8 | 3 | 500 nM | 68.8 | 6 | 4 |
| 120 μM | 91.57 | 1.1 | 3 | 5 μM | 64.9 | 8.1 | 4 |
| | | | | 50 μM | 63 | 6 | 6 |

| | |
|---|---|
| PKC EC₅₀ = μM | PKC EC₅₀ = 200 nM-100 μM |

Effect of PKA Activators on Rat Primary Hepatocytes

| 8-cpt cAMP | Mean | STD | n | Forskolin | Mean | STD | n |
|---|---|---|---|---|---|---|---|
| Primary | 58.8 | 4.8 | 4 | Primary | 57.6 | 3.4 | 7 |
| hepatocytes | | | | hepatocytes | | | |
| DMSO | 58.2 | 4.6 | 4 | DMSO | 61.8 | 4.1 | 6 |
| 2 μM | 61.2 | 4.3 | 4 | 4 μM | 70.6 | 5.2 | 6 |
| 20 μM | 59.3 | 2.4 | 2 | 40 μM | 68.5 | 7.5 | 4 |
| 200 μM | 59.6 | 3.4 | 4 | 120 μM | 69 | 0.8 | 4 |
| EC₅₀ PKA = 2 μM | | | | AC EC₅₀ = 4 μM | | | |
| Glucagon | Mean | STD | n | Isoproterenol | Mean | STD | n |
| Primary | 61.9 | 0.9 | 4 | Primary | 63.2 | 6.2 | 4 |
| hepatocytes | | | | hepatocytes | | | |
| 5% acetic | 64.3 | 4.4 | 4 | DMSO | 67.4 | 7.8 | 4 |
| acid | | | | | | | |
| 1 nM | 62.2 | 0.9 | 4 | 1 μM | 60.6 | 1.9 | 2 |
| 10 nM | 59.5 | 1.4 | 2 | 10 μM | 68.1 | 1.7 | 4 |
| 100 nM | 58.3 | 3.9 | 4 | 100 μM | 72.3 | 2.9 | 4 |

| | |
|---|---|
| AC EC₅₀ = 0.1-100 nM | AC EC₅₀ = 1 nM-100 μM |

TABLE 4

| Sample | ApoB [ ] (ng) Run 1 | ApoB [ ] (ng) Run 2 | ApoB [ ] (ng) AVG. |
|---|---|---|---|
| NS | 814 | 1036 | 925 |
| W2 | 624 | 795 | 709 |
| H3 | 700 | 596 | 648 |
| H2 | 514 | 653 | 584 |

### Example 16

### ACF Regulation of apoB mRNA

Fatty liver disease (hepatosteatosis) is due to the accumulation of fatty infiltrates in the liver and are characteristic of the hyperinsulinemic and insulin-resistant state. The accumulation of lipid in the liver can arise from increased hepatic uptake of fatty acids due to elevated lipolysis in adipose tissue and a suppressed ability of the liver to clear lipid by processing lipids into lipoproteins and secreting them into the circulation. Progression to liver cirrhosis (steatohepatitis) can involve enhanced susceptibility to oxidative stress, suppressed immune response and liver inflammation. This can be compounded by cyclic patterns of rapid weight loss/weight gain and long term or morbid obesity seen increasingly in cultures consuming western diets. This is because excess lipid released from adipose tissue and dietary lipid can acutely or chronically exceed the liver's metabolic capacity and cause the accumulation of lipid droplets in the liver. During fatty acid oxidative catabolism, peroxides are produced which cause liver damage through oxidative stress. Obesity induced, insulin-resistance and type II diabetes exacerbate both the metabolic and physiological stress.

In fact, 14% to 24% of the US population suffers from obesity. Fatty liver disease is being observed in children and young adults (Steinberger, J., and Daniels, S. R. “Obesity, insulin resistance, diabetes, and cardiovascular risk in children: an American Heart Association scientific statement from the Atherosclerosis, Hypertension, and Obesity in the Young Committee (Council on Cardiovascular Disease in the Young) and the Diabetes Committee (Council on Nutrition, Physical Activity, and Metabolism). Circulation 107:1448-53 (2003). High fat content and high fructose sweeteners in the western diet are thought to contribute to the insulin resistant phenotype 9 (Bray, G. A. et al. Am J Clin Nutr 79:537-43 (2004); Heini, A. F., and Weinsier, R. L. “Am J Med 102:259-64 (1997)). Frequently patients with this condition progress from simple hepatic steatosis to cirrhosis and require liver transplantation (Browning, J. D., and Horton, J. D. J Clin Invest 114:147-52 (2004); Festi, D., et al. Obes Rev 5:27-42 (2004)).

Therapeutic intervention to mobilize lipid from fatty livers in humans induces serum hyperlipidemia, ultimately raising serum levels of low density lipoprotein particles or LDL and increasing the risk of development of cardiovascular diseases. Changes in dietary habits and the identification of cellular pathways and factors that determine hepatic lipid clearance and serum lipoprotein metabolism can be used as therapeutic targets for the prevention and treatment of fatty liver disease. As disclosed herein, obesity-induced, insulin-resistance impairs a regulatory process in which ACF phosphorylation normally regulates lipid clearance in the liver through its ability to bind and retain apoB mRNA in the nucleus and thereby modulate the availability of cytoplasmic apoB mRNA for translation during VLDL assembly and secretion.

ACF regulation of apoB mRNA APOBEC-1 Complementation Factor (ACF) trafficks between the cytoplasm and nucleus (Blanc, V. et al. J Biol. Chem. (2003); Chester, A. et al. “Embo J22:3971-82 (2003); Sowden, M. P. et al. J Cell Science 115:027-1039 (2002)) and accumulates in the nucleus in response to insulin or ethanol stimulation. Nuclear accumulation of ACF is regulated by insulin-dependent serine phosphorylation and ACF export to the cytoplasm can be regulated through dephosphorylation by protein phosphatase I (Lehmann, D. M. et al. Nucleic Acids Res 34:3299-308 (2006)). ACF binds to apoB mRNA with high affinity and specificity for the 11 nt mooring sequence within exon 26 (Mehta, A., Driscoll, D. M. RNA 8:69-82 (2002)). This interaction is independent of a C or U at the editing site, the subcellular source of ACF (Sowden et al. (2002); Harris et al. (1993); Sowden, M. P. et al. J. Biol. Chem. 278, 197-206 (2004)) or its phosphorylation (Lehmann, D. et al. (2006); Mehta, A. et al. (2002), Blanc, V. et al. J Biol Chem 276:46386-93 (2001); Driscoll, D. M., et al. Mol Cell Biol 13:7288-94 (1993); Galloway, C. A. et al. Biotechniques 34:524-6, 528-530 (2003); Mehta, A. et al. J Biol Chem 271:28294-9 (1996)). ACF is bound to the mooring sequence of apoB mRNA in polysomes (Chen, X et al. J Biol Chem 268:21007-13 (1993)) and protects edited apoB mRNA from Nonsense Codon Mediated Decay in the cytoplasm (Chester, A. et al. (2003)). ACF trafficking to the nucleus does not require the editing enzyme APOBEC-1 as data demonstrate nuclear accumulation of ACF in apobec-1 knockout mice. Trafficking of ACF has therefore retained its association with apoB mRNA during nuclear export. Alterations in ACF nuclear export due to its phosphorylation is mechanistically important for controlling the amount of apoB mRNA available in the cytoplasm for translation. Metabolic perturbations that modulate this process can have quantitative effects on hepatic assembly and secretion of VLDL. In fact, it has been suggested that enhanced hepatic ACF phosphorylation and nuclear retention is associated with reduced intracellular and secreted ApoB. Dysregulation of ACF phosphorylation leads to enhanced apoB mRNA nuclear export to the cytoplasm contributing to the well documented hepatic overproduction and secretion of VLDL that gives rise to dyslipidemia in the insulin resistant state.

ACF was Discovered through its Role in apoB mRNA Editing.

ACF was first described as an RNA binding protein that selectively bound to the mooring sequence 3′ of the edited site in apolipoprotein B (apoB) mRNA (Harris, S. G. et al. J. Biol. Chem. 268:7382-92 (1993); Shah, R. et al. J Biol Chem 266:16301-4 (1991)). The apolipoprotein B (ApoB) protein is a non-exchangeable structural component of intestinally derived chylomicrons and of liver derived VLDL. ApoB is translated from a 14 kb mRNA that is transcribed from a single copy gene located on human chromosome (Bray, G. A., et al. (2004); Scott, J. Mol Biol Med 6:65-80 (1989)). Large (ApoB100) and small (ApoB48) isoforms of ApoB lipoprotein were known for several years before post-transcriptional RNA editing was discovered as the means for their expression (Chen, S. H. et al. Science 238:363-6 (1987); Powell, L. M. et al. Cell 50:831-40 (1987)). ApoB mRNA editing is one example of several forms of mammalian mRNA editing (Wedekind, J. E. et al. Trends Genet. 19: 207-16 (2003)) and involves a site-specific hydrolytic deamination of cytidine at nucleotide 6666 (C6666) to form uridine, thereby creating an in-frame translation stop codon, UAA, from a glutamine codon, CAA (Chen, S. H., et al. (1987)) Powell, L. M., et al. (1987)). The mammalian small intestine constitutively edits >85% of apoB mRNA and stores ApoB48 for the assembly and secretion of chylomicrons containing dietary lipids (Chen, S. H., et al. (1987)) Powell, L. M., et al. (1987)) Backus, J. W. et al. Biochem Biophys Res Commun 170:513-8 (1990); Greeve, J., et al. J Lipid Res 34:1367-83 (1993)). In several mammals, with the important exception of humans (Fujino, T. et al. Genomics 47: 266-75 (1998)), apoB mRNA editing also occurs in liver (Greeve, J. et al. (1993)). Rodent liver regulates apoB mRNA editing thereby modulating the proportion of edited apoB mRNA and the proportion of B48 versus B100 secreted as VLDL (Greeve, J., et al.(1993); Sparks, C. E. et al. Can J Biochem 59:693-9 (1981)). In contrast to intestine, hepatic ApoB protein is not stored and VLDL are assembled co-translationally with ApoB48 or ApoB100 and are secreted into the blood from where they are cleared by peripheral tissues and the liver (Shelness, G. S., and Ledford, A. S. Curr Opin Lipidol 16, 325-32 (2005)).

Significantly, apoB mRNA editing in mammalian liver reduces the VLDL+LDL to HDL ratio (Greeve, J. et al. (1993), a condition associated with reduced atherogenesis. B100 VLDL are converted by peripheral lipases to low density lipoprotein particles (LDL) (an atherogenic risk factor (Corsetti, J. P. et al. Athersclerosis 171: In Press (2003)), whereas B48 VLDL lacking the LDL receptor binding domain are cleared more rapidly via chylomicron remnant or apoE surface receptors before conversion to LDL. Hence the mechanism and factors involved in hepatic editing became of interest a potential therapeutic target for reducing the risk of atherosclerosis (Scott, J. (1989); Shelness, G. S, and Ledfored, A. S. (2005); Sparks, C. E., and Marsh, J. B. J Lipid Res 22:519-27” (1981)).

The catalytic subunit that carries out C to U apoB mRNA editing is APOBEC-1 which is a cytidine deaminase that is uniquely required for apoB mRNA editing. The enzyme alone cannot edit apoB mRNA unless expressed in an appropriate cell line (expressing ACF) or complemented in vitro by cell extracts (Hadjiagapiou, C. et al. Nucleic Acids Res 22:1874-9 (1994); Lau, P. P. et al. Proc. Natl. Acad. Sci. USA 91:8522-8526 (1994); Nakamuta, M. et al. J Biol Chem 270:13042-56 (1995); Teng, B. et al. J. Biol. Chem. 269:29395-29404 (1994); Teng, B. et al. Science 260:1816-1819 (1993); Yamanaka, S et al.; J Biol Chem 269:21725-34 (1994). Molecular cloning of ACF demonstrated that ACF was necessary and sufficient to complement APOBEC-1 (Lellek, H. et al. J Biol Chem 275:19848-56 (2000); Mehta, A. et al. Mol Cell Biol 20:1846-54 (2000)). Hence the current understanding of the minimal editosome is APOBEC-1 binds to ACF and is thereby targeted to edit C6666 by the interaction of ACF with the mooring sequence (Sowden, M. P. et al. (2002). Several alternatively spliced variants of ACF are expressed in human and rodent tissues (Sowden, M. P. et al. (2004); Blanc, V. et al. (2001); Blanc, V. et al. Mol Cell Biol 25:7260-9 (2005); Dance, G. S. C. et al. J. Biol. Chem. 277:12703-12709 (2002); Dur, S. et al. Biochim Biophys Acta 1680:11-23 (2004). The 64 kDa form of ACF (referred to throughout this proposal as ACF) has been most extensively studied as it is expressed ≧10-fold higher abundance than the other variants. All variants studied to date have equivalent or less complementation and apoB mRNA binding activity compared to ACF64.

What is the cellular regulation of ACF?

ACF is distributed in the cytoplasm (where it localizes to the exterior of the endoplasmic reticulum) and the cell nucleus (the site of apoB mRNA editing) (Blanc, V. et al. (2003); Chester, A. et al. (2003); Sowden, M. P. (2002); Sowden, M. P. et al. (2004); Yang, Y. et al. J Biol Chem 275:22663-9 (2000)). Metabolic studies in rats and rat primary hepatocytes demonstrated that the proportion of cellular ACF in the nucleus increased when apoB mRNA editing was stimulated by ethanol or insulin and returned to basal, predominantly cytoplasmic distribution, when the stimuli were withdrawn (Sowden, P. et al. (2002); Yang, Y. et al. (2000)). A unique nuclear localization signal in ACF and its interaction with transportin-2 (Blanc, V. (2003)) enables ACF to traffick between the cytoplasm and nucleus (Blanc, V. et al. (2003); Chester, A. et al. (2003)). Insulin (and ethanol) promote retention of ACF in the nucleus and a stable interaction with APOBEC-1 through regulated serine phosphorylation (Lehmann, D. M. et al. (2006); ) Lehmann, D. M. et al. Biochim Biophys Acta 1773:408-18 (2007)). Protein phosphatase 1 activity has been implicated in ACF dephosphorylation, the disassembly of nuclear editosomes and ACF export. While researchers agree that ACF and APOBEC-1 are trafficking proteins, it is not clear what governs APOBEC-1 trafficking. The majority of evidence suggests that APOBEC-1 requires ACF for its nuclear localization (Blanc, V. et al. (2003); Lehmann, D. M. et al. (2006); Lehmann, D. M. et al. (2007); Yang, Y. et al. Exp. Cell Res. 267:153-164 (2001); Yang, Y., and Smith, H. C. Proc Natl Acad Sci USA 94:13075-80 (1997)). Insulin-dependent ACF phosphorylation and nuclear retention have been observed in human primary hepatocytes and apobec-1 knockout mice showing that APOBEC-1 is not required for ACF trafficking to the nucleus and also that ACF trafficking is evolutionarily conserved.

Mouse knockout studies demonstrated that ACF is an essential gene, affecting embryo viability at the preimplantation stage (Blanc, V. et al. (2005)). In the same study, RNAi knockdown of ACF in McArdle rat hepatoma cells to 70% of control cell levels resulted in apoptosis. These findings were somewhat unanticipated because although apoB itself is an essential gene whose gene product is required for lipoprotein assembly and transport within the embryo yolk sack endoderm as well as in adult tissues (Farese, R. V., Jr. et al. Proc Natl Acad Sci USA 92:1774-8 (1995); Veniant, M. M. et al. J Nutr 129:451 S-455S (1999), APOBEC-1 is not an essential gene (Fujino, T. et al. 1998); Nakamuta, M. et al. J. Biol. Chem. 271:25981-25988 (1996); ) Xie, Y. et al. Am J Physiol Gastrointest Liver Physiol 285:G735-46 (2003)). Apobec-1−/− knockout mice expressed only ApoB100 in their intestine and liver resulting in elevated serum LDL levels, low hepatic triglyceride secretion rates and larger lipoprotein size (Nakamuta, M. et al. (1996), Hirano, K. I. et al. J. Biol. Chem. 271:9887-9890 (1996); ) Xu, Y. Cell Death Differ 10:400-3 (2003). Control ratios of B48:B100 in serum VLDL and the proportion of serum VLDL and LDL lipoproteins was restored by apobec-1 gene transfer without adverse side effects (Teng, B. et al (1994) Nakamuta, M. et al. (1996); Hughes, S. D. et al. Hum Gene Ther 7:39-49 (1996); Qian, X. et al. Arterioscler Thromb Vase Biol 18:1013-20 (1998)). A function other than APOBEC-1 complementation has been suspected given that human liver does not express APOBEC-1 but has maintained ACF expression (Fujino, T. et al. (1998); Dance, G. S. C. et al. (2002)) along with its nuclear-cytoplasmic trafficking and phosphorylation.

Is Lipoprotein Metabolism Regulated by Insulin?

Obesity-induced insulin-resistance leads to hyperinsulinemic state of type 2 diabetes, lipid dysregulation, fatty liver disease (hepatosteatosis) and liver cirrhosis. ApoB metabolism and the regulation of VLDL synthesis and secretion by insulin have been reviewed (Adiels, M. et al. Curr Opin Lipidol 17:238-246 (2006); Sparks, J. D., and Sparks, C. E. Biochim Biophys Acta 1215:9-32 (1994)). Following a meal, insulin is rapidly released into the portal vein in response to rising serum glucose followed by a progressive increase in insulin secretion until serum glucose levels fall. The hepatic response to first phase insulin release plays an important role in conditioning liver enzymes for carbohydrate and lipid homeostasis. In rat primary hepatocytes acute insulin stimulation inhibits VLDL triglyceride (TG) secretion in an insulin receptor-dependent manner (Durrington, P. N. et al. J Clin Invest 70:63-73 (1982); ) Patsch, W. et al. J Biol Chem 261:9603-6 (1986)). Studies by Patsch et al. demonstrated that insulin inhibited not only TG secretion, but also ApoB secretion. High levels of insulin reduce hepatic lipoprotein secretion during periods of peak intestinal fat absorption and formation of intestinal chylomicrons (CMs), making the liver a temporary storage site for newly synthesized lipids (Duerden, J. M. et al. Biochem J 272:583-7 (1990); Gibbons, G. F., and Burnham, F. J. Biochem J 275 (Pt 1):87-92 (1991)). The first-phase of insulin release is hypothesized to signal the ‘fed state’ to the liver, interrupting hepatic gluconeogenesis and stimulating glycogen storage from incoming nutrients. Overall, the role of short-term, high levels of insulin represents a regulatory mechanism that modulates levels of plasma TG during periods of increased hepatic lipogenesis (Adiels, M. et al. (2006); Durrington, P. N. et al. (1982); Duerden, J. M. et al. (1990)).

Insulin-resistance is characterized by aberrations in the insulin signaling pathway leading to depressed glucose uptake, elevated levels of serum triglycerides that are due to hepatic VLDL over production, low HDL levels, elevated small dense LDL and dysregulation of a host of enzymes involved in intermediary metabolism (Adeli, K. et al. Trends Cardiovasc Med 11:170-6 (2001; Carpentier, A. et al. J Biol Chem 277:28795-802 (2002); Ginsberg, H. N. J Clin Invest 106:453-8 (2000); Lewis, G. F. et al. Endocr Rev 23:201-29 ((2002)); Sparks, J. D. et al. Biochem J261:83-8 (1989)). Insulin resistance in liver, and the associated failure of insulin to reduce the production and secretion of VLDL, is promoted by the increased release of free fatty acids in peripheral and abdominal adipose tissue due to dysregulation of lipolysis and esterification (Carpentier, A. et al. (2002); Ginsberg, H. N. (2000); Lewis, G. F. et al. (2002); Lewis, G. F., and Steiner, G. Diabetes Metab Rev 12:37-56 (1996). Hormones and cytokines (adipokines) produced by adipose tissue suppress insulin signaling in liver (Klover, P. J. et al. Endocrinology 146:3417-27 (2005); Klover, P. J. et al. Diabetes 52:2784-9 (2003)). Elevated IL6 production in insulin-resistant animals induces hepatic expression of the Suppressor of Cytokine Signaling protein 3 (SOCS3) that in turn inhibits the autophosphorylation of the insulin receptor and dependent signaling pathways (Senn, J. J. et al. J Biol Chem 278, 13740-6 (2003)). Insulin-resistance increases hepatic Protein Tyrosine Phosphatase IB activity that also dephosphorylates the insulin receptor (Carpentier, A. et al. (2002)). Insulin binding to its cognate receptor tyrosine kinase results in the rapid stimulation of multiple serine/threonine protein kinases for example PKC, GSK3β, PI-dependent protein kinase, PI3K, B/AKT, MAPK, p90RSK and p70 S6 kinase (Czech, M. P. et al. J Biol Chem 263: 11017-20 (1988); Werner, E. D. et al. J Biol Chem 279:35298-305 (2004)). Protein phosphatases are also activated by insulin (Shi, K., et al. J Biochem (Tokyo) 136:89-96 (2004); Ugi, S. et al. Mol Cell Biol 24: 8778-89 (2004)). These facts indicate a complex pattern in which site-specific phosphorylation or dephosphorylation is finely orchestrated to regulate cell signaling, gene expression and protein function. Phosphorylation of proteins can either activate or inactivate these processes and frequently proteins are phosphorylated at multiple sites (hyper-phosphorylated), revealing that many exist in a range of hyper- or hypo-phosphorylated states.

Over production of hepatic VLDL in the insulin resistant state can also be attributed to dysregulation of the insulin receptor signaling-dependent transcriptional control of the Microsomal Triglyceride transfer Protein (MTP) gene (Carpentier, A. et al. (2002)). Acute insulin receptor signaling leads to transcriptional suppression of the MTP promoter (Lin, M. C. et al. J Lipid Res 36:1073-81 (1995)) and a reduction in intrahepatic and secreted ApoB (Chirieac, D. V. et al. Am J Physiol Endocrinol Metab 279: E1003-11 (2000); Gordon, D. A., and Janil, H. Biochim Biophys Acta 1486:72-83 (2000)). Insulin inhibition of hepatic VLDL secretion is due in part to reduced intracellular association of lipids with ApoB thereby limiting assembly of VLDL. This process is dependent upon endosomal translocation of ApoB (Sparks, J. D. et al. Biochem J 313 (Pt 2):567-74 (1996)), activation of phosphatidylinositol 3-kinase signaling (Sparks, J. D. et al. (1996); Brown, A. M., and Gibbons, G. F. Arterioscler Thromb Vasc Biol 21, 1656-61 (2001)) and also involves degradation of ApoB through endosomal and re-uptake dependent pathways (Fisher, E. A. et al. J Biol Chem 276, 27855-63 (2001)). MTP (Liao, W. et al. J Lipid Res 44, 978-85 (2003); Wetterau, J. R. et al. Biochim Biophys Acta 1345, 136-50 (1997)) and fatty acid-binding protein (L-FABP) (Bass, N. M. Mol Cell Biochem 98, 167-76 (1990)); Raabe, M. et al. Proc Natl Acad Sci USA 95, 8686-91 (1998); Schroeder, F. et al. Chem Phys Lipids 92, 1-25 (1998) are essential for the co-translational assembly of the lipid core upon ApoB in the formation of nascent VLDL (Rustaeus, S. et al. J Biol Chem 273, 5196-203 (1998); Taghibiglou, C. et al. J Biol Chem 275:8416-25 (2000)) and accordingly insulin-dependent degradation of ApoB occurs primarily on newly translated ApoB (Sparks, J. D., and Sparks, C. E. J Biol Chem 265:8854-62 (1990)). Hepatic overexpression of MTP in insulin-resistance stabilizes ApoB in nascent VLDL and enhances VLDL secretion.

What is the Regulatory Function of Leptin in Normal and Insulin-Resistant States.

Leptin, the protein product of the oh gene, is a 16 kDa circulating hormone or adipokine (Zhang, Y. et al. Nature 372:425-32 (1994): Katoh, M., and Katoh, M. Int J Mol Med 19:273-8 (2007)). Leptin is almost exclusively secreted by white adipose tissue (WAT) and elevated levels of this tissue type, as observed in obese individuals, correlate well with high plasma levels of the hormone (Banks, W. A. Peptides 25:331-8 (2004); Fruhbeck, G. Proc Nutr Soc 60:301-18 (2001); Fruhbeck, G. et al. Clin Physiol 18:399-419 (1998)). The mechanism of leptin signaling has been most extensively studied in brain, specifically in the hypothalamus, where it confers the sensation of satiety through binding to its cytokine like receptor Ob-Rb, one of its several alternatively spliced isoforms (Tartaglia, L. A. et al. Cell 83:1263-71 (1995)). Intracellular signaling occurs through the JAK-STAT signaling cascade resulting in immediate alterations in gene transcription, through STATs, as well as cross-talk with other signaling cascades through Janus kinase activation (Katoh, M., and Katoh, M. (2007); Piessevaux, J. et al. J Biol Chem 281:32953-66 (2006)). Negative regulation of Ob-R is achieved through SOCS-3 inhibition of Ob-R phosphorylation (Bjorbak, C. et al. J Biol Chem 275:40649-57 (2000)) and enhanced protein phosphatase 1B (PTP1B) activity on Janus kinase (Zabolotny, J. M. et al. Dev Cell 2:489-95 (2002)). Obesity and insulin-resistance induced by high-fat diet-feeding is associated with overexpression of hepatic PTP1B (Lam, N. T. et al. J Mol Endocrinol 36:163-74 (2006)). Important to this proposal leptin signaling has been observed to increase insulin sensitivity through activation of IRS elements and PI3K (Niswender, K. D., and Schwartz, M. W. Front Neuroenclocrinol 24:1-10 (2003)). Diet-induced obesity in rats was observed to have a deleterious effect on the synergistic relationship between leptin and insulin signaling by reducing the basal levels of IRS-1pY, IRS-1p85, IRS-2pY, and IRS-2p85 to 20-25% of lean controls (Brabant, G. et al. Faseb J19, 1048-50 (2005)). Authors also noted a significant decrease of JAK2p in the obese state, further diminishing crosstalk between signaling cascades, leading to insulin-resistance. Collective these studies suggest an interplay between leptin and insulin signaling pathways where in perturbations of one pathway are likely to impact signaling in the other in the chronic obese state.

Dietary Model

The majority of studies of obesity induced insulin resistance have used high fat feeding (60% more saturated fat relative to standard chow) to study the relationship between hepatic fat accumulation and insulin action (reviewed in (Gauthier, M. S. et al. Br J Nutr 95:273-81 (2006); Lin, J. et al. Cell 120:261-73 (2005); Samuel, V. T. et al. J Biol Chem 279:32345 53 (2004)). Disclosed herein is a model in conjunction with the ob/ob mouse model described below. Fat feeding involves a diet consisting of for example a safflower oil-based high-fat diet that supplies 59% of the calories as fat and 20% of the calories as carbohydrate compared to a normal chow diet that provides 10% of the calories as fat and 65% as carbohydrate (Chalkley, S. M. et al. Am J Physiol Endocrinol Metab 282:E1231-8 (2002)). This diet induces whole body insulin-resistance in animals maintained on it for a minimum of 6-8 weeks (Chalkley, S. M. et al (2002); Kusunoki, M. et al. Diabetes 44:718-20 (1995); Pedersen, O. et al. Endocrinology 129:771-7 (1991); Storlien, L. H. et al. Am J Physiol 251:E576-83 (1986)). At this stage insulin-resistance is largely due to impaired insulin-stimulated glucose transport in skeletal muscle and adipocytes (with the latter showing decreased expression of insulin-responsive glucose transporter-4 Glut 4 (Pedersen, O. et al. 7 (1991)), glucocorticoid-induced insulin-resistance in skeletal muscle (Kusunoki, M. et al. (1995)) as well as the increased production of pro-inflammatory cytokines and cytokine signaling pathways (Adiels, M. et al. (2006); Klover, P. J. et al. (2005); Klover, P. J. et al. (2003); Senn, J. J. et al. (2003) Adiels, M. et al. Diabetologia 49:755-65 (2006)). Serum FA (from adipose tissue) and serum VLDL (from the liver) become significantly elevated relative to lean controls by 12-16 weeks of continued maintenance on the high-fat diet (Park S. Y et al. Diabetes 54:3530-40 (2005)). At this time hepatic insulin-resistance (as measured by hyperinsulinemic-euglycemic clamp) can be measured and lipid droplet accumulation in the liver was evident, marking overt hepatosteatosis (Park S. Y et al. (2005).

Feeding fructose-enriched diets (60% more fructose relative to standard mouse chow) (Bray, G. A. et al. Am J Clin Nutr 79:537-43 (2004); Huang, Y. J. et al. Metabolism 46:1252-8 (1997); Podolin, D. A. et al. Am J Physiol 274: R840-8 (1998)) rapidly induces whole body insulin-resistance (within 3-4 weeks) and a metabolic syndrome associated with obesity, hyperinsulinemia, hepatic MTP overexpression (Lin, M. C. et al. (1995)), enhanced intrahepatic stability and assembly of ApoB containing lipoproteins, leading to VLDL over secretion (Taghibiglou, C. et al. (2000)), hypertriglyceridemia, diminished insulin signaling in liver (Au, C. S. et al. Metabolism 53, 228-35 (2004)), and increased circulating free fatty acids (Kelley, G. L. et al. Endocrinology 145, 548-55 (2004). This model has demonstrated significant down-regulation of hepatic insulin signaling in the fructose-fed, insulin-resistant hamster as evidenced by decreased phosphorylation of the insulin receptor, IRS-1, and IRS-2, decreased activity of phosphotyrosine-associated PI3-kinase, reduced phosphorylation of Akt/PKB and overexpression and hyper activity of PTP-1B (Tartaglia, L. A. et al. (1995)). This dietary model is relevant to the study of insulin-resistance, consumption of fat in the U.S. remains high (Heini, A. F., and Weinsier, R. L. (1997).

Genetic Model

An important genetic model is the ob/ob or db/db mice that do not express leptin or are a knockout of the leptin receptor gene respectively (Gavrilova, O. et al. Nature 403:850; discussion 850-1 (2000); Shimomura, I. et al. Nature 401:73-6 (1999)). Ob/ob mice are incorporated into several protocols but always in comparison to responses in diet-induced obese mice. Leptin is released from adipose tissues and has insulin-sensitizing effects mediated primarily through the hypothalamus with direct actions on peripheral tissues such as skeletal muscle and liver (Elmquist, J. K. et al. Nat Neurosci 1, 445-50 (1998); Kahn, B. B., and Flier, J. S. J Clin Invest 106:473-81 (2000)). In lipoatrophic models, insulin resistance due to leptin deficiency is independent of food intake and adipose mass. Leptin administration completely or partially corrects insulin resistance in the lipoatrophic models (Gavrilova, O. et al. (2000); Shimomura, I. et al. (1999)). Effects of leptin on the liver in vivo include enhancement of insulin's suppression of glycogenolysis and hepatic glucose production (Gavrilova, O. et al. (2000)) and in primary hepatocyte culture (Nemecz, M. et al. Hepatology 29:166-72 (1999)).

Prior studies have_demonstrated insulin-dependent regulation of apobec-1 mRNA expression in the Zucker fatty rat (fa/fa) (Shafrir, E. Diabetes Metab Rev 8:179-208 (1992); Zucker, L. M., and Antoniades, H. N. Endocrinology 90:1320-30 (1972). These rats are hypertriglyceridemic, hyperinsulinemic and are insulin resistant with impaired glucose tolerance. Zucker rats are resistant to insulin's inhibitory effect on hepatic glucose production (Zucker, L. M., and Antoniades, H. N. (1972); Jonescu, E. et al. Am J Physiol 248: E500-6 (1985); Terrettaz, J., and Jeanrenaud, B. Endocrinology 112:1346-51 (1983)). Insulin resistance at the level of the adipocyte results in the inability to suppress the release of free FA (Frayn, K. N. et al. Metabolism 42:504-10 (1993)), and high FA levels favor increased hepatic production and secretion of hepatic TG-rich lipoproteins (Tonescu, E. et al. (1985); Azain, M. J. et al. J Biol Chem 260:174-81 (1985); Schonfeld, G., and Pfleger, B. J Lipid Res 12:614-21 (1971)). In hepatocytes derived from Zucker obese rats, insulin did not inhibit ApoB secretion or reduce ApoB in cells to any significant extent (Sparks, J. D., and Sparks, C. E (1994)) showing that the insulin inhibitory action on ApoB is attenuated in insulin-resistant state. The attenuation of insulin action on hepatic B100 production in chronic hyperinsulinemia has recently been demonstrated in humans (Lewis, G. F. et al. Diabetes 42:833-42 (1993)). Using short-term hyperinsulinemia produced by euglycemic hyperinsulinemic clamps, hepatic B100 production is decreased by 50% in controls, but not in obese individuals with chronic hyperinsulinemia (Lewis, G. F. et al. (1993)).

ACF is Localized in the Cytoplasm and Nucleus of Hepatocytes in Situ.

ACF is distributed in both the nucleus and cytoplasm of hepatocytes. It has been shown that ACF is expressed in sufficient abundance in rat hepatoma cells (McArdle 7777 cells) and rat liver for immunological detection with a peptide-specific rabbit polyclonal antibody that binds to a sequence within the amino terminus of ACF (NT-Ab) (Sowden, M. P. et al. (2002)). Immunofluorescence of McArdle cells revealed homogeneous nuclear ACF staining and a granular staining pattern for ACF in the cytoplasm (FIG. 38, left pair of panels). Higher resolution analysis afforded by immunoelectron microscopy of rat liver demonstrated nuclear (Nu) ACF in a perichromatin distribution and the bulk of cytoplasmic ACF localized to the cytoplasmic surface of the endoplasmic reticulum (ER) (FIG. 38, right panel, red arrows point to NT-Ab reactivity). Biochemical fractionation of rat liver into nuclear and cytoplasmic extracts and western blotting with the NT-Ab confirmed this dual localization (FIG. 37A, upper panel, Control pair of C, cytoplasmic and N, nuclear extract). On a per ug protein basis more ACF appeared to be recovered in the nuclear extract. The PAGE were loaded with equal μg of extract protein but given that there is ˜15-times more total volume of cytoplasmic extract, the amount of ACF in the nucleus and cytoplasm (N/C ratio) was calculated to be approximately 1:3. Ultraviolet light (UV) induces covalent cross-links between proteins that bind to RNAs and this system has been developed to demonstrate selective binding of ACF to ³²P-radiolabeled apoB mRNA containing the mooring sequence (Smith, H. C. Methods 15:27-39 (1998)). selective apoB mRNA binding by ACF in nuclear or cytoplasmic extracts (Sowden, M. P. et al. (2002)) has been demonstrated (FIG. 37A, lower panel).

An important premise (Lehmann, D. M. et al. (2006); Lehmann, D. M. (2007)) is that ACF trafficking and apoB mRNA editing are regulated by insulin-dependent phosphorylation of serine in ACF. The fasted and high-sugar refed rat is a metabolic model of the respective acute hypoinsulinemic and acute hyperinsulinemic state (induced by refeeding with a high-sugar, low-fat diet (Funahashi, T. F. et al. J Lipid Res. 36:414-428 (1995))). Trafficking of hepatic ACF to the nucleus is impaired in the fasted rat and robustly re-stimulated by refeeding (FIG. 37A, upper panel, fasted and refed C and N pairs respectively). ACF RNA binding activity detected in the cytoplasm is markedly increased in the hypoinsulinemic state and markedly reduced in the hyperinsulinemic state commensurate with changes in ACF abundance (FIG. 37A, lower panel fasted and refed C and N pairs respectively). Western bolt analysis of ACF subcellular distribution in apobec-1 knockout mice demonstrated that ACF trafficking to the nucleus is not dependent on APOBEC-1 expression (FIG. 37B), a finding that is consistent with the absence of a strong nuclear localization signal in APOBEC-1 (Yang, Y., and Smith, H. C. (1997)). The subcellular fractionation was evaluated for cross-contamination using antibodies reactive with Ku70 (predominantly nuclear RNA binding protein) and actin (cytoplasmic protein). Taken together, these data are interpreted as indicating that ACF trafficking is a regulated process and that regardless of its intracellular localization, ACF binds to apoB mRNA. Under acute insulin stimulation, nuclear retention of ACF can reduce apoB mRNA export from the nucleus to the cytoplasm and thereby decrease the amount of apoB mRNA available for translation. On the other hand, excess ACF in the cytoplasm, as seen in the ob/ob mouse liver (FIG. 41), is predicted to hyper-stabilize apoB mRNA in the cytoplasm contributing to the hyper-expression of ApoB protein such as is characteristic of the insulin-resistant state.

ACF is a Phosphoprotein and as such, is Restricted to the Nucleus of Hepatocytes.

ACF was shown to contain metabolically regulated serine phosphorylation through ³²P pulse radiolabeling studies in rats and in rat primary hepatocyte cultures (Lehmann, D. M. et al. (2006); Lehmann, D. M. (2007)). Both ethanol and insulin induced hepatic ACF phosphorylation. The function of ACF phosphorylation was revealed through subcellular fractionation. Only nuclear ACF contained radiolabeled phosphoserine and this form of ACF was quantitatively recovered in 27S complexes containing apoB mRNA and APOBEC-1. Cytoplasmic ACF, was recovered as 60S complexes with apoB mRNA but did not contain radiolabel, and APOBEC-1 could not be co-immunoprecipitated with ACF from cytoplasmic extracts (Lehmann, D. M. et al. (2006); Lehmann, D. M. (2007)). Phosphoamino acid specific antibodies revealed however that both nuclear and cytoplasmic ACF had low turnover (constitutive) phosphothreonine (Lehmann, D. M. (2007)).

The role of phospho-ACF in the nucleus was further delineated by ACF immunoprecipitation using extracts from rat primary hepatocytes treated with kinase activators or phosphatase inhibitors. The data demonstrated that ACF phosphorylation was not required for nuclear import, but was required for ACF nuclear retention. Protein Kinase C (PKC) isoforms zeta, beta II and alpha were identified as likely candidates involved in ACF phosphorylation as their activities uniquely promoted high levels of ACF phosphorylation, ACF nuclear retention and activation of apoB mRNA editing activity (Lehmann, D. M. (2007)). These studies also implicated a role for protein phosphatase 1 (PP1) in regulating ACF dephosphorylation, nuclear export of ACF and down-regulation of apoB mRNA editing activity (Lehmann, D. M. et al. (2006)).

Site directed mutation of computationally predicted PKC sites demonstrated that serine to alanine substitutions at residues 154 and 368 uniquely inhibited editing activity and had a dominant negative effect on the ability of apoB mRNA editing to be metabolically regulated. In contrast, serine to aspartic acid substitutions (scrine phosphorylation mimic) at residues 154 and 368 uniquely and fully activated editing activity (Lehmann, D. M. (2007)).

ACF trafficking to the nucleus and phosphorylation occurs in rodent and human liver, and it was therefore asked whether ACF is a phosphoprotein in mice. The question was addressed in apobec-1 knockout mouse liver where the intrinsic capacity of ACF to be phosphorylated can be evaluated in the absence of concerns for its regulation in apoB mRNA editing. Primary hepatocyte cultures (containing insulin 0.1 nM) from apobec-1 KO mice were pulse ³²P radiolabeled in the P.I.'s lab for 4 h and nuclear extracts where immunoprecipitated with ACF CT-Ab and transferred to nylon membrane. Radiolabeled phosphoamino acids were evaluated by autoradiography (FIG. 41) of 2D thin layer chromatography of acid hydrolyzates of ACF excised from nylon membranes as described recently (Lehmann, D. M. (2007)). Consistent with what has been observed for rat liver ACF, phosphorylation of mouse liver ACF occurred on both serine and threonine.

Cytoplasmic ACF Co-Fractionates with apoB mRNA.

ACF is associated with apoB mRNA throughout its life span in the cell and that factors which affect nuclear export affect apoB mRNA export and hence the capacity of the liver to express ApoB protein, assembly and secrete VLDL (clear lipid) (Phung, T. L. et al. Metabolism 45, 1056-8 (1996)). Immunolocalization and RNA binding (FIG. 37) analyses support this. If apoB mRNA remains associated with ACF during nuclear export, it is anticipated that cytoplasmic ACF should biochemically co-fractionate with cytoplasmic polysomes containing apoB mRNA.

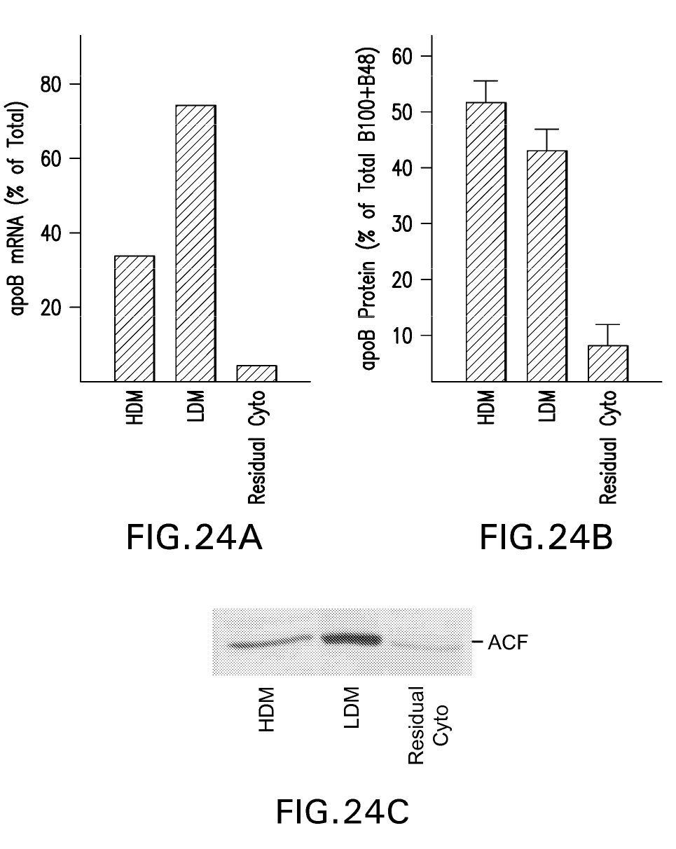

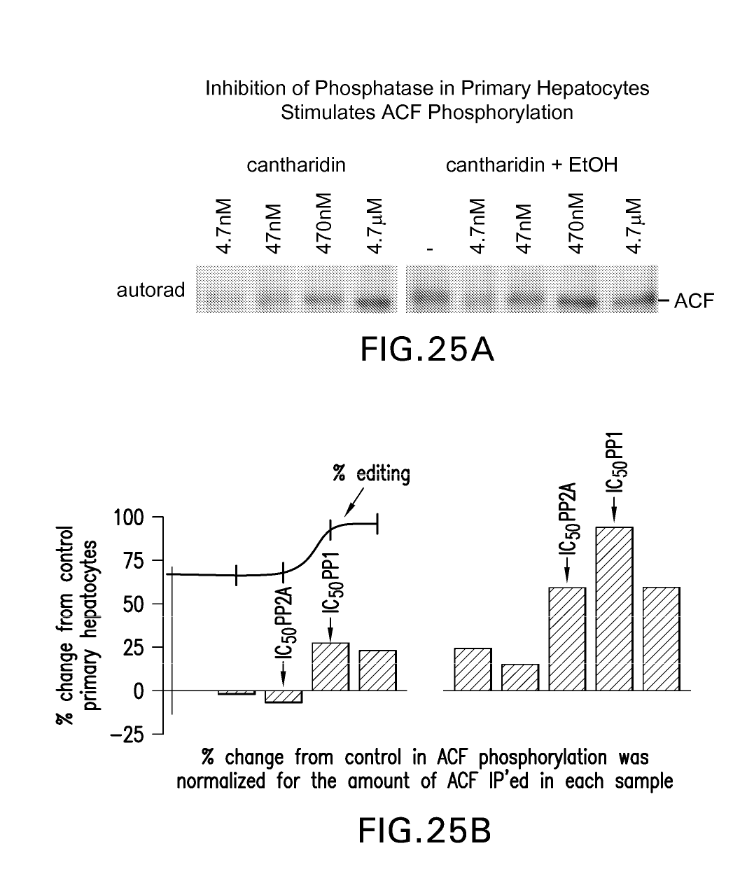

Polysomal apoB mRNA co-sediments with the low density microsomal membrane fraction (LDM) rather than the high density membrane fraction (HDM). The LDM fraction contains the bulk of the endoplasmic reticulum as well as membrane bound polysomes (Phung, T. L. et al. (1996). The aberrant sedimentation of apoB mRNA polysomes is due to the co-translational assembly of nascent ApoB protein with membrane lipids in the formation of VLDL (discussed in (Phung, T. L. et al. (1996)) through the endoplasmic reticulum luminal protein known as microsomal transfer protein, MTP (Shelness, G. S., and Ledford, A. S. (2005)). Quantitative northern slot blot analysis with apoB mRNA specific probes (courtesy of Dr. J. Sparks,) demonstrated that the bulk of apoB mRNA was recovered in the LDM fraction (FIG. 24A). In contrast, ApoB100 and ApoB48 translation products were recovered in the HDM fraction, characteristic of secreted proteins (FIG. 24B). To validate that ACF was associated with apoB mRNA translation complexes, LDM and HDM fractions of rat liver were western blotted for ACF. Western blots of equal amounts of protein as HDM, LDM and residual cytoplasmic fractions reacted with ACF CT-Ab demonstrated an enrichment of ACF in the LDM fraction (FIG. 24C). Given that cytoplasmic ACF preferentially localized to the cytoplasmic surface of the endoplasmic reticulum (Sowden, M. P. et al. (2002)), bound selectively to the mooring sequence of apoB mRNA (regardless of the editing status of the mRNA (Harris, S. G. et al. J. Biol. Chem. 268:7382-92 (1993)) and protects edited apoB mRNA from NMD (Chester, A. et al. Embo J 22:3971-82 (2003), it appears that ACF remains associated with apoB mRNA during nuclear export and tracks with apoB mRNA to the site of translation. Phosphorylation of ACF can therefore serve as a mechanism for modulating the flux of apoB mRNA from the cell nucleus into the cytoplasm for translation. Precedent for this is the regulation of the phosphorylation of SR alternative splicing factors determining their alternate roles as RNA splicing factors or nuclear export chaperones of associated RNA through interaction with exportin proteins (Huang, Y. et al. Proc Natl Acad Sci USA 101, 9666-70 (2004)).

Development of ACF RNAi

RNAi suppression of ACF expression has been proposed for the analysis of ACF-dependent nuclear export of apoB mRNA and VLDL secretion and the role of ACF phosphorylation in this process. Dharmacon RNA interference SMARTpool technology (Dharmacon, website publication) can be used to knockdown human ACF protein expressed in human HepG2 hepatoma cells while not affecting the expression of the rat acf transgene or the control luciferase transcript. By comparative acf cDNA analysis, rodent-specific RNAi were designed and three RNAi (H2, H3 and W2) sequences were cloned into the pAdEasy vector (Stratagene) to produce Adenovirus expressing both RNAi under the control of the U6 promoter and also GFP under the control of the CMV promoter as a marker of infection efficiency. A scrambled RNAi sequence (NS) was prepared as a control for the effect of adenovirus infection on cell viability and the transcriptome. McArdle rat hepatoma cells were used as a system for titering the adenovirus infection efficiency and monitoring the knockdown efficacy of each construct. Cultures demonstrating >90% expression of GFP were harvested at 24 hours post infection and evaluated by western blotting for ACF and actin as described above (FIG. 39). For these studies, blots were sequentially probed for ACF, actin and eGFP (a marker for equivalent viral infection and expression). Scanning densitometric quantification of actin and ACF signals from a light autoradiographic exposure were used to calculate ACF/actin ratios to quantify knockdown efficiency. Cells infected with H2 had ≧70% ACF knockdown relative to that seen with NS and uninfected cells (NV) (FIG. 39). H3 induced ˜30% knockdown whereas W2 produced low or no knockdown.

The effect of ACF knockdown in rat primary hepatocytes was evaluated. These cultures are known to retain the phenotype of differentiated liver when plated as confluent monolayers onto collagen coated surfaces in serum-free media supplemented with 0.1 nM insulin (Berry, M. N. et al. Cell Biol Toxicol 13, 223-33 (1997); omez-Lechon et al. Chem Biol Interact 165, 106-16 (2007); Gomez-Lechon, M. J. et al. Prog Mol Subcell Biol 25, 89-104 (2000); Neufeld, D. S. (1997). Isolation of rat liver hepatocytes. Methods Mol Biol 75, 145-51; O'Brien, P. J. et al. Chem Biol Interact 150, 97-114 (2004)). Therefore RNAi knockdown of ACF in these cells was likely to yield results that are more relevant to liver cell biology. Hepatocytes appeared to be resistant to the potential toxic effects of adenovirus infection or ACF knockdown as little or no attrition of cells from monolayers was observed with ˜100% the cells showing high levels of GFP fluorescence (FIG. 38) for 2-3 days. The infected hepatocytes retained their flattened morphology and contained sharply contrasting nucleoli and organelles (characteristic of actively metabolizing liver cells) that appear contrasted against the background of GFP fluorescence distributed throughout the cell (FIG. 38). In primary hepatocytes infected with NS and W2, fluorescent-opaque, lipid filled endosomal vesicles (dark peri-nuclear region) were readily apparent; the abundance of which is a hallmark of hepatocyte lipoprotein assembly and active secretion of ApoB-containing very low density lipoproteins (VLDL). Within the same time frame, very few hepatocytes contained these vesicles when infected with H2 (H3 was not as effective as H2) and those that did, were markedly less opaque (suggesting less lipid content). This response has been observed in three separate preparations of primary hepatocytes treated with ACF RNAi and showed that H2 knockdown of ACF affects lipid processing and storage in hepatocytes.

One possible explanation for this observation is that ACF knockdown affected ApoB protein availability for lipoprotein assembly through changes in apoB mRNA stability and/or ApoB translation. Given that there is one ApoB protein per VLDL, quantification of VLDL is routinely carried by ApoB radioimmunoassay. Consequently, culture supernatants were collected over a 24 h period following Adenovirus infection and subjected to RIA. The VLDL secreted by rat primary hepatocytes into culture media was reduced in cells infected with H2 (and to a lesser extent H3) compared to that secreted from cells infected with NS (Table 5, two separate experiments). This shows strong support for a functional link between ACF expression and the ability of liver to clear lipid through the VLDL secretion pathway.

Protein Phosphatase 1 Activity Modulates ACF Phosphorylation, apoB mRNA Editing and ApoB Protein Abundance.

Recently published data demonstrated that protein kinase activators and protein phosphatase inhibitors (indolactam V, PKC activator and cantharidin, PP1 inhibitor) uniquely stimulated ACF phosphorylation and markedly stimulated the amount of edited apoB mRNA in rat primary hepatocytes (Gomez-Lechon et al. Chem Biol Interact 165, 106-16 (2007)). Maximum recovery of phosphorylated ACF IP'ed from rat hepatocyte nuclear extracts was from cells treated with 5 μM cantharidin to completely inhibit PP1 (IC₅₀ of PP1, 470 nM) (Mehta, A. et al (2002)). Similarly, apoB mRNA editing was maximally stimulated by cantharidin treatment of primary hepatocytes at ≧the IC₅₀ of PP1.

If alterations in ACF phosphorylation are consequently to ApoB metabolism, then intracellular ApoB protein abundance should be responsive to inhibition of PP1. This was evaluated in treated primary hepatocytes by quantifying ApoB protein in hepatocyte cytoplasmic extracts and secreted into the media as VLDL using ApoB radioimmunoassay and expressed as ng ApoB protein per mg total protein (FIG. 13). For these experiments insulin was withdrawn from control and cantharidin-treated cultures 24 hours prior to the addition of cantharidin in order to isolate the effect of PP I inhibition in the absence of insulin stimulated kinase activity. A slight reduction in intracellular (cell) and secreted (media) ApoB (22% and 17% respectively) was apparent at the IC₅₀ of PP1 (0.5 μM) relative to that observed in untreated hepatocytes (0 μM). A marked reduction in intracellular (‘cell’) ApoB (≧60%) was apparent in cells where PP1 was predicted to be nearly completely inhibited (10-times the IC₅₀ or 5 μM). Secreted ApoB (‘media’) was also reduced (≧30%) from cells treated with 5 PM cantharidin. This is the same dose response that was reported for maximal phospho-ACF recovery Mehta, A. et al (2002)). Taken together the data suggest ACF and ACF phosphorylation play an important role in the regulation of hepatic VLDL assembly and lipid clearance.

Ob/ob Mouse Liver has a Defect in ACF Trafficking.

The ob/ob obese mouse model was observed for alterations in ACF trafficking. The mice are insulin-resistant as evident in elevated blood glucose (180-220 mg/dl after fasting) and lack a significant response to challenge when injected with 1.5 IU of insulin/kg BW. It has been shown that apobec-1 gene expression and apoB mRNA editing is markedly stimulated by insulin (Sowden, M. P. et al. (2002); Funahashi, T. F. et al (1995); Phung, T. L. et al. Metabolism 45, 1056-1058 (1996); von Wronski, M. A. et al. Metabolism 47, 869-73 (1998)). As a further indicator of the insulin-resistant state of these animals, it is apparent that hepatic apoB mRNA editing is not stimulated compared to cogenic lean mice (FIG. 40). The results from three fed animals as lean, ob/ob and 4 h Insulin-treated ob/ob mice are shown. Editing was evaluated using the established poisoned-primer extension assay for quantifying edited apoB mRNA where % editing is calculated from PhosphorImager scans as the counts in the edited band, divided by the sum of the counts in the edited plus unedited bands X 100. The nuclear-cytoplasmic distribution of ACF was next assessed in lean and ob/ob mouse liver without or with insulin challenge (4 h following 1.5 IU insulin/kg BW). FIG. 41 shows western blots of representative aliquots of cytoplasmic and nuclear extracts probed sequentially with antibodies reactive with ACF, actin and histone H1. While the hepatic ACF nuclear retention in lean controls increased following insulin stimulation (4-fold change), ACF nuclear retention was markedly insulin-resistant in ob/ob mouse liver and did not respond to insulin (0.9-fold change). For comparison between animals and treatment groups, the densitometric scans of ACF signals were normalized in the cytoplasm for the actin signal and in the nucleus for the histone H1 signal. The fold-nuclear retention of total cellular ACF was calculated from these values and is provided at the bottom of the image. It was also suggested from these western blots (which are representative images of the results from three separate animals in each group) that there can be more total ACF in the cytoplasm and nucleus of the ob/ob liver compared to the lean liver.

To quantify this observation, whole liver from lean and ob/ob mice were processed for protein and western blotted to quantify ACF (using actin as a cell normalization control) or processed for RNA and mRNA abundance quantified for acf, apoB and apobec-1 by qRT-PCR (using actin as cell normalization control). FIG. 42 (left panel) shows that, as anticipated from the literature, the abundance of apoB mRNA per hepatocyte is not significantly different in the lean and ob/ob mice. Consistent with the apoB mRNA editing activity, ob/ob hepatic apobec-1 mRNA abundance was not significantly different than that quantified in the lean mouse liver, suggesting the possibility that apobec-1 gene expression in the ob/ob mouse liver was insulin-resistant. In contrast, hepatic acf mRNA abundance was markedly elevated in ob/ob mice compared to that quantified in lean mice and this corresponded to a marked elevation in the abundance of ACF protein (FIG. 42, right panel) in these animals (p<0.05 and p<0.01, respectively). Therefore, in addition to insulin-resistant trafficking of ACF in ob/ob liver, ACF expression also is deregulated.

The marked overexpression of ACF in ob/ob mice raised the question of whether there is a threshold capacity of PKC to phosphorylate ACF. It appears that ACF nuclear restriction due to phosphorylation could become saturated when ACF expression exceeds the threshold. The possibility was addressed through in vivo ³²P pulse labeling of ob/ob and age-matched lean controls during the refeeding phase of the fasting-refeeding protocol known to induce hyper-phosphorylation and nuclear retention of ACF in lean animals. Nuclear and cytoplasmic extracts were immunoprecipitated with the ACF CT-Ab, transferred to PVDF nylon membranes, PhosphorImaged to obtain the ³²P signal and western blotted with ACF NT-Ab to validate that the ³²P signal aligned with ACF (FIG. 42a). Densitometer scanning of light exposures of the ACF western was used to obtain a relative quantification of ACF recovered from each fraction by IP. The data are representative of determinations carried out on 3 animals in each group and demonstrated that (i) ACF is phosphorylated in the liver of ob/ob mice, (ii) only nuclear ACF had detectable ³²P labeled ACF however (iii) the specific activity of ACF recovered from ob nuclear extracts is markedly reduced compared to that of the lean control. These data are important in that they suggest that while the intracellular site of ACF phosphorylation (the nucleus) is unchanged by obesity, the bulk of ACF in the nucleus of ob/ob livers is not phosphorylated despite hyperinsulinemia and dietary stimuli resulting from the post prandial state. It appears that the calculated reduced specific activity of phophoACF in ob/ob nuclei is due to more hypo-phosphorylated ACF in the IP, and this can result from ACF overexpression having exceeded the substrate saturation threshold for the kinase. It is therefore believed that the bulk of nuclear export of ACF and apoB mRNA in ob/ob liver is unregulated. Accordingly, the model for the role of ACF in fatty liver disease incorporates persistent ACF overexpression as a significant contributor to dyslipidemia.

Dysregulation of ACF in High-Fat Diet Obese Mice

As described above, ob/ob mice are morbidly obese due to long term over eating induced by the absence of leptin. The data in FIG. 41 raised the question as to whether dysregulation of ACF expression is simply a response to the absence of leptin and therefore may not be seen in other models for obesity? Put another way, would cogenic mice with normal capacity to express leptin demonstrate similar ACF dysregulation when they became obese? This was addressed by high-fat diet feeding cogenic wild type mice for 2 months at which point they showed signs of hyperglycemia and had a reduced response to an insulin challenge (insulin resistance). The animals gained 20%-30% in body mass relative to animals maintained on normal mouse chow. Blood glucose levels in the high-fat diet fed mice after a 4 h fast were 100-150 mg/dl whereas age-matched, lean control mouse fasted blood glucose levels were 65-125 mg/dl.

A representative western blot from a subcellular fractionation study performed on liver from fed animals on normal chow and the high-fat diet (n=3) (FIG. 43) was carried out as described above and Lehmann, D. M. et al. (2006) and Lehmann, D. M. (2007). At first glance, the data suggest that ACF subcellular distribution in high-fat diet fed induced obese animals (DIO) is comparable to that of the lean control mouse liver. However, when the signals were adjusted for the relative amount of actin cross-contamination in the nuclear fractions (indicative of cytoplasmic contamination) there was >2-fold more nuclear ACF in the DIO mouse liver. These data clearly differed from those obtained from the fed ob/ob liver fractionation and suggested that the characteristic of insulin-dependent ACF nuclear retention (and ACF phosphorylation) was retained in the livers of mice made acutely obese despite the onset of insulin-resistance as measured by fasting blood glucose levels and insulin challenge.

The hepatic mRNAs of lean control and DIO mice (n=3, each group) were not significantly different for apoB, but apobec-1 mRNA was reduced (p<0.05, Student t-test), consistent with an insulin-resistant state (FIG. 44, left panel). Importantly, ACF protein levels were significantly elevated in DIO compared to lean controls (FIG. 44, right panel) (p<0.02, Student t-test). This shows that the dysregulation of ACF can begin with overexpression of ACF and that this can progress to disrupted insulin-dependent ACF nuclear retention such as seen in the ob/ob mouse liver.

Computational Predictions of the Cis-Acting Regulatory Elements in the ACF Promoter.

Liver specific acf promoter elements have been mapped to the first 2 kb upstream of the human acf gene (Dance, G. S. C. et al. (2002)) and therefore are in a position to pursue the possibility that leptin, FA, cytokine or adipokine signaling leads to ACF transcriptional suppression. FIG. 45 shows a cartoon of the positions of predicted transcription factor bindings sites within the minus 2 kb region of the mouse acf gene using the transcription factor binding site search engines TESS (http://www.cbil.upenn.edu/cgi-bin/tess/tess?RQ=WELCOME) and PROMO v3 (http://alggen.isi upc.es/cqi-bin/promo v3/promo/promoinit.cqi?dirDB=TF 8.3).

Predicted sites were restricted to 100% consensus matches then filtered by predictive confidence, in TESS by La (likelihood)/length of site being >1.5 (2.0 is max) and in PROMO by random occurrence of the site <1 in the length of sequence tested. These criteria yield probability sites in the upper most likely quartile. Two binding sites for STAT4 and one STAT1 site were predicted. These are of interest to the proposed research as they are known to be regulated by Janus kinase activation following leptin binding to the ObR and STAT1 homodimers have been implicated in suppression of the PPARγ2 expression (Hogan, J. C., and Stephens, J. M. Biochem Biophys Res Commun 287, 484-92 (2001)). Leptin signaling has been implicated in activation of MAP kinase pathways, especially those involving ERK1/2 (Fruhbeck, G. Biochem J 393, 7-20 (2006)). Substrates in these cascades can include ETS containing factors (PU.1 and ELK-1), c-myc or CEBPc/p, all with binding sites represented in acf promoter. Other sites of note include those for PPARα and RXR-α known to control the expression of genes involved in lipid metabolism and fatty acid oxidation as reviewed in (Touyz, R. M., and Schiffrin, E. L. Vascul Pharmacol 45, 19-28 (2006)). This computational promoter analysis shows that acf gene expression can be regulated by signaling pathways active in obese and insulin-resistant animals.

### Quantify Hepatic ACF Abundance in the Nucleus and Cytoplasm of Insulin-Resistant Obese Animal Models

The analysis begins by evaluating 8-12 week ob/ob male mice fed normal mouse chow. As described above, these animals do not express leptin from birth, and this genetic basis for obesity and insulin-resistance can differ mechanistically from that induced by diet. Consequently, lean cogenic mice (age-matched) are initiated on the high-fat diet feeding protocol for 8, 12, and 24 weeks and weight gain recorded relative to age-matched lean mice fed normal mouse chow for the same period.

Animals are euthanized by CO₂ asphyxiation and blood is drawn by cardiac puncture. Livers are perfused in situ with 5 mls of ice cold 0.25M-STM buffer (50 mM Tris pH 7.2, 5 mM MgCl₂ containing, 10 mM NaF and Complete™ proteinase inhibitors (Roche)) and processed for nuclear and cytoplasmic extracts as established (Sowden, M. P. et al. (2002)). Extract proteins are quantified with the BioRad Reagent and proteins from an equivalent proportion of each fraction is resolved by SDS PAGE and western blotted. The C-terminal ACF peptide-specific antibody is reacted with each blot, followed by reaction with peroxidase conjugated secondary antibody and chemiluminescence substrate (ECL, Perkin Elmer). The amount of ACF detected in each fraction is determined by densitometric scans of light X-ray film exposures and the total amount of ACF in each fraction calculated by correcting these values for the proportion of each fraction that was loaded onto the gel as described previously. To compare the ACF signal between treatment groups, blots are stripped and sequentially reacted with anti-actin and anti-histone H1 antibodies in order to normalize the respective cytoplasmic and nuclear ACF densitometric signals. All animal models are evaluated in triplicate and the experiment is performed three times. Data is analyzed for means and standard deviations and analyzed for significance using ANOVA. Differences are considered significant at P≦0.05.

### Quantify Hepatic ACF Phosphorylation in the Nucleus and Cytoplasm of Insulin-Resistant Obese Animal Models.

All animals used for phosphorylation analyses are injected IP with 2 mCi of ³²Pi in Hepes buffered saline without or with 1.5 IU/kg porcine insulin (insulin challenge). Four hours of in vivo labeling is used and this was determined to yield the maximum recovery of phosphoACF in rats (Lehmann, D. M. et al (2006)). The occurrence of phosphoACF in the nucleus and cytoplasm is determined by immunoprecipitating ACF from nuclear and cytoplasmic extracts and quantifying the recovery of phosphoACF in each fraction by PhosphorImager scanning densitometry of PAGE resolved material. The ACF C-terminal antibody is used for the IP as it selects for ACF64 over the less abundant short variants. Phosphoaminio acid analysis of immunopurified ACF is by 2D thin layer chromatography as described (Yang, Y. et al. (2001)).

Quantify Hepatic apoB mRNA in the Nucleus and Cytoplasm of Insulin-Resistant Obese Animal Models.

For RNA analysis, liver segments (0.1 gm wet weight) from the same animals described above are placed into 1 ml of TriReagent and flash frozen in liquid nitrogen. Aliquots of whole cytoplasmic and nuclear extracts from are also placed into TriReagent and flash frozen for the quantification of the relative distribution of apoB mRNA with the hepatocyte fractions. RNA is purified and DNase I digested to remove contaminating genomic DNA. cDNA is reverse transcribed by oligo dT priming and qRT-PCR carried out using apoB-specific primers and Taqman® technology. Actin mRNA in all RNA samples are subjected to qRT-PCR as the cell number normalization control and the abundance of apoB mRNA calculated from the amplification critical threshold values (CT) normalized for actin CT value. Quantification is carried out in triplicate from three separate animals in each treatment group. Analysis of variance is determined as described above.

Quantify acf mRNA (qRT-PCR) and ACF Protein Abundance (Western Blotting) in Samples of Whole Liver within the Obese Animal Models to Evaluate the Generality of the Dysregulation of ACF Expression.

Acf cDNA is reverse transcribed from oligo dT primed total hepatic RNA isolated from the obese animal models and lean controls and subjected to qRT-PCR with acf-specific primer pairs located with the 3′ terminal exon of mRNA encoding full length ACF along with actin mRNA as cell input normalization controls as described above. ACF protein expression is evaluated semi-quantitatively using western blots of equal amounts of total liver protein from each animal in the obese model and lean controls used above and reacted with the C-terminal ACF peptide-specific antibody. Blots are stripped and reacted with monoclonal antibody reactive with beta actin. Antibody reactivity is detected by enhanced chemiluminescence (ECL) and low density X-ray film exposure and quantified by scanning densitometry. The normalized ACF abundance in each treatment group (expressed as the ACF to actin signal ratio) provides the basis for comparison as a ‘fold change’ from lean control values. The number of determinations and statistical treatment of the data is described above.

Three mice are evaluated every other week from each group over an 24 week period for: (1) body weight, (2) gonadal fat pad mass, (3) response to an insulin challenge, (4) fasting blood glucose, (5) acf apoB, apobec-1, actin mRNA abundance, (6) apoB mRNA C/N ratio (7) ACF and ApoB protein abundance, (8) ACF protein C/N ratio and (9) ACF phosphorylation. Obesity-induced, insulin-resistance is monitored on an every other week basis on each animal in each group by tail vein blood analysis for fasting glucose and insulin levels. In addition, insulin tolerance tests (1.5 IU insulin/kg BW for DIO and ob/ob and 0.75 IU/kg for leans) and insulin levels are determined at the time of sacrifice on representative animals in the respective treatment groups. Animals are fasted 4 hours prior to sacrifice and analysis.

Analyses 1 and 2 assess weight gain and provide and metric for WAT mass. Analyses 3 and 4 determine onset and severity of insulin-resistance. Given that the duration of feeding (up to 24 weeks), all animals gain weight but fat-fed animals show a greater rate of weight gain and greater end weight. Fasted, blood glucose values are determined on blood from cardiac puncture. Individual values will vary between 80-120 mg/dl but not change significantly over the course of the feeding period for mice maintained on normal mouse chow. Fasting blood glucose values of mice fed the high-fat diet initially are indistinguishable from that in the control group but as the animals become insulin-resistant, the values rise to >120 mg/dl (range of 120-150 mg/dl by 10 weeks). Analyses 5 and 7 are determined using two small pieces of liver (0.1 gm/sample/animal) from each animal. The pieces of liver are processed immediately with (i) TriReagent for RNA extraction or (ii) homogenized with 0.25M STM plus proteinase inhibitors for total cellular protein extraction. Analyses 6 and 8 are determined on the remainder of the liver from each animal following cytoplasmic and nuclear extract preparation. qRT-PCR quantification of RNA, western blotting quantification of protein is determined for nuclear and cytoplasmic fractions. Analysis 9 on radiolabeled animals is done separately but validation of ACF phosphorylation sites by mass spec is carried out on ACF immunopurified from samples collected from unlabeled animals.

There are 60 samples for analysis by qRT-PCR and western blotting. Two people can process and analyze these samples in two weeks. An addition technical consideration is that all 60 samples should be analyzed in triplicate at the level of qRT-PCR and western blotting. Precision in the analysis is important for reliable comparison between diets. Variation between samples (due to plate-to-plate variation in the qRT-PCR and experimental variation in protein quantification and PAGE or western analyses) can be minimized when all 60 samples are processed simultaneous rather than assaying them in batches of 6 per week over a 24 week period. Samples from 60 animals is processed and the data evaluated for trends in diet-related weight gain, changes in fasted blood glucose over time and changes in ACF and ApoB quantification.

Demonstrate the Requirement for ACF in Regulating Hepatic apoB mRNA and Secretion of VLDL.

Factors that affect the regulation of ACF can modulate apoB mRNA translation and assembly of lipids as VLDL. It has been suggested that insulin-dependent nuclear restriction of ACF may promote lipid retention in response to hyperinsulinemia during the early stages of disease but ACF nuclear retention can become insulin-resistant and/or leptin-resistant, leading eventually to increased production of VLDL, though this VLDL production level can be insufficient to export the lipid load.

Demonstrate that apoB mRNA Nuclear Export is ACF-Dependent using qRT-PCR to Quantify apoB mRNA Changes in the Relative Recovery of apoB mRNA from Nuclei and the Cytoplasm in Hepatocytes wherein ACF Abundance has been Reduced by RNAi Knockdown.

To test whether ACF is essential for apoB mRNA nuclear export, qRT-PCR quantification of apoB mRNA (both total cellular and as N/C ratios) is carried out using primary hepatocytes isolated from lean animals following suppression of ACF by RNAi as described above. Acf H2 RNAi and control NS RNAi have already been identified and subcloned into adenovirus expression vectors and viral packaging system with the AdEasy kit (Invitrogen) and stored as titered stocks using the AdenoX-titer immunological reagent from BD.

Primary hepatocytes cultures (60 mm dishes containing 1.5-2.0 million hepatocytes) are infected with 10 m.o.i. and monitored for infection in living cells through viral expressed eGFP fluorescence. Cell viability is monitored by assaying for the induction of Caspases or DNA strand breakage using apoptosis detection kits (Promega). The efficiency of ACF knockdown by H2 RNAi relative to that seen with NS RNAi is assessed by western blotting total cellular ACF with the ACF CT-Ab.

ACF rescue experiments are necessary for demonstrating that changes in apoB mRNA abundance and/or nuclear export are due to ACF suppression and not the result of unforeseen effects on other proteins (off-target effects of RNAi). Mouse primary hepatocytes treated with H2 acf RNAi or NS RNAi is transduced simultaneously with adenovirus expressing human V5 epitope-tagged ACF cDNA to accomplish the rescue. Cells from each condition in six well clusters are scraped into phosphate buffered saline containing proteinase inhibitors and one third of the cells from each well is pelleted and resuspended in TriReagent for RNA extraction and qRT-PCR of apoB mRNA. The remaining cells from each well is pelleted and fractionated into cytoplasmic and nuclear extracts using the NePer kit (Pierce) as described previously (Lehmann, D. M. et al (2006); Lehmann, D. M. (2007)) and RNA extracted with TriReagent. The expression of exogenous human ACF is evaluated on the same western blots using V5-reactive antibodies. The procedure is repeated on separate cell cultures at 12 hour intervals up to 48 h post-transduction. The knockdown/rescue strategy is performed in triplicate and apoB mRNA is quantified from in sample three times.

Demonstrate that the Abundance of Intracellular ApoB and ApoB Secreted as VLDL is Modulated by ACF Trafficking through ApoB-Specific Radioimmunoassay Quantification ApoB in Hepatocyte Cytoplasmic Extracts and Culture Media from Cell Cultures Subjected to the Knockdown/Rescue Strategy.

Changes in VLDL secretion in response to changes in ACF abundance are evaluated in mouse primary hepatocytes subjected to the knockout/rescue strategy. The experimental design is identical to that above except that the endpoint is quantification of intracellular and secreted ApoB protein. For protein analyses, primary hepatocytes treated as described above are harvested in Reporter Lysis buffer (Promega) for the quantification of intracellular ApoB protein and their culture supernatants (harvested at 12 hour intervals and stored at 4° C.) are used in the quantification of secreted ApoB as VLDL. Cell extracts and media from each hepatocyte culture at different time points are adjusted to a density of 1.019 g/ml with NaBr and ultracentrifuged to ‘float’ VLDL and separate them from the infranatant containing LDL and HDL (Sparks, J. D., and Sparks, C. E. (1990)). Given that there is only one apoB protein molecule per VLDL (or LDL), immunological quantification of apoB protein in the gradient fractions of secreted lipoproteins is a reliable measure of the amount of VLDL produced. Relative to a standard curve composed of mouse apoB VLDL, the amount of VLDL secreted in each culture is quantified by radioimmunoassay based on I¹²⁵-conjugated goat anti-rabbit (Amersham) secondary antibody and ApoB N-terminal reactive primary antibody (Santa Cruz) using the Department of Biochemistry's gamma counter.

Identify the Sites of ACF Phosphorylation and Demonstrate their Role in Regulating apoB mRNA Nuclear Export and VLDL Secretion.

Specific serine and/or threonine residues are targeted in ACF for insulin-dependent phosphorylation. Understanding the effect of insulin-resistance at the level of the utilization of these sites provides mechanistic insight as to how changes in the regulation of ACF site-specific phosphorylation may modulate ACF and apoB mRNA nuclear export and contribute to the dyslipidemia associated with insulin-resistance.

Identify the Sites of Insulin-Dependent Mouse Hepatic ACF Phosphorylation by Phosphopeptide Mass Spec Sequencing of Protein Isolated from In Vivo Insulin-Stimulated Lean Control Mice.

ACF from lean mouse liver with and without and insulin challenge, is immunoprecipitated from nuclear and cytoplasmic extracts (four test samples in total) as recently described (Lehmann, D. M. et al (2006)), resolved by SDS PAGE, excised from gels after a brief staining with Commassie blue and sent to 21st Century Biochemicals for mass spectrophotometric sequencing of phosphopeptides. Detection and localization of phosphorylation sites are performed using nanospray ionization techniques that allow efficient detection of peptides and proteins without the use of radiolabeling, coupled to a triple quadrupole orthogonal time-of-flight or Q-oTOF mass spectrometer (QStar™; Applied Biosystems, Fostor City, Calif.). This instrument performs experiments which include molecular mass determination and assessment of chemical modifications through mass increases as well as fragmentation experiments (for sequence analysis) with very high resolution and high mass accuracy (10 ppm) minimizing the possibilities for incorrect peptide and phosphorylation assignments. The Q-oTOF enables performance of collision-induced dissociation for a peptide ion of interest to gain structural information.

For long proteins such as ACF, experiments using capillary liquid chromatography (100-300 mm reversed-phase columns) interfaced to the Q-oTOF are utilized and extensive structural information is obtained. This step is essential for detection of some phosphorylation sites as often phosphopeptides are suppressed during ionization relative to unmodified peptides requiring chromatographic separation (Perlman, D. H. et al. Proc Natl Acad Sci USA (2005)). To further aid characterization, affinity purified ACF in solution or as an Immobilon-P bound protein is digested with trypsin (or an alternative sequence specific protease) and sent to the facility. Phosphorylated peptides derived from TPCK-treated trypsin digested ACF, is analyzed in a capillary LC/MS experiment with continuous selection of individual ions for fragmentation throughout the data acquisition using a defined set of parameters (mass range, charge state and signal intensity). Therefore knowing the sequence of ACF and its predicted sites of phosphorylation can greatly facilitate identification of phosphoamino acid containing peptides from a set of peptide mass ions. Web-based algorithms such as MS-Fit, PepFrag and/or Peptide Search can be used to “fit” the mass profile fingerprint with ACF. Programs such as GP-MAW (Odensse, Denmark) are also available at the facility to “digest” in silico known protein sequences to predict theoretical peptide digest mass ions and modification sites and therefore the optimal digestion conditions.

Demonstrate the Dependence of apoB mRNA Nuclear Export on Site-Specific ACF Phosphorylation in Cells Subjected to the ACF Knockdown/Rescue Strategy and Expressing ACF Phosphorylation Site Mutants that Mimic ACF Constitutively Dephosphorylated (ser/thr to ala mutants) or Phosphorylated (ser/thr to asp mutants) using qRT-PCR to Quantify Changes in apoB mRNA Relative Abundance in Nuclear and Cytoplasmic Fractions.

Given the conservation of ser154 and ser368 and the observation that mouse ACF contains phosphoserine, the rescue studies with human ACF containing ala or asp mutations at these sites and are used, and additional ACF mutations are adopted into the protocol as they become apparent from mass spec sequencing of mouse ACF. Expression of wild type ACF and ACF with mutations at sites that do not affect ACF function (as measured by complementation of editing activity (Lehmann, D. M. (2007)) expressed in untreated or RNAi-treated primary hepatocytes serve as controls for nonspecific effects of the expression of ACF mutants on apoB mRNA nuclear export.

Demonstrate the Dependence of Intracellular and Secreted VLDL ApoB on ACF Phosphorylation using ApoB-Specific Radioimmunoassay to Quantify VLDL in Cell Extracts and Culture Supernatants from Cells in the ACF Knockdown/Rescue Strategy

The dependence of VLDL assembly and secretion on ACF phosphorylation is determined by expressing ACF phosphorylation site-specific ala/asp mutants as described above in the knockdown/rescue strategy described above and quantifying intracellular and secreted ApoB as described above. Controls are primary hepatocytes treated with non-relevant ACF site-specific mutants and/or treated with NS RNAi. Cell extracts and culture supernatants collected are analyzed for endpoints.

Evaluate the Relative Contributions of Obesity, Insulin-Resistance and Leptin to ACF Dysregulation by Quantifying the Induction of Changes in ACF Endpoints in the Obese Animal Models and Mouse Primary Hepatocytes.

Obesity-induced, insulin-resistance arises progressively in response to changes and imbalances of multiple metabolic and hormonal factors. The relative contributions of obesity, insulin-resistance and leptin signaling that lead to dysregulation of ACF involve an interplay of these factors.

Quantify Changes ACF Endpoints in ob/ob Mice given a Single Bolus of Leptin (10 mg/kg) over a Time Course of 0-24 Hours to Determine, Prior to Weight Loss, whether Leptin alone can Suppress ACF Overexpression and its Dysregulation.

The leptin study of Pelleymounter et al. (Science 269, 540-3 (1995)) involved daily IP injections of recombinant leptin and was conducted over a 26 day period. This study demonstrated a leptin-induced reduction in body weight and WAT mass along with a reduction in insulin-resistance that was both a dose- and time-dependent. A dose of 10 mg/kg per day was required to bring about these effects with ˜20% decrease in weight determined at the conclusion of the study. Relevant to the experimental design is the finding that no significant changes in any of the measured gross physiological endpoints were observed for the first two days of treatment. To address the possibility of leptin as a principle factor regulating acf gene expression, the experimental design focuses on the immediate early phase of the response (with the first 48 hours).

Ob/ob mice are maintained on normal mouse chow and injected IP with 0.2 ml of saline (control) or 10 mg/kg leptin (Sigma-Aldrich, MO). Six animals are placed in each treatment group. For the initial analysis, total liver acf mRNA and ACF protein is determined on liver samples recovered from animals sacrificed following 4 h, 12 h, 24 h and 48 hr of treatment. Liver samples for RNA and protein analysis are collected by flash freezing in liquid nitrogen over the course of the study and processed simultaneously once all samples in the protocol have been collected. At the time of sacrifice, fasting blood glucose levels and gonadal fat pad masses are determined. If there is a significant difference in acf mRNA and ACF protein (ANOVA test, p≦0.05) between treatment groups then a study with an expanded treatment and time course is conducted. In these studies acf mRNA and ACF protein is quantified in ob/ob mouse liver (+/−leptin treatment) in an appropriately focused time course (i.e. 1 h, 2 h, 4 h and 6 h or 12 h, 24 h, 36 h and 48 h). Once the time frame for maximum suppression of acf mRNA and ACF protein expression has been established, varying leptin doses are evaluated in order to establish the maximum effective dose. Once conditions for a maximal leptin response have been established, the effect of leptin treatment on acf mRNA and ACF protein expression in lean controls (maintained on normal chow) and high-fat diet induced obese mice are determined.

Evaluate Key Metabolic and Hormonal Factors that are know to affect Liver Metabolism in the Obese State (i.e. Free Fatty Acids, Glucose, Adipokines (Leptin, Resistin) and Cytokines (IL-6, TNF-α) for their Ability to Modulate ACF (Aim A.1.a) and apoB mRNA (A.1.b) Endpoints in Mouse Primary Hepatocyte Cultures.

To evaluate the basis of ACF dysregulation observed in obesity, mouse primary hepatocyte cultures derived form lean control animals are exposed to key metabolic and hormonal factors and assayed for changes in the ACF endpoints. Primary hepatocytes are isolated from lean control mice, allowed to equilibrate to culture for a 24 h period and treated with varying stimuli for periods of 1 h, 4 h, 6 h, 12 h and 24 h. The treatments are performed in triplicate and whole cell mRNA and protein harvested. The culture media consists of MEM, 0.25% fatty acid free BSA, streptomycin-penicillin-hygromycin and 0.1 nM porcine insulin. Metabolic stimuli that are added to this media include: (i) treatment with free fatty acids, using a 1 mM 1:1 (palmitate/oleic) mixture as the EC50 and/or (ii) 5 to 20 mM glucose treatment (glucose-free MEM is used as the basal media in which the concentration of glucose is varied). Adipokine dose response is assessed across a two log range above and below the basal plasma levels of 1 ng/mL or 4 ng/mL for leptin or resistin, respectively. Cytokine treatments includes doses ranging from 100-fold above and below the basal plasma levels of 10-50 pg/ml for TNF-α or IL-6. The ACF and ApoB endpoints have been described above. Primary hepatocytes in standard non-supplemented culture media and treated with vehicle alone are maintained for the duration of treatment and serve as a source of ACF and apoB mRNA in control analyses. Statistically significant changes from control values in the expression of ACF at either the message or protein level is assessed using a ANOVA test.

Determine how Factors in Aim A.4.b Affect acf Transcription using mouse acf Promotor Reporter Constructs and Mutants Thereof Expressed in Mouse Cell Lines.

ACF (Accession # AY566863) is encoded on mouse chromosome 19 within 80 lib is predicted to include 15 exons plus (Blanc, V. et al. (2005); Dur, S. et al. (2004)). Transcription is initiated from a TATA-less promoter from multiple sites within a cluster of Sp1 sites (Dur, S. et al. (2004)). DNA from Incyte Pharmaceuticals BAC genomic clone # 26879 has been obtained which harbors the entire ACF locus from the SV129 mouse strain. The design of the experiments is to transfect mouse cell lines (e.g. AML12, liver or NIH 3T3, fibroblast) with acf promoter-reporter constructs consisting of up to minus 2 kb of the acf promoter driving transcription of the firefly luciferase gene. Once baseline expression has been established in the recommended media for each cell type, the conditions described above are tested for the ability (alone and in combination) to stimulate or suppress luciferase expression. Cultures are monitored for luciferase activity up to 48 h post-transfection relative to a transfectants with a promoter-less control construct by quantifying cell extracts for luminescence using a commercially available kit (Promega). Once signaling factors have been identified that can affect acf promoter activity, more precise quantification is conducted using different doses and durations of treatment. Cells are co-transfected with the acf promoter firefly luciferase gene and the Renilla luciferase gene driven by the CMV promoter. Sequential quantification of these luciferase signals using the Dual Luciferase Reporter® Assay (Promega) in each sample enables normalization of the changes in firefly luciferase signal for transfection efficiency based on the Renilla luciferase signals.

TABLE 5
Effect of ACF RNAi on VLDL Secreted
from Primary Hepatocytes

| | [apoB] | [apoB] | Average |
|---|---|---|---|
| RNAi Treatment | (ng) exp 1 | (ng) exp 2 | [apoB] (ng) |
| NS | 814 | 1036 | 925 |
| H2 | 514 | 653 | 584 |
| H3 | 700 | 596 | 648 |

### REFERENCES

Adeli, K., Taghibiglou, C., Van Iderstine, S. C., and Lewis, G. F. “Mechanisms of hepatic very low-density lipoprotein overproduction in insulin resistance.” Trends Cardiovasc Med 11:170-6 (2001).
Adeli, K., and Theriault, A. “Insulin modulation of human apolipoprotein B mRNA translation: studies in an in vitro cell-free system from HepG2 cells.” Biochem Cell Biol 70:1301-12. (1992).
Adiels, M., Olofsson, S. O., Taskinen, M. R., and Boren, J. “Diabetic dyslipidaemia.” Curr Opin Lipidol 17:238-246 (2006).
Adiels, M., Taskinen, M. R., Packard, C., Caslake, M. J., Soro-Paavonen, A., Westerbacka, J., Vehkavaara, S., Hakkinen, A., Olofsson, S. O., Yki-Jarvinen, H., and Boren, J. “Overproduction of large VLDL particles is driven by increased liver fat content in man.” Diabetologia 49, 755-65 2006).
Allister, E. M., Borradaile, N. M., Edwards, J. Y., and Huff, M. W. “Inhibition of microsomal triglyceride transfer protein expression and apolipoprotein b100 secretion by the citrus flavonoid naringenin and by insulin involves activation of the mitogen-activated protein kinase pathway in hepatocytes”. Diabetes 54:1676-83 (2005).
Anant and Davidson, “Identification and regulation of protein components of the apolipoprotein B mRNA editing enzyme. A complex event.” Trends Cardiovasc Med 12:311-7 (2002).
Anant et al. “Evolutionary origins of the mammalian apolipoproteinB RNA editing enzyme, apobec-1: structural homology inferred from analysis of a cloned chicken small intestinal cytidine deaminase.” Biol Chem 379:1075-81 (1998).
Araya, J., Rodrigo, R., Videla, L. A., Thielemann, L., Orellana, M., Pettinelli, P., and Poniachik, J. “Increase in long-chain polyunsaturated fatty acid n−6/n−3 ratio in relation to hepatic steatosis in patients with non-alcoholic fatty liver disease.” Clin Sci (Lond) 106:635-43 (2004).
Arraiano, C. M., and Maquat, L. E. “Post-transcriptional control of gene expression: effectors of mRNA decay.” Mol Microbiol 49:267-76 (2003).
Au, C. S., Wagner, A., Chong, T., Qiu, W., Sparks, J. D., and Adeli, K. “Insulin regulates hepatic apolipoprotein B production independent of the mass or activity of Akt1/PKBalpha.” Metabolism 53: 228-35 (2004).
Avogaro, A., Watanabe, R. M., Dall'Arche, A., De Kreutzenberg, S. V., Tiengo, A., and Pacini, G. “Acute alcohol consumption improves insulin action without affecting insulin secretion in type 2 diabetic subjects: Diabetes Care 27, 1369-74 (2004).
Azain, M. J., Fukuda, N., Chao, F. F., Yamamoto, M., and Ontko, J. A. “Contributions of fatty acid and sterol synthesis to triglyceride and cholesterol secretion by the perfused rat liver in genetic hyperlipemia and obesity. J Biol Chem 260:174-81 (1985).
Backus, J. W., and Smith, H. C. “Three distinct RNA sequence elements are required for efficient apolipoprotein B (apoB) RNA editing in vitro.” Nucleic Acids Res 20:6007-14 (1992)
Backus and Smith, “Apolipoprotein B mRNA sequences 3′ of the editing site are necessary and sufficient for editing and editosome assembly.” Nucleic Acids Research 19:6781-6 (1991).
Backus, J. W., Eagleton, M. J., Harris, S. G., Sparks, C. E., Sparks, J. D., and Smith, H. C. “Quantitation of endogenous liver apolipoprotein B mRNA editing.” Biochem Biophys Res Commun 170:513-8 (1990).
Banks, W. A. “The many lives of leptin.” Peptides 25, 331-8 (2004).
Bass, N. M. “Fatty acid-binding protein expression in the liver: its regulation and relationship to the zonation of fatty acid metabolism.” Mol Cell Biochem 98:167-76 (1990).
Beg, A. A., Ruben, S. M., Scheinman, R. I., Haskill, S., Rosen, C. A., and Baldwin, A. S., Jr. “I kappa B interacts with the nuclear localization sequences of the subunits of NF-kappa B: a mechanism for cytoplasmic retention.” Genes Dev 6:1899-913 (1992).
Berry, M. N., Grivell, A. R., Grivell, M. B., and Phillips, J. W. “Isolated hepatocytes—past, present and future.” Cell Biol Toxicol 13:223-33 (1997).
Bjorbak, C., Layery, H. J., Bates, S. H., Olson, R. K., Davis, S. M., Flier, J. S., and Myers, M. G., Jr. “SOCS3 mediates feedback inhibition of the leptin receptor via Tyr985.” J Biol Chem 275:40649-57 (2000).
Blanc, V., Henderson, J. O., Newberry, E. P., Kennedy, S., Luo, J., and Davidson, N. O. “Targeted deletion of the murine apobec-1 complementation factor (acf) gene results in embryonic lethality.” Mol Cell Biol 25:7260-9 (2005).
Blanc, V., Kennedy, S. M., and Davidson, N. O. “A novel nuclear localization signal in the auxiliary domain of apobec-1 complementation factor (ACF) regulates nucleo-cytoplasmic import and shuttling.” J Biol. Chem. (2003).
Blanc, V., Henderson, J. O., Kennedy, S., and Davidson, N. O. “Mutagenesis of apobec-1 complementation factor reveals distinct domains that modulate RNA binding, protein-protein interaction with apobec-1, and complementation of C to U RNA-editing activity.” J Biol Chem 276:46386-93 (2001).
Brabant, G., Muller, G., Horn, R., Anderwald, C., Roden, M., and Nave, H. “Hepatic leptin signaling in obesity. Faseb J 19:1048-50 (2005).
Bray, G. A., Nielsen, S. J., and Popkin, B. M. “Consumption of high-fructose corn syrup in beverages may play a role in the epidemic of obesity.” Am J Clin Nutr 79:537-43 (2004).
Brown, A. M., and Gibbons, G. F “Insulin inhibits the maturation phase of VLDL assembly via a phosphoinositide 3-kinase-mediated event.” Arterioscler Thromb Vasc Biol 21:1656-61. (2001).
Browning, J. D., and Horton, J. D. “Molecular mediators of hepatic steatosis and liver injury.” J Clin Invest 114:147-52 (2004).
Carpentier, A., Taghibiglou, C., Leung, N., Szeto, L., Van Iderstine, S. C., Uffelman, K. D., Buckingham, R., Adeli, K., and Lewis, G. F. “Ameliorated hepatic insulin resistance is associated with normalization of microsomal triglyceride transfer protein expression and reduction in very low density lipoprotein assembly and secretion in the fructose-fed hamster.” J Biol Chem 277:28795-802 (2002).
Chalkley, S. M., Hettiarachchi, M., Chisholm, D. J., and Kraegen, E. W. “Long-term high-fat feeding leads to severe insulin resistance but not diabetes in Wistar rats.” Am J Physiol Endocrinol Metab 282: E1231-8 (2002).
Chen, Z., Eggerman, T. L., Patterson, A. P. (2001). Phosphorylation is a regulatory mechanism in apolipoprotein B mRNA editing. Biochem. J. 357, 661-672.
Chen, X., Sparks, J. D., Yao, Z., and Fisher, E. A. “Hepatic polysomes that contain apoprotein B mRNA have unusual physical properties.” J Biol Chem 268:1007-13 (1993).
Chen, S. H., Habib, G., Yang, C. Y., Gu, Z. W., Lee, B. R., Weng, S. A., Silberman, S. R., Cai, S. J., Deslypere, J. P., Rosseneu, M., and et al. “Apolipoprotein B-48 is the product of a messenger RNA with an organ-specific in-frame stop codon.” Science 238:363-6 (1987).
Chester et al. “Optimization of apolipoprotein B mRNA editing by APOBEC1 apoenzyme and the role of its auxiliary factor, ACF.” RNA 10: 1399-411 (2004).
Chester, A., Somasekaram, A., Tzimina, M., Jarnuz, A., Gisbourne, J., O'Keefe, R., Scott, J., and Navaratnam, N. “The apolipoprotein B mRNA editing complex performs a multifunctional cycle and suppresses nonsense-mediated decay.” Embo J22: 3971-82 (2003).
Chester et al. “RNA editing: cytidine to uridine conversionin apolipoprotein B mRNA. Biochim. Biophys Acta 1494:1-13 (2000)
Chireac, D. V., Collins, H. L., Clanci, J., Sparks, J. D., and Sparks, C. E. “Altered triglyceride-rich lipoprotein production in Zucker Diabetic Fatty rats.” Amer. J. Physiol. 287:E42-49 (2004).
Chirieac, D. V., Chirieac, L. R., Corsetti, J. P., Clanci, J., Sparks, C. E., and Sparks, J. D. “Glucose-stimulated insulin secretion suppresses hepatic triglyceride-rich lipoprotein and apoB production.” Am J Physiol Endocrinol Metab 279:E1003-11 (2000).
Corsetti, J. P., Wojciech, Z., Moss, A. J., Ridker, P. M., Marder, V. J., Rainwater, D. L and Sparks, C. E. “Metabolic Syndrome Best Defines the Multivariate Distribution of Blood Variables in Postinfarction Patients. Athersclerosis 171:In Press (2003).
Czech, M. P., Klarlund, J. K., Yagaloff, K. A., Bradford, A. P., and Lewis, R. E. “Insulin receptor signaling. Activation of multiple serine kinases.” J Biol Chem 263:11017-20 (1988).
Dance, G. S. C., Sowden, M. P., Cartegni, L., Cooper, E., Kramer, A. R., Smith, H. C. “Two proteins essential for apolipoprotein B mRNA editing are expressed from a single gene through alternative splicing.” J. Biol. Chem. 277:12703-12709 (2002).
Dashti, N., Williams, D. L., and Alaupovic, P. “Effects of oleate and insulin on the production rates and cellular mRNA concentrations of apolipoproteins in HepG2 cells.” J Lipid Res 30:1365-73 (1989).
Dreon, D. M. a. K., R. M “Alcohol, Lipids and Liporoteins,” National Institute on Alcohol Abuse and Acoholism, Bethesda, Md. (1996).
Driscoll, D. M., Lakhe-Reddy, S., Oleksa, L. M., and Martinez, D. “Induction of RNA editing at heterologous sites by sequences in apolipoprotein B mRNA.” Mol Cell Biol 13:7288-94 (1993).
Dur, S., Krause, K., Pluntke, N., and Greeve, J. “Gene structure and expression of the mouse APOBEC-1 complementation factor: multiple transcriptional initiation sites and a spliced variant with a premature stop translation codon.” Biochim Biophys Acta 1680:11-23 (2004).
Duerden, J. M., and Gibbons, G. F. “Storage, mobilization and secretion of cytosolic triacylglycerol in hepatocyte cultures. The role of insulin.” Biochem J272:583-7 (1990).
Durrington, P. N., Newton, R. S., Weinstein, D. B., and Steinberg, D. “Effects of insulin and glucose on very low density lipoprotein triglyceride secretion by cultured rat hepatocytes.” J Clin Invest 70: 63-73 (1982).
Elmquist, J. K., Maratos-Flier, E., Saper, C. B., and Flier, J. S. “Unraveling the central nervous system pathways underlying responses to leptin.” Nat Neurosci 1:445-50 (1998).
Farese, R. V., Jr., Ruland, S. L., Flynn, L. M., Stokowski, R. P., and Young, S. G. “Knockout of the mouse apolipoprotein B gene results in embryonic lethality in homozygotes and protection against diet-induced hypercholesterolemia in heterozygotes.” Proc Natl Acad Sci USA 92, 1774-8 (1995).
Festi, D., Colecchia, A., Sacco, T., Bondi, M., Roda, E., and Marchesini, G. “Hepatic steatosis in obese patients: clinical aspects and prognostic significance.” Obes Rev 5:27-42 (2004).
Fisher, E. A., Pan, M., Chen, X., Wu, X., Wang, H., Jamil, H., Sparks, J. D., and Williams, K. J. “The triple threat to nascent apolipoprotein B. Evidence for multiple, distinct degradative pathways. J Biol Chem 276:27855-63 (2001).
Frayn, K. N., Coppack, S. W., Humphreys, S. M., Clark, M. L., and Evans, R. D. Periprandial regulation of lipid metabolism in insulin-treated diabetes mellitus. Metabolism 42:504-10 (1993).
Fruhbeck, G. “Intracellular signalling pathways activated by leptin.” Biochem J393:7-20 (2006).
Fruhbeck, G. “A heliocentric view of leptin.” Proc Nutr Soc 60:301-18 (2001).
Fruhbeck, G., Jebb, S. A., and Prentice, A. M. “Leptin: physiology and pathophysiology.” Clin Physiol 18:399-419 (1998).
Fujino, T., Navaratnam, N., and Scott, J. “Human apolipoprotein B RNA editing deaminase gene (APOBEC1).” Genomics 47:266-75 (1998).
Funahashi, T. F., Giannoni, A. M., DePaoli, S. F., Skarosi and N. O., Davidson “Tissue-specific, developmental and nutritional regulation of the gene encoding the catalytic subunit of the rat apoB mRNA editing enzyme: functional role in the modulation of apoB mRNA editing.” J. Lipid Res. 36: 414-428 (1995).
Fung, P. A., Labrecque, R., and Pederson, T. “RNA-dependent phosphorylation of a nuclear RNA binding protein.” Proc Natl Acad Sci USA 94:1064-8 (1997).
Galloway, C. A., Sowden, M. P., and Smith, H. C. “Increasing the yield of soluble recombinant protein expressed in E. coli by induction during late log phase.” Biotechniques 34:524-6, 528, 530 (2003).
Gao, R., McCormick, C. J., Arthur, M. J., Ruddell, R., Oakley, F., Smart, D. E., Murphy, F. R., Harris, M. P., and Mann, D. A. “High efficiency gene transfer into cultured primary rat and human hepatic stellate cells using baculovirus vectors.” Liver 22:15-22 (2002).
Gauthier, M. S., Favier, R., and Lavoie, J. M. “Time course of the development of non-alcoholic hepatic steatosis in response to high-fat diet-induced obesity in rats.” Br J Nutr 95:273-81 (2006).
Gavrilova, O., Marcus-Samuels, B., Leon, L. R., Vinson, C., and Reitman, M. L. “Leptin and diabetes in lipoatrophic mice.” Nature 403:850; discussion 850-1 (2000).
Giangreco, A., Sowden, M. P., Mikityansky, I., and Smith, H. C “Ethanol stimulates apolipoprotein B mRNA editing in the absence of de novo RNA or protein synthesis.” Biochem Biophys Res Commun 289:1162-7. (2001).
Gibbons, G. F., and Burnham, F. J. “Effect of nutritional state on the utilization of fatty acids for hepatitic triacylglycerol synthesis and secretion as very-low-density lipoprotein.” Biochem J275 (Pt 1):87-92 (1991).
Ginsberg, H. N. “Insulin resistance and cardiovascular disease.” J Clin Invest 106:453-8 (2000).
Gomez-Lechon, M. J., Donato, M. T., Martinez-Romero, A., Jimenez, N., Castell, J. V., and O'Connor, J. E. “A human hepatocellular in vitro model to investigate steatosis.” Chem Biol Interact 165:106-16 (2007).
Gomez-Lechon, M. J., Jover, R., Donato, T., Ponsoda, X., and Castell, J. V. “Expression of liver specific-genes in hepatocytes cultured in collagen gel matrix.” Prog Mol Subcell Biol 25:89-104 (2000).
Gordon, D. A., and Jamil, H. “Progress towards understanding the role of microsomal triglyceride transfer protein in apolipoprotein-B lipoprotein assembly.” Biochim Biophys Acta 1486:72-83 (2000).
Gott and Emeson, Functions and mechanisms of RNA editing. Annu Rev Genet. 34:499-531 (2000).
Greeve, J., Altkemper, I., Dieterich, J. H., Greten, H., and Windler, E. “Apolipoprotein B mRNA editing in 12 different mammalian species: hepatic expression is reflected in low concentrations of apoB-containing plasma lipoproteins.” J Lipid Res 34:1367-83 (1993).
Hadjiagapiou, C., Giannoni, F., Funahashi, T., Skarosi, S. F., and Davidson, N. O. “Molecular cloning of a human small intestinal apolipoprotein B mRNA editing protein.” Nucleic Acids Res 22:1874-9 (1994).
Hanley, A. J., Williams, K., Festa, A., Wagenknecht, L. E., D'Agostino, R. B., Jr., Kempf, J., Zinman, B., and Haffner, S. M. “Elevations in markers of liver injury and risk of type 2 diabetes: the insulin resistance atherosclerosis study.” Diabetes 53, 2623-32 (2004).
Harris et al. “RNA editing enzyme APOBEC I and some of its homologs can act as DNA mutators.” Mol Cell 10:1247-53 (2002)
Harris et al. Extract-specific heterogeneity in high-order complexes containing apolipoprotein B mRNA editing activity and RNA-binding proteins. J. Biol. Chem. 268:7382-92 (1993)
Heini, A. F., and Weinsier, R. L. “Divergent trends in obesity and fat intake patterns: the American paradox.” Am J Med 102:259-64 (1997).
Hirano, K. I., Young, S. G., Farese, R. V., Ng, J., Sande, E., Warburton, C., Powell-Braxton, L. M., Davidson, N. O. “Targeted disruption of the mouse apobec-1 gene abolishes apolipoprotein B mRNA editing and eliminates apolipoprotein B48. J. Biol. Chem. 271:9887-9890 (1996).
Hogan, J. C., and Stephens, J. M. “The identification and characterization of a STAT 1 binding site in the PPARgamma2 promoter.” Biochem Biophys Res Commun 287: 484-92 (2001).
Huang, Y. J., Fang, V. S., Juan, C. C., Chou, Y. C., Kwok, C. F., and Ho, L. T. “Amelioration of insulin resistance and hypertension in a fructose-fed rat model with fish oil supplementation.” Metabolism 46:1252-8 (1997).
Huang, Y., Yario, T. A., and Steitz, J. A. “A molecular link between SR protein dephosphorylation and mRNA export.” Proc Natl Acad Sci USA 101:9666-70 33 (2004).
Hughes, S. D., Rouy, D., Navaratnam, N., Scott, J., and Rubin, E. M. “Gene transfer of cytidine deaminase apoBEC-1 lowers lipoprotein(a) in transgenic mice and induces apolipoprotein B editing in rabbits.” Hum Gene Ther 7:39-49 (1996).
Imbert, V., Rupec, R. A., Livolsi, A., Pahl, H. L., Traenckner, E. B., Mueller-Dieckmann, C., Farahifar, D., Rossi, B., Auberger, P., Baeuerle, P. A., and Peyron, J. F. “Tyrosine phosphorylation of I kappa B-alpha activates NF-kappa B without proteolytic degradation of I kappa B-alpha.” Cell 86:787-98 (1996).
Inui, Y., Giannoni, F., Funahashi, T., Davidson, N. O. “REPR and complementation factor(s) interact to modulate rat apolipoprotein B mRNA editing in response to alterations in cellular cholesterol flux.” J. Lipid Res. 35 (1994).
Ionescu, E., Sauter, J. F., and Jeanrenaud, B. “Abnormal oral glucose tolerance in genetically obese (fa/fa) rats.” Am J Physiol 248:E500-6 (1985).
Ishigaki, Y., Li, X., Serin, G., and Maquat, L. E. “Evidence for a pioneer round of mRNA translation: mRNAs subject to nonsense-mediated decay in mammalian cells are bound by CBP80 and CBP20.” Cell 106:607-17 (2001).
Jackson, T. K., Salhanick, A. I., Elovson, J., Deichman, M. L., and Amatruda, J. M. “Insulin regulates apolipoprotein B turnover and phosphorylation in rat hepatocytes.” J Clin Invest 86:1746-51 (1990).
Jin, X., Brooks, A., Chen, H., Bennett, R., Reichman, R. and Smith, H. “APOBEC3G/CEM15 (hA3G) mRNA levels associate inversely with human immunodeficiency virus viremia.” Virol 79:11513-6 (2005).
Johnson et al. “The mechanism for apo-B mRNA editing is deamination.” Biochem Biophys Res Commun 195:1204-10 (1993) Kahn, B. B., and Flier, J. S. “Obesity and insulin resistance.” J Clin Invest 106:473-81 (2000).
Kaptein, A., Roodenburg, L., and Princen, H. M. “Butyrate stimulates the secretion of apolipoprotein (apo) A-1 and apo B100 by the human hepatoma cell line Hep G2. Induction of apo A-1 mRNA with no change of apo B100 mRNA.” Biochem J 278 (Pt 2):557-64. (1991).
Katoh, M., and Katoh, M. “STAT3-induced WNT5A signaling loop in embryonic stem cells, adult normal tissues, chronic persistent inflammation, rheumatoid arthritis and cancer (Review).” Int J Mol Med 19:273-8 (2007).
Keegan, A., Martini, R., and Batey, R. “Ethanol-related liver injury in the rat: a model of steatosis, inflammation and pericentral fibrosis.” J Hepatol 23:591-600 (1995).
Kelley, G. L., Allan, G., and Azhar, S. “High dietary fructose induces a hepatic stress response resulting in cholesterol and lipid dysregulation.” Endocrinology 145:548-55 (2004).
Kim, J. B., and Sharp, P. A “Positive transcription elongation factor B phosphorylates hSPT5 and RNA polymerase II carboxyl-terminal domain independently of cyclin-dependent kinase-activating kinase.” J Biol Chem 276:12317-23. (2001).
Klover, P. J., Clementi, A. H., and Mooney, R. A. “Interleukin-6 depletion selectively improves hepatic insulin action in obesity.” Endocrinology 146:3417-27 (2005).
Klover, P. J., Zimmers, T. A., Koniaris, L. G., and Mooney, R. A. “Chronic exposure to interleukin-6 causes hepatic insulin resistance in mice.” Diabetes 52:2784-9 (2003).
Kraus, L. M., Traxinger, R., and Kraus, A. P. “Uremia and insulin resistance: N-carbamoyl-asparagine decreases insulin-sensitive glucose uptake in rat adipocytes.” Kidney Int 65:881-7 (2004).
Kusunoki, M., Cooney, G. J., Hara, T., and Storlien, L. H. “Amelioration of high-fat feeding-induced insulin resistance in skeletal muscle with the antiglucocorticoid RU486.” Diabetes 44:718-20. (1995).
Lam, N. T., Covey, S. D., Lewis, J. T., Oosman, S., Webber, T., Hsu, E. C., Cheung, A. T., and Kieffer, T. J “Leptin resistance following over-expression of protein tyrosine phosphatase 1B in liver.” J Mol Endocrinol 36:163-74. (2006).
Lau and Chan, Involvement of a chaperone regulator, Bcl2-associated Athanogene-4 (BAG-4), in apolipoprotein B mRNA editing. J Biol. Chem. 278:52988-96 (2003).
Lau et al. DnaJ protein, Apobec-1-binding protein-2, modulates apolipoprotein B mRNA editing. J. Biol. Chem. 276:46445-46452 (2001).
Lau, P. P., H.-J., Zhu, H A., Baldini, C., Charnsangavej, L., Chan. “Dimeric structure of a human apo B mRNA editing protein and cloning and chromosomal localization of its gene.” Proc. Natl. Acad. Sci. USA 91:8522-8526 (1994).
Le Hir, H., Izaurralde, E., Maquat, L. E., and Moore, M. J. “The spliceosome deposits multiple proteins 20-24 nucleotides upstream of mRNA exon-exon junctions.” Embo J 19:6860-9 (2000).
Lehmann, D. M., Galloway, C. A., Macelrevey, C., Sowden, M. P., Wedekind, J. E., and Smith, H. C. “Functional characterization of APOBEC-1 complementation factor phosphorylation sites.” Biochim Biophys Acta 1773:408-18 (2007).
Lehmann, D. M., Galloway, C. A., Sowden, M. P., and Smith, H. C. “Metabolic regulation of apoB mRNA editing is associated with phosphorylation of APOBEC-1 complementation factor.” Nucleic Acids Res 34:3299-308 (2006).
Lejeune, F., Li, X., Maquat, L. E. “Nonsense-Mediated mRNA Decay in Mammalian Cells Involves Decapping, Deadenylating, and Exonuclelolytic Activities.” Mol Cell 12, 675-687 (2003).
Lejeune, F., Ishigaki, Y., Li, X., and Maquat, L. E. “The exon junction complex is detected on CBP80-bound but not eIF4E-bound mRNA in mammalian cells: dynamics of mRNP remodeling.” Embo J21:3536-45. (2002).
Lellek, H., Kirsten, R., Diehl, I., Apostel, F., Buck, F., and Greeve, J. “Purification and molecular cloning of a novel essential component of the apolipoprotein B mRNA editing enzyme-complex.” J Biol Chem 275:19848-56 (2000).
Lewis, G. F., Carpentier, A., Adeli, K., and Giacca, A. “Disordered fat storage and mobilization in the pathogenesis of insulin resistance and type 2 diabetes.” Endocr Rev 23:201-29 (2002).
Lewis, G. F., and Steiner, G. “Hypertriglyceridemia and its metabolic consequences as a risk factor for atherosclerotic cardiovascular disease in non-insulin-dependent diabetes mellitus.” Diabetes Metab Rev 12:37-56 (1996).
Lewis, G. F., Uffelman, K. D., Szeto, L. W., and Steiner, G. “Effects of acute hyperinsulinemia on VLDL triglyceride and VLDL apoB production in normal weight and obese individuals.” Diabetes 42:833-42 (1993).
Li, J., Meyer, A. N., and Donoghue, D. J. “Nuclear localization of cyclin B1 mediates its biological activity and is regulated by phosphorylation.” Proc Natl Acad Sci USA 94:502-7 (1997).
Li, X., Shou, W., Kloc, M., Reddy, B. A., and Etkin, L. D. “Cytoplasmic retention of Xenopus nuclear factor 7 before the mid blastula transition uses a unique anchoring mechanism involving a retention domain and several phosphorylation sites.” J Cell Biol 124:7-17 (1994).
Liao, W., Hui, T. Y., Young, S. G., and Davis, R. A. “Blocking microsomal triglyceride transfer protein interferes with apoB secretion without causing retention or stress in the ER.” J Lipid Res 44:978-85 (2003).
Lin, J., Yang, R., Tarr, P. T., Wu, P. H., Handschin, C., Li, S., Yang, W., Pei, L., Uldry, M., Tontonoz, P., Newgard, C. B., and Spiegelman, B. M. “Hyperlipidemic effects of dietary saturated fats mediated through PGC-1beta coactivation of SREBP.” Cell 120:261-73 (2005).
Lin, M. C., Gordon, D., and Wetterau, J. R. “Microsomal triglyceride transfer protein (MTP) regulation in HepG2 cells: insulin negatively regulates MTP gene expression.” J Lipid Res 36:1073-81 (1995).
Manning, G., Whyte, D. B., Martinez, R., Hunter, T., and Sudarsanam, S. “The protein kinase complement of the human genome.” Science 298:1912-34 (2002).
Mayrand, S. H., Dwen, P., and Pederson, T. “Serine/threonine phosphorylation regulates binding of C hnRNP proteins to pre-mRNA.” Proc Natl Acad Sci USA 90:7764-8 (1993).
Mehta, A., Driscoll, D. M. “Identification of Domains in APOBEC-1 Complementation Factor Required for RNA Binding and Apolipoprotein B mRNA editing.” RNA 8:69-82 (2002).
Mehta, A., Kinter, M. T., Sherman, N. E., and Driscoll, D. M. “Molecular cloning of apobec-1 complementation factor, a novel RNA-binding protein involved in the editing of apolipoprotein B mRNA.” Mol Cell Biol 20:1846-54 (2000).
Mehta, A., and Driscoll, D. M. “A sequence-specific RNA-binding protein complements apobec-1 To edit apolipoprotein B mRNA.” Mol Cell Biol 18:4426-32 (1998).
Mehta, A., Banerjee, S., and Driscoll, D. M. “Apobec-1 interacts with a 65-kDa complementing protein to edit apolipoprotein-B mRNA in vitro.” J Biol Chem 271, 28294-9 (1996).
Mendell, J. T., Medghalchi, S. M., Lake, R. G., Noensie, E. N., and Dietz, H. C. “Novel Upf2p orthologues suggest a functional link between translation initiation and nonsense surveillance complexes.” Mol Cell Biol 20, 8944-57 (2000).
Mian et al. “Statistical modelling and phylogenetic analysis of a deaminase domain.” J Comput Biol 5:57-72 (1998).
Moberly, J. B., Cole, T. G., Alpers, D. H., and Schonfeld, G. “Oleic acid stimulation of apolipoprotein B secretion from HepG2 and Caco-2 cells occurs post-transcriptionally.” Biochim Biophys Acta 1042:70-80 (1990).
Mokuda, O., Tanaka, H., Hayashi, T., Ooka, H., Okazaki, R., and Sakamoto, Y. “Ethanol Stimulates Glycogenolysis and Inhibits both Glycogenesis via Gluconeogenesis and from Exogenous Glucose in Perfused Rat Liver.” Ann Nutr Metab 48:276-280 (2004).
Nakamuta, M., Chang, B. H. J., Zsigmond, E., Kobayashi, K., Lei, H., Ishida, B. Y., Oka, K., Li, E., Chan, L. “Complete phenotypic characterization of the apobec-1 knockout mice with a wild-type genetic background and a human apolipoprotein B transgenic background, and restoration of apolipoprotein B mRNA editing by somatic gene transfer of Apobec-1.” J. Biol. Chem. 271:25981-25988 (1996).
Nakamuta, M., Oka, K., Krushkal, J., Kobayashi, K., Yamamoto, M., Li, W. H., and Chan, L. “Alternative mRNA splicing and differential promoter utilization determine tissue-specific expression of the apolipoprotein B mRNA-editing protein (Apobec1) gene in mice. Structure and evolution of Apobec1 and related nucleoside/nucleotide deaminases.” J Biol Chem 270:13042-56 (1995).
Negro, F. “Hepatitis C virus and liver steatosis: when fat is not beautiful.” J Hepatol 40:533-5 (2004).
Nemecz, M., Preininger, K., Englisch, R., Furnsinn, C., Schneider, B., Waldhausl, W., and Roden, M. “Acute effect of leptin on hepatic glycogenolysis and gluconeogenesis in perfused rat liver. Hepatology 29:166-72 (1999).
Neufeld, D. S. “Isolation of rat liver hepatocytes.” Methods Mol Biol 75:145-51 (1997) (1997).
Newberry, E. P., Xie, Y., Kennedy, S., Han, X., Buhman, K. K., Luo, J., Gross, R. W., and Davidson, N. O. “Decreased hepatic triglyceride accumulation and altered fatty acid uptake in mice with deletion of the liver fatty acid-binding protein gene.” J Biol Chem 278:51664-72 (2003).
Niswender, K. D., and Schwartz, M. W. “Insulin and leptin revisited: adiposity signals with overlapping physiological and intracellular signaling capabilities.” Front Neuroenclocrinol 24:1-10 (2003).
O'Brien, P. J., Chan, K., and Silber, P. M. “Human and animal hepatocytes in vitro with extrapolation in vivo.” Chem Biol Interact 150:97-114 (2004).
Ohnishi, T., Yamashita, A., Kashima, I., Schell, T., Anders, K. R., Grimson, A., Hachiya, T., Hentze, M. W., Anderson, P., and Ohno, S. “Phosphorylation of hUPF1 induces formation of mRNA surveillance complexes containing hSMG-5 and hSMG-7.” Mol Cell 12: 1187-200 (2003).
Pan, M., Cederbaum, A. I., Zhang, Y. L., Ginsberg, H. N., Williams, K. J., and Fisher, E. A. “Lipid peroxidation and oxidant stress regulate hepatic apolipoprotein B degradation and VLDL production.” J Clin Invest 113:1277-87 (2004).
Park S. Y., Cho, Y. R., Kim, H. J., Higashimori, T., Danton, C., Lee, M. K., Dey, A., Rothermel, B., Kim, Y. B., Kalinowski, A., Russell, K. S., Kim, J. K. “Unraveling the temporal pattern of diet-induced insulin resistance in individual organs and cardiac dysfunction in C57BL/6 mice.” Diabetes 54:3530-40 (2005)
Patsch, W., Gotto, A. M., Jr., and Patsch, J. R. “Effects of insulin on lipoprotein secretion in rat hepatocyte cultures. The role of the insulin receptor.” J Biol Chem 261:9603-6 (1986).
Patsch, W., Franz, S., and Schonfeld, G “Role of insulin in lipoprotein secretion by cultured rat hepatocytes.” J Clin Invest 71:1161-74. (1983).
Pedersen, O., Kahn, C. R., Flier, J. S., and Kahn, B. B. “High fat feeding causes insulin resistance and a marked decrease in the expression of glucose transporters (Glut 4) in fat cells of rats.” Endocrinology 129, 771-7 (1991).
Pei, Y., Hausmann, S., Ho, C. K., Schwer, B., and Shuman, S. “The length, phosphorylation state, and primary structure of the RNA polymerase II carboxyl-terminal domain dictate interactions with mRNA capping enzymes.” J Biol Chem 276, 28075-82 (2001).
Pelleymounter, M. A., Cullen, M. J., Baker, M. B., Hecht, R., Winters, D., Boone, T., and Collins, F. “Effects of the obese gene product on body weight regulation in ob/ob mice.” Science 269:540-3 (1995).
Perlman, D. H., Berg, E. A., O'Connor P, B., Costello, C. E., and Hu, J. “Reverse transcription-associated dephosphorylation of hepadnavirus nucleocapsids.” Proc Natl Acad Sci USA (2005).
Petersen-Mahrt et al. In vitro deamination of cytosine to uracil in single-stranded DNA by apolipoprotein B editing complex catalytic subunit 1 (APOBEC1). J Biol Chem 278:19583-6 (2003). Phung, T. L., M. P., Sowden, J. D., Sparks, C. E., Sparks, H. C., Smith “Regulation of hepatic apoB RNA editing in the genetically obese Zucker rat.” Metabolism 45, 1056-1058 (1996).
Phung, T. L., Sowden, M. P., Sparks, J. D., Sparks, C. E., and Smith, H. C. “Regulation of hepatic apolipoprotein B RNA editing in the genetically obese Zucker rat.” Metabolism 45:1056-8 (1996).
Piessevaux, J., Lavens, D., Montoye, T., Wauman, J., Catteeuw, D., Vandekerckhove, J., Belsham, D., Peelman, F., and Tavernier, J. “Functional cross-modulation between SOCS proteins can stimulate cytokine signaling.” J Biol Chem 281:32953-66 (2006).
Podolin, D. A., Gayles, E. C., Wei, Y., Thresher, J. S., and Pagliassotti, M. J. Menhaden oil prevents but does not reverse sucrose-induced insulin resistance in rats.” Am J Physiol 274:R840-8 (1998).
Pontrelli, L., Sidiropoulos, K. G., and Adeli, K. “Translational control of apolipoprotein B mRNA: regulation via cis elements in the 5′ and 3′ untranslated regions.” Biochemistry 43:6734-44 (2004).
Powell, L. M., Wallis, S. C., Pease, R. J., Edwards, Y. H., Knott, T. J., and Scott, J. A novel form of tissue-specific RNA processing produces apolipoprotein-B48 in intestine. Cell 50:831-40 (1987).
Pullinger, C. R., North, J. D., Teng, B. B., Rifici, V. A., Ronhild de Brito, A. E., and Scott, J. “The apolipoprotein B gene is constitutively expressed in HepG2 cells: regulation of secretion by oleic acid, albumin, and insulin, and measurement of the mRNA half-life.” J Lipid Res 30:1065-77 (1989).
Qian, X., Balestra, M. E., Yamanaka, S., Boren, J., Lee, I., and Innerarity, T. L. “Low expression of the apolipoprotein B mRNA-editing transgene in mice reduces LDL levels but does not cause liver dysplasia or tumors.” Arterioscler Thromb Vasc Biol 18:1013-20 (1998).
Raabe, M., Flynn, L. M., Zlot, C. H., Wong, J. S., Veniant, M. M., Hamilton, R. L., and Young, S. G. “Knockout of the abetalipoproteinemia gene in mice: reduced lipoprotein secretion in heterozygotes and embryonic lethality in homozygotes.” Proc Natl Acad Sci USA 95:8686-91 (1998).
Ro, S., Hwang, S. J., Ordog, T. and Sanders, K. M. “Adenovirus-based short hairpin RNA vectors containing an EGFP marker and mouse U6, human H1, or human U6 promoter.” Biotechniques 38:625-7 (2005).
Rochon, J “Application of GEE procedures for sample size calculations in repeated measures experiments.” Stat Med 17:1643-58. (1998).
Rubin, R., Seiler, A. and Rooney, T. “Ethanol-Induced Inhibition of Insulin-Like Growth Factor I Signaling,” National Institutes of Alocohol Abuse and Alcoholism, Bethesda, Md. (2000).
Rustaeus, S., Stillemark, P., Lindberg, K., Gordon, D., and Olofsson, S. O. “The microsomal triglyceride transfer protein catalyzes the post-translational assembly of apolipoprotein B-100 very low density lipoprotein in McA-RH7777 cells.” J Biol Chem 273:5196-203 (1998).
Samuel, V. T., Liu, Z. X., Qu, X., Elder, B. D., Bilz, S., Befroy, D., Romanelli, A. J., and Shulman, G. I. “Mechanism of hepatic insulin resistance in non-alcoholic fatty liver disease. J Biol Chem 279:32345-53 (2004).
Schonfeld, G., and Pfleger, B. “Utilization of exogenous free fatty acids for the production of very low density lipoprotein triglyceride by livers of carbohydrate-fed rats. J Lipid Res 12:614-21 (1971).
Schroeder, F., Jolly, C. A., Cho, T. H., and Frolov, A. “Fatty acid binding protein isoforms: structure and function.” Chem Phys Lipids 92:1-25 (1998).
Scott, J. “The molecular and cell biology of apolipoprotein-B.” Mol Biol Med 6, 65-80 (1989).
Senn, J. J., Klover, P. J., Nowak, I. A., Zimmers, T. A., Koniaris, L. G., Furlanetto, R. W., and Mooney, R. A. “Suppressor of cytokine signaling-3 (SOCS-3), a potential mediator of interleukin-6-dependent insulin resistance in hepatocytes.” J Biol Chem 278:13740-6 (2003).
Shafrir, E. “Animal models of non-insulin-dependent diabetes.” Diabetes Metab Rev 8:179-208 (1992).
Shah, R. R., Knott, T. J., Legros, J. E., Navaratnam, N., Greeve, J. C., and Scott, J. “Sequence requirements for the editing of apolipoprotein B mRNA.” J Biol Chem 266:16301-4 (1991).
Shelness, G. S., and Ledford, A. S. “Evolution and mechanism of apolipoprotein B-containing lipoprotein assembly.” Curr Opin Lipidol 16:325-32 (2005).
Shi, K., Egawa, K., Maegawa, H., Nakamura, T., Ugi, S., Nishio, Y., and Kashiwagi, A. “Protein-tyrosine phosphatase 1B associates with insulin receptor and negatively regulates insulin signaling without receptor internalization.” J Biochem (Tokyo) 136:89-96 (2004).
Shimomura, I., Hammer, R. E., Ikemoto, S., Brown, M. S., and Goldstein, J. L. “Leptin reverses insulin resistance and diabetes mellitus in mice with congenital lipodystrophy.” Nature 401:73-6 (1999).
Siddiqui et al. “Disproportionate relationship between APOBEC-1 expression and apolipoprotein B mRNA editing activity.” Exp Cell Res 252:154-64 (1999).
Singh, K., Batuman, 0. A., Akman, H. O., Kedees, M. H., Vakil, V., and Hussain, M. M. “Differential, tissue-specific, transcriptional regulation of apolipoprotein B secretion by transforming growth factor!” Jounial of Biological Chemistry 277:39515-39524 (2002).
Smith, H. C. “Analysis of protein complexes assembled on apolipoprotein B mRNA for mooring sequence-dependent RNA editing.” Methods 15:27-39 (1998).
Smith et al. “A guide to RNA editing.” Rna 3:1105-23 (1997) Smith, H. C. “Apolipoprotein B mRNA editing: the sequence to the event.” Semin Cell Biol 4:267-78 (1993).
Smith et al. “In vitro apolipoprotein B mRNA editing: identification of a 27S editing complex.” Proc Natl Acad Sci USA 88:1489-93 (1991).
Sowden, M. P., Lehmann, D. M., Lin, X., Smith C. O. and Smith, H. C. “Identification of Novel Alternative Splice Variants of APOBEC-1 Complementation Factor with Different Capacities to Support ApoB mRNA Editing.” J. Biol. Chem. 278:197-206 (2004).
Sowden, M. P., Ballatori, N., de Mesy Jensen, K. L., Hamilton Reed, L., Smith, H. C. “The editosome for cytidine to uridine mRNA editing has a native complexity of 27S: identification of intracellular domains containing active and inactive editing factors.” J Cell Science 115:1027-1039 (2002). Sowden and Smith., “Commitment of apolipoprotein B RNA to the splicing pathway regulates cytidine-to-uridine editing-site utilization.” Biochem J 359:697-705 (2001).
Sowden, M. P., Collins, H. L., Smith, H. C., Garrow, T. A., Sparks, J. D., and Sparks, C. E. “Apolipoprotein B mRNA and lipoprotein secretion are increased in McArdle RH-7777 cells by expression of betaine-homocysteine S-methyltransferase.” Biochem J 341 (Pt 3):639-45 (1999).
Sowden et al. “Apolipoprotein B RNA sequence 3′ of the mooring sequence and cellular sources of auxiliary factors determine the location and extent of promiscuous editing. “Nucleic Acids Research 26:1644-52 (1998).
Sowden et al. “Overexpression of APOBEC-1 results in mooring sequence-dependent promiscuous RNA editing.” J Biol Chem 271:3011-7 (1996).
Sowden et al. “Identification of Novel Alternative Splice Variants of APOBEC-1 Complementation Factor with Different Capacities to Support ApoB mRNA Editing.” J. Biol. Chem. 278:197-206 (2004).
Sparks, J. D., Phung, T. L., Bolognino, M., and Sparks, C. E. “Insulin-mediated inhibition of apolipoprotein B secretion requires an intracellular trafficking event and phosphatidylinositol 3-kinase activation: studies with brefeldin A and wortmannin in primary cultures of rat hepatocytes.” Biochem J313 (Pt 2):567-74 (1996).
Sparks, J. D., and Sparks, C. E. “Insulin regulation of triacylglycerol-rich lipoprotein synthesis and secretion.” Biochim Biophys Acta 1215:9-32 (1994).
Sparks, J. D. and Sparks, C. E. “Obese Zucker (fa/fa) rats are resistant to insulin's inhibitory effect on hepatic apo B secretion.” Biochem. Biophys. Res. Commun. 205:417-422 (1994).
Sparks, J. D., Zolfaghari, R., Sparks, C. E., Smith, H. C., and Fisher, E. A. Impaired hepatic apolipoprotein B and E translation in streptozotocin diabetic rats. J Clin Invest 89:1418-30 (1992).
Sparks, J. D., and Sparks, C. E. “Insulin modulation of hepatic synthesis and secretion of apoB by rat hepatocytes.” J Biol Chem 265, 8854-62 (1990).
Sparks, J. D., Sparks, C. E., and Miller, L. L. “Insulin effects on apolipoprotein B production by normal, diabetic and treated-diabetic rat liver and cultured rat hepatocytes.” Biochem J 261:83-8 (1989).
Sparks, J. D., Sparks, C. E., Bolognino, M., Roncone, A. M., Jackson, T. K., and Amatruda, J. M. “Effects of nonketotic streptozotocin diabetes on apolipoprotein B synthesis and secretion by primary cultures of rat hepatocytes.” J. Clin. Invest. 82:37-43 (1988).
Sparks, C. E., and Marsh, J. B. “Metabolic heterogeneity of apolipoprotein B in the rat.” J Lipid Res 22:519-27 (1981).
Sparks, C. E., Hnatiuk, O., and Marsh, J. B. “Hepatic and intestinal contribution of two forms of apolipoprotein B to plasma lipoprotein fractions in the rat.” Can J Biochem 59:693-9 (1981).
Standridge, J. B., Zylstra, R. G., and Adams, S. M. “Alcohol consumption: an overview of benefits and risks.” South Med J97:664-72 (2004).
Steinburg et al. “Tissue-specific differences in the role of RNA 3′ of the apo B mRNA mooring sequence in editosome assembly.” Biochem. Biophys. Res. Commun. 263:81-86 (1999).
Steinberger, J., and Daniels, S. RObesity, insulin resistance, diabetes, and cardiovascular risk in children: an American Heart Association scientific statement from the Atherosclerosis, Hypertension, and Obesity in the Young Committee (Council on Cardiovascular Disease in the Young) and the Diabetes Committee (Council on Nutrition, Physical Activity, and Metabolism). Circulation 107:1448-53 (2003).
Storlien, L. H., James, D. E., Burleigh, K. M., Chisholm, D. J., and Kraegen, E. W. “Fat feeding causes widespread in vivo insulin resistance, decreased energy expenditure, and obesity in rats.” Am J Physiol 251:E576-83 (1986).
Tacke, R., Chen, Y., and Manley, J. L. “Sequence-specific RNA binding by an SR protein requires RS domain phosphorylation: creation of an SRp40-specific splicing enhancer.” Proc Natl Acad Sci USA 94:1148-53 (1997).
Taghibiglou, C., Carpentier, A., Van Iderstine, S. C., Chen, B., Rudy, D., Aiton, A., Lewis, G. F., and Adeli, K. “Mechanisms of hepatic very low density lipoprotein overproduction in insulin resistance. Evidence for enhanced lipoprotein assembly, reduced intracellular ApoB degradation, and increased microsomal triglyceride transfer protein in a fructose-fed hamster model.” J Biol Chem 275, 8416-25 (2000).
Tartaglia, L. A., Dembski, M., Weng, X., Deng, N., Culpepper, J., Devos, R., Richards, G. J., Campfield, L. A., Clark, F. T., Deeds, J., Muir, C., Sanker, S., Moriarty, A., Moore, K. J., Smutko, J. S., Mays, G. G., Wool, E. A., Monroe, C. A., and Tepper, R. I. “Identification and expression cloning of a leptin receptor, OB-R.” Cell 83, 1263-71 (1995).
Teng et al. “Mutational analysis of apolipoprotein B mRNA editing enzyme (APOBEC1). structure-function relationships of RNA editing and dimerization.” J Lipid Res 40:623-35 (1999)
Teng, B., S. Blumenthal, T. Forte, N. Navaratnam, J. Scott, A. M. Gotto Jr., L. Chan “Adenovirus-mediated gene transfer of rat apolipoprotein B mRNA editing protein in mice virtually eliminates apolipoprotein B-100 and normal low density lipoprotein production.” J. Biol. Chem. 269:29395-29404 (1994).
Teng, B., Burant, C. F., and Davidson, N. O. “Molecular cloning of an apolipoprotein B messenger RNA editing protein.” Science 260:1816-1819 (1993).
Terrettaz, J., and Jeanrenaud, B. “In vivo hepatic and peripheral insulin resistance in genetically obese (fa/fa) rats.” Endocrinology 112:1346-51 (1983).
Theriault, A., Cheung, R., and Adeli, K. “Expression of apolipoprotein B in vitro in cell-free lysates of HepG2 cells: evidence that insulin modulates ApoB synthesis at the translational level.” Clin Biochem 25, 321-3 (1992).
Tilg, H., and Moschen, A. R. “Adipocytokines: mediators linking adipose tissue, inflammation and immunity.” Nat Rev Immunol 6:772-83 (2006).
Touyz, R. M., and Schiffrin, E. L. “Peroxisome proliferator-activated receptors in vascular biology-molecular mechanisms and clinical implications.” Vascul Pharmacol 45:19-28 (2006).
Tu, X. M., Kowalski, J., Zhang, J., Lynch, K. G., and Crits-Christoph, P. “Power analyses for longitudinal trials and other clustered designs.” Stat Med 23:2799-815 (2004).
Ugi, S., Imamura, T., Maegawa, H., Egawa, K., Yoshizaki, T., Shi, K., Obata, T., Ebina, Y., Kashiwagi, A., and Olefsky, J. M. “Protein phosphatase 2A negatively regulates insulin's metabolic signaling pathway by inhibiting Akt (protein kinase B) activity in 3T3-L1 adipocytes.” Mol Cell Biol 24:8778-89 (2004).
van de Wiel, A. “Diabetes mellitus and alcohol”. Diabetes Metab Res Rev 20, 263-7 (2004).
Van Mater, D., Sowden, M. P., Clanci, J. Sparks, J. D., Sparks C. E., Ballatori, N. & Smith, H. C. “Ethanol Increases Apob Mma Editing In Rat Primary Hepatocyte And Mcardle Cells.” Biochem. Biophys Res. Commun. 252:334-9 (1989).
Veniant, M. M., Kim, E., McCormick, S., Boren, J., Nielsen, L. B., Raabe, M., and Young, S. G. “Insights into apolipoprotein B biology from transgenic and gene-targeted mice.” J Nutr 129:451 S-455S (1999).
von Wronski, M. A., Hirano, K. I., Cagen, L. M., Wilcox, H. G., Raghow, R., Thorngate, F. E., Heimberg, M., Davidson, N. O., and Elam, M. B. “Insulin increases expression of apobec-1, the catalytic subunit of the apolipoprotein B mRNA editing complex in rat hepatocytes.” Metabolisn 47:869-73 (1998).
Wang, T. W. M., Byrne, C. D., and Hales, C. N. “Effect of ethanol on hepatic apolipoprotein B synthesis and secretion in vitro.” Biochimica et Biophysica Acta 1211:234-238 (1994).
Wedekind, J. E., Dance, G. S., Sowden, M. P., and Smith, H. C. “Messenger RNA editing in mammals: new members of the APOBEC family seeking roles in the family business.” Trends Genet. 19:207-16 (2003).
Werner, E. D., Lee, J., Hansen, L., Yuan, M., and Shoelson, S. E. “Insulin resistance due to phosphorylation of insulin receptor substrate-1 at serine 302.” J Biol Chem 279:35298-305 (2004).
Wetterau, J. R., Lin, M. C., and Jamil, H. “Microsomal triglyceride transfer protein.” Biochim Biophys Acta 1345:136-50 (1997).
Wilson, G. M., Lu, J., Sutphen, K., Sun, Y., Huynh, Y., and Brewer, G. “Regulation of A+U-rich element-directed mRNA turnover involving reversible phosphorylation of AUFI.” J Biol Chem 278:33029-38 (2003).
Xiao, S. H., and Manley, J. L. “Phosphorylation of the ASF/SF2 RS domain affects both protein-protein and protein-RNA interactions and is necessary for splicing.” Genes Dev 11:334-44 (1997).
Xie, Y., Nassir, F., Luo, J., Buhman, K., and Davidson, N. O. “Intestinal lipoprotein assembly in apobec-1−/− mice reveals subtle alterations in triglyceride secretion coupled with a shift to larger lipoproteins.” Am J Physiol Gastrointest Liver Physiol 285, G735-46 (2003)
Xu, Y. “Regulation of p53 responses by post-translational modifications.” Cell Death Differ 10:400-3 (2003).
Yagmur, E., Trautwein, C., Gressner, A. M., and Tacke, F. “Resistin serum levels are associated with insulin resistance, disease severity, clinical complications, and prognosis in patients with chronic liver diseases.” Am J Gastroenterol 101: 1244-52 (2006).
Yamanaka et al. “Hyperediting of multiple cytidines of apolipoprotein B mRNA by APOBEC-1 requires auxiliary protein(s) but not a mooring sequence motif.” J. Biol. Chem. 271:11506-11510 (1996)
Yamanaka et al. “Apolipoprotein B mRNA-editing protein induces hepatocellular carcinoma and dysplasia in transgenic animals.” Proc Natl Acad Sci USA 92:8483-7 (1995).
Yamanaka et al. “A novel translational repressor mRNA is edited extensively in livers containing tumors caused by the transgene expression of the apoB mRNA-editing enzyme. Genes Dev 11:321-33 (1996).
Yamanaka, S., P'oksay, K. S., Balestra, M. E., Zeng, G. Q., and Innerarity, T. L. “Cloning and mutagenesis of the rabbit ApoB mRNA editing protein. A zinc motif is essential for catalytic activity, and noncatalytic auxiliary factor(s) of the editing complex are widely distributed.” J Biol Chem 26:21725-34 (1994).
Yang et al. “Apolipoprotein B mRNA editing and the reduction in synthesis and secretion of the atherogenic risk factor, apolipoprotein B100 can be effectively targeted through TAT-mediated protein transduction.” Mol Pharmacol 61:269-76 (2002).
Yang, Y., M. P., Sowden Y., Yang, H. C., Smith “Intracellular Trafficking Determinants in APOBEC-1, the Catalytic Subunit for Cytidine to Uridine Editing of Apolipoprotein B mRNA.” Exp. Cell Res. 267:153-164 (2001).
Yang, Y., Sowden, M. P., and Smith, H. C. “Induction of cytidine to uridine editing on cytoplasmic apolipoprotein B mRNA by overexpressing APOBEC-1.” J Biol Chem 275:22663-9 (2000).
Yang, Y., and Smith, H. C. “Multiple protein domains determine the cell type-specific nuclear distribution of the catalytic subunit required for apolipoprotein B mRNA editing.” Proc Natl Acad Sci USA 94:13075-80 (1997).
Yang et al. “Partial characterization of the auxiliary factors involved in apolipoprotein B mRNA editing through APOBEC-1 affinity chromatography.” J Biol Chem 272:27700-6 (1997).
Yao, Z., and McLeod, R. S. “Synthesis and secretion of hepatic apolipoprotein B-containing lipoproteins.” Biochim Biophys Acta 1212:152-66 (1994).
Yi, S. J., and Jhun, B. H. “Ethanol impairs insulin's actions through phosphatidylinositol 3-kinase.” J Med Food 7:24-30 (2004).
Zabolotny, J. M., Bence-Hanulec, K. K., Stricker-Krongrad, A., Haj, F., Wang, Y., Minokoshi, Y., Kim, Y. B., Elmquist, J. K., Tartaglia, L. A., Kahn, B. B., and Neel, B. G. “PTP1B regulates leptin signal transduction in vivo.” Dev Cell 2:489-95 (2002).
Zhang et al. “At least one intron is required for the nonsense-mediated decay of triosephosphate isomerase mRNA: a possible link between nuclear splicing and cytoplasmic translation.” Mol Cell Biol 18:5272-83 (1998).
Zhang, J., Sun, X., Qian, Y., and Maquat, L. E. (Intron function in the nonsense-mediated decay of beta-globin mRNA: indications that pre-mRNA splicing in the nucleus can influence mRNA translation in the cytoplasm.” Rna 4:801-15 (1998).
Zhang, Y., Proenca, R., Maffei, M., Barone, M., Leopold, L., and Friedman, J. M. “Positional cloning of the mouse obese gene and its human homologue”. Nature 372, 425-32 (1994).
Zhu, B., and Gulick, T “Phosphorylation and alternative pre-mRNA splicing converge to regulate myocyte enhancer factor 2C activity.”Mol Cell Biol 24:8264-75 (2004).
Zucker, L. M., and Antoniades, H. N. “Insulin and obesity in the Zucker genetically obese rat “fatty”.” Endocrinology 90:1320-30 (1972).

What is claimed is:

1. A polypeptide comprising an ACF sequence, a secretion sequence and a transduction sequence.

2. The polypeptide of claim 1, wherein the ACF sequence is selected from the group of mRNA spliced variants consisting of an ACF65 sequence, an ACF64 sequence, an ACF45 sequence, and an ACF43 sequence, wherein the ACF65 sequence comprises the amino acid sequence SEQ ID NO:17, an amino acid sequence at least about 90% identical to the amino acid sequence of SEQ ID NO:17, the amino acid sequence SEQ ID NO:17 having one or more conservative amino acid substitutions, or the amino acid sequence SEQ ID NO:17 having one or more mutations;
wherein the ACF64 sequence comprises the amino acid sequence SEQ ID NO:1, an amino acid sequence at least about 90% identical to the amino acid sequence of SEQ ID NO:1, the amino acid sequence SEQ ID NO:1 having one or more conservative amino acid substitutions, or the amino acid sequence SEQ ID NO:1 having one or more mutations;
wherein the ACF45 sequence comprises the amino acid sequence SEQ ID NO:18, an amino acid sequence at least about 90% identical to the amino acid sequence of SEQ ID NO:18, the amino acid sequence SEQ ID NO:18 having one or more conservative amino acid substitutions, or the amino acid sequence SEQ ID NO:18 having one or more mutations; and
wherein the ACF43 sequence comprises the amino acid sequence SEQ ID NO:19, an amino acid sequence at least about 90% identical to the amino acid sequence of SEQ ID NO:19, the amino acid sequence SEQ ID NO:19 having one or more conservative amino acid substitutions, or the amino acid sequence SEQ ID NO:19 having one or more mutations.

3. The polypeptide of claim 1, wherein the ACF sequence has a mutation at one or more sites where ACF is phosphorylated.

4. The polypeptide of claim 1, wherein the ACF sequence has a serine to alanine or aspartic acid substitution at one or more sites where ACF is phosphorylated.

5. The polypeptide of claim 4, wherein the amino acid substitution comprises a serine to alanine or aspartic acid substitution at the amino acid residue corresponding to amino acid residue 154 in SEQ ID NO:1.

6. The polypeptide of claim 4, wherein the amino acid substitution comprises a serine to alanine or aspartic acid substitution at the amino acid residue corresponding to amino acid residue 368 in SEQ ID NO:1.

7. The polypeptide of claim 6, wherein the ACF has a second amino acid substitution, wherein the second amino acid substitution comprises a serine to alanine or aspartic acid substitution at the amino acid residue corresponding to amino acid residue 154 in SEQ ID NO:1.

8. The polypeptide of claim 1, wherein the transduction sequence is a TAT sequence.

9. The polypeptide of claim 8, wherein the TAT sequence comprises SEQ ID NO:23.

10. The polypeptide of claim 1, wherein the secretion sequence is an albumin signal sequence.

11. The polypeptide of claim 10, wherein the albumin signal sequence comprises SEQ ID NO:24.

12. The polypeptide of claim 1, wherein, when a cell produces the polypeptide, the polypeptide is secreted from the cell, and the polypeptide transduces a second cell, thereby delivering the polypeptide to the second cell.

13. The polypeptide of claim 12, wherein delivery of the polypeptide to the second cell increases transport of ApoB mRNA from the nucleus to the cytoplasm of the second cell.

14. A composition comprising the polypeptide of claim 1.

15. A nucleic acid encoding the polypeptide of claim 1.

16. A vector comprising the nucleic acid of claim 15.

17. A cell comprising the vector of claim 16.

18. A method of expressing ACF in a cell, comprising bringing into contact a cell and a vector comprising a nucleic acid, wherein the nucleic acid encodes a polypeptide comprising an ACF sequence, a secretion sequence and a transduction sequence; whereby the nucleic acid produces the polypeptide, thereby expressing ACF in the cell.

19. The method of claim 18, wherein the polypeptide is secreted from the cell, and wherein the polypeptide transduces a second cell, thereby delivering the polypeptide to the second cell.

20. The method of claim 19, wherein delivery of the polypeptide to the second cell increases transport of ApoB mRNA from the nucleus to the cytoplasm of the second cell.

21. The method of any claim 20, wherein the polypeptide is delivered via transduction.

22. The method of claim 21, wherein expression of ApoB protein in the cytoplasm of the second cell is increased.

23. The method of claim 22, wherein increased expression of ApoB protein leads to a reduction in the level of lipid in the second cell.

24. The method of claim 18, wherein the cell is a liver cell.

25. The method of claim 18, wherein the cell is in a subject.

26. The method of claim 25, wherein the subject has one or more of the following:
nonalcoholic fatty liver disease (steatohepatitis), alcoholic fatty liver disease (alcoholic hepatic steatosis), viral induced steatohepatitis (hepatitis B infection), liver cirosis chronic hyperinsulinemia, type II diabetes, or obesity.

27. The method of claim 18, wherein the ACF sequence is selected from the group consisting of an ACF65 sequence, an ACF64 sequence, an ACF45 sequence, and an ACF43 sequence.

28. The method of claim 27, wherein the ACF65 sequence comprises the amino acid sequence SEQ ID NO:17, an amino acid sequence at least about 90% identical to the amino acid sequence of SEQ ID NO:17, the amino acid sequence SEQ ID NO:17 having one or more conservative amino acid substitutions, or the amino acid sequence SEQ ID NO:17 having one or more mutations;
wherein the ACF64 sequence comprises the amino acid sequence SEQ ID NO:1, an amino acid sequence at least about 90% identical to the amino acid sequence of SEQ ID NO:1, the amino acid sequence SEQ ID NO:1 having one or more conservative amino acid substitutions, or the amino acid sequence SEQ ID NO:1 having one or more mutations;
wherein the ACF45 sequence comprises the amino acid sequence SEQ ID NO:18, an amino acid sequence at least about 90% identical to the amino acid sequence of SEQ ID NO:18, the amino acid sequence SEQ ID NO:18 having one or more conservative amino acid substitutions, or the amino acid sequence SEQ ID NO:18 having one or more mutations; and
wherein the ACF43 sequence comprises the amino acid sequence SEQ ID NO:19, an amino acid sequence at least about 90% identical to the amino acid sequence of SEQ ID NO:19, the amino acid sequence SEQ ID NO:19 having one or more conservative amino acid substitutions, or the amino acid sequence SEQ ID NO:19 having one or more mutations.

29. The method of claim 28, wherein the ACF sequence has a mutation at one or more sites where ACF is phosphorylated.

30. The method of claim 28, wherein the ACF sequence has a serine to alanine or aspartic acid substitution at one or more sites where ACF is phosphorylated.

31. The method of claim 30, wherein the amino acid substitution comprises a serine to alanine or aspartic acid substitution at the amino acid residue corresponding to amino acid residue 154 in SEQ ID NO:1.

32. The method of claim 30, wherein the amino acid substitution comprises a serine to alanine or aspartic acid substitution at the amino acid residue corresponding to amino acid residue 368 in SEQ ID NO:1.

33. The method of claim 32, wherein the ACF has a second amino acid substitution, wherein the second amino acid substitution comprises a serine to alanine or aspartic acid substitution at the amino acid residue corresponding to amino acid residue 154 in SEQ ID NO:1.

34. The method of claim 18, wherein the transduction sequence is a TAT sequence.

35. The method of claim 34, wherein the TAT sequence comprises SEQ ID NO:23.

36. The method of claim 18, wherein the secretion sequence is an albumin signal sequence.

37. The method of claim 36, wherein the albumin signal sequence comprises SEQ ID NO:24.

38. The method of claim 18, wherein the nucleic acid encoding the polypeptide is operably linked to an expression control sequence.

39. The method of claim 18, wherein the vector further comprises a second nucleic acid, wherein the second nucleic acid encodes a second polypeptide, wherein the second polypeptide comprises an APOBEC-1 sequence, a second secretion sequence and a second transduction sequence; whereby the nucleic acid produces the second polypeptide, thereby expressing APOBEC-1 in the cell.

40. The method of claim 18 further comprising bringing into contact the cell and a second vector, wherein the second vector comprises a second nucleic acid, wherein the second nucleic acid encodes a second polypeptide, wherein the second polypeptide comprises an APOBEC-1 sequence, a second secretion sequence and a second transduction sequence; whereby the nucleic acid produces the second polypeptide, thereby expressing APOBEC-1 in the cell.

41. The method of claim 39, wherein the second polypeptide is secreted from the cell, and wherein the second polypeptide transduces a second cell, thereby delivering the second polypeptide to the second cell.

42. The method of claim 39, wherein the APOBEC-1 sequence comprises the amino acid sequence SEQ ID NO:8, an amino acid sequence at least about 90% identical to the amino acid sequence of SEQ ID NO:8, or the amino acid sequence SEQ ID NO:8 having one or more conservative amino acid substitutions.

43. A vector comprising a nucleic acid, wherein the nucleic acid encodes a polypeptide comprising an ACF sequence, a secretion sequence and a transduction sequence.

44. The vector of claim 43, wherein the ACF sequence is selected from the group consisting of an ACF65 sequence, an ACF64 sequence, an ACF45 sequence, and an ACF43 sequence, wherein the ACF65 sequence comprises the amino acid sequence SEQ ID NO:17, an amino acid sequence at least about 90% identical to the amino acid sequence of SEQ ID NO:17, the amino acid sequence SEQ ID NO:17 having one or more conservative amino acid substitutions, or the amino acid sequence SEQ ID NO:17 having one or more mutations;
wherein the ACF64 sequence comprises the amino acid sequence SEQ ID NO:1, an amino acid sequence at least about 90% identical to the amino acid sequence of SEQ ID NO:1, the amino acid sequence SEQ ID NO:1 having one or more conservative amino acid substitutions, or the amino acid sequence SEQ ID NO:1 having one or more mutations;
wherein the ACF45 sequence comprises the amino acid sequence SEQ ID NO:18, an amino acid sequence at least about 90% identical to the amino acid sequence of SEQ ID NO:18, the amino acid sequence SEQ ID NO:18 having one or more conservative amino acid substitutions, or the amino acid sequence SEQ ID NO:18 having one or more mutations; and
wherein the ACF43 sequence comprises the amino acid sequence SEQ ID NO:19, an amino acid sequence at least about 90% identical to the amino acid sequence of SEQ ID NO:19, the amino acid sequence SEQ ID NO:19 having one or more conservative amino acid substitutions, or the amino acid sequence SEQ ID NO:19 having one or more mutations.

45. The vector of claim 43, wherein the ACF sequence has a mutation at one or more sites where ACF is phosphorylated.

46. The vector of claim 45, wherein the ACF sequence has a serine to alanine or aspartic acid substitution at one or more sites where ACF is phosphorylated.

47. The vector of claim 46, wherein the amino acid substitution comprises a serine to alanine or aspartic acid substitution at the amino acid residue corresponding to amino acid residue 154 in SEQ ID NO:1.

48. The vector of claim 46, wherein the amino acid substitution comprises a serine to alanine or aspartic acid substitution at the amino acid residue corresponding to amino acid residue 368 in SEQ ID NO:1.

49. The vector of claim 48, wherein the ACF has a second amino acid substitution, wherein the second amino acid substitution comprises a serine to alanine or aspartic acid substitution at the amino acid residue corresponding to amino acid residue 154 in SEQ ID NO:1.

50. The vector of claim 46, wherein the vector further comprises a second nucleic acid, wherein the second nucleic acid encodes a second polypeptide, wherein the second polypeptide comprises an APOBEC-1 sequence, a second secretion sequence and a second transduction sequence; whereby the nucleic acid produces the second polypeptide, thereby expressing APOBEC-1 in the cell.

51. The vector of claim 50, wherein the second polypeptide is secreted from the cell, and wherein the second polypeptide transduces a second cell, thereby delivering the second polypeptide to the second cell.

52. The vector of claim 46, wherein the APOBEC-1 sequence comprises the amino acid sequence SEQ ID NO:8, an amino acid sequence at least about 90% identical to the amino acid sequence of SEQ ID NO:8, or the amino acid sequence SEQ ID NO:8 having one or more conservative amino acid substitutions.

53. A composition comprising the vector of claim 46.

54. A cell comprising the vector of claim 46.

55. The cell of claim 54, wherein the cell is a hepatocyte.

56. The vector of claim 46, wherein, when the vector is brought into contact with a cell, the nucleic acid produces the polypeptide, the polypeptide is secreted from the cell, and the polypeptide transduces a second cell, thereby delivering the polypeptide to the second cell.

57. The vector of claim 56, wherein delivery of the polypeptide to the second cell increases transport of ApoB mRNA from the nucleus to the cytoplasm of the second cell.

58. A method comprising administering to a cell a polypeptide comprising an ACF sequence and a transduction sequence,
wherein the ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated.

59. The method of claim 58, wherein the ACF sequence has a serine to alanine or aspartic acid substitution at one or more sites where ACF is phosphorylated.

60. The method of claim 59, wherein the amino acid substitution comprises a serine to alanine or aspartic acid substitution at the amino acid residue corresponding to amino acid residue 154 in SEQ ID NO:1.

61. The method of claim 60, wherein the amino acid substitution comprises a serine to alanine or aspartic acid substitution at the amino acid residue corresponding to amino acid residue 368 in SEQ ID NO:1.

62. The method of claim 61, wherein the ACF has a second amino acid substitution, wherein the second amino acid substitution comprises a serine to alanine or aspartic acid substitution at the amino acid residue corresponding to amino acid residue 154 in SEQ ID NO:1.

63. The method of claim 61, wherein the polypeptide increases transport of ApoB mRNA from the nucleus to the cytoplasm of the cell.

64. A method of expressing ACF in a cell, comprising bringing into contact a cell and a vector comprising a nucleic acid, wherein the nucleic acid encodes a polypeptide comprising an ACF sequence; whereby the nucleic acid produces the polypeptide, thereby expressing ACF in the cell,
wherein the ACF sequence comprises the amino acid sequence SEQ ID NO:17 having one or more mutations at one or more sites where ACF is phosphorylated, the amino acid sequence SEQ ID NO:1 having one or more mutations at one or more sites where ACF is phosphorylated, the amino acid sequence SEQ ID NO:18 having one or more mutations at one or more sites where ACF is phosphorylated, or the amino acid sequence SEQ ID NO:19 having one or more mutations at one or more sites where ACF is phosphorylated.

65. The method of claim 64, wherein expression of the polypeptide in the cell increases transport of ApoB mRNA from the nucleus to the cytoplasm of the cell.

66. The method of claim 64, wherein expression of ApoB protein in the cytoplasm of the cell is increased.

67. The method of claim 66, wherein increased expression of ApoB protein leads to a reduction in the level of lipid in the cell.

68. The method of claim 64 further comprising bringing into contact the cell and a second vector, wherein the second vector comprises a second nucleic acid, wherein the second nucleic acid encodes a second polypeptide, wherein the second polypeptide comprises an APOBEC-1 sequence; whereby the nucleic acid produces the second polypeptide, thereby expressing APOBEC-1 in the cell.

69. The method of claim 68, wherein the second polypeptide further comprises a secretion sequence and a transduction sequence.

70. The method of claim 68, wherein the second polypeptide is secreted from the cell, and wherein the second polypeptide transduces a second cell, thereby delivering the second polypeptide to the second cell.

71. The method of claim 68, wherein the APOBEC-1 sequence comprises the amino acid sequence SEQ ID NO:8, an amino acid sequence at least about 90% identical to the amino acid sequence of SEQ ID NO:8, or the amino acid sequence SEQ ID NO:8 having one or more conservative amino acid substitutions.

72. A method of screening for a compound that modulates phosphorylation of ACF, comprising:
contacting a cell expressing ACF with a test compound,
detecting the level of phosphorylated ACF using ACF phosphorylation site-specific antibodies,
wherein a change in the level of phosphorylated ACF compared to the level of phosphorylated ACF in a control cell expressing ACF not exposed to the test compound indicates that the test compound is a compound that modulates phosphorylation of ACF.

73. The method of claim 72, wherein the test compound is a phosphatase inhibitor.

74. The method of claim 72, wherein a plurality of test compounds are contacted with ACF in a high throughput cell-based assay system.

75. The method of claim 74, wherein the high throughput assay system comprises an immobilized array of cells.

76. The method of claim 72, wherein the cells are hepatocytes.

77. The method of claim 72, wherein the level of phosphorylated ACF in the cells expressing ACF contacted with the test compound is decreased compared to the level of phosphorylated ACF in the control cells, thereby identifying the test compound as a compound that decreases the level of phosphorylated ACF.

78. The method of claim 72 further comprising producing the compound identified as modulating phosphorylation of ACF.

79. A compound identified by the method of claim 78.

80. A method of screening for a compound that increases expression of ACF, comprising:
contacting a cell with a test compound,
detecting the level of ACF expression in the cell, wherein an increased level of ACF expression compared to the level of ACF expression in a control cell not exposed to the test compound indicates that the test compound is a compound that increases expression of ACF.

81. The method of claim 80, wherein the cell is a hepatocyte.

82. The method of claim 80, wherein the level of ACF expression in the cell is detected by detecting the level of ACF in the cell.

83. The method of claim 80, wherein the cell comprises a nucleic acid sequence comprising ACF expression control sequences operably linked to a sequence encoding a marker, wherein the level of ACF expression in the cell is detected by detecting the level of the marker.

84. The method of claim 80 further comprising producing the compound identified as increasing expression of ACF.

85. A compound identified by the method of claim 80.


SEQUENCE LISTING (38 sequences)

SEQ ID NO: 1 - protein, 589 aa, Artificial Sequence
  Description of Artificial Sequence; note = synthetic construct
MESNHKSGDGLSGTQKEAALRALVQRTGYSLVQENGQRKYGGPPPGWDAAPPERGCEIFIGKLPRDLFEDELIPLCEKIGKIYEMRMMMDFNGNNRGYAFVTFSNKVEAKNAIKQLNNYEIRNGRLLGVCASVDNCRLFVGGIPKTKKREEILSEMKKVTNEGVVDVIVYPSAADKTKNRGFAFVEYESHRTAAMARRKLLPGRIQLWGHGIAVDWAEPEVEVDEDTMSSVKILYVRNLMLSTSEEMIEKEFNNIKPGAVERVKKIRDYAFVHFSNRKDAVEAMKALNGKVLDGSPIEVTLAKPVDKDSYVRYTRGTGGRGTMLQGEYTYSLGQVYDPTTTYLGAPVFYAPQTYAAIPSLHFPATKGHLSNRAIIRAPSVRGAAGVRGLGGRGYLAYTGLGRGYQVKGDKREDKLYDILPGMELTPMNPVTLKPQGIKLAPQILEEICQKNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 2 - protein, 589 aa, Artificial Sequence
  Description of Artificial Sequence; note = synthetic construct
MESNHKSGDGLSGTQKEAALRALVQRTGYSLVQENGQRKYGGPPPGWDAAPPERGCEIFIGKLPRDLFEDELIPLCEKIGKIYEMRMMMDFNGNNRGYAFVTFSNKVEAKNAIKQLNNYEIRNGRLLGVCASVDNCRLFVGGIPKTKKREEILAEMKKVTNEGVVDVIVYPSAADKTKNRGFAFVEYESHRTAAMARRKLLPGRIQLWGHGIAVDWAEPEVEVDEDTMSSVKILYVRNLMLSTSEEMIEKEFNNIKPGAVERVKKIRDYAFVHFSNRKDAVEAMKALNGKVLDGSPIEVTLAKPVDKDSYVRYTRGTGGRGTMLQGEYTYSLGQVYDPTTTYLGAPVFYAPQTYAAIPSLHFPATKGHLSNRAIIRAPSVRGAAGVRGLGGRGYLAYTGLGRGYQVKGDKREDKLYDILPGMELTPMNPVTLKPQGIKLAPQILEEICQKNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 3 - protein, 589 aa, Artificial Sequence
  Description of Artificial Sequence; note = synthetic construct
MESNHKSGDGLSGTQKEAALRALVQRTGYSLVQENGQRKYGGPPPGWDAAPPERGCEIFIGKLPRDLFEDELIPLCEKIGKIYEMRMMMDFNGNNRGYAFVTFSNKVEAKNAIKQLNNYEIRNGRLLGVCASVDNCRLFVGGIPKTKKREEILSEMKKVTNEGVVDVIVYPSAADKTKNRGFAFVEYESHRTAAMARRKLLPGRIQLWGHGIAVDWAEPEVEVDEDTMSSVKILYVRNLMLSTSEEMIEKEFNNIKPGAVERVKKIRDYAFVHFSNRKDAVEAMKALNGKVLDGSPIEVTLAKPVDKDSYVRYTRGTGGRGTMLQGEYTYSLGQVYDPTTTYLGAPVFYAPQTYAAIPSLHFPATKGHLANRAIIRAPSVRGAAGVRGLGGRGYLAYTGLGRGYQVKGDKREDKLYDILPGMELTPMNPVTLKPQGIKLAPQILEEICQKNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 4 - protein, 589 aa, Artificial Sequence
  Description of Artificial Sequence; note = synthetic construct
MESNHKSGDGLSGTQKEAALRALVQRTGYSLVQENGQRKYGGPPPGWDAAPPERGCEIFIGKLPRDLFEDELIPLCEKIGKIYEMRMMMDFNGNNRGYAFVTFSNKVEAKNAIKQLNNYEIRNGRLLGVCASVDNCRLFVGGIPKTKKREEILAEMKKVTNEGVVDVIVYPSAADKTKNRGFAFVEYESHRTAAMARRKLLPGRIQLWGHGIAVDWAEPEVEVDEDTMSSVKILYVRNLMLSTSEEMIEKEFNNIKPGAVERVKKIRDYAFVHFSNRKDAVEAMKALNGKVLDGSPIEVTLAKPVDKDSYVRYTRGTGGRGTMLQGEYTYSLGQVYDPTTTYLGAPVFYAPQTYAAIPSLHFPATKGHLANRAIIRAPSVRGAAGVRGLGGRGYLAYTGLGRGYQVKGDKREDKLYDILPGMELTPMNPVTLKPQGIKLAPQILEEICQKNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 5 - DNA, 1945 bp, Artificial Sequence
  Description of Artificial Sequence; note = synthetic construct
gataatcaaggaaaccttttccgggtggggatctctgaaattactcagatacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgaacagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgaaaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctatt

SEQ ID NO: 6 - protein, 598 aa, Artificial Sequence
  Description of Artificial Sequence; note = synthetic construct
RKKRRQRRRMESNHKSGDGLSGTQKEAALRALVQRTGYSLVQENGQRKYGGPPPGWDAAPPERGCEIFIGKLPRDLFEDELIPLCEKIGKIYEMRMMMDFNGNNRGYAFVTFSNKVEAKNAIKQLNNYEIRNGRLLGVCASVDNCRLFVGGIPKTKKREEILSEMKKVTNEGVVDVIVYPSAADKTKNRGFAFVEYESHRTAAMARRKLLPGRIQLWGHGIAVDWAEPEVEVDEDTMSSVKILYVRNLMLSTSEEMIEKEFNNIKPGAVERVKKIRDYAFVHFSNRKDAVEAMKALNGKVLDGSPIEVTLAKPVDKDSYVRYTRGTGGRGTMLQGEYTYSLGQVYDPTTTYLGAPVFYAPQTYAAIPSLHFPATKGHLSNRAIIRAPSVRGAAGVRGLGGRGYLAYTGLGRGYQVKGDKREDKLYDILPGMELTPMNPVTLKPQGIKLAPQILEEICQKNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 7 - protein, 236 aa, Artificial Sequence
  Description of Artificial Sequence; note = synthetic construct
MTSEKGPSTGDPTLRRRIEPWEFDVFYDPRELRKEACLLYEIKWGMSRKIWRSSGKNTTNHVEVNFIKKFTSERDFHPSISCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 8 - DNA, 711 bp, Artificial Sequence
  Description of Artificial Sequence; note = synthetic construct
atgacttctgagaaaggtccttcaaccggtgaccccactctgaggagaagaatcgaaccctgggagtttgacgtcttctatgaccccagagaacttcgtaaagaggcctgtctgctctacgaaatcaagtggggcatgagccggaagatctggcgaagctcaggcaaaaacaccaccaatcacgtggaagttaattttataaaaaaatttacgtcagaaagagattttcacccatccatcagctgctccatcacctggttcttgtcctggagtccctgctgggaatgctcccaggctattagagagtttctgagtcggcaccctggtgtgactctagtgatctacgtagctcggcttttttggcacatggatcaacaaaatcggcaaggtctcagggaccttgttaacagtggagtaactattcagattatgagagcatcagagtattatcactgctggaggaattttgtcaactacccacctggggatgaagctcactggccacaatacccacctctgtggatgatgttgtacgcactggagctgcactgcataattctaagtcttccaccctgtttaaagatttcaagaagatggcaaaatcatcttacatttttcagacttcatcttcaaaactgccattaccaaacgattccgccacacatccttttagctacagggctgatacatccttctgtggcttggagatga

SEQ ID NO: 9 - protein, 596 aa, Artificial Sequence
  Description of Artificial Sequence; note = synthetic construct
MESNHKSGDGLSGTQKEAALRALVQRTGYSLVQENGQRKYGGPPNPGWDAAPPERGCEIFIGKLPRDLFEDELIPLCEKIGKIYEMRMMMDFNGNNRGYAFVTNFSNKVEAKNAIKQLNNYEIRNGRLLGVCASVDNCRLFVGGIPKTKKREEILSEMKKVTNEGVVDVIVYPSAADKTKNRGFAFVEYESHRAAAMARRKLLPGRIQLWGHGIAVDWAEPNEVEVDEDTMSSVKILYVRNLMLSTSEEMIEKEFNNIKPGAVERVKKIRDYAFVHFSNRNEDAVEAMKALNGKVLDGSPIEVTLAKPVDKDSYVRYTRGTGGRGTMLQGEYTYSLGQVNYDPTTTYLGAPVFYAPQTYAAIPSLHFPATKGHLSNRAIIRAPSVRGAAGVRGLGGRGNYLAYTGLGRGYQVKGDKREDKLYDILPGMELTPMNPVTLKPQGIKLAPQILEEICQKNNNWGQPVYQLHSAIGQDQRQLFLYKITIPALASQNPAIHPFTPPKLSAFVDEAKTYAAENYTLQTLGIPTDGGDGTMATAAAAATAFPGYAVPNATAPVSAAQLKQAVTLGQDLAAYTNTYEVYPTFAVTARGDGYGTF

SEQ ID NO: 10 - protein, 113 aa, Artificial Sequence
  Description of Artificial Sequence; note = synthetic construct
PIEVTLAKPVDKDSYVRYTRGTGGRNTMLQEYTYPLSHVYDPTTTYLGAPVFYTPQAYAANIPSLHFPATKGHLSNRALIRTPSVRGCSRTPSIYLCFLTAVHAGVHHIHVQN

SEQ ID NO: 11 - protein, 389 aa, Artificial Sequence
  Description of Artificial Sequence; note = synthetic construct
MESNHKSGDGLSGTQKEAALRALVQRTGYSLVQENGQRKYGGPPNPGWDTTPPERGCEIFIGKLPRDLFEDELIPLCEKIGKIYEMRMMMDFNGNNRGYAFVTNFSNKQEAKNAIKQLNNYEIRNGRLLGVCASVDNCRLFVGGIPKTKKREEILSEMKKVTNEGVVDVIVYPSAADKTKNRGFAFVEYESHRAAAMARRRLLPGRIQLWGHPIAVDWAEPNEVEVDEDTMSSVKILYVRNLMLSTSEEMIEKEFNSIKPGAVERVKKIRDYAFVHFSNRNEDAVEAMKALNGKVLDGSPIEVTLAKPVDKDSYVRYTRGTGGRNTMLQEYTYPLSHVYNDPTTTYLGAPVFYAPQAYAAIPSLHFPATKGHLSNRALIRTPSVRGNIS

SEQ ID NO: 12 - DNA, 1770 bp, Artificial Sequence
  Description of Artificial Sequence; note = synthetic construct
ctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatg

SEQ ID NO: 13 - DNA, 1394 bp, Artificial Sequence
  Description of Artificial Sequence; note = synthetic construct
gaattcaccataatcaagaaaccttttccgggtgggggaacctcagaaattactcagtgagcaaccctaattaacctgattttttgctgataatcactctcaatggaatcaaatcacaaatccggggatggattgagcggcacccagaaggaagcagcactccgcgcactggtccagcgcacaggatatagcttggtccaggaaaatggacaaagaaaatatggtggtcctccaccaggctgggatactacacccccagaaaggggctgcgagattttcattgggaaacttccccgggacctttttgaggatgaactcataccattgtgtgaaaaaattggtaaaatttatgaaatgagaatgatgatggatttcaatgggaacaacagaggctatgcatttgtaaccttctcaaataagcaggaagccaagaatgcaatcaagcaacttaataattatgaaattcggaatggccgtctcctgggcgtctgtgccagtgtggacaactgccggttgtttgtggggggaatccccaaaaccaaaaagagagaagaaatcttgtcagagatgaaaaaggtcactgaaggagttgttgatgtcattgtctacccaagcgctgccgataaaaccaaaaaccgggggtttgcctttgtggaatatgagagtcaccgcgcagccgccatggctaggcggaggctgctgccaggaagaattcagttgtggggacatcctatcgcagtagactgggcagagccagaagtcgaagttgacgaagacacaatgtcttccgtgaaaatcctgtacgtaaggaaccttatgctgtctacctcggaagagatgattgagaaggaattcaacagtattaaaccaggtgctgtggaacgggtgaagaagattcgagactatgcttttgtgcatttcagtaaccgagaagatgcagttgaagccatgaaggctttgaatggcaaggtgctggatggttccccaatagaagtgaccttggccaagccagtggacaaggacagttacgttaggtacacccggggcaccgggggcaggaacaccatgctgcaagaatacacctaccctctgagccatgtttatgaccctaccacaacctaccttggagctcctgtcttctatgctccccaagcctacgcagccattccaagtcttcatttcccagctaccaaaggacatctcagcaacagagctctcatccggaccccttctgtcagaggctgctcaagaactcccagcatctacctttgcttcctgactgctgtacatgcaggtgtgcaccacatccatgtgcagtaactttgaaaaaacaaatcaaacaaggagattaatattgctttagcataatggaaaccaaataaactaaaaaaaaa

SEQ ID NO: 14 - DNA, 1916 bp, Artificial Sequence
  Description of Artificial Sequence; note = synthetic construct
gaattcaccataatcaagaaaccttttccgggtgggggaacctcagaaattactcagtgagcaaccctaattaacctgattttttgctgataatcactctcaatggaatcaaatcacaaatccggggatggattgagcggcacccagaaggaagcagcactccgcgcactggtccagcgcacaggatatagcttggtccaggaaaatggacaaagaaaatatggtggtcctccaccaggctgggatactacacccccagaaaggggctgcgagattttcattgggaaacttccccgggacctttttgaggatgaactcataccattgtgtgaaaaaattggtaaaatttatgaaatgagaatgatgatggatttcaatgggaacaacagaggctatgcatttgtaaccttctcaaataagcaggaagccaagaatgcaatcaagcaacttaataattatgaaattcggaatggccgtctcctgggcgtctgtgccagtgtggacaactgccggttgtttgtggggggaatccccaaaaccaaaaagagagaagaaatcttgtcagagatgaaaaaggtcactgaaggagttgttgatgtcattgtctacccaagcgctgccgataaaaccaaaaaccgggggtttgcctttgtggaatatgagagtcaccgcgcagccgccatggctaggcggaggctgctgccaggaagaattcagttgtggggacatcctatcgcagtagactgggcagagccagaagtcgaagttgacgaagacacaatgtcttccgtgaaaatcctgtacgtaaggaaccttatgctgtctacctcggaagagatgattgagaaggaattcaacagtattaaaccaggtgctgtggaacgggtgaagaagattcgagactatgcttttgtgcatttcagtaaccgagaagatgcagttgaagccatgaaggctttgaatggcaaggtgctggatggttccccaatagaagtgaccttggccaagccagtggacaaggacagttacgttaggtacacccggggcaccgggggcaggaacaccatgctgcaagaatacacctaccctctgagccatgtttatgaccctaccacaacctaccttggagctcctgtcttctatgctccccaagcctacgcagccattccaagtcttcatttcccagctaccaaaggacatctcagcaacagagctctcatccggaccccttctgtcagaggtaacattagctagctcttgtctctccataactccaaatttcaaatgactaataaacgagtttaaatgaactataaactatacttcagtagccttaaatagattggatggaatcactgggagaaaaaaaagagtagaaaactgttaaaaaatcaactatatcagtatctcttgtcatataggaatctattttttcacattacaactggttattgaaaaatacttgtcagaacgtgatatgaatgagcccagtccataagtgagattcttttagtatataaattggatccacaaaatatgcacacaatataaccttgctcttataaccattgcctcttcctccttattaaaccaactagaccagttacagaactttcaagggagagcattggggcctaatgggacctgcctcaaatgaggcattctcatataacctctctcctggatatgtgtgctatacctttgctagcccatgtgtttgtctgtatgttggtgacaagggcttactatatattccaggctgctcaagaactcccagcatctacctttgcttcctgactgctgtacatgcaggtgtgcaccacatccatgtgcagtaactttgaaaaaacaaatcaaacaaggagattaatattgctttagcataatggaaaccaaataaactaaaaaaaaaaaaa

SEQ ID NO: 15 - protein, 9 aa, Artificial Sequence
  Description of Artificial Sequence; note = synthetic construct
RKKRRQRRR

SEQ ID NO: 16 - protein, 594 aa, Artificial Sequence
  Description of Artificial Sequence; note = synthetic construct
MESNHKSGDGLSGTQKEAALRALVQRTGYSLVQENGQRKYGGPPPGWDTTPPERGCEIFIGKLPRDLFEDELIPLCEKIGKIYEMRMMMDFNGNNRGYAFVTFSNKQEAKNAIKQLNNYEIRNGRLLGVCASVDNCRLFVGGIPKTLKREEILSEMKLVTEGVVDVIVYPSAADKTKNRGFAFVEYESHRAAAMARRRLLPGRIQLWGHPIAVDWAEPEVEVDEDTMSSVKILYVRNLMLSTSEEMIEKEFNSIKPGAVERVKKIRDYAFVHFSNREDAVEAMKALNGKVLDGSPIEVTLAKPVDKDSYVRYTRGTGGRNTMLQEYTYPLSHVYDPTTTYLGAPVFYTPQAYAAIPSLHFPATKGHLSNRALIRTPSVREIYMNVPVGAAGVRGLGGRGYLAYTGLGRGYQVKGDKRQDKLYDLLPGMELTPMNTISLKPQGVKLAPQILEEICQKNNWGQPVYQLHSAIGQDQRQLFLYKVTIPALASQNPAIHPFTPPKLSAYVDEAKRYAAEHTLQTLGIPTEGGDAGTTAPTATSATVFPGYAVPSATAPVSTAQLKQAVTLGQDLAAYTTYEVYPTFAVTTRGDGYGTF

SEQ ID NO: 17 - DNA, 1785 bp, Artificial Sequence
  Description of Artificial Sequence; note = synthetic construct
atggaatcaaatcacaaatccggggatggattgagcggcacccagaaggaagcagcactccgcgcactggtccagcgcacaggatatagcttggtccaggaaaatggacaaagaaaatatggtggtcctccaccaggctgggatactacacccccagaaaggggctgcgagattttcattgggaaacttccccgggacctttttgaggatgaactcataccattgtgtgaaaaaattggtaaaatttatgaaatgagaatgatgatggatttcaatgggaacaacagaggctatgcatttgtaaccttctcaaataagcaggaagccaagaatgcaatcaagcaacttaataattatgaaattcggaatggccgtctcctgggcgtctgtgccagtgtggacaactgccggttgtttgtggggggaatccccaaaaccaaaaagagagaagaaatcttgtcagagatgaaaaaggtcactgaaggagttgttgatgtcattgtctacccaagcgctgccgataaaaccaaaaaccgggggtttgcctttgtggaatatgagagtcaccgcgcagccgccatggctaggcggaggctgctgccaggaagaattcagttgtggggacatcctatcgcagtagactgggcagagccagaagtcgaagttgacgaagacacaatgtcttccgtgaaaatcctgtacgtaaggaaccttatgctgtctacctcggaagagatgattgagaaggaattcaacagtattaaaccaggtgctgtggaacgggtgaagaagatccgagactatgcttttgtgcatttcagtaaccgagaagatgcagttgaagccatgaaggctttgaatggcaaggtgctggatggttccccaatagaagtgaccttggccaagccagtggacaaggacagttacgttaggtacacccggggcaccgggggcaggaacaccatgctgcaagaatacacctaccctctgagccatgtttatgaccctaccacaacctaccttggagctcctgtcttctatactccccaagcctacgcagccattccaagtcttcatttcccagctaccaaaggacatctcagcaacagagctctcatccggaccccttctgtcagagaaatttacatgaatgtccctgtaggggctgcgggcgtgagaggactgggcggccgtgggtatttggcatatacaggcctgggtcgaggataccaggtcaaaggagacaagagacaagacaaactctatgaccttctgcctgggatggagctcaccccgatgaatactatctctttaaaaccacaaggagttaaacttgctcctcagatattagaagaaatctgtcagaaaaataactggggacagccagtgtaccagctgcactctgccattggacaagaccaaagacagttattcctatacaaagtaactatcccagcgctggccagccagaatcctgcgatccaccctttcacacccccaaagctaagcgcctacgtggatgaagcaaagaggtacgccgcagagcacaccctacagacactaggcatccccacagaaggaggggacgctgggactacagcacccactgccacatccgccactgtgtttccaggatacgctgtccccagtgccaccgctcctgtgtctacagcccagctcaagcaagcagtgacacttggacaagacttagcagcatatacaacctatgaggtctaccctacttttgcagtgaccacccgaggtgatggatatggcaccttctga

SEQ ID NO: 18 - protein, 586 aa, Artificial Sequence [sequence omitted]

SEQ ID NO: 19 - DNA, 1761 bp, Artificial Sequence
  Description of Artificial Sequence; note = synthetic construct
atggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgaaaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctga

SEQ ID NO: 20 - protein, 229 aa, Artificial Sequence [sequence omitted]

SEQ ID NO: 21 - DNA, 689 bp, Artificial Sequence
  Description of Artificial Sequence; note = synthetic construct
atgagttccgasacaggccctgtagctgttgatcccactctgaggagaagaattgagccccacgagtttgaagtcttctttgacccccgggaacttcggaaagagacctgtctgctgtatgagatcaactgggaggaaggcacagcatctggcgacacacgagccaaaacaccaacaaacacgttgaagtcaatttcatagaaaaatttactacagaaagatacttttgtccaaacaccagatgctccattacctggttcctgtcctggagtccctgtggggagtgctccagggccattacagaatttttgagccgatacccccatgtaactctgtttatttatatagcacggctttatcaccacgcagatcctcgaaatcggcaaggactcagggaccttattagcagcggtgttactatccagatcatgacggagcaagagtctggctactgctggaggaattttgtcaactactccccttcgaatgaagctcattggccaaggtacccccatctgtgggtgaggctgtacgtactggaactctactgcatcattttaggacttccaccctgtttaaatattttaagaagaaaacaacctcaactcacgtttttcacgattgctcttcaaagctgccattaccaaaggctaccaccccacatcctgtgggccacagggttgaaatga

SEQ ID NO: 22 - protein, 229 aa, Artificial Sequence
  Description of Artificial Sequence; note = synthetic construct
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 23 - DNA, 689 bp, Artificial Sequence
  Description of Artificial Sequence; note = synthetic construct
atgagttccgagacaggccctgtagctgttgatcccactctgaggagaagaattgagccccacgagtttgaagtcttctttgacccccgggagcttcggaaagagacctgtctgctgtatgagatcaactggggtggaaggcacagtgtctggcgacacacgagccaaaacaccagcaaccacgttgaagtcaacttcttagaaaaatttactacagaaagatactttcgtccgaacaccagatgctccattacctggttcctgtcctggagtccctgcggggagtgctccagggccattacagagtttctgagccgacacccctatgtactctgtttatttacatagcacggctttatcaccacacggatcagcgaaaccgccaaggactcagggaccttattagcagcggtgtgactatccagatcatgacagagcaagagtattgttactgctggaggaatttcgtcaactaccccccttcaaacgaagcttattggccaaggtacccccatctgtgggtgaaactgtatgtattggagctctactgcatcattttaggacttccaccctgtttaaaaattttaagaagaaagcaacctcaactcacgtttttcacaattactcttcaaacctgccattaccaaaggataccaccccatctcctttgggctacagggttgaaatga

SEQ ID NO: 24 - DNA, 27 bp, Artificial Sequence
  Description of Artificial Sequence; note = synthetic construct
agaaaaaaaagaagacaaagaagaaga

SEQ ID NO: 25 - protein, 14 aa, Artificial Sequence
  Description of Artificial Sequence; note = synthetic construct
NHKSGDGLSGTQKE

SEQ ID NO: 26 - protein, 14 aa, Artificial Sequence
  Description of Artificial Sequence; note = synthetic construct
HTLQTLGIPTEGGD

SEQ ID NO: 27 - protein, 9 aa, Artificial Sequence
  Description of Artificial Sequence; note = synthetic construct
EIYMNVPVG

SEQ ID NO: 28 - protein, 25 aa, Artificial Sequence
  Description of Artificial Sequence; note = synthetic construct
CSRTPSIYLCFLTAVHAGVHHIHVQ

SEQ ID NO: 29 - DNA, 25 bp, Artificial Sequence
  Description of Artificial Sequence; note = synthetic construct
tcaatgtgaagtataacgaagatgg

SEQ ID NO: 30 - DNA, 20 bp, Artificial Sequence
  Description of Artificial Sequence; note = synthetic construct
atgtccagatgagcctctcc

SEQ ID NO: 31 - DNA, 19 bp, Artificial Sequence
  Description of Artificial Sequence; note = synthetic construct
gaccggttctgtcatgtcg

SEQ ID NO: 32 - DNA, 24 bp, Artificial Sequence
  Description of Artificial Sequence; note = synthetic construct
acctggttcatcatcactaatcac

SEQ ID NO: 33 - protein, 12 aa, Artificial Sequence
  Description of Artificial Sequence; note = synthetic construct
TKKREEILSEMK

SEQ ID NO: 34 - protein, 10 aa, Artificial Sequence
  Description of Artificial Sequence; note = synthetic construct
GHLSNRALIR

SEQ ID NO: 35 - RNA, 11 nt, Artificial Sequence
  Description of Artificial Sequence; note = synthetic construct
ugaucaguaua

SEQ ID NO: 36 - protein, 16 aa, Artificial Sequence
  Description of Artificial Sequence; note = synthetic construct
PQYPPLWMMLYLALEL

SEQ ID NO: 37 - protein, 9 aa, Artificial Sequence
  Description of Artificial Sequence; note = synthetic construct
KGHLSNRAL

SEQ ID NO: 38 - protein, 9 aa, Artificial Sequence
  Description of Artificial Sequence; note = synthetic construct
EEILSEMKK